(19) United States
(12) Patent Application Publication
(10) Pub. No.: US 2016/0326157 A1
(43) Pub. Date: Nov. 10, 2016

(54) MONOBACTAMS AND METHODS OF THEIR SYNTHESIS AND USE

(71) Applicant: PRESIDENT AND FELLOWS OF HARVARD COLLEGE, Cambridge, MA (US)

(72) Inventors: Andrew G. Myers, Boston, MA (US); lan Bass Seiple, San Francisco, CA (US); Robin Judith Sussman, Cambridge, MA (US)

(73) Assignee: President and Fellows of Harvard College, Cambridge, MA (US)

(21) Appl. No.: 15/109,843

(22) PCT Filed: Jan. 6, 2015
(86) PCT No.: PCT/US15/10247

§ 371 Date: Jul. 6, 2016

Related U.S. Application Data
(60) Provisional application No. 61/924,162, filed on Jan. 6, 2014.

(51) Int. Cl. C07D 417/12

(52) U.S. Cl. CPC C07D 417/12

(57) ABSTRACT

Described herein are monobactam antibiotics of Formula (I), (I′), (II), and (II′), along with methods and intermediates for preparing these compounds. Pharmaceutical compositions and methods of treating infectious diseases using the monobactams are also provided.

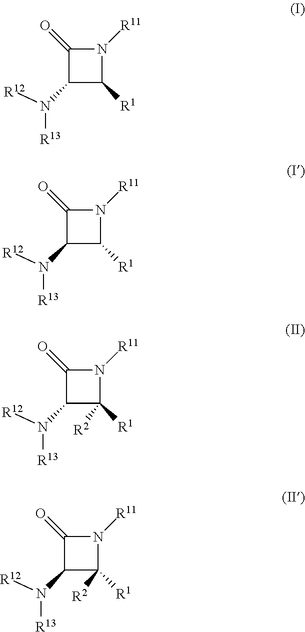

### RELATED APPLICATIONS

The present application claims priority under 35 U.S.C. §119(e) to U.S. provisional patent application, U.S. Ser. No. 61/924,162, filed Jan. 6, 2014, which is incorporated herein by reference.

### BACKGROUND OF THE INVENTION

Emerging resistance to existing antibiotics is rapidly developing as a crisis of global proportions. Pathogenic bacteria can transmit genes coding for antibiotic resistance both vertically (to their progeny) and horizontally (to neighboring bacteria of different lineages), and as a result antibiotic resistance can evolve quickly, particularly in nosocomial (hospital) settings. See, e.g., Wright, Chem. Commun. (2011) 47:4055-4061. CDC estimates that in the United States, more than two million people are sickened every year with antibiotic-resistant infections, with at least 23,000 dying as a result. The estimates are based on conservative assumptions and are likely minimum estimates. See, e.g., et al. CDC Report on Antibiotic Resistance Threats in the United States (2013) 1-114. The current crisis is exacerbated by the fact that most major pharmaceutical companies have essentially abandoned research in the development of new antibiotics. See, e.g., Projan Drug Discovery Today (2008) 13: 279-280. The current rate of introduction of new antibiotics does not adequately address growing resistance, and with the ease of international travel and increasing population densities, the need for innovation in the field of antibiotic development has never been higher.

### SUMMARY OF THE INVENTION

Described herein are new monobactams along with new methods for their synthesis and intermediates in the synthesis of these compounds. Pharmaceutical compositions of these monobactams are provided that enable the delivery of a therapeutically effective dose of a monobactam described herein to a patient in need thereof. The monobactams and pharmaceutical compositions thereof may be administered to a subject orally or parenterally for the treatment or prevention of infection. Methods of use of monobactams and pharmaceutical compositions thereof for the prevention or treatment of an infectious disease wherein the etiologic agent is a bacterium are also presented. Examples of such agents include, but are not limited to, Gram positive bacteria such as Staphylococcus aureus, or Gram negative bacteria such as Neisseria meningitidis. The monobactams described herein are useful as therapeutics for numerous diseases or disorders, examples of which include, but are not limited to, Pneumococcal pneumonia, Streptococcal pharyngitis, Meningococcal meningitis, or Listeriosis. The monobactams described herein represent a substantial advancement over the limited field of known monobactam antibiotics (see Scheme 1).

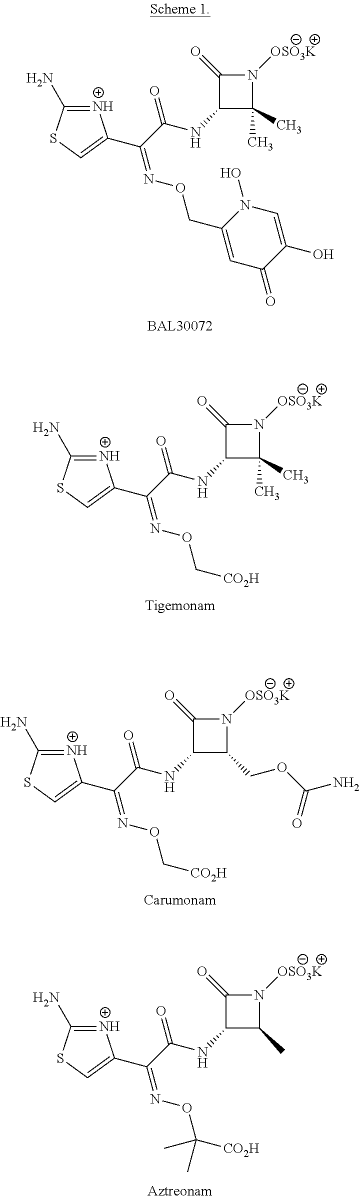

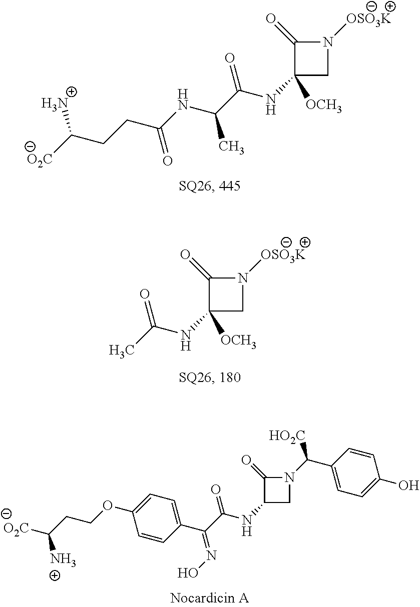

Specifically, the synthetic methods described herein to access monobactams enable the efficient incorporation of a broad range of functionality than could previously be realized with existing synthetic methodologies prior to this disclosure. Whereas previous methods often relied upon racemic syntheses of monobactams, followed by requiste chiral purification, the methods disclosed herein lead directly to the monobactams in optically pure form, often in high enantiomeric or diastereomeric excess. Furthermore, the capacity for facile integration of ketone substrates into synthetic methods described herein promotes the accessibility of monobactams with differentially substituted asymmetric quaternary centers, a group of compounds not previously explored in the field of monobactam antibiotics.

One aspect provides compounds of Formula (I) or (I′):

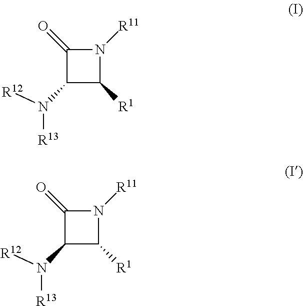

or pharmaceutically acceptable salts, stereoisomers, polymorphs, isotopes, solvates, hydrates, tautomers, or prodrugs thereof, where R¹, R¹¹, R¹², and R¹³ are defined herein.

In another aspect, provided are compounds of Formula (II) or (II′):

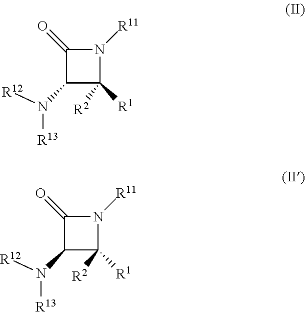

or pharmaceutically acceptable salts, stereoisomers, polymorphs, isotopes, solvates, hydrates, tautomers, or prodrugs thereof, where R¹, R², R¹¹, R¹², and R¹³ are defined herein.

En route to the monobactams, the synthetic methodology described herein utilizes a diastereoselective glycine aldol reaction with aldehyde and ketone substrates to generate diastereomerically enriched amino amides and derivatives thereof with high diastereoselectivity (see Scheme 2). This reaction is also described in PCT Publication No. WO 2014/165792, published Oct. 9, 2014, which is incorporated herein by reference.

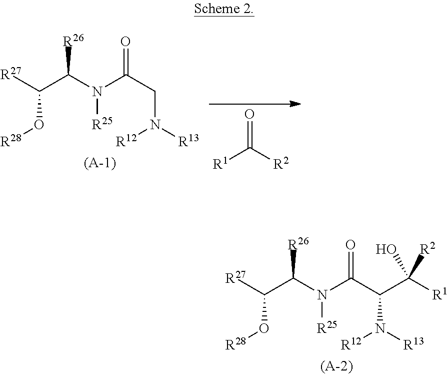

The synthetic methodology as described herein is both useful in the synthesis of known monobactams, and in the synthesis and development of new monobactams as described herein. New monobactams, and pharmaceutical compositions thereof, are contemplated to be useful in the treatment of various diseases such as, for example, the treatment and prevention of infectious diseases, such as bacterial infections.

Further provided are compounds of Formula (I), (I′), (II), and (II′), and pharmaceutically acceptable salts, solvates, hydrates, polymorphs, co-crystals, tautomers, stereoisomers, isotopes, prodrugs, and compositions thereof. The compounds of Formula (I), (I′), (II), and (II′), and pharmaceutically acceptable salts, solvates, hydrates, polymorphs, co-crystals, tautomers, stereoisomers, isotopes, prodrugs, and compositions thereof, may inhibit the growth of a bacterium. In certain embodiments, the bacterium is Gram positive. In certain embodiments, the bacterium is Gram negative. Further provided are methods of using the monobactams, and pharmaceutically acceptable salts, solvates, hydrates, polymorphs, co-crystals, tautomers, stereoisomers, isotopes, prodrugs, and compositions thereof, as therapeutics for the prevention and treatment of a bacterial infections. In certain embodiments, the bacterial etiologic agent is Enterobacteriaceae spp., Pseudomonas aeruginosa, Haemophilus influenzae, or Neisseria meningitidis. In certain embodiments, the monobactams are used to treat a drug-resistant bacterial infections.

The details of certain embodiments of the invention are set forth in the Detailed Description of Certain Embodiments, as described below. Other features, objects, and advantages of the invention will be apparent from the Examples, and Claims.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds may be depicted as different tautomers. It should also be understood that when compounds have tautomeric forms, all tautomeric forms are intended to be included in the scope of the present invention, and the naming of any compound described herein does not exclude any tautomer form.

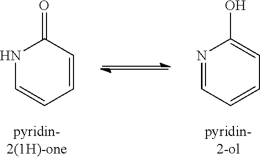 [SMILES: O=c1cccc[nH]1 ; Oc1ccccn1]

Unless otherwise stated, structures depicted herein include compounds that differ only in the presence of one or more isotopes of any given atom within any described molecule. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools, probes in biological assays, imaging agents, or radiopharmaceuticals (i.e. “isotopic labels” or “isotopically labeled” compounds). In some embodiments, the molecule is “enriched” in a given isotope, which is defined as a state in which an increased amount of an isotope of interest is present within the final composition relative to a composition derived from unenriched synthetic compounds, intermediates, or reagents.

The term “aliphatic,” as used herein, includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃10 carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₋₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or polycyclic (e.g., bicyclic or tricyclic) 4n+2 aromatic ring system (e.g., having 6 or 10 it electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-10 membered heteroaryl. In certain embodiments, the heteroaryl group is substituted 5-10 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Aralkyl” is a subset of “alkyl” and refers to an alkyl group, as defined herein, substituted by an aryl group, as defined herein, wherein the point of attachment is on the alkyl moiety.

“Heteroaralkyl” is a subset of “alkyl” and refers to an alkyl group, as defined herein, substituted by a heteroaryl group, as defined herein, wherein the point of attachment is on the alkyl moiety.

“Heterocyclylalkyl” is a subset of “alkyl” and refers to an alkyl group, as defined herein, substituted by a heterocycle, as defined herein, wherein the point of attachment is on the alkyl moiety.

“Carbocyclylalkyl” is a subset of “alkyl” and refers to an alkyl group, as defined herein, substituted by a carbocycle, as defined herein, wherein the point of attachment is on the alkyl moiety.

“Fused” or “ortho-fused” are used interchangeably herein, and refer to two rings that have two atoms and one bond in common, e.g., napthalene

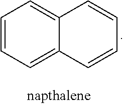

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

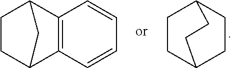 [SMILES: c1ccc2c(c1)C1CCC2C1 ; C1CC2CCC1CC2]

“Spiro” or “Spiro-fused” refers to a group of atoms which connect to the same atom of a carbocyclic or heterocyclic ring system (geminal attachment), thereby forming a ring, e.g.,

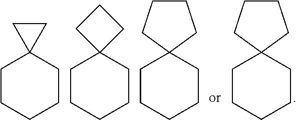

Spiro-fusion at a bridgehead atom is also contemplated.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” aliphatic, “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}C₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-10 membered heterocyclyl, C₆₋₁₄ aryl, and 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀alkenyl, C₂₋₁₀ alkynyl, C₃₋₁₀ carbocyclyl, 3-10 membered heterocyclyl, C₆₋₁₄ aryl, and 5-10 membered heteroaryl, or two R^{aa} groups are joined to form a 3-10 membered heterocyclyl or 5-10 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from —H, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-10 membered heterocyclyl, C₆14 aryl, and 5-10 membered heteroaryl, or two R^{bb} groups are joined to form a 3-10 membered heterocyclyl or 5-10 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from —H, C₁₋₁₀ alkyl, C₁₋₁₀ perhaloalkyl, C₂₋₁₀ alkenyl, C₂₋₁₀ alkynyl, C₃₋₁₀ carbocyclyl, 3-10 membered heterocyclyl, C₆₋₁₄ aryl, and 5-10 membered heteroaryl, or two R^{cc} groups are joined to form a 3-10 membered heterocyclyl or 5-10 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆10 aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from —H, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₄ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

In certain embodiments, a substituent is selected from the group consisting of halogen, —CN, —NO₂, —N₃, —OH, —OR^{aa}, —N(R^{bb})₂, —SH, —SR^{aa}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —C(═O)SR^{aa}, C₁₋₁₀ alkyl, C₁₋₁₀ perhaloalkyl, C₂₋₁₀ alkenyl, C₂₋₁₀ alkynyl, C₃₋₁₀ carbocyclyl, 3-10 membered heterocyclyl, C₆₋₁₄ aryl, and 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, —H, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-10 membered heterocyclyl, C₁₋₄ aryl, and 5-10 membered heteroaryl, or two R^{cc} groups attached to a nitrogen atom are joined to form a 3-10 membered heterocyclyl or 5-10 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-10 membered heterocyclyl, C₆₋₁₄ aryl, and 5-10 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1, 1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, tosylate (Ts), and and t-butyl carbonate (BOC).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W.

Greene and P. G. M. Wuts, 3^{rd} edition, John Wiley & Sons, 1999, incorporated herein by reference.

The term “acyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

“Acidic” refers to a medium containing an organic or inorganic chemical substance, an aqueous solution of which is characterized by a pH of less than 7. The chemical substance may be a Brønsted acid, wherein the chemical substance adds protons to the medium, or a Lewis acid, wherein the chemical substance can accept an electron lone pair from another molecule in the medium, thereby forming a stable molecular complex.

“Basic” refers to a medium containing an organic or inorganic chemical substance, an aqueous solution of which is characterized by a pH of greater than 7. The chemical substance may be a Brønsted base, wherein the chemical substance adds hydroxide molecules to the medium, or a Lewis base, wherein the chemical substance can donate an electron lone pair to another molecule in the medium, thereby forming a stable molecular complex.

The “aldol reaction” is defined as a reaction between two carbonyl-containing compounds, at least one of which possesses an enolizable carbon, wherein one compound acts as a nucleophile, and the other an electrophile resulting in the formation of a carbon-carbon bond between the enolizable carbon of one carbonyl-containing compound, and the carbonyl carbon of the other. The product of the reaction is a beta-hydroxy ketone or aldehyde, the trivial name of latter being “aldol”, a term which is used more generally to identify the reaction class.

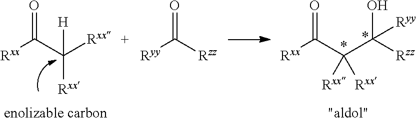

The reaction proceeds through the intermediacy of a nucleophilic enol or enolate which is formed under a variety of conditions, often in the presence of a base.

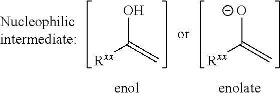

The reaction conditions are closely associated with the stereochemical outcome of the reaction, and may be controlled to obtain a composition enriched in one or more of these products.

Possible Stereochemical Outcomes:

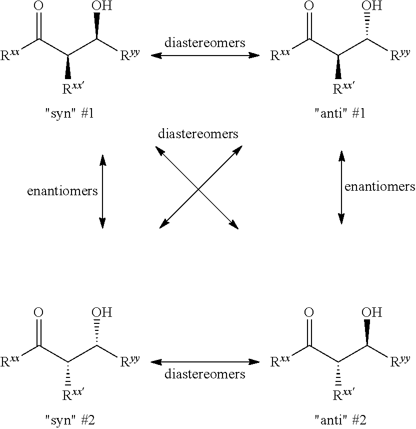

See, for example, March Advanced Organic Chemistry 4th ed. (937-944).

### OTHER DEFINITIONS

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the monobactams of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like.

Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or other non-human animals, for example mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In certain embodiments, the non-human animal is a mammal. The non-human animal may be a male or female and at any stage of development. A non-human animal may be a transgenic animal.

“Disease,” “disorder,” and “condition” are used interchangeably herein.

As used herein, and unless otherwise specified, the terms “treat,” “treating” and “treatment” contemplate an action that occurs while a subject is suffering from the specified infectious disease, which reduces the severity of the infectious disease, or retards or slows the progression of the infectious disease (“therapeutic treatment”), and also contemplates an action that occurs before a subject begins to suffer from the specified infectious disease (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of an infectious disease, or to delay or minimize one or more symptoms associated with the infectious disease. The term “therapeutically effective amount” can encompass an amount that cures the infection by killing all of, or a proportion of the etiologic pathogen or pathogens. The term “therapeutically effective amount” can encompass an amount that cures the infection by limiting the growth or reproduction of all of, or a proportion of the etiologic pathogen or pathogens. A therapeutically effective amount of a compound means an amount of therapeutic agent, alone or in combination with other therapies, which provides a therapeutic benefit in the treatment of the infectious disease. The term “therapeutically effective amount” can encompass an amount that improves overall therapy, reduces or avoids symptoms or causes of infectious disease, or enhances the therapeutic efficacy of another therapeutic agent.

As used herein, and unless otherwise specified, a “prophylactically effective amount” of a compound is an amount sufficient to prevent an infectious disease, or one or more symptoms associated with the infectious disease, or prevent its recurrence. A prophylactically effective amount of a compound means an amount of a therapeutic agent, alone or in combination with other agents, which provides a prophylactic benefit in the prevention of the infectious disease. The term “prophylactically effective amount” can encompass an amount that improves overall prophylaxis or enhances the prophylactic efficacy of another prophylactic agent.

The term “prodrugs” refer to compounds, including derivatives of the compounds of Formula (I), (I′), (II), or (II′), which have cleavable groups and become by solvolysis or under physiological conditions the compounds of Formula (I), (I′), (II), or (II′) which are pharmaceutically active in vivo. Such examples include, but are not limited to, ester, thioester, carbonate, carbamate, amide, thioamide, imide, thioimide, acetal, ketal, aminal, aminol, thioacetal, thioketal, thioaminal, phosphonate ester, phosphate, sulfonate ester, sulfate, N-alkyl, O-alkyl derivatives and the like. Other derivatives of the compounds described herein have activity in both their free and prodrug forms, but the prodrug form often offers advantages of solubility, tissue compatibility, and/or delayed release in the mammalian organism (see, Bundgard, H., Design of Prodrugs, pp. 7-9, 21-24, Elsevier, Amsterdam 1985). Prodrugs include acid derivatives well known to those skilled in the art, such as esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are exemplary prodrugs. In certain embodiments, the prodrug is an ester type prodrug. In certain embodiments, the prodrug is an ester type prodrug including one cleavable ester group. In certain embodiments, the prodrug is an ester type prodrug including two cleavable ester groups. In certain embodiments, the prodrug is a double ester type prodrug, such as an (acyloxy)alkyl ester or ((alkoxycarbonyl)oxy)alkyl ester. In certain embodiments, the prodrug is a C₁₋₈ alkyl ester, C₂₋₈ alkenyl ester, C₂₋₈ alkynyl ester, aryl ester, C₇₋₁₂ substituted aryl ester, or C₇₋₁₂ arylalkyl ester of a compound of Formula (I), (I′), (II), or (II′).

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general Formula R.xH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂O)). The term “solvate” encompasses the aforementioned terminology, but implies a broader application to a compound that is associated with any solvent. Such solvates can involve inorganic solvents (e.g. ammonia), but typically refer to organic solvents (e.g. dimethylsulfoxide, methylene chloride, dimethylformamide, acetone, ethyl acetate, ethanol).

As used herein, a “leaving group”, or “LG”, is a term understood in the art to refer to a molecular fragment that departs with a pair of electrons upon heterolytic bond cleavage, wherein the molecular fragment is an anion or neutral molecule. See, for example, March Advanced Organic Chemistry 6th ed. (501-502). Examples of suitable leaving groups include, but are not limited to, halides (such as chloride, bromide, or iodide), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, haloformates, —NO₂, trialkylammonium, and aryliodonium salts. In some embodiments, the leaving group is a sulfonic acid ester. In some embodiments, the sulfonic acid ester comprises the formula —OSO₂R^{LG1} wherein R^{LG1} is selected from the group consisting alkyl optionally, alkenyl optionally substituted, heteroalkyl optionally substituted, aryl optionally substituted, heteroaryl optionally substituted, arylalkyl optionally substituted, and heterarylalkyl optionally substituted. In some embodiments, R^{LG1} is substituted or unsubstituted C₁-C₆ alkyl. In some embodiments, R^{LG1} is methyl. In some embodiments, R^{LG1} is —CF₃. In some embodiments, R^{LG1} is substituted or unsubstituted aryl. In some embodiments, R^{LG1} is substituted or unsubstituted phenyl. In some embodiments R^{LG1} is:

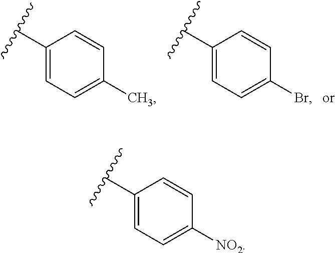 [SMILES: *c1ccc(C)cc1 ; *c1ccc(Br)cc1 ; *c1ccc([N+](=O)[O-])cc1]

“Polymorph” refers to a particular polymorphic variant of a given compound. Polymorphism is the ability of a solid substance of a given chemical composition to exist in more than one form or crystalline structure. Polymorphism can exist as a result of differences in crystal packing (packing polymorphism), conformational differences (conformational polymorphism), or changes due to co-crystalization with other chemical entities (pseudopolymorphism). Polymorphism is an important aspect of pharmaceutical development, in which case drugs typically receive regulatory approval for only a single form. Distinct polymorphic forms frequently vary considerably in terms of their physical properties. Altered dissolution rates, thermal stability, and hygroscopicity are frequently observed.

“Infectious disease” refers to an infection with a pathogen, such as a fungus, bacteria, virus, or a parasite. In certain embodiments, the infectious disease is caused by a pathogen resistant to other treatments. In certain embodiments, the infectious disease is caused by a pathogen that is multi-drug tolerant or resistant, e.g., the infectious disease is caused by a pathogen that neither grows nor dies in the presence of or as a result of other treatments. Bacterial species that are of importance to clinical practice include, but are not limited to the following: Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Prevotella melaninogenica, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis, and Yersinia pseudotuberculosis.

### DETAILED DESCRIPTION OF CERTAIN EMBODIMENTS OF THE INVENTION

Known monobactams are active against Gram-negative aerobic bacteria. Aztreonam is the only monobactam antibiotic that is currently avaliable on the market. It is inactive against Gram-positive or anaerobic bacteria. Aztreonam readily penetrates the outer membrane of Gram-negative species, and it is resistant to hydrolysis by most class A plasmid and chromosomal β-lactamases and class B enzymes. It is inactivated by class A carbapenemases, extended spectrum β-lactamases, and many class C β-lactamases, especially when these are overexpressed. Aztreonam inhibits most Enterobacteriaceae at concentrations less than 0.5 g/mL; some P. aeruginosa, E. cloacae, and C. freundii strains are resistant. Most P. aeruginosa organisms are inhibited by less than 16 g/mL. Most B. cepacia and S. maltophilia and many Acinetobacter spp. are resistant.

Aztreonam is rarely used alone empirically because its spectrum of activity is limited entirely to aerobic Gram-negative bacteria. Aztreonam has been used safely and effectively in conjunction with clindamycin, erythromycin, metronidazole, penicillins, and vancomycin. Its greatest utility is for treatment of infections caused by Gram-negative aerobic bacteria in the patient who has severe allergy to penicillin or other β-lactams. It may also have a role in combination therapy of infections caused by metallo-β-lactamase producing Gram negatives, although these strains often produce other β-lactamases that are active against aztreonam.

Aztreonam is extremely well tolerated. Skin rashes can occur. Aztreonam is the one β-lactam that can be used safely in patients with rashes or immediate hypersensitivity reactions to penicillins or other β-lactams because cross-reactivity does not occur. Hematologic, gastrointestinal, nephrotoxic, or neurotoxic reactions are rare.

Aztreonam is not absorbed from the gastrointestinal tract and is renally excreted. In adults with normal renal and hepatic function, the elimination half-life is approximately 2 hours.

Given the therapeutic profile of aztreonam, the development of new monobactams with improved anti-bacterial efficacy against a broader range of Gram negative or positive species while maintaining the safety and tolerability of existing agents would be ideal.

Furthermore, improvement of ADME properties, such as enhanced oral bioavailability and extended half-life, would considerably benefit patients and prescribing physicians.

The inventors have discovered a new synthetic route to monobactams allowing for the synthesis of novel agents from this class. This enabling technology provides access to derivatives that may exhibit several of the aforementioned benefits over traditional monobactam agents.

### COMPOUNDS

The inventors have discovered novel monobactams antibiotics accessible by the synthetic routes described herein.

In one aspect, provided is a compound of Formula (I):

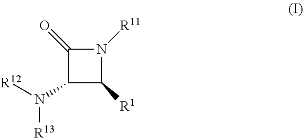

or pharmaceutically acceptable salts, stereoisomers, polymorphs, isotopes, solvates, hydrates, tautomers, or prodrugs thereof,
wherein:

R¹ is selected from C₁₋₁₀ alkyl, C₁₋₁₀ haloalkyl, C₂₋₁₀ alkenyl, C₂₋₁₀ alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, or 3-10 membered carbocyclylalkyl, each of which is optionally substituted with 0, 1, 2, 3, 4, or 5 occurrences of R⁵;

R⁵ is independently selected from halogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷ or —SR⁷;

R⁷ is independently selected from hydrogen, acyl, optionally substituted C₁₋₁₀ alkyl, optionally substituted C₆₋₁₀ aryl, optionally substituted 5-10 membered heteroaryl, optionally substituted 4-10 membered heterocyclyl, or optionally substituted 3-10 membered carbocyclyl, or two R⁷ groups are taken together with any intervening atoms to form an optionally substituted heterocyclic ring.

R¹¹ is hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵; and

R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), a nitrogen protecting group, or R¹² and R¹³ are taken together with any intervening atoms to form an optionally substituted heteroaryl, or optionally substituted heterocyclic ring, each of which is optionally substituted with 0, 1, 2, 3, 4, or 5 occurrences of R⁵.

In another aspect, provided is a compound of Formula (I′):

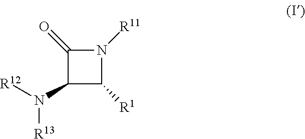

or pharmaceutically acceptable salts, stereoisomers, polymorphs, isotopes, solvates, hydrates, tautomers, or prodrugs thereof, wherein:

R¹ is selected from C₁₋₁₀ alkyl, C₁₋₁₀ haloalkyl, C₂₋₁₀ alkenyl, C₂₋₁₀ alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, or 3-10 membered carbocyclylalkyl, each of which is optionally substituted with 0, 1, 2, 3, 4, or 5 occurrences of R⁵;

R⁵ is independently selected from halogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷ or —SR⁷;

R⁷ is independently selected from hydrogen, acyl, optionally substituted C₁₋₁₀ alkyl, optionally substituted C₆₋₁₀ aryl, optionally substituted 5-10 membered heteroaryl, optionally substituted 4-10 membered heterocyclyl, or optionally substituted 3-10 membered carbocyclyl, or two R⁷ groups are taken together with any intervening atoms to form an optionally substituted heterocyclic ring.

R¹¹ is hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵; and

R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, or R¹² and R¹³ are taken together with any intervening atoms to form an optionally substituted heteroaryl, or optionally substituted heterocyclic ring, each of which is optionally substituted with 0, 1, 2, 3, 4, or 5 occurrences of R⁵.

While the methods described herein may be applied to the synthesis of known monobactams, the compounds are not for any intents or purposes inclusive of known monobactams. In some embodiments, the compounds of Formula (I) or (I′) do not include the known antibiotic aztreonam. In some embodiments, the compounds of Formula (I) or (I′) do not include the known antibiotic nocardicin A. In some embodiments, the compounds of Formula (I) or (I′) do not include the known antibiotic BAL 30072. In some embodiments, the compounds of Formula (I) or (I′) do not include the known antibiotic tigemonam. In some embodiments, the compounds of Formula (I) or (I′) do not include the known antibiotic carumonam. In some embodiments, the compounds of Formula (I) or (I′) do not include the known antibiotic SQ26,445. In some embodiments, the compounds of Formula (I) or (I′) do not include the known antibiotic SQ26,180. In some embodiments of Formula (I) or (I′), R¹ does not include unsubstituted pyridine. In some embodiments of Formula (I) or (I′), R¹ does not include substituted pyridine. In some embodiments of Formula (I) or (I′), R¹ does not include unsubstituted phenyl. In some embodiments of Formula (I) or (I′), R¹ does not include phenyl substituted with 1 occurrance of R⁵. In some embodiments of Formula (I) or (I′), R¹ does not include phenyl substituted with 2 occurances of R⁵. In some embodiments of Formula (I) or (I′), R¹ unsubstituted cyclohexyl. In some embodiments of Formula (I) or (I′), R¹ substituted cyclohexyl. In some embodiments of Formula (I) or (I′), R¹ does not include unsubstituted thiophene. In some embodiments of Formula (I) or (I′), R¹ does not include substituted thiophene. In some embodiments of Formula (I) or (I′), R¹ does not include unsubstituted furan. In some embodiments of Formula (I) or (I′), R¹ does not include substituted furan. In some embodiments of Formula (I) or (I′), R¹ does not include unsubstituted isoxazole. In some embodiments of Formula (I) or (I′), R¹ does not include substituted isoxazole. In some embodiments of Formula (I) or (I′), R¹ does not include unsubstituted pyrazole. In some embodiments of Formula (I) or (I′), R¹ does not include substituted pyrazole. In some embodiments of Formula (I) or (I′), R¹ does not include dioxolane. In some embodiments of Formula (I) or (I′), R¹ does not include unsubstituted alkyl. In some embodiments of Formula (I) or (I′), R¹ does not include substituted alkyl.

In some embodiments, the compound of Formula (I) is of Formula (I-a):

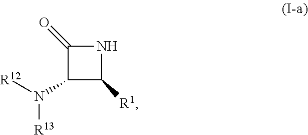

or pharmaceutically acceptable salts, stereoisomers, polymorphs, isotopes, solvates, hydrates, tautomers, or prodrugs thereof.

In some embodiments, the compound of Formula (I′) is of Formula (I-a′):

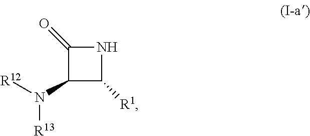

or pharmaceutically acceptable salts, stereoisomers, polymorphs, isotopes, solvates, hydrates, tautomers, or prodrugs thereof.

In some embodiments, the compound of Formula (I) is of Formula (I-b):

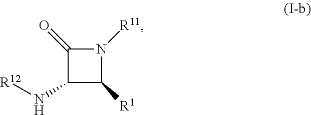

or pharmaceutically acceptable salts, stereoisomers, polymorphs, isotopes, solvates, hydrates, tautomers, or prodrugs thereof.

In some embodiments, the compound of Formula (I′) is of Formula (I-b′):

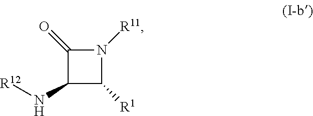

or pharmaceutically acceptable salts, stereoisomers, polymorphs, isotopes, solvates, hydrates, tautomers, or prodrugs thereof.

In some embodiments, the compound of Formula (I) is of Formula (I-c):

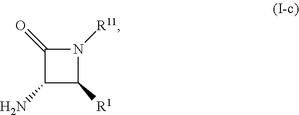

or pharmaceutically acceptable salts, stereoisomers, polymorphs, isotopes, solvates, hydrates, tautomers, or prodrugs thereof.

In some embodiments, the compound of Formula (I′) is of Formula (I-c′):

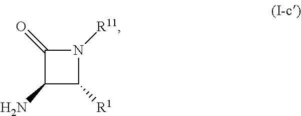

or pharmaceutically acceptable salts, stereoisomers, polymorphs, isotopes, solvates, hydrates, tautomers, or prodrugs thereof.

In another aspect, provided is a compound of Formula (II):

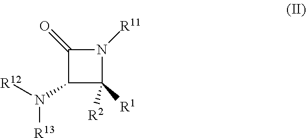

or pharmaceutically acceptable salts, stereoisomers, polymorphs, isotopes, solvates, hydrates, tautomers, or prodrugs thereof,
wherein:

R¹ and R² are independently selected from C₁₋₁₀ alkyl, C₁₋₁₀ haloalkyl, C₂₋₁₀ alkenyl, C₂₋₁₀ alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, or 3-10 membered carbocyclylalkyl, each of which is optionally substituted with 0, 1, 2, 3, 4, or 5 occurrences of R⁵, or R¹ and R² are taken together with any intervening atoms to form an optionally substituted aryl, optionally substituted heteroaryl, optionally substituted carbocyclic, or optionally substituted heterocyclic ring;

R⁵ is independently selected from halogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷;

R⁷ is independently selected from hydrogen, acyl, optionally substituted C₁₋₁₀ alkyl, optionally substituted C₆₋₁₀ aryl, optionally substituted C₆₋₁₀ aryl, optionally substituted 5-10 membered heteroaryl, optionally substituted 4-10 membered heterocyclyl, or optionally substituted 3-10 membered carbocyclyl, or two R⁷ groups are taken together with any intervening atoms to form an optionally substituted heterocyclic ring.

R¹¹ is hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵; and

R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, or R¹² and R¹³ are taken together with any intervening atoms to form an optionally substituted heteroaryl, or optionally substituted heterocyclic ring, each of which is optionally substituted with 0, 1, 2, 3, 4, or 5 occurrences of R⁵.

In another aspect, provided is a compound of Formula (II′):

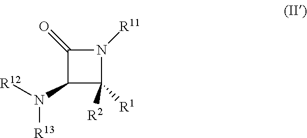

or pharmaceutically acceptable salts, stereoisomers, polymorphs, isotopes, solvates, hydrates, tautomers, or prodrugs thereof,
wherein:

R¹ and R² are independently selected from C₁₋₁₀ alkyl, C₁₋₁₀ haloalkyl, C₂₋₁₀ alkenyl, C₂₋₁₀ alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, or 3-10 membered carbocyclylalkyl, each of which is optionally substituted with 0, 1, 2, 3, 4, or 5 occurrences of R⁵, or R¹ and R² are taken together with any intervening atoms to form an optionally substituted aryl, optionally substituted heteroaryl, optionally substituted carbocyclic, or optionally substituted heterocyclic ring;

R⁵ is independently selected from halogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷;

R⁷ is independently selected from hydrogen, acyl, optionally substituted C₁₋₁₀ alkyl, optionally substituted C₆₋₁₀ aryl, optionally substituted C₆₋₁₀ aryl, optionally substituted 5-10 membered heteroaryl, optionally substituted 4-10 membered heterocyclyl, or optionally substituted 3-10 membered carbocyclyl, or two R⁷ groups are taken together with any intervening atoms to form an optionally substituted heterocyclic ring.

R¹¹ is hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵; and

R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, or R¹² and R¹³ are taken together with any intervening atoms to form an optionally substituted heteroaryl, or optionally substituted heterocyclic ring, each of which is optionally substituted with 0, 1, 2, 3, 4, or 5 occurrences of R⁵.

While the methods described herein may be applied to the synthesis of known monobactams, the compounds are not for any intents or purposes inclusive of known monobactams. Compounds of Formula (II) or (II′) do not include the known antibiotic aztreonam, for example. Other compounds excluded from Formula (II) or (II′) include:

TABLE 1
Known Monobactams

| | CAS Registry |
|---|---|
| Structure | Number |
| 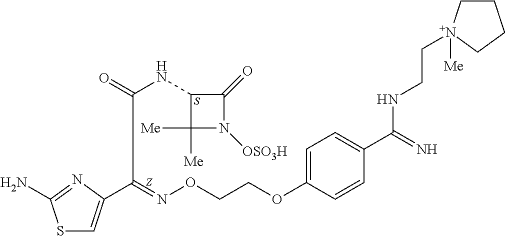 | 1448675-20-4 |
| 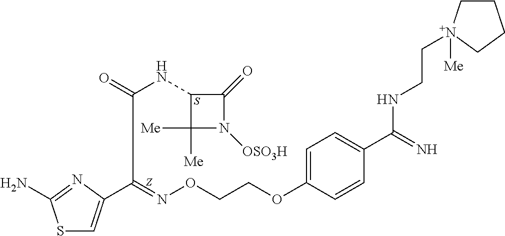 | 1448675-20-4 |
| 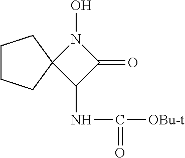 | 1448673-13-9 |
| 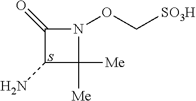 | 1448673-12-8 |
| 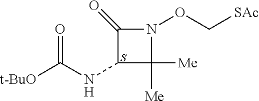 | 1448673-11-7 |
| 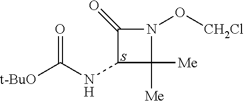 | 1448673-10-6 |
| 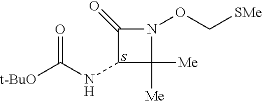 | 1448673-09-3 |
| 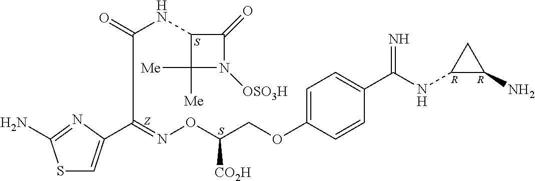 | 1448672-92-1 |
| 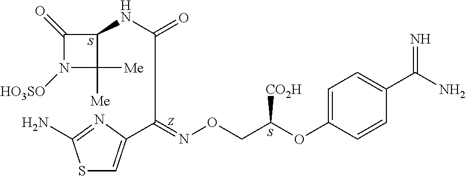 | 1448672-91-0 |
| 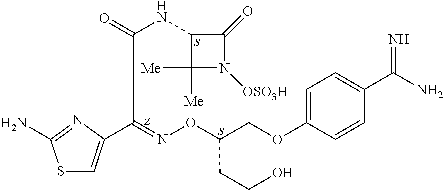 | 1448672-90-9 |
| 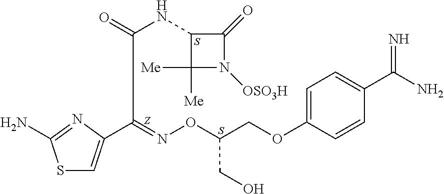 | 1448672-89-6 |
| 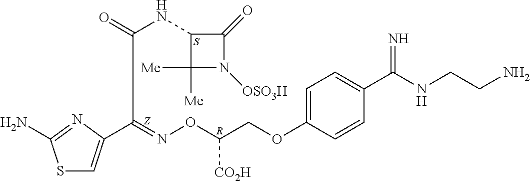 | 1448672-88-5 |
| 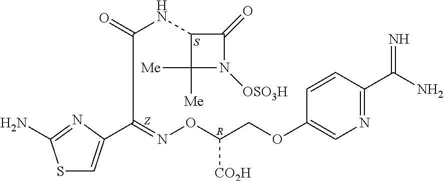 | 1448672-87-4 |
| 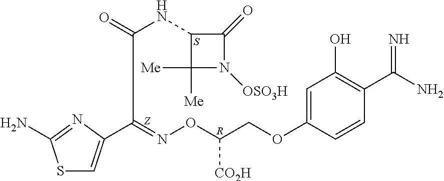 | 1448672-86-3 |
| 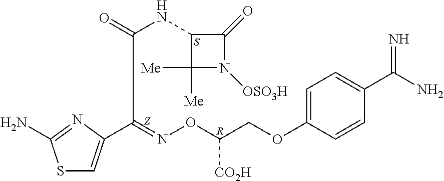 | 1448672-85-2 |
| 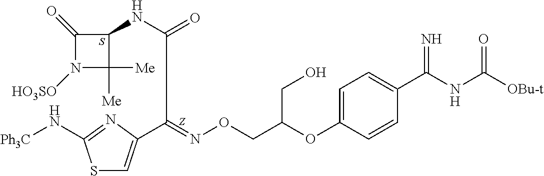 | 1448672-83-0 |
| 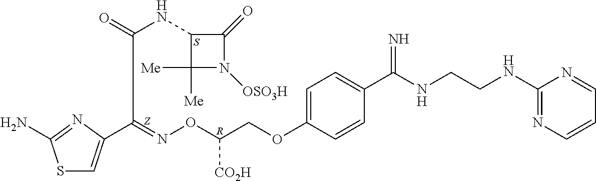 | 1448672-82-9 |
| 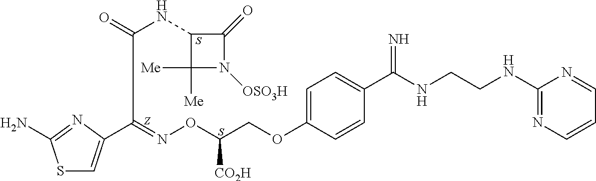 | 1448672-81-8 |
| 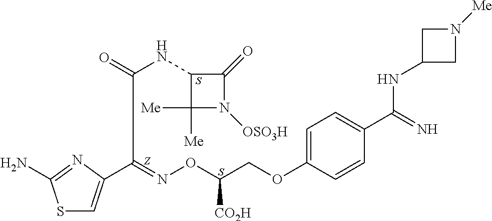 | 1448672-80-7 |
| 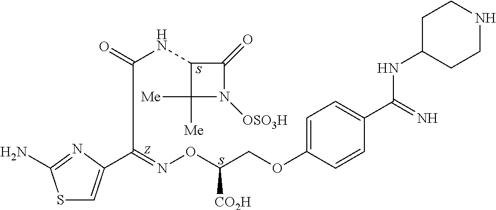 | 1448672-79-4 |
| 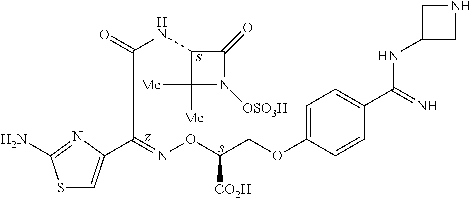 | 1448672-78-3 |
| 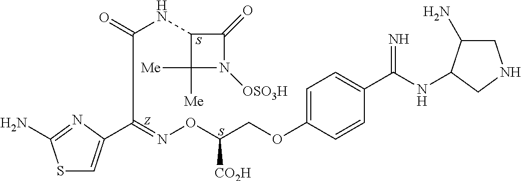 | 1448672-77-2 |
| 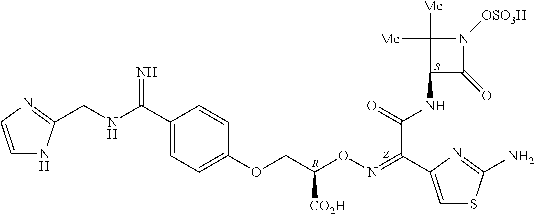 | 1448672-76-1 |
| 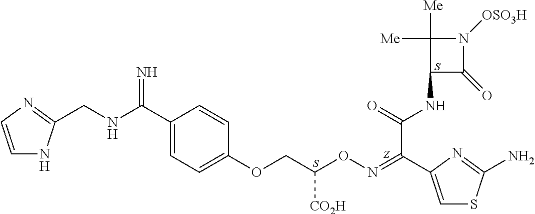 | 1448672-75-0 |
| 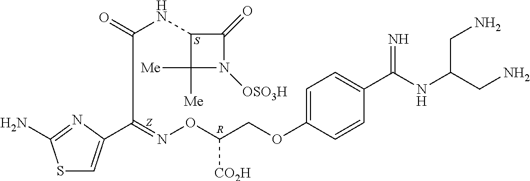 | 1448672-74-9 |
| 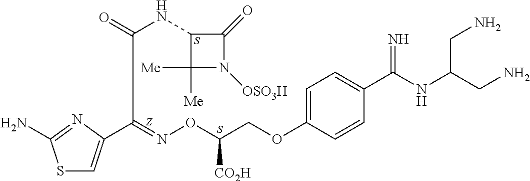 | 1448672-73-8 |
| 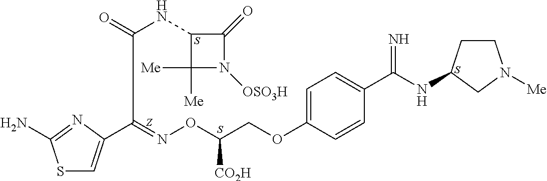 | 1448672-72-7 |
| 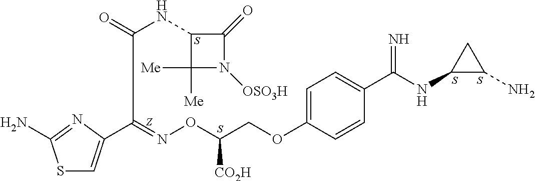 | 1448672-71-6 |
| 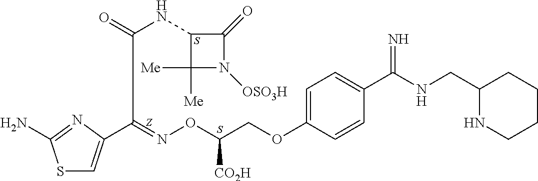 | 1448672-70-5 |
| 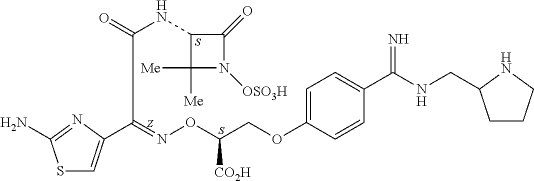 | 1448672-69-2 |
| 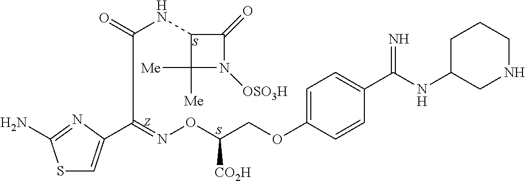 | 1448672-68-1 |
| 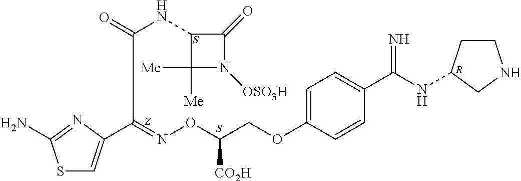 | 1448672-67-0 |
| 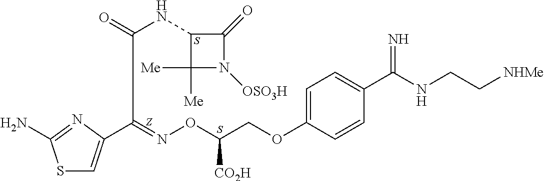 | 1448672-66-9 |
| 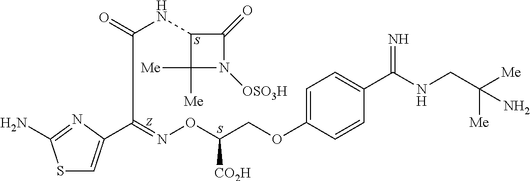 | 1448672-65-8 |
| 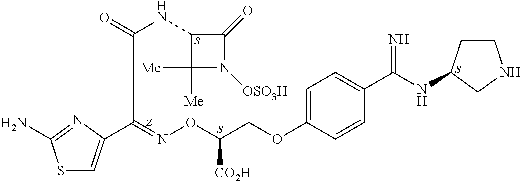 | 1448672-64-7 |
| 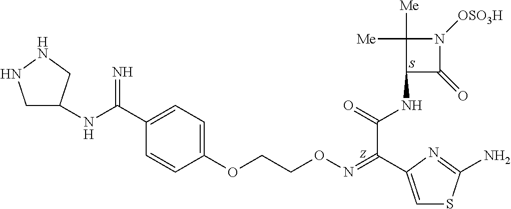 | 1448672-63-6 |
| 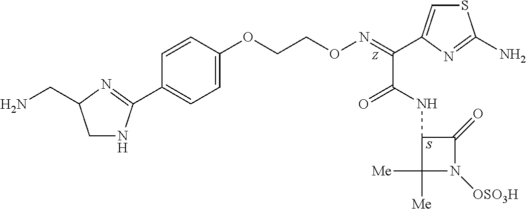 | 1448672-62-5 |
| 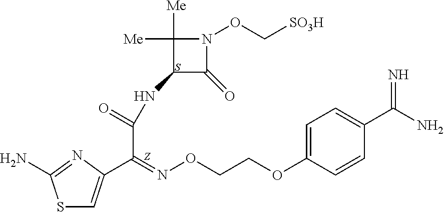 | 1448672-61-4 |
| 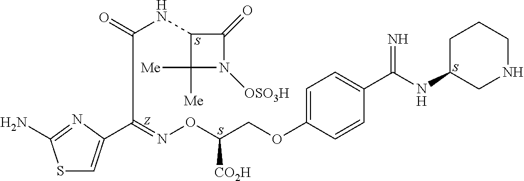 | 1448672-60-3 |
| 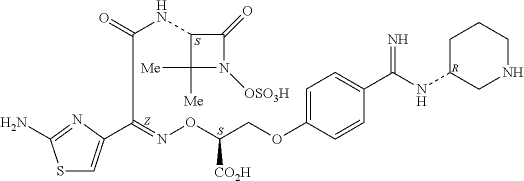 | 1448672-59-0 |
| 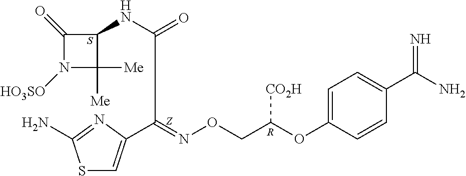 | 1448672-58-9 |
| 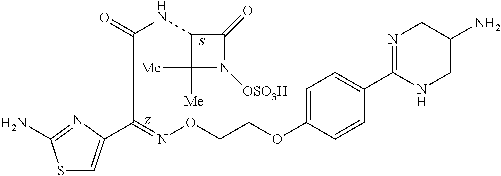 | 1448672-57-8 |
| 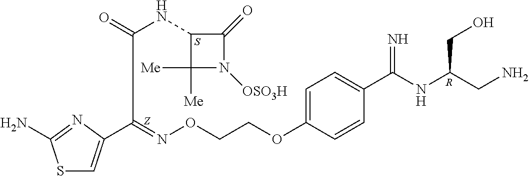 | 1448672-56-7 |
| 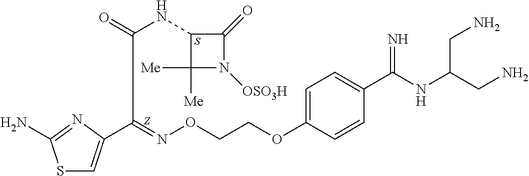 | 1448672-55-6 |
| 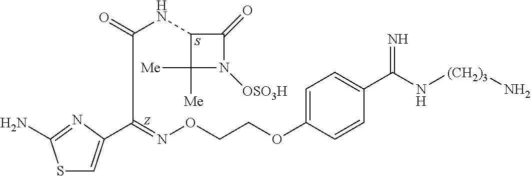 | 1448672-54-5 |
| 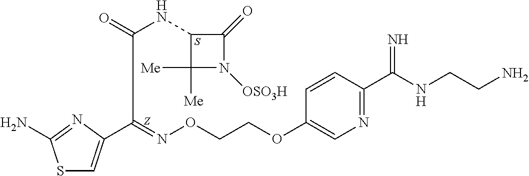 | 1448672-53-4 |
| 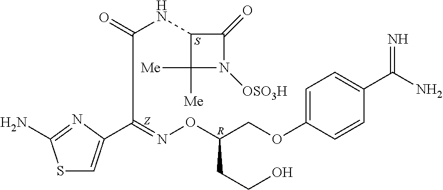 | 1448672-52-3 |
| 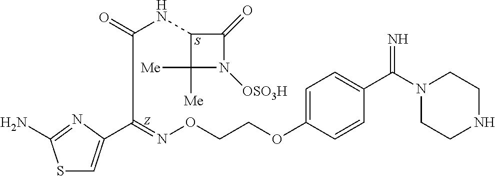 | 1448672-51-2 |
| 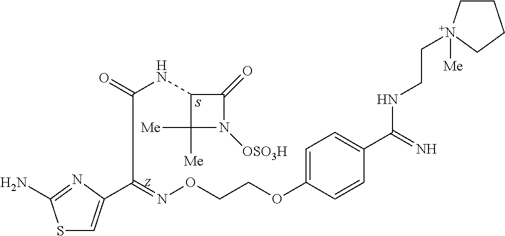 | 1448672-50-1 |
| 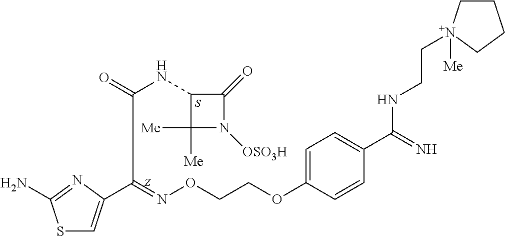 | 1448672-49-8 |
| 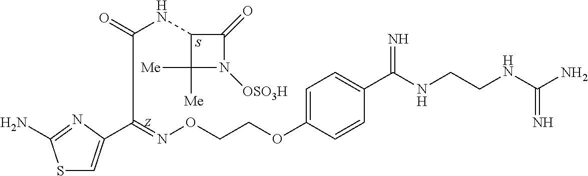 | 1448672-48-7 |
| 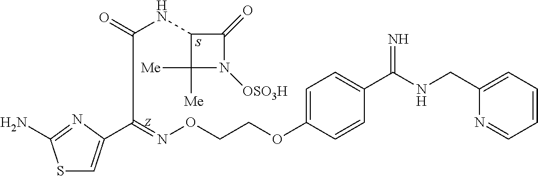 | 1448672-46-5 |
| 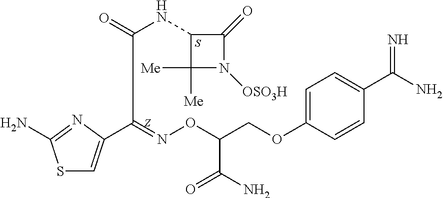 | 1448672-45-4 |
| 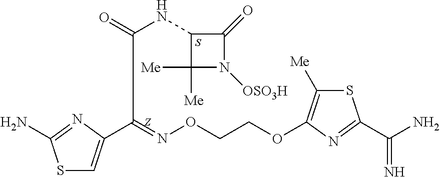 | 1448672-44-3 |
| 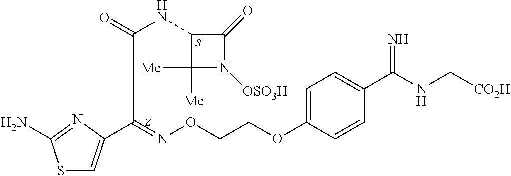 | 1448672-43-2 |
| 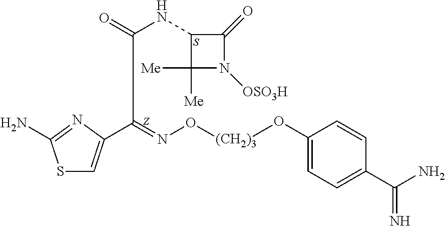 | 1448672-28-3 |
| 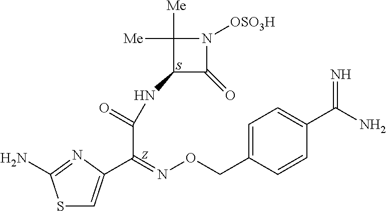 | 1448672-27-2 |
| 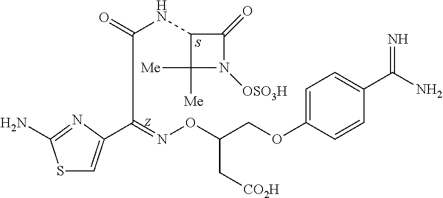 | 1448672-16-9 |
| 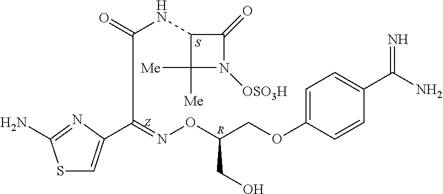 | 1448672-15-8 |
| 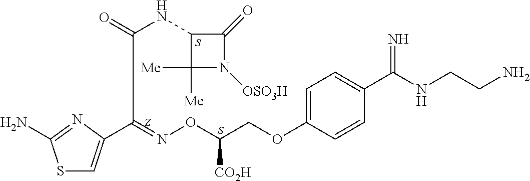 | 1448672-14-7 |
| 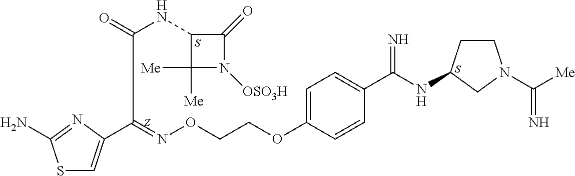 | 1448672-13-6 |
| 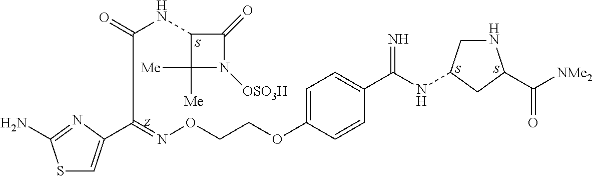 | 1448672-11-4 |
| 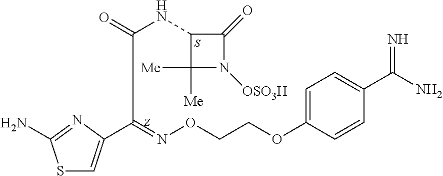 | 1448672-09-0 |
| 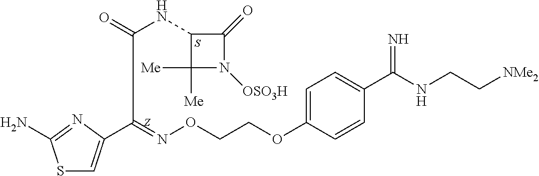 | 1448672-08-9 |
| 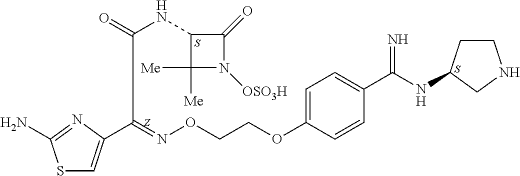 | 1448672-07-8 |
| 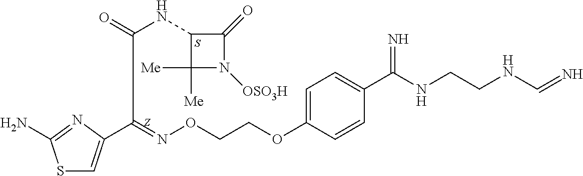 | 1448672-06-7 |
| 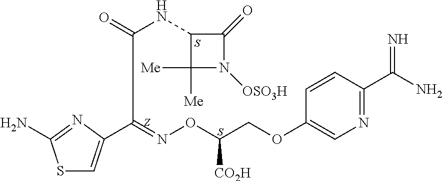 | 1448672-03-4 |
| 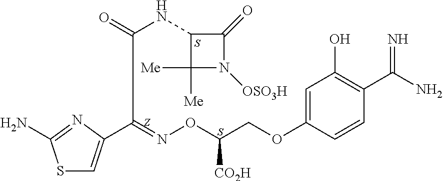 | 1448671-98-4 |
| 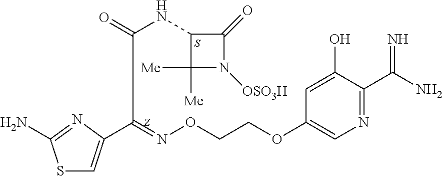 | 1448671-96-2 |
| 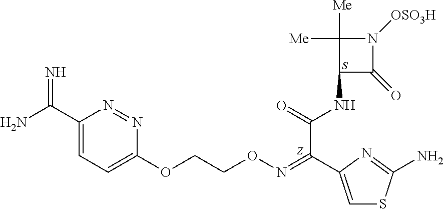 | 1448671-88-2 |
| 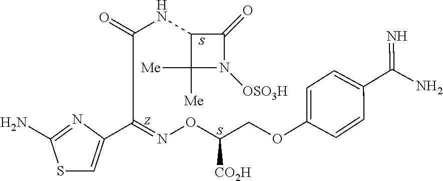 | 1448671-85-9 |
| 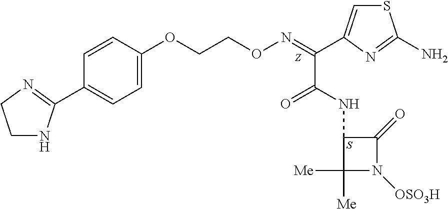 | 1448671-83-7 |
| 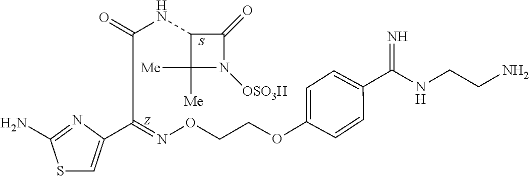 | 1448671-82-6 |
| 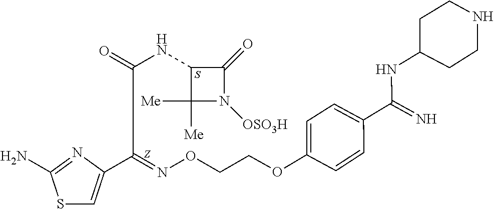 | 1448671-81-5 |
| 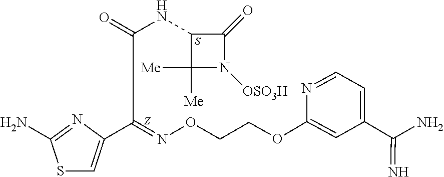 | 1448671-80-4 |
| 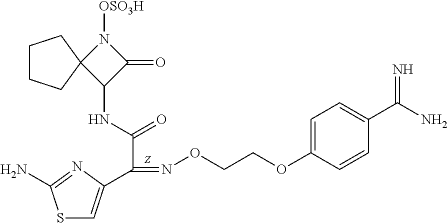 | 1448671-74-6 |
| 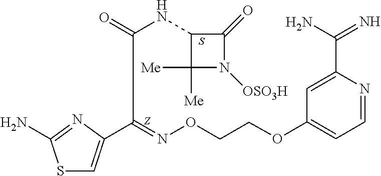 | 1448671-73-5 |
| 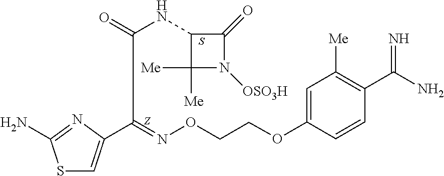 | 1448671-71-3 |
| 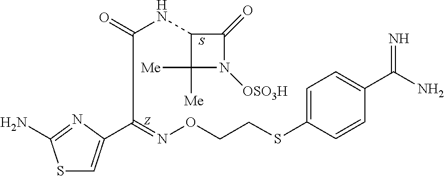 | 1448671-65-5 |
| 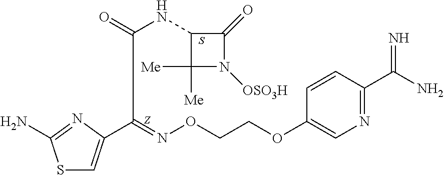 | 1448671-62-2 |
| 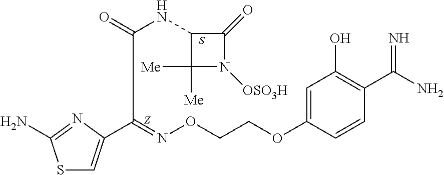 | 1448671-59-7 |
| 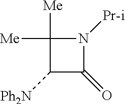 | 1446695-36-8 |
| 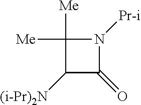 | 1279084-27-3 |
| 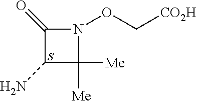 | 1216954-30-1 |
| 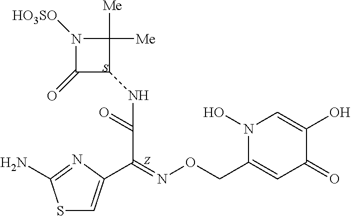 | 1064505-72-1 |
| 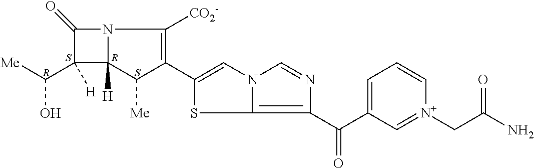 | |
| 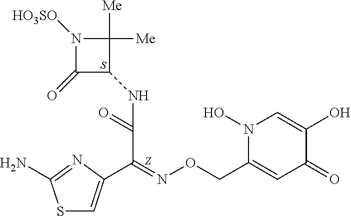 | 1064505-71-0 |
| 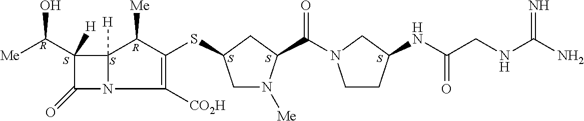 | |
| 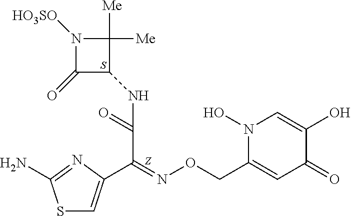 | 1064505-66-3 |
| 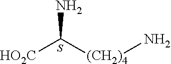 | |
| 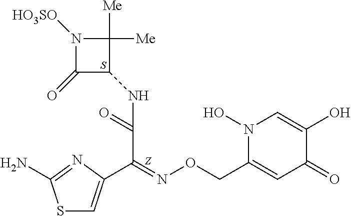 | 1064505-61-8 |
| 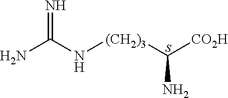 | |
| 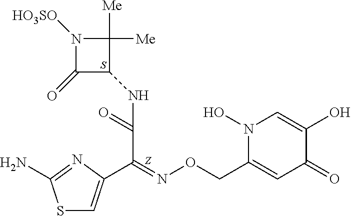 | 1064505-59-4 |
| 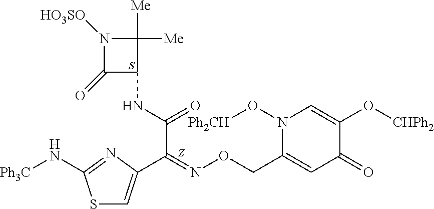 | 1064505-54-9 |
| 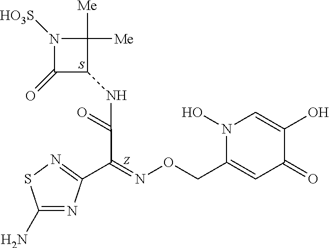 | 941285-29-6 |
| 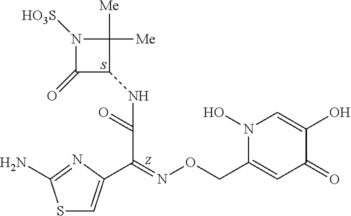 | 941285-28-5 |
| 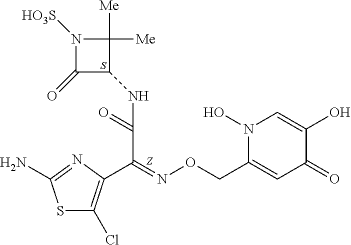 | 941285-27-4 |
| 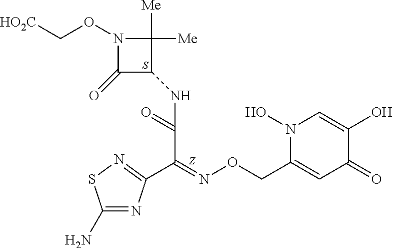 | 941285-24-1 |
| 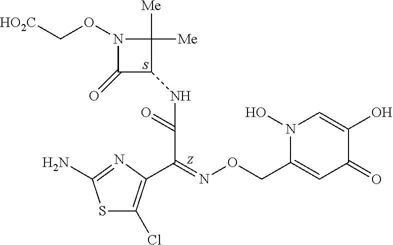 | 941285-23-0 |
| 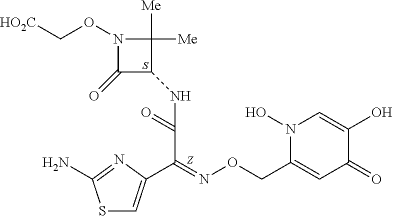 | 941285-22-9 |
| 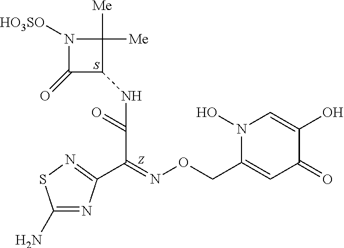 | 941285-21-8 |
| 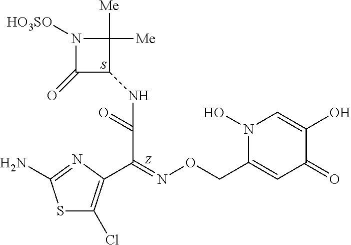 | 941285-20-7 |
| 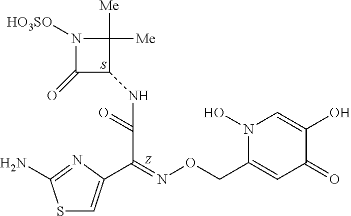 | 941285-15-0 |
| 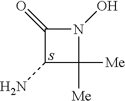 | 820253-42-7 |
| 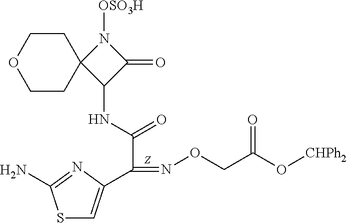 | 793627-56-2 |
| 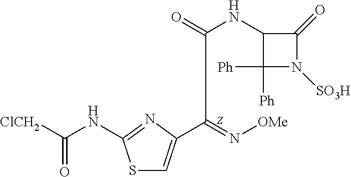 | 792861-65-5 |
| 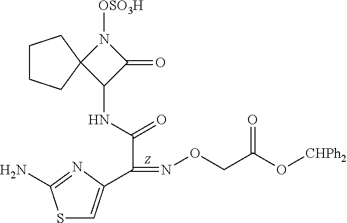 | 791022-44-1 |
| 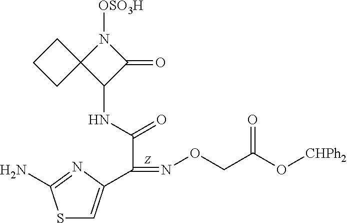 | 784099-50-9 |
| 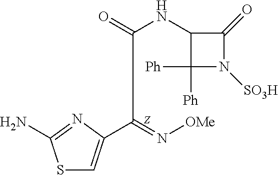 | 783259-43-8 |
| 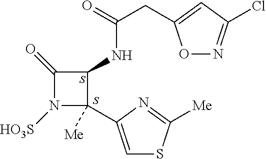 | 779302-30-6 |
| 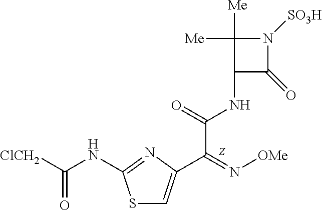 | 777012-46-1 |
| 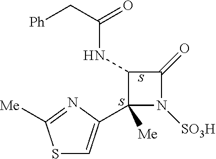 | 776271-84-2 |
| 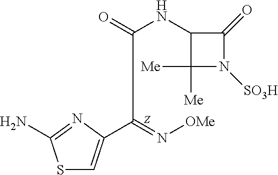 | 761355-82-2 |
| 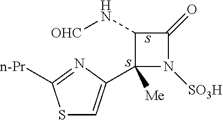 | 757164-87-7 |
| 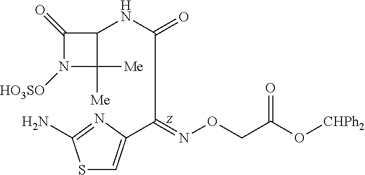 | 756778-23-1 |
| 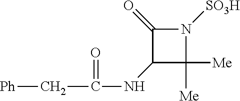 [SMILES: CC1(C)C(NC(=O)Cc2ccccc2)C(=O)N1S(=O)(=O)O] | 754138-75-5 |
| 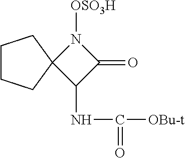 | 751440-92-3 |
| 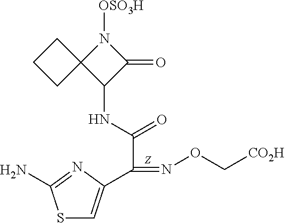 | 742022-04-4 |
| 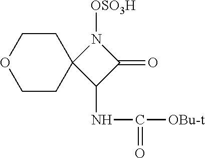 | 740047-72-7 |
| 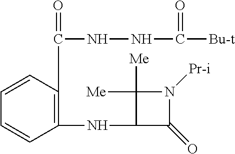 | 634589-54-1 |
| 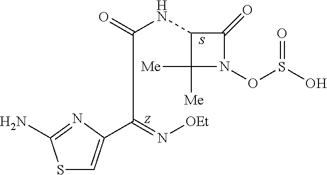 | 469868-49-3 |
| 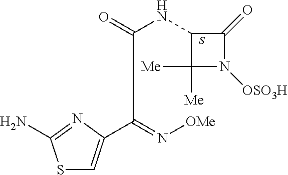 | 415708-54-2 |
| 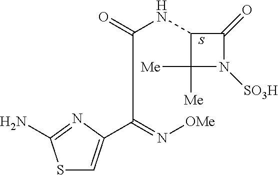 | 405940-67-2 |
| 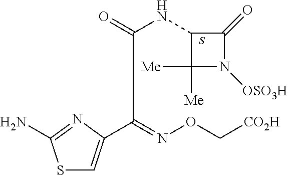 | 405940-56-9 |
| 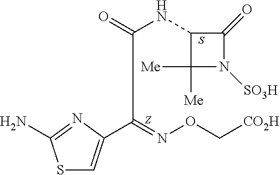 | 370099-06-2 |
| 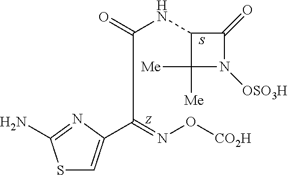 | 270597-80-3 |
| 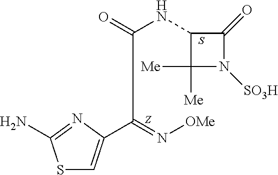 | 270597-79-0 |
| 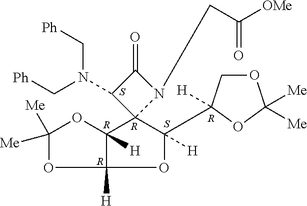 | 197657-15-1 |
| 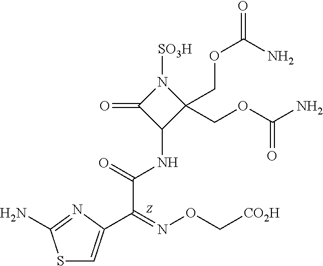 | 188881-42-7 |
| 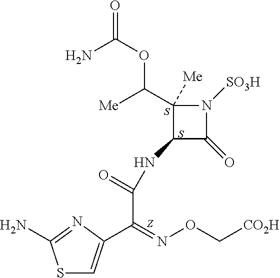 | 188881-41-6 |
| 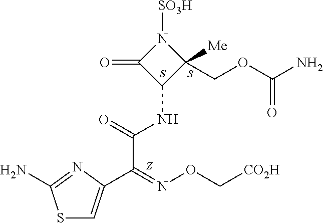 | 188881-39-2 |
| 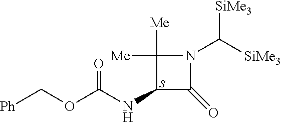 | 187332-77-0 |
| 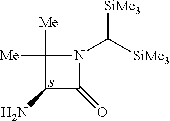 | 187332-76-9 |
| 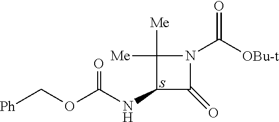 | 176217-49-5 |
| 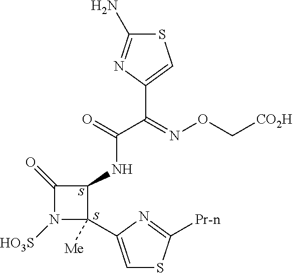 | 172544-37-5 |
| 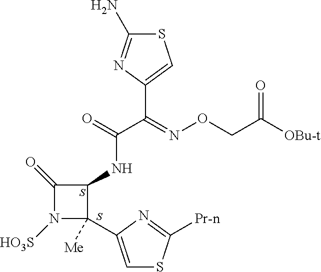 | 172544-36-4 |
| 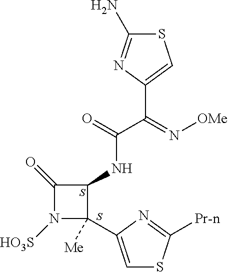 | 172544-35-3 |
| 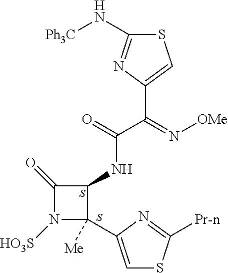 | 172543-73-6 |
| 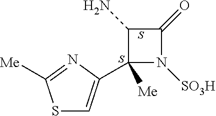 | 172543-72-5 |
| 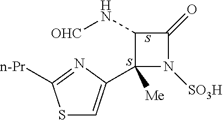 | 172543-71-4 |
| 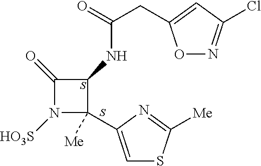 | 172543-70-3 |
| 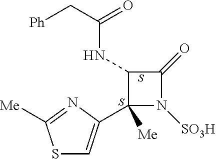 | 172543-69-0 |
| 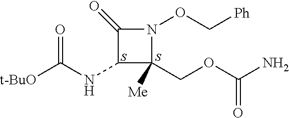 | 167256-82-8 |
| 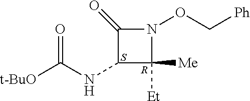 | 167256-81-7 |
| 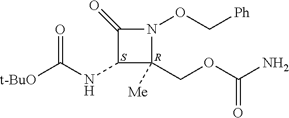 | 167102-87-6 |
| 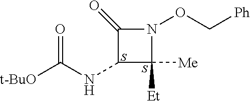 | 167102-78-5 |
| 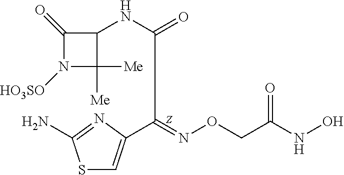 | 126374-19-4 |
| 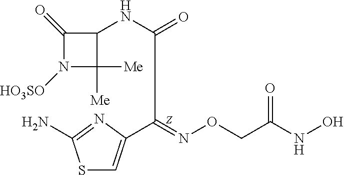 | 126374-18-3 |
| 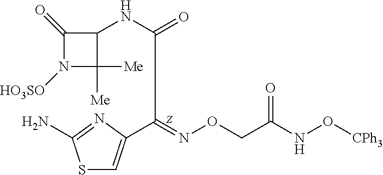 | 126268-87-9 |
| 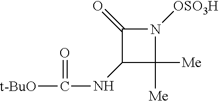 | 126268-86-8 |
| 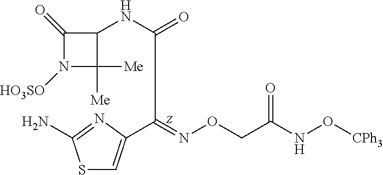 | 126268-84-6 |
| 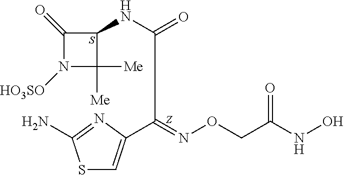 | 126268-83-5 |
| 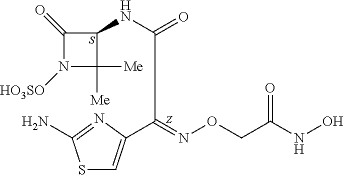 | 126268-82-4 |
| 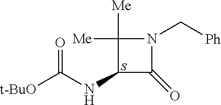 | 124716-69-4 |
| 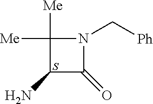 | 124716-66-1 |
| 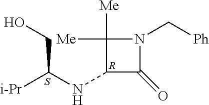 | 124716-65-0 |
| 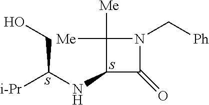 | 124716-64-9 |
| 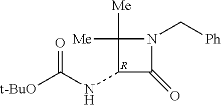 | 124716-59-2 |
| 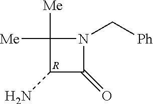 | 124716-53-6 |
| 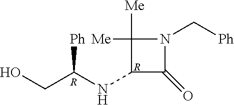 | 124716-52-5 |
| 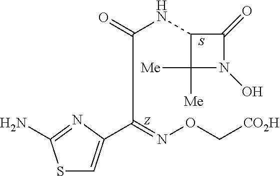 | 123176-21-6 |
| 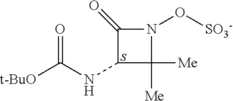 | 123176-14-7 |
| 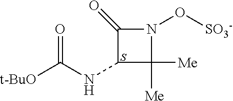 | 123176-13-6 |
| 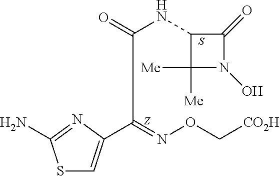 | 123176-12-5 |
| 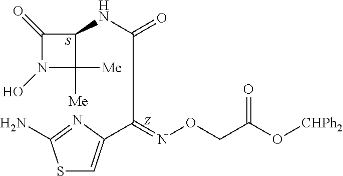 | 123176-11-4 |
| 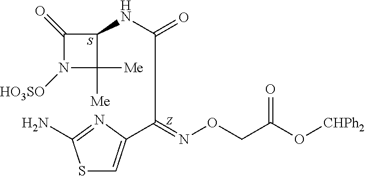 | 123176-10-3 |
| 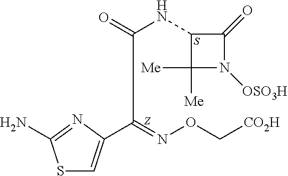 | 116208-58-3 |
| 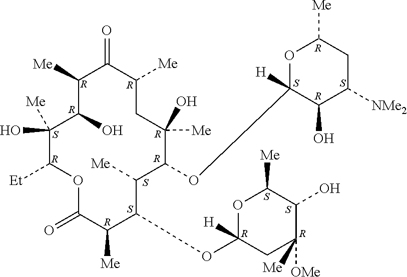 | |
| 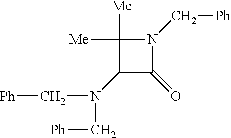 [SMILES: CC1(C)C(N(Cc2ccccc2)Cc2ccccc2)C(=O)N1Cc1ccccc1] | 114693-32-2 |
| 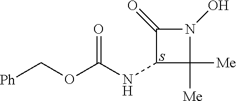 | 112110-91-5 |
| 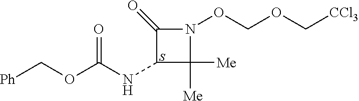 | 112110-90-4 |
| 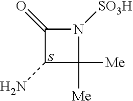 | 111556-12-8 |
| 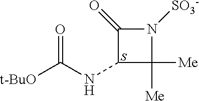 | 111556-11-7 |
| 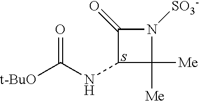 | 111556-10-6 |
| 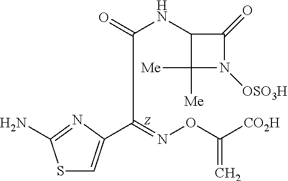 | 111556-09-3 |
| 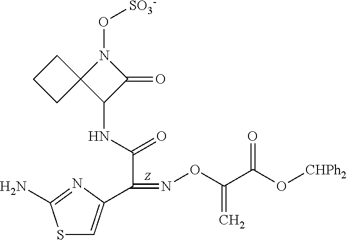 | 111491-10-2 |
| 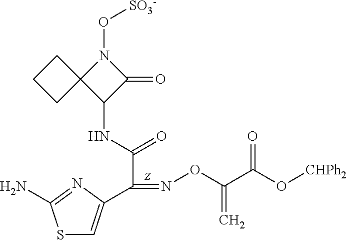 | 111491-09-9 |
| 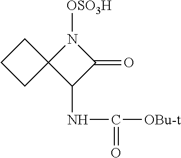 | 111491-08-8 |
| 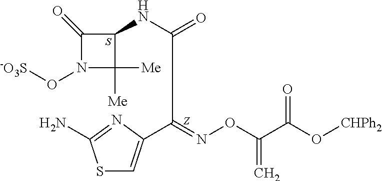 | 111491-06-6 |
| 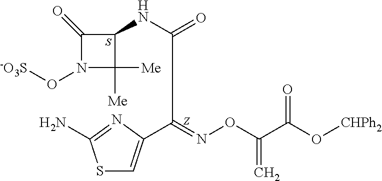 | 111491-05-5 |
| 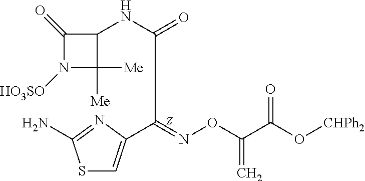 | 111491-03-3 |
| 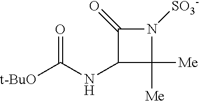 | 111491-02-2 |
| 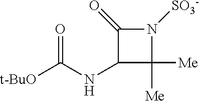 | 111491-01-1 |
| 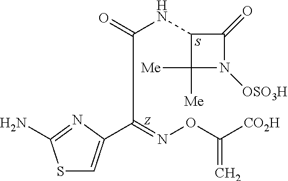 | 111490-95-0 |
| 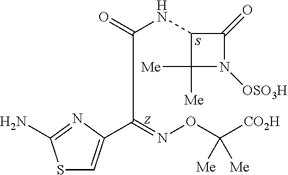 | 109885-32-7 |
| 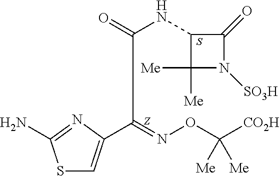 | 109885-30-5 |
| 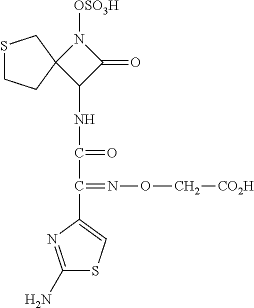 | 105928-93-6 |
| 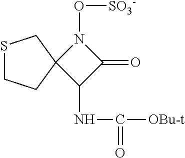 | 105885-49-2 |
| 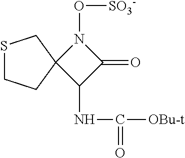 | 105885-48-1 |
| 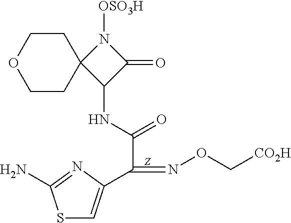 | 105871-74-7 |
| 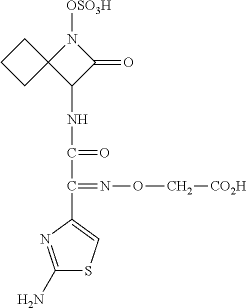 | 105871-73-6 |
| 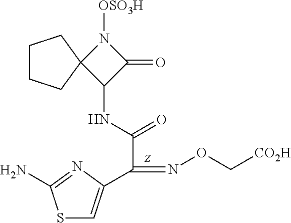 | 105871-72-5 |
| 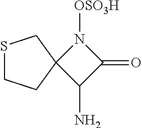 | 105871-71-4 |
| 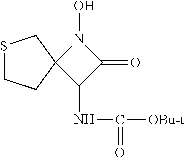 | 105871-70-3 |
| 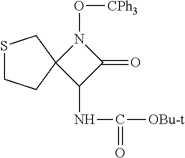 | 105871-69-0 |
| 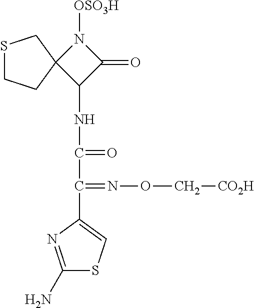 | 105871-65-6 |
| 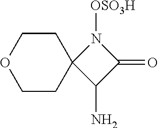 | 105871-63-4 |
| 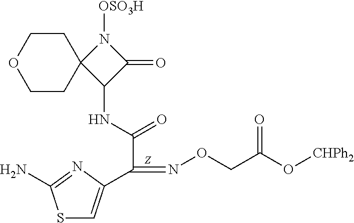 | 105871-62-3 |
| 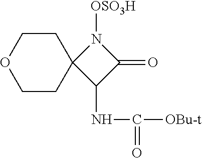 | 105871-61-2 |
| 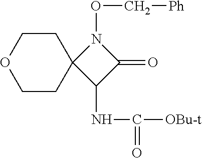 | 105871-60-1 |
| 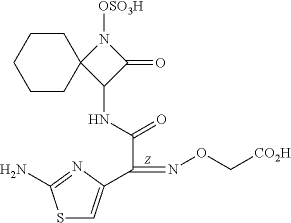 | 105871-59-8 |
| 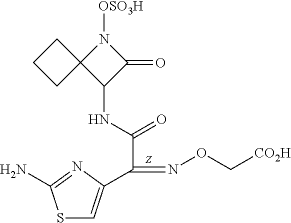 | 105871-58-7 |
| 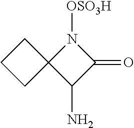 | 105871-57-6 |
| 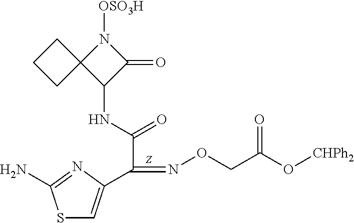 | 105871-56-5 |
| 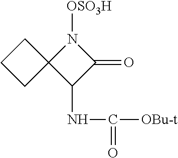 | 105871-55-4 |
| 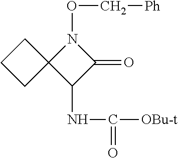 | 105871-54-3 |
| 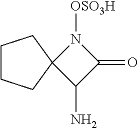 | 105871-50-9 |
| 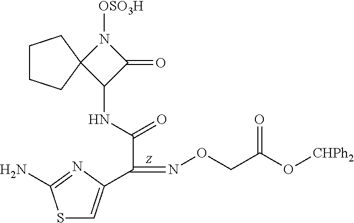 | 105871-49-6 |
| 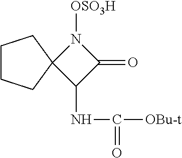 | 105871-48-5 |
| 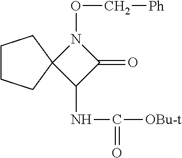 | 105871-47-4 |
| 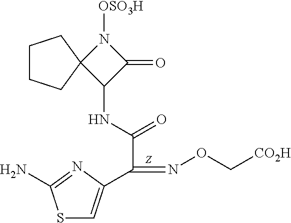 | 105871-43-0 |
| 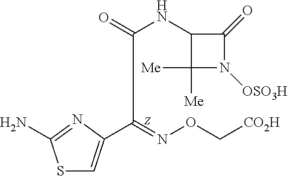 | 105615-86-9 |
| 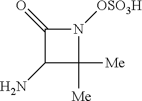 | 105615-57-4 |
| 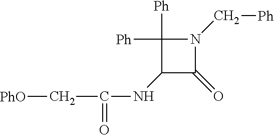 [SMILES: O=C(COc1ccccc1)NC1C(=O)N(Cc2ccccc2)C1(c1ccccc1)c1ccccc1] | 105602-12-8 |
| 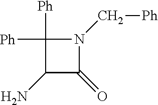 [SMILES: NC1C(=O)N(Cc2ccccc2)C1(c1ccccc1)c1ccccc1] | 105602-11-7 |
| 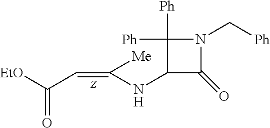 [SMILES: CCOC(=O)/C=C(/C)NC1C(=O)N(Cc2ccccc2)C1(c1ccccc1)c1ccccc1] | 105602-10-6 |
| 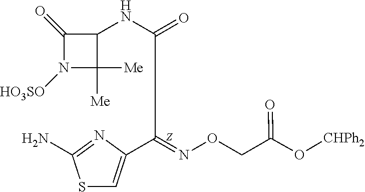 | 105561-90-8 |
| 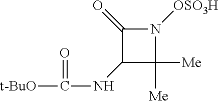 | 105561-89-5 |
| 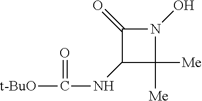 [SMILES: CC(C)(C)OC(=O)NC1C(=O)N(O)C1(C)C] | 105561-88-4 |
| 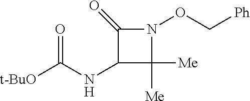 [SMILES: CC(C)(C)OC(=O)NC1C(=O)N(OCc2ccccc2)C1(C)C] | 105561-87-3 |
| 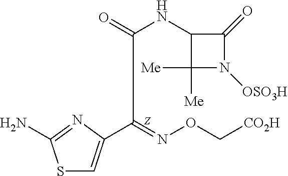 | 105561-85-1 |
| 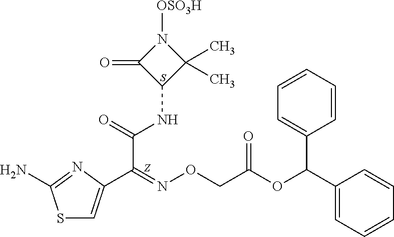 | 105504-77-6 |
| 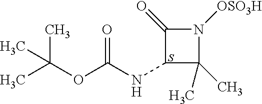 | 105504-76-5 |
| 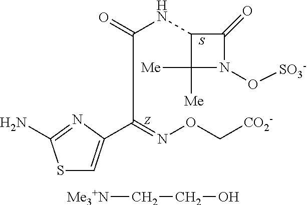 | 102916-21-2 |
| 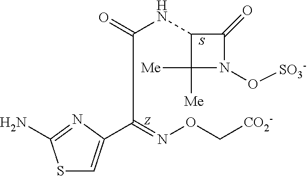 | 102868-95-1 |
| 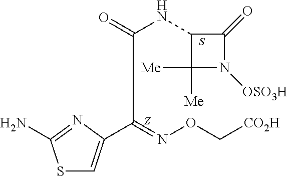 | 102522-04-3 |
| 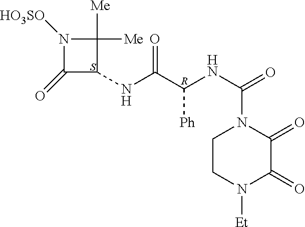 | 102522-03-2 |
| 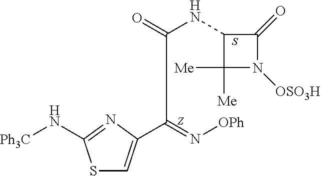 | 102507-95-9 |
| 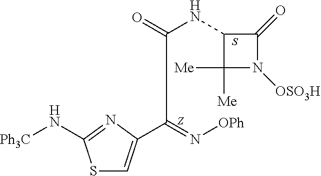 | 102507-94-8 |
| 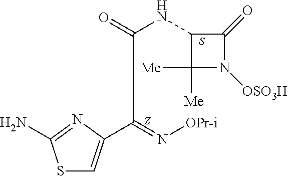 | 102507-93-7 |
| 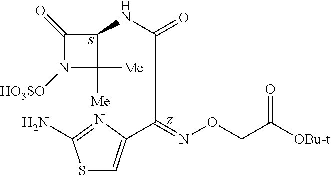 | 102507-87-9 |
| 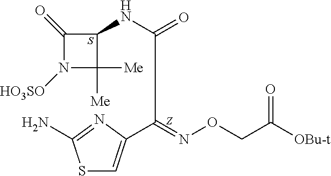 | 102507-86-8 |
| 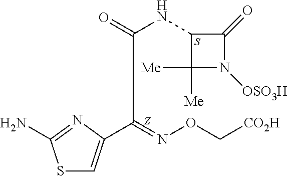 | 102507-71-1 |
| 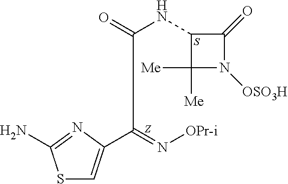 | 102507-67-5 |
| 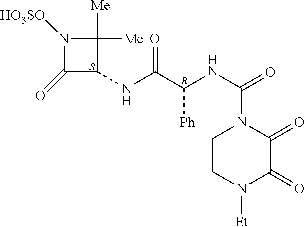 | 102507-55-1 |
| 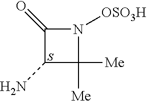 | 102507-49-3 |
| 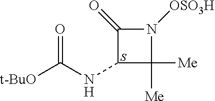 | 102507-38-0 |
| 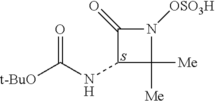 | 102507-37-9 |
| 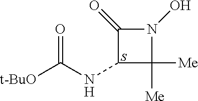 | 102507-31-3 |
| 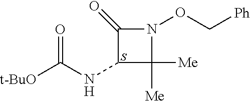 | 102507-25-5 |
| 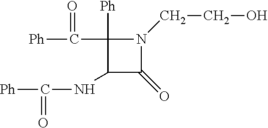 [SMILES: O=C(NC1C(=O)N(CCO)C1(C(=O)c1ccccc1)c1ccccc1)c1ccccc1] | 97577-27-0 |
| 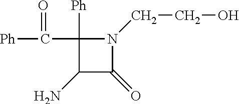 [SMILES: NC1C(=O)N(CCO)C1(C(=O)c1ccccc1)c1ccccc1] | 97577-26-9 |
| 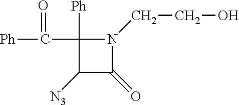 [SMILES: [N-]=[N+]=NC1C(=O)N(CCO)C1(C(=O)c1ccccc1)c1ccccc1] | 97577-25-8 |
| 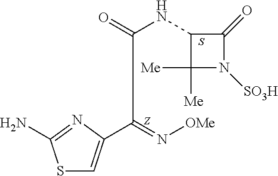 | 88825-06-3 |
| 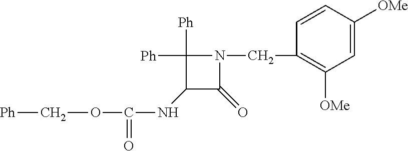 [SMILES: COc1ccc(CN2C(=O)C(NC(=O)OCc3ccccc3)C2(c2ccccc2)c2ccccc2)c(OC)c1] | 85573-96-2 |
| 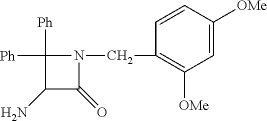 [SMILES: COc1ccc(CN2C(=O)C(N)C2(c2ccccc2)c2ccccc2)c(OC)c1] | 85573-95-1 |
| 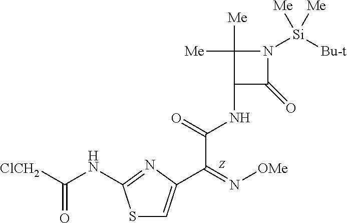 | 85573-93-9 |
| 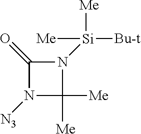 | 85573-92-8 |
| 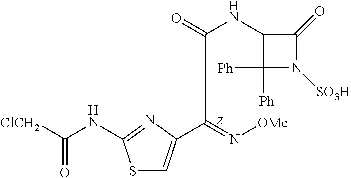 | 85573-90-6 |
| 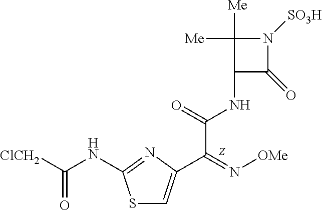 | 85573-89-3 |
| 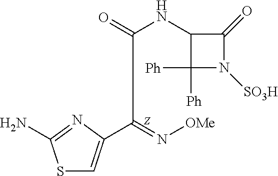 | 85573-88-2 |
| 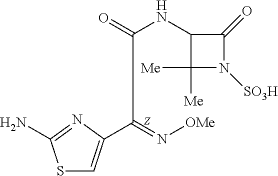 | 85573-87-1 |
| 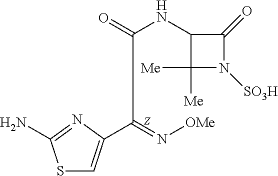 | 80560-02-7 |
| 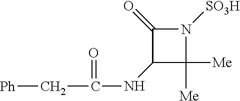 [SMILES: CC1(C)C(NC(=O)Cc2ccccc2)C(=O)N1S(=O)(=O)O] | 80541-94-2 |
| 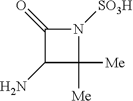 [SMILES: CC1(C)C(N)C(=O)N1S(=O)(=O)O] | 80541-93-1 |
| 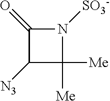 | 80541-92-0 |
| 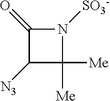 | 80541-91-9 |
| 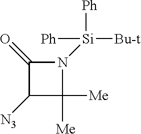 | 80541-89-5 |
| 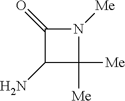 | 55758-04-8 |
| 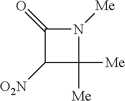 [SMILES: CN1C(=O)C([N+](=O)[O-])C1(C)C] | 33259-64-2 |

In some embodiments, the compound of Formula (II) is of Formula (II-a):

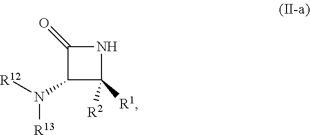

or pharmaceutically acceptable salts, stereoisomers, polymorphs, isotopes, solvates, hydrates, tautomers, or prodrugs thereof.

In some embodiments, the compound of Formula (II′) is of Formula (II-a′):

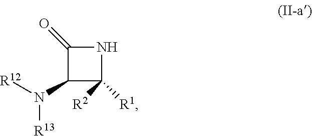

or pharmaceutically acceptable salts, stereoisomers, polymorphs, isotopes, solvates, hydrates, tautomers, or prodrugs thereof.

In some embodiments, the compound of Formula (II) is of Formula (II-b):

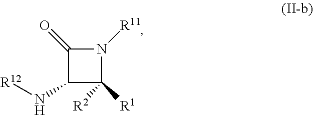

or pharmaceutically acceptable salts, stereoisomers, polymorphs, isotopes, solvates, hydrates, tautomers, or prodrugs thereof.

In some embodiments, the compound of Formula (II′) is of Formula (II-b′):

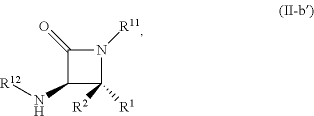

or pharmaceutically acceptable salts, stereoisomers, polymorphs, isotopes, solvates, hydrates, tautomers, or prodrugs thereof.

In some embodiments, the compound of Formula (II) is of Formula (II-c):

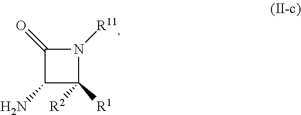

or pharmaceutically acceptable salts, stereoisomers, polymorphs, isotopes, solvates, hydrates, tautomers, or prodrugs thereof.

In some embodiments, the compound of Formula (II′) is of Formula (II-c′):

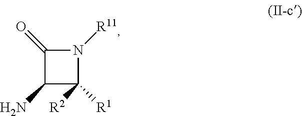

or pharmaceutically acceptable salts, stereoisomers, polymorphs, isotopes, solvates, hydrates, tautomers, or prodrugs thereof.
Groups R¹ and R²

In some embodiments of Formula (I), (I′), (II), or (II′), R¹ is substituted or unsubstituted C₁₋₁₀ alkyl. In some embodiments of Formula (I), (I′), (II), or (II′), R¹ is substituted or unsubstituted C₁₋₃ alkyl. In some embodiments of Formula (I), (I′), (II), or (II′), R¹ is substituted or unsubstituted C₁₋₆ alkyl. In some embodiments of Formula (I), (I′), (II), or (II′), R¹ is branched or unbranched substituted or unsubstituted C₁₋₆ alkyl. In some embodiments of Formula (I), (I′), (II), or (II′), R¹ is substituted or unsubstituted methyl. In some embodiments of Formula (I), (I′), (II), or (II′), R¹ is substituted or unsubstituted ethyl. In some embodiments of Formula (I), (I′), (II), or (II′), R¹ is substituted or unsubstituted propyl. In some embodiments of Formula (I), (I′), (II), or (II′), R¹ is substituted or unsubstituted butyl. In some embodiments of Formula (I), (I′), (II), or (II′), R¹ is substituted or unsubstituted pentyl. In some embodiments of Formula (I), (I′), (II), or (II′), R¹ is substituted or unsubstituted C₁₋₁₀ haloalkyl. In some embodiments of Formula (I), (I′), (II), or (II′), R¹ is substituted or unsubstituted C₁₋₃ haloalkyl. In some embodiments of Formula (I), (I′), (II), or (II′), R¹ is substituted or unsubstituted fluoroalkyl. In some embodiments of Formula (I), (I′), (II), or (II′), R¹ is trifluoromethyl. In some embodiments of Formula (I), (I′), (II), or (II′), R¹ is substituted or unsubstituted trifluoroethyl. In some embodiments of Formula (I), (I′), (II), or (II′), R¹ is substituted or unsubstituted C₂₋₁₀ alkenyl. In some embodiments of Formula (I), (I′), (II), or (II′), R¹ is substituted or unsubstituted vinyl. In some embodiments of Formula (I), (I′), (II), or (II′), R¹ is substituted or unsubstituted allyl. In some embodiments of Formula (I), (I′), (II), or (II′), R¹ is substituted or unsubstituted propenyl. In some embodiments of Formula (I), (I′), (II), or (II′), R¹ is substituted or unsubstituted butenyl. In some embodiments of Formula (I), (I′), (II), or (II′), R¹ is substituted or unsubstituted C₂₋₁₀ alkynyl. In some embodiments of Formula (I), (I′), (II), or (II′), R¹ is substituted or unsubstituted acetylenyl. In some embodiments of Formula (I), (I′), (II), or (II′), R¹ is substituted or unsubstituted propargyl. In some embodiments of Formula (I), (I′), (II), or (II′), R¹ is substituted or unsubstituted butynyl. In some embodiments of Formula (I), (I′), (II), or (II′), R¹ is substituted or unsubstituted C₆₋₁₀ aryl. In some embodiments of Formula (I), (I′), (II), or (II′), R¹ is substituted or unsubstituted phenyl. In some embodiments of Formula (I), (I′), (II), or (II′), R¹ is substituted or unsubstituted C₆₋₁₀ aralkyl. In some embodiments of Formula (I), (I′), (II), or (II′), R¹ is substituted or unsubstituted C₁₋₁₀ alkoxy. In some embodiments of Formula (I), (I′), (II), or (II′), R¹ is substituted or unsubstituted C₁₋₆ alkoxy. In some embodiments of Formula (I), (I′), (II), or (II′), R¹ is CH₂—OMe. In some embodiments of Formula (I), (I′), (II), or (II′), R¹ is CH₂-OEt. In some embodiments of Formula (I), (I′), (II), or (II′), R¹CH₂—OPr. In some embodiments of Formula (I), (I′), (II), or (II′), R¹ is CH₂—OCF₃. In some embodiments of Formula (I), (I′), (II), or (II′), R¹ is substituted or unsubstituted CH₂—OR⁷. In some embodiments of Formula (I), (I′), (II), or (II′), R¹ is substituted or unsubstituted 5-10 membered heteroaryl. In some embodiments of Formula (I), (I′), (II), or (II′), R¹ is substituted or unsubstituted 5-10 membered heteroaralkyl. In some embodiments of Formula (I), (I′), (II), or (II′), R¹ is substituted or unsubstituted 4-10 membered heterocyclyl. In some embodiments of Formula (I), (I′), (II), or (II′), R¹ is substituted or unsubstituted 4-10 membered heterocyclylalkyl. In some embodiments of Formula (I), (I′), (II), or (II′), R¹ is substituted or unsubstituted 3-10 membered carbocyclyl. In some embodiments of Formula (I), (I′), (II), or (II′), R¹ is substituted or unsubstituted 3-6 membered carbocyclyl. In some embodiments of Formula (I), (I′), (II), or (II′), R¹ is substituted or unsubstituted 3-10 membered carbocyclylalkyl. In some embodiments of Formula (I), (I′), (II), or (II′), R¹ is optionally substituted with 5 occurrences of R⁵. In some embodiments of Formula (I), (I′), (II), or (II′), R¹ is optionally substituted with 4 occurrences of R⁵. In some embodiments of Formula (I), (I′), (II), or (II′), R¹ is optionally substituted with 3 occurrences of R⁵. In some embodiments of Formula (I), (I′), (II), or (II′), R¹ is optionally substituted with 2 occurrences of R⁵. In some embodiments of Formula (I), (I′), (II), or (II′), R¹ is optionally substituted with 1 occurrence of R⁵. In some embodiments of Formula (I), (I′), (II), or (II′), R¹ is optionally substituted with 0 occurrences of R⁵

In some embodiments of Formula (II) or (II′), R² is substituted or unsubstituted C₁₋₁₀ alkyl. In some embodiments of Formula (II) or (II′), R² is substituted or unsubstituted C₁₋₆ alkyl. In some embodiments of Formula (II) or (II′), R² is substituted or unsubstituted C₁₋₃alkyl. In some embodiments of Formula (II) or (II′), R² is branched or unbranched substituted or unsubstituted C₁₋₆ alkyl. In some embodiments of Formula (II) or (II′), R² is substituted or unsubstituted methyl. In some embodiments of Formula (II) or (II′), R² is substituted or unsubstituted ethyl. In some embodiments of Formula (II) or (II′), R² is substituted or unsubstituted propyl. In some embodiments of Formula (II) or (II′), R² is substituted or unsubstituted butyl. In some embodiments of Formula (II) or (II′), R² is substituted or unsubstituted pentyl. In some embodiments of Formula (II) or (II′), R² is substituted or unsubstituted C₁₋₁₀ haloalkyl. In some embodiments of Formula (II) or (II′), R² is substituted or unsubstituted C₁₋₃ haloalkyl. In some embodiments of Formula (II) or (II′), R² is substituted or unsubstituted fluoroalkyl. In some embodiments of Formula (II) or (II′), R² is trifluoromethyl. In some embodiments of Formula (II) or (II′), R² is substituted or unsubstituted trifluoroethyl. In some embodiments of Formula (II) or (II′), R² is substituted or unsubstituted C₂₋₁₀ alkenyl. In some embodiments of Formula (II) or (II′), R² is substituted or unsubstituted vinyl. In some embodiments of Formula (II) or (II′), R² is substituted or unsubstituted allyl. In some embodiments of Formula (II) or (II′), R² is substituted or unsubstituted propenyl. In some embodiments of Formula (II) or (II′), R² is substituted or unsubstituted butenyl. In some embodiments of Formula (II) or (II′), R² is substituted or unsubstituted C₂₋₁₀ alkynyl. In some embodiments of Formula (II) or (II′), R² is substituted or unsubstituted acetylenyl. In some embodiments of Formula (II) or (II′), R² is substituted or unsubstituted propargyl. In some embodiments of Formula (II) or (II′), R² is substituted or unsubstituted butynyl. In some embodiments of Formula (II) or (II′), R² is substituted or unsubstituted C₆₋₁₀ aryl. In some embodiments of Formula (II) or (II′), R² is substituted or unsubstituted phenyl. In some embodiments of Formula (II) or (II′), R² is substituted or unsubstituted C₆₋₁₀ aralkyl. In some embodiments of Formula (II) or (II′), R² is substituted or unsubstituted C₁₋₁₀ alkoxy. In some embodiments of Formula (II) or (II′), R² is substituted or unsubstituted C₁₋₆ alkoxy. In some embodiments of Formula (II) or (II′), R² is CH₂—OMe. In some embodiments of Formula (II) or (II′), R² is CH₂—OEt. In some embodiments of Formula (II) or (II′), R²CH₂—OPr. In some embodiments of Formula (II) or (II′), R² is CH₂—OCF₃. In some embodiments of Formula (II) or (II′), R² is substituted or unsubstituted CH₂—OR⁷. In some embodiments of Formula (II) or (II′), R² is substituted or unsubstituted 5-10 membered heteroaryl. In some embodiments of Formula (II) or (II′), R² is substituted or unsubstituted 5-10 membered heteroaralkyl. In some embodiments of Formula (II) or (II′), R² is substituted or unsubstituted 4-10 membered heterocyclyl. In some embodiments of Formula (II) or (II′), R² is substituted or unsubstituted 4-10 membered heterocyclylalkyl. In some embodiments of Formula (II) or (II′), R² is substituted or unsubstituted 3-10 membered carbocyclyl. In some embodiments of Formula (II) or (II′), R² is substituted or unsubstituted 3-6 membered carbocyclyl. In some embodiments of Formula (II) or (II′), R² is substituted or unsubstituted 3-10 membered carbocyclylalkyl. In some embodiments of Formula (II) or (II′), R² is optionally substituted with 5 occurrences of R⁵. In some embodiments of Formula (II) or (II′), R² is optionally substituted with 4 occurrences of R⁵. In some embodiments of Formula (II) or (II′), R² is optionally substituted with 3 occurrences of R⁵. In some embodiments of Formula (II) or (II′), R² is optionally substituted with 2 occurrences of R⁵. In some embodiments of Formula (II) or (II′), R² is optionally substituted with 1 occurrence of R⁵. In some embodiments of Formula (II) or (II′), R² is optionally substituted with 0 occurrences of R⁵

In some embodiments of Formula (II) or (II′), R¹ is substituted or unsubstituted C₁₋₃ alkyl; and R² is substituted or unsubstituted C₁₋₃ alkyl. In some embodiments of Formula (II) or (II′), R¹ is substituted or unsubstituted C₁₋₃ alkyl; and R² is substituted or unsubstituted C₁₋₃ haloalkyl. In some embodiments of Formula (II) or (II′), R¹ is substituted or unsubstituted C₁₋₃ alkyl; and R² is substituted or unsubstituted C₆ aryl. In some embodiments of Formula (II) or (II′), R¹ is substituted or unsubstituted C₁₋₃ alkyl; and R² is substituted or unsubstituted C₁₋₆ alkoxy. In some embodiments of Formula (II) or (II′), R¹ is substituted or unsubstituted C₁₋₃ alkyl; and R² is substituted or unsubstituted 5-10 membered heteroaryl. In some embodiments of Formula (II) or (II′), R¹ is substituted or unsubstituted C₁₋₃ alkyl; and R² is substituted or unsubstituted 4-10 membered heterocyclyl. In some embodiments of Formula (II) or (II′), R¹ is substituted or unsubstituted C₁₋₃ alkyl; and R² is substituted or unsubstituted 3-6 membered carbocyclyl. In some embodiments of Formula (II) or (II′), R¹ is substituted or unsubstituted C₁₋₃ haloalkyl; and R² is substituted or unsubstituted C₁₋₃ alkyl. In some embodiments of Formula (II) or (II′), R¹ is substituted or unsubstituted C₁₋₃ haloalkyl; and R² is substituted or unsubstituted C₁₋₃ haloalkyl. In some embodiments of Formula (II) or (II′), R¹ is substituted or unsubstituted C₁₋₃ haloalkyl; and R² is substituted or unsubstituted C₆ aryl. In some embodiments of Formula (II) or (II′), R¹ is substituted or unsubstituted C₁₋₃haloalkyl; and R² is substituted or unsubstituted C₁₋₆ alkoxy. In some embodiments of Formula (II) or (II′), R¹ is substituted or unsubstituted C₁₋₃ haloalkyl; and R² is substituted or unsubstituted 5-10 membered heteroaryl. In some embodiments of Formula (II) or (II′), R¹ is substituted or unsubstituted C₁₋₃ haloalkyl; and R² is substituted or unsubstituted 4-10 membered heterocyclyl. In some embodiments of Formula (II) or (II′), R¹ is substituted or unsubstituted C₁₋₃ haloalkyl; and R² is substituted or unsubstituted 3-6 membered carbocyclyl. In some embodiments of Formula (II) or (II′), R¹ is substituted or unsubstituted C₆ aryl; and R² is substituted or unsubstituted C₁₋₃ alkyl. In some embodiments of Formula (II) or (II′), R¹ is substituted or unsubstituted C₆ aryl; and R² is substituted or unsubstituted C₁₋₃ haloalkyl. In some embodiments of Formula (II) or (II′), R¹ is substituted or unsubstituted C₆ aryl; and R² is substituted or unsubstituted C₆ aryl. In some embodiments of Formula (II) or (II′), R¹ is substituted or unsubstituted C₆ aryl; and R² is substituted or unsubstituted C₁₋₆ alkoxy. In some embodiments of Formula (II) or (II′), R¹ is substituted or unsubstituted C₆ aryl; and substituted or unsubstituted R² is 5-10 membered heteroaryl. In some embodiments of Formula (II) or (II′), R¹ is substituted or unsubstituted C₆ aryl; and R² is substituted or unsubstituted 4-10 membered heterocyclyl. In some embodiments of Formula (II) or (II′), R¹ is substituted or unsubstituted C₆ aryl; and R² is substituted or unsubstituted 3-6 membered carbocyclyl. In some embodiments of Formula (II) or (II′), R¹ is substituted or unsubstituted C₁₋₆ alkoxy; and R² is substituted or unsubstituted C₁₋₃ alkyl. In some embodiments of Formula (II) or (II′), R¹ is substituted or unsubstituted C₁₋₆ alkoxy; and R² is substituted or unsubstituted C₁₋₃ haloalkyl. In some embodiments of Formula (II) or (II′), R¹ is substituted or unsubstituted C₁₋₆ alkoxy; and R² is substituted or unsubstituted C₆ aryl. In some embodiments of Formula (II) or (II′), R¹ is C₁₋₆ alkoxy; and R² is C₁₋₆ alkoxy. In some embodiments of Formula (II) or (II′), R¹ is substituted or unsubstituted C₁₋₆alkoxy; and R² is substituted or unsubstituted 5-10 membered heteroaryl. In some embodiments of Formula (II) or (II′), R¹ is substituted or unsubstituted C₁₋₆ alkoxy; and R² is substituted or unsubstituted 4-10 membered heterocyclyl. In some embodiments of Formula (II) or (II′), R¹ is substituted or unsubstituted C₁₋₆ alkoxy; and R² is substituted or unsubstituted 3-6 membered carbocyclyl. In some embodiments of Formula (II) or (II′), R¹ is substituted or unsubstituted 5-10 membered heteroaryl; and R² is substituted or unsubstituted C₁₋₃ alkyl. In some embodiments of Formula (II) or (II′), R¹ is substituted or unsubstituted 5-10 membered heteroaryl; and R² is substituted or unsubstituted C₁₋₃ haloalkyl. In some embodiments of Formula (II) or (II′), R¹ is substituted or unsubstituted 5-10 membered heteroaryl; and R² is substituted or unsubstituted C₆ aryl. In some embodiments of Formula (II) or (II′), R¹ is substituted or unsubstituted 5-10 membered heteroaryl; and R² is substituted or unsubstituted C₁₋₆ alkoxy. In some embodiments of Formula (II) or (II′), R¹ is substituted or unsubstituted 5-10 membered heteroaryl; and R² is substituted or unsubstituted 5-10 membered heteroaryl. In some embodiments of Formula (II) or (II′), R¹ is substituted or unsubstituted 5-10 membered heteroaryl; and R² is substituted or unsubstituted 4-10 membered heterocyclyl. In some embodiments of Formula (II) or (II′), R¹ is substituted or unsubstituted 5-10 membered heteroaryl; and R² is substituted or unsubstituted 3-6 membered carbocyclyl. In some embodiments of Formula (II) or (II′), R¹ is substituted or unsubstituted 4-10 membered heterocyclyl; and R² is substituted or unsubstituted C₁₋₃ alkyl. In some embodiments of Formula (II) or (II′), R¹ is substituted or unsubstituted 4-10 membered heterocyclyl; and R² is substituted or unsubstituted C₁₋₃ haloalkyl. In some embodiments of Formula (II) or (II′), R¹ is substituted or unsubstituted 4-10 membered heterocyclyl; and R² is substituted or unsubstituted C₆ aryl. In some embodiments of Formula (II) or (II′), R¹ is substituted or unsubstituted 4-10 membered heterocyclyl; and R² is substituted or unsubstituted C₁₋₆ alkoxy. In some embodiments of Formula (II) or (II′), R¹ is substituted or unsubstituted 4-10 membered heterocyclyl; and R² is substituted or unsubstituted 5-10 membered heteroaryl. In some embodiments of Formula (II) or (II′), R¹ is substituted or unsubstituted 4-10 membered heterocyclyl; and R² is substituted or unsubstituted 4-10 membered heterocyclyl. In some embodiments of Formula (II) or (II′), R¹ is substituted or unsubstituted 4-10 membered heterocyclyl; and R² is substituted or unsubstituted 3-6 membered carbocyclyl. In some embodiments of Formula (II) or (II′), R¹ is substituted or unsubstituted 3-6 membered carbocyclyl; and R² is substituted or unsubstituted C₁₋₃ alkyl. In some embodiments of Formula (II) or (II′), R¹ is substituted or unsubstituted 3-6 membered carbocyclyl; and R² is substituted or unsubstituted C₁₋₃ haloalkyl. In some embodiments of Formula (II) or (II′), R¹ is substituted or unsubstituted 3-6 membered carbocyclyl; and R² is substituted or unsubstituted C₆ aryl. In some embodiments of Formula (II) or (II′), R¹ is substituted or unsubstituted 3-6 membered carbocyclyl; and R² substituted or unsubstituted is C₁₋₆ alkoxy. In some embodiments of Formula (II) or (II′), R¹ is substituted or unsubstituted 3-6 membered carbocyclyl; and R² is substituted or unsubstituted 5-10 membered heteroaryl. In some embodiments of Formula (II) or (II′), R¹ is substituted or unsubstituted 3-6 membered carbocyclyl; and R² is substituted or unsubstituted 4-10 membered heterocyclyl. In some embodiments of Formula (II) or (II′), R¹ is substituted or unsubstituted 3-6 membered carbocyclyl; and R² is substituted or unsubstituted 3-6 membered carbocyclyl.

In some embodiments of Formula (II) or (II′), R¹ is identical to R². In some embodiments of Formula (II) or (II′), R¹ and R² are both hydrogen. In some embodiments of Formula (II) or (II′), neither R¹ nor R² are methyl. In some embodiments of Formula (II) or (II′), neither R¹ nor R² are substituted or unsubstituted phenyl. In some embodiments of Formula (II) or (II′), the ring formed by R¹ and R² is fused to a substituted or unsubstituted C₆ aryl ring. In some embodiments of Formula (II) or (II′), the ring formed by R¹ and R² is fused to a substituted or unsubstituted 5 or 6-membered heteroaryl ring. In some embodiments of Formula (II) or (II′) the ring formed by R¹ and R² is fused to a substituted or unsubstituted 4 to 7-membered heterocyclic ring. In some embodiments of Formula (II) or (II′), the ring formed by R¹ and R² is fused to a substituted or unsubstituted 4 to 7-membered carbocyclic ring. In some embodiments of Formula (II) or (II′), the ring formed by R¹ and R² comprises a substituted or unsubstituted bridged ring system in which at least one ring is a 4 to 8-membered heterocycle. In some embodiments of Formula (II) or (II′), the ring formed by R¹ and R² comprises a substituted or unsubstituted bridged ring system in which at least one ring is a 4 to 8-membered carbocycle. In some embodiments of Formula (II) or (II′), the ring formed by R¹ and R² comprises a substituted or unsubstituted bridged ring system in which both rings are 4 to 8-membered heterocycles. In some embodiments of Formula (II) or (II′), the ring formed by R¹ and R² comprises a substituted or unsubstituted bridged ring system in which both rings are 4 to 8-membered carbocycles. In some embodiments of Formula (II) or (II′), the ring formed by R¹ and R² is not substituted or unsubstituted cyclopentyl. In some embodiments of Formula (II) or (II′), the ring formed by R¹ and R² is not substituted or unsubstituted cyclobutyl. In some embodiments of Formula (II) or (II′), the ring formed by R¹ and R² is not substituted or unsubstituted pyran. In some embodiments of Formula (II) or (II′), the ring formed by R¹ and R² is not substituted or unsubstituted tetrahydrothiophene. In some embodiments of Formula (II) or (II′), the ring formed by R¹ and R² is not substituted or unsubstituted heterocyclic. In some embodiments of Formula (II), or (II′), the ring formed by R¹ and R² is not a substituted or unsubstituted carbocycle.

In some embodiments of Formula (II) or (II′), the ring formed by R¹ and R² comprises a substituted or unsubstituted spirocyclic ring system in which at least one ring is a 4 to 7-membered heterocycle. In some embodiments of Formula (II) or (II′), the ring formed by R¹ and R² comprises a substituted or unsubstituted spirocyclic ring system in which at least one ring is a 3 to 7-membered carbocycle. In some embodiments of Formula (II) or (II′), the ring formed by R¹ and R² comprises a substituted or unsubstituted spirocyclic ring system in which both rings are 4 to 7-membered heterocycles. In some embodiments of Formula (II) or (II′), the ring formed by R¹ and R² comprises a substituted or unsubstituted spirocyclic ring system in which both rings are 3 to 7-membered carbocycles.

In some embodiments of Formula (I), (I′), (II), or (II′), R¹ or R² is a carbon-linked substituted or unsubstituted 5-10 membered heteroaryl ring that includes, but is not limited to, one of the following: 1,6-dihydropyrrolo[2,3-b]pyrrole, 1,5-dihydropyrrolo[3,4-b]pyrrole, 1,6-dihydropyrrolo[2,3-c]pyrazole, 2,4-dihydropyrrolo[3,2-c]pyrazole, 3,4-dihydropyrrolo[2,3-d]imidazole, 1,5-dihydropyrrolo[3,4-d]imidazole, 1,4-dihydropyrrolo[2,3-d]imidazole, 1,6-dihydropyrazolo[3,4-c]pyrazole, 1,4-dihydropyrazolo[4,3-c]pyrazole, 1,5-dihydropyrazolo[3,4-c]pyrazole, 1,5-dihydropyrazolo[4,3-c]pyrazole, 1,6-dihydroimidazo[4,5-c]pyrazole, 1,4-dihydroimidazo[4,5-c]pyrazole, 2,6-dihydroimidazo[4,5-c]pyrazole, 2,4-dihydroimidazo[4,5-c]pyrazole, 3,4-dihydropyrrolo[2,3-d][1,2,3]triazole, 2,4-dihydropyrrolo[2,3-d][1,2,3]triazole, 1,4-dihydropyrrolo[2,3-d][1,2,3]triazole, 1,5-dihydropyrrolo[3,4-d][1,2,3]triazole, 3,4-dihydropyrazolo[3,4-d][1,2,3]triazole, 1,4-dihydropyrazolo[3,4-d][1,2,3]triazole, 1,6-dihydroimidazo[4,5-d][1,2,3]triazole, 1,4-dihydroimidazo[4,5-d][1,2,3]triazole, furo[2,3-b]furan, furo[3,4-b]furan, thieno[2,3-b]furan, thieno[3,2-b]furan, furo[3,2-b]furan, thieno[3,4-b]furan, thieno[2,3-b]thiophene, thieno[3,2-b]thiophene, thieno[3,4-b]thiophene, 6H-furo[2,3-b]pyrrole, 1H-furo[3,4-b]pyrrole, 6H-pyrrolo[3,2-d]isoxazole, 4H-pyrrolo[3,2-c]isoxazole, 4H-pyrrolo[3,2-d]oxazole, 5H-pyrrolo[3,4-d]oxazole, 4H-pyrrolo[2,3-d]oxazole, 6H-pyrazolo[4,3-d]isoxazole, 4H-pyrazolo[3,4-d]isoxazole, 5H-pyrazolo[4,3-d]isoxazole, 5H-pyrazolo[3,4-d]isoxazole, 1H-pyrazolo[4,3-d]oxazole, 1H-pyrazolo[3,4-d]oxazole, 2H-pyrazolo[4,3-d]oxazole, 2H-pyrazolo[3,4-d]oxazole, 3H-furo[2,3-d][1,2,3]triazole, 2H-furo[2,3-d][1,2,3]triazole, 1H-furo[2,3-d][1,2,3]triazole, 1H-furo[3,4-d][1,2,3]triazole, 3H-isoxazolo[4,5-d][1,2,3]triazole, 1H-isoxazolo[4,5-d][1,2,3]triazole, 3H-oxazolo[4,5-d][1,2,3]triazole, 1H-oxazolo[4,5-d][1,2,3]triazole, 6H-imidazo[4,5-d]isoxazole, 4H-imidazo[4,5-d]isoxazole, 6H-imidazo[4,5-d]oxazole, 4H-imidazo[4,5-d]oxazole, 4H-pyrrolo[2,3-c][1,2,5]oxadiazole, 4H-pyrazolo[3,4-c][1,2,5]oxadiazole, 4H-imidazo[4,5-c][1,2,5]oxadiazole, furo[3,2-d]isoxazole, furo[3,2-c]isoxazole, furo[3,2-d]oxazole, furo[3,4-d]oxazole, furo[2,3-d]oxazole, isoxazolo[4,5-d]isoxazole, isoxazolo[5,4-d]isoxazole, isoxazolo[5,4-c]isoxazole, isoxazolo[4,5-c]isoxazole, oxazolo[4,5-d]isoxazole, oxazolo[5,4-d]isoxazole, oxazolo[5,4-c]isoxazole, oxazolo[4,5-c]isoxazole, furo[2,3-c][1,2,5]oxadiazole, isoxazolo[4,5-c][1,2,5]oxadiazole, oxazolo[4,5-c][1,2,5]oxadiazole, oxazolo[4,5-d]isoxazole, oxazolo[4,5-d]oxazole, oxazolo[5,4-d]oxazole, 6H-thieno[2,3-b]pyrrole, 1H-thieno[3,4-b]pyrrole, 6H-pyrrolo[3,2-d]isothiazole, 4H-pyrrolo[3,2-c]isothiazole, 4H-pyrrolo[3,2-d]thiazole, 5H-pyrrolo[3,4-d]thiazole, 4H-pyrrolo[2,3-d]thiazole, 6H-pyrazolo[4,3-d]isothiazole, 4H-pyrazolo[3,4-d]isothiazole, 5H-pyrazolo[4,3-d]isothiazole, 5H-pyrazolo[3,4-d]isothiazole, 1H-pyrazolo[4,3-d]thiazole, 1H-pyrazolo[3,4-d]thiazole, 2H-pyrazolo[4,3-d]thiazole, 2H-pyrazolo[3,4-d]thiazole, 3H-thieno[2,3-d][1,2,3]triazole, 2H-thieno[2,3-d][1,2,3]triazole, 1H-thieno[2,3-d][1,2,3]triazole, 1H-thieno[3,4-d][1,2,3]triazole, 3H-isothiazolo[4,5-d][1,2,3]triazole, 1H-isothiazolo[4,5-d][1,2,3]triazole, 3H-thiazolo[4,5-d][1,2,3]triazole, 1H-thiazolo[4,5-d][1,2,3]triazole, 6H-imidazo[4,5-d]isothiazole, 4H-imidazo[4,5-d]isothiazole, 6H-imidazo[4,5-d]thiazole, 4H-imidazo[4,5-d]thiazole, 4H-pyrrolo[2,3-c][1,2,5]thiadiazole, 4H-pyrazolo[3,4-c][1,2,5]thiadiazole, 4H-imidazo[4,5-c][1,2,5]thiadiazole, thieno[3,2-d]isothiazole, thieno[3,2-c]isothiazole, thieno[3,2-d]thiazole, thieno[3,4-d]thiazole, thieno[2,3-d]thiazole, isothiazolo[4,5-d]isothiazole, isothiazolo[5,4-d]isothiazole, isothiazolo[5,4-c]isothiazole, isothiazolo[4,5-c]isothiazole, thiazolo[4,5-d]isothiazole, thiazolo[5,4-d]isothiazole, thiazolo[5,4-c]isothiazole, thiazolo[4,5-c]isothiazole, thieno[2,3-c][1,2,5]thiadiazole, isothiazolo[4,5-c][1,2,5]thiadiazole, thiazolo[4,5-c][1,2,5]thiadiazole, thiazolo[4,5-d]isothiazole, thiazolo[4,5-d]thiazole, thiazolo[5,4-d]thiazole, thieno[3,2-d]isoxazole, thieno[3,2-c]isoxazole, thieno[3,2-d]oxazole, thieno[3,4-d]oxazole, thieno[2,3-d]oxazole, isothiazolo[4,5-d]isoxazole, isothiazolo[5,4-d]isoxazole, isothiazolo[4,3-d]isoxazole, isothiazolo[3,4-d]isoxazole, isothiazolo[4,5-d]oxazole, isothiazolo[5,4-d]oxazole, isothiazolo[4,3-d]oxazole, isothiazolo[3,4-d]oxazole, thieno[2,3-c][1,2,5]oxadiazole, isothiazolo[4,5-c][1,2,5]oxadiazole, thiazolo[4,5-c][1,2,5]oxadiazole, thiazolo[4,5-d]isoxazole, thiazolo[4,5-d]oxazole, thiazolo[5,4-d]oxazole, furo[3,2-d]isothiazole, furo[3,2-c]isothiazole, furo[3,2-d]thiazole, furo[3,4-d]thiazole, furo[2,3-d]thiazole, thiazolo[4,5-d]isoxazole, thiazolo[5,4-d]isoxazole, isothiazolo[5,4-c]isoxazole, isothiazolo[4,5-c]isoxazole, thiazolo[5,4-c]isoxazole, thiazolo[4,5-c]isoxazole, isothiazolo[4,5-d]oxazole, thiazolo[4,5-d]oxazole, thiazolo[5,4-d]oxazole, furo[2,3-c][1,2,5]thiadiazole, isoxazolo[4,5-c][1,2,5]thiadiazole, oxazolo[4,5-c][1,2,5]thiadiazole, pyrrolo[2,1-b]oxazole, pyrrolo[2,1-b]thiazole, pyrrolo[2,1-b][1,3,4]oxadiazole, pyrrolo[1,2-d][1,2,4]oxadiazole, imidazo[2,1-b]oxazole, imidazo[5, 1-b]oxazole, pyrazolo[5, 1-b]oxazole, pyrrolo[2,1-b][1,3,4]thiadiazole, pyrrolo[1,2-d][1,2,4]thiadiazole, imidazo[2,1-b]thiazole, imidazo[5,1-b]thiazole, pyrazolo[5,1-b]thiazole, imidazo[2,1-b][1,3,4]oxadiazole, imidazo[1,5-d][1,2,4]oxadiazole, pyrazolo[1,5-d][1,2,4]oxadiazole, imidazo[2,1-b][1,3,4]thiadiazole, imidazo[1,5-d][1,2,4]thiadiazole, pyrazolo[1,5-d][1,2,4]thiadiazole, 1H-pyrrole, 1H-pyrazole, 2H-1,2,3-triazole, 2H-tetrazole, 1H-1,2,4-triazole, 1H-imidazole, furan, thiophene, isoxazole, 1,2,4-oxadiazole, oxazole, 1,2,5-oxadiazole, 1,3,4-oxadiazole, isothiazole, 1,2,4-thiadiazole, thiazole, 1,2,5-thiadiazole, 1,3,4-thiadiazole, pyridine, pyridazine, pyrimidine, pyrazine, 1,2,3-triazine, 1,3,5-triazine, 1,2,4-triazine, 1,2,4,5-tetrazine, 1H-indole, 2H-isoindole, 1H-pyrrolo[2,3-b]pyridine, 6H-pyrrolo[3,4-b]pyridine, 1H-pyrrolo[3,2-b]pyridine, 1H-pyrrolo[2,3-c]pyridine, 2H-pyrrolo[3,4-c]pyridine, 1H-pyrrolo[3,2-c]pyridine, 1H-indazole, 1H-benzo[d]imidazole, 2H-indazole, pyrazolo[1,5-a]pyridine, pyrrolo[1,2-c]pyrimidine, pyrrolo[1,2-b]pyridazine, imidazo[1,5-a]pyridine, imidazo[1,2-a]pyridine, pyrrolo[1,2-a]pyrimidine, pyrrolo[1,2-a]pyrazine, 1H-pyrazolo[3,4-b]pyridine, 2H-pyrazolo[3,4-b]pyridine, 1H-pyrazolo[4,3-b]pyridine, 2H-pyrazolo[4,3-b]pyridine, 3H-imidazo[4,5-b]pyridine, 2H-pyrazolo[4,3-c]pyridine, 1H-pyrazolo[4,3-c]pyridine, 2H-pyrazolo[3,4-c]pyridine, 1H-pyrazolo[3,4-c]pyridine, 5H-pyrrolo[2,3-b]pyrazine, 7H-pyrrolo[2,3-c]pyridazine, 5H-pyrrolo[3,2-c]pyridazine, 1H-imidazo[4,5-c]pyridine, 6H-pyrrolo[3,4-b]pyrazine, 7H-pyrrolo[2,3-d]pyrimidine, 5H-pyrrolo[3,2-d]pyrimidine, 1H-pyrrolo[2,3-d]pyridazine, 6H-pyrrolo[3,4-c]pyridazine, 6H-pyrrolo[3,4-d]pyrimidine, [1,2,3]triazolo[1,5-a]pyridine, [1,2,4]triazolo[1,5-a]pyridine, pyrazolo[1,5-a]pyrimidine, pyrazolo[1,5-a]pyrazine, pyrazolo[1,5-c]pyrimidine, pyrazolo[1,5-b]pyridazine, [1,2,4]triazolo[4,3-a]pyridine, imidazo[1,5-a]pyrimidine, imidazo[1,5-a]pyrazine, imidazo[1,5-c]pyrimidine, imidazo[1,5-b]pyridazine, imidazo[1,2-a]pyrimidine, imidazo[1,2-c]pyrimidine, imidazo[1,2-b]pyridazine, imidazo[1,2-a]pyrazine, pyrrolo[2,1-c][1,2,4]triazine, pyrrolo[1,2-a][1,3,5]triazine, pyrrolo[1,2-b][1,2,4]triazine, pyrrolo[1,2-d][1,2,4]triazine, pyrrolo[2,1-f][1,2,4]triazine, 1H-pyrazolo[3,4-b]pyrazine, 1H-pyrazolo[3,4-d]pyrimidine, 1H-pyrazolo[3,4-c]pyridazine, 1H-[1,2,3]triazolo[4,5-b]pyridine, 1H-[1,2,3]triazolo[4,5-c]pyridine, 3H-[1,2,3]triazolo[4,5-c]pyridine, 1H-pyrazolo[4,3-d]pyrimidine, 3H-[1,2,3]triazolo[4,5-b]pyridine, 2H-[1,2,3]triazolo[4,5-b]pyridine, 2H-[1,2,3]triazolo[4,5-c]pyridine, 9H-purine, 7H-imidazo[4,5-c]pyridazine, 7H-purine, 1H-pyrazolo[3,4-d]pyridazine, 1H-pyrazolo[4,3-c]pyridazine, 1H-imidazo[4,5-d]pyridazine, 5H-imidazo[4,5-c]pyridazine, 1H-imidazo[4,5-b]pyrazine, pyrazolo[5,1-c][1,2,4]triazine, pyrazolo[1,5-a][1,3,5]triazine, pyrazolo[1,5-b][1,2,4]triazine, pyrazolo[1,5-d][1,2,4]triazine, pyrazolo[5,1-f][1,2,4]triazine, pyrazolo[1,5-c][1,2,3]triazine, tetrazolo[1,5-a]pyridine, imidazo[2,1-c][1,2,4]triazine, [1,2,3]triazolo[5,1-c][1,2,4]triazine, [1,2,4]triazolo[5,1-c][1,2,4]triazine, imidazo[5,1-c][1,2,4]triazine, imidazo[1,5-a][1,3,5]triazine, imidazo[1,5-b][1,2,4]triazine, imidazo[1,5-d][1,2,4]triazine, imidazo[5, 1-f][1,2,4]triazine, imidazo[1,5-c][1,2,3]triazine, imidazo[1,2-a][1,3,5]triazine, imidazo[1,2-b][1,2,4]triazine, imidazo[1,2-d][1,2,4]triazine, imidazo[2,1-f][1,2,4]triazine, imidazo[1,2-c][1,2,3]triazine, [1,2,3]triazolo[1,5-a][1,3,5]triazine, [1,2,3]triazolo[1,5-b][1,2,4]triazine, [1,2,3]triazolo[1,5-d][1,2,4]triazine, [1,2,3]triazolo[5,1-f][1,2,4]triazine, [1,2,3]triazolo[1,5-c][1,2,3]triazine, [1,2,4]triazolo[1,5-a][1,3,5]triazine, [1,2,4]triazolo[1,5-b][1,2,4]triazine, [1,2,4]triazolo[1,5-d][1,2,4]triazine, [1,2,4]triazolo[5,1-f][1,2,4]triazine, [1,2,4]triazolo[1,5-c][1,2,3]triazine, [1,2,4]triazolo[4,3-a]pyrimidine, [1,2,4]triazolo[4,3-a]pyrazine, [1,2,4]triazolo[4,3-c]pyrimidine, [1,2,4]triazolo[4,3-b]pyridazine, benzo[b]thiophene, benzofuran, benzo[c]thiophene, isobenzofuran, furo[2,3-b]pyridine, furo[3,4-b]pyridine, furo[3,2-b]pyridine, furo[2,3-c]pyridine, furo[3,4-c]pyridine, furo[3,2-c]pyridine, benzo[d]isoxazole, benzo[d]oxazole, benzo[c]isoxazole, isoxazolo[5,4-b]pyridine, isoxazolo[3,4-b]pyridine, isoxazolo[4,5-b]pyridine, isoxazolo[4,3-b]pyridine, oxazolo[5,4-b]pyridine, oxazolo[4,5-b]pyridine, isoxazolo[4,3-c]pyridine, isoxazolo[4,5-c]pyridine, isoxazolo[3,4-c]pyridine, isoxazolo[5,4-c]pyridine, furo[2,3-b]pyrazine, furo[2,3-c]pyridazine, furo[3,2-c]pyridazine, oxazolo[5,4-c]pyridine, oxazolo[4,5-c]pyridine, furo[3,4-b]pyrazine, furo[2,3-d]pyrimidine, furo[2,3-d]pyridazine, furo[3,4-c]pyridazine, furo[3,4-d]pyrimidine, furo[3,2-d]pyrimidine, isoxazolo[4,5-b]pyrazine, isoxazolo[5,4-d]pyrimidine, isoxazolo[5,4-c]pyridazine, isoxazolo[4,5-d]pyrimidine, isoxazolo[4,5-d]pyridazine, isoxazolo[4,5-c]pyridazine, [1,2,3]oxadiazolo[4,5-b]pyridine, [1,2,3]oxadiazolo[4,5-c]pyridine, [1,2,3]oxadiazolo[5,4-c]pyridine, [1,2,3]oxadiazolo[5,4-b]pyridine, [1,2,5]oxadiazolo[3,4-b]pyridine, [1,2,5]oxadiazolo[3,4-c]pyridine, oxazolo[5,4-d]pyrimidine, oxazolo[5,4-c]pyridazine, oxazolo[4,5-d]pyrimidine, oxazolo[4,5-d]pyridazine, oxazolo[4,5-c]pyridazine, oxazolo[4,5-b]pyrazine, thieno[2,3-b]pyridine, thieno[3,4-b]pyridine, thieno[3,2-b]pyridine, thieno[2,3-c]pyridine, thieno[3,4-c]pyridine, thieno[3,2-c]pyridine, benzo[d]isothiazole, benzo[d]thiazole, benzo[c]isothiazole, isothiazolo[5,4-b]pyridine, isothiazolo[3,4-b]pyridine, isothiazolo[4,5-b]pyridine, isothiazolo[4,3-b]pyridine, thiazolo[5,4-b]pyridine, thiazolo[4,5-b]pyridine, isothiazolo[4,3-c]pyridine, isothiazolo[4,5-c]pyridine, isothiazolo[3,4-c]pyridine, isothiazolo[5,4-c]pyridine, thieno[2,3-b]pyrazine, thieno[2,3-c]pyridazine, thieno[3,2-c]pyridazine, thiazolo[5,4-c]pyridine, thiazolo[4,5-c]pyridine, thieno[3,4-b]pyrazine, thieno[3,2-d]pyrimidine, thieno[2,3-d]pyridazine, thieno[2,3-d]pyrimidine, thieno[3,4-c]pyridazine, thieno[3,4-d]pyrimidine, isothiazolo[4,5-b]pyrazine, isothiazolo[5,4-d]pyrimidine, isothiazolo[5,4-c]pyridazine, [1,2,3]thiadiazolo[4,5-b]pyridine, [1,2,3]thiadiazolo[4,5-c]pyridine, [1,2,3]thiadiazolo[5,4-c]pyridine, isothiazolo[4,5-d]pyrimidine, [1,2,3]thiadiazolo[5,4-b]pyridine, [1,2,5]thiadiazolo[3,4-b]pyridine, [1,2,5]thiadiazolo[3,4-c]pyridine, thiazolo[5,4-d]pyrimidine, thiazolo[5,4-c]pyridazine, thiazolo[4,5-d]pyrimidine, isothiazolo[4,5-d]pyridazine, isothiazolo[4,5-c]pyridazine, thiazolo[4,5-d]pyridazine, thiazolo[4,5-c]pyridazine, thiazolo[4,5-b]pyrazine, thieno[3,4-d]pyridazine, 1H-1,2,3-triazole, 4H-1,2,4-triazole, 1H-tetrazole, indolizine, quinoline, isoquinoline, cinnoline, phthalazine, quinazoline, quinoxaline, 1,5-naphthyridine, 1,6-naphthyridine, 1,7-naphthyridine, 1,8-naphthyridine, 2,6-naphthyridine, pyrido[2,3-c]pyridazine, pyrido[2,3-d]pyridazine, pyrido[2,3-d]pyrimidine, pyrido[2,3-b]pyrazine, pyrido[3,4-c]pyridazine, pyrido[4,3-c]pyridazine, pyrido[3,2-c]pyridazine, pyrido[3,4-d]pyridazine, pyrido[3,4-d]pyrimidine, pyrido[4,3-d]pyrimidine, pyrido[3,2-d]pyrimidine, pyrido[3,4-b]pyrazine, benzo[d][1,2,3]triazine, benzo[e][1,2,4]triazine, pyrido[3,2-e][1,2,4]triazine, pyrazino[2,3-d]pyridazine, pyrimido[4,5-d]pyrimidine, pyrazino[2,3-b]pyrazine, pyrido[4,3-e][1,2,4]triazine, pyrido[3,4-e][1,2,4]triazine, pyrido[2,3-e][1,2,4]triazine, pyrimido[4,5-d]pyridazine, pteridine, pyrido[2,3-d][1,2,3]triazine, pyrido[3,4-d][1,2,3]triazine, pyrido[4,3-d][1,2,3]triazine, pyrido[3,2-d][1,2,3]triazine, pyrazino[2,3-c]pyridazine, pyrimido[5,4-c]pyridazine, pyridazino[4,5-d]pyridazine, pyridazino[4,3-e][1,2,4]triazine, pyrazino[2,3-d][1,2,3]triazine, pyridazino[4,5-e][1,2,4]triazine, pyridazino[3,4-e][1,2,4]triazine, pyrazino[2,3-e][1,2,4]triazine, pyrimido[4,5-d][1,2,3]triazine, pyrimido[5,4-e][1,2,4]triazine, pyrimido[5,4-d][1,2,3]triazine, pyridazino[4,3-d][1,2,3]triazine, pyrimido[4,5-e][1,2,4]triazine, pyridazino[4,5-d][1,2,3]triazine, pyridazino[3,4-d][1,2,3]triazine, or 2,7-naphthyridine, wherein the heteroaryl ring is optionally substituted with 0, 1, 2, 3, 4, or 5 R⁵ groups as valency permits.

In some embodiments of Formula (I), (I′), (II), or (II′), R¹ or R² is a carbon-linked substituted or unsubstituted 4-10 membered heterocyclyl ring that includes, but is not limited to, one of the following: oxetane, tetrahydrofuran, tetrahydro-2H-pyran, oxepane, oxocane, 2-oxabicyclo[2.1.0]pentane, 2-oxabicyclo[2.2.0]hexane, 3-oxabicyclo[3.1.0]hexane, 6-oxabicyclo[3.2.0]heptane, hexahydro-2H-cyclopenta[b]furan, 2-oxabicyclo[4.1.0]heptane, 7-oxabicyclo[4.2.0]octane, octahydrocyclopenta[b]pyran, octahydro-2H-chromene, 4-oxabicyclo[5.1.0]octane, 8-oxabicyclo[5.2.0]nonane, octahydro-2H-cyclohepta[b]furan, 4-oxabicyclo[6.1.0]nonane, 9-oxabicyclo[6.2.0]decane, 1,4-dioxane, 1,4-dioxepane, 1,5-dioxocane, 1,4-dioxocane, azetidine, pyrrolidine, piperidine, azepane, azocane, piperazine, 1,4-diazepane, 1,5-diazocane, 1,4-diazocane, thietane, tetrahydrothiophene, tetrahydro-2H-thiopyran, thiepane, thiocane, 1,4-dithiane, 1,4-dithiepane, 1,5-dithiocane, 1,4-dithiocane, morpholine, 1,4-oxazepane, 1,5-oxazocane, 1,4-oxazocane, thiomorpholine, 1,4-thiazepane, 1,5-thiazocane, 1,4-thiazocane, 1,4-oxathiane, 1,4-oxathiepane, 1,5-oxathiocane, 1,4-oxathiocane, 2-oxabicyclo[3.1.0]hexane, 3-oxabicyclo[4.1.0]heptane, 3-oxabicyclo[5.1.0]octane, 3-oxabicyclo[6.1.0]nonane, 2-oxabicyclo[5.1.0]octane, 2-oxabicyclo[6.1.0]nonane, 2,5-dioxabicyclo[5.1.0]octane, 2,6-dioxabicyclo[5.1.0]octane, 2,5-dioxabicyclo[6.1.0]nonane, 2,6-dioxabicyclo[6.1.0]nonane, 2,7-dioxabicyclo[6.1.0]nonane, 2,5-dioxabicyclo[2.2.0]hexane, 3-oxabicyclo[3.2.0]heptane, 2-oxabicyclo[4.2.0]octane, 2-oxabicyclo[5.2.0]nonane, 2-oxabicyclo[6.2.0]decane, 2-oxabicyclo[3.2.0]heptane, 3,6-dioxabicyclo[3.2.0]heptane, 2,6-dioxabicyclo[3.2.0]heptane, 3-oxabicyclo[4.2.0]octane, 2,7-dioxabicyclo[4.2.0]octane, 3,7-dioxabicyclo[4.2.0]octane, 2,5-dioxabicyclo[4.2.0]octane, 3-oxabicyclo[5.2.0]nonane, 4-oxabicyclo[5.2.0]nonane, 8-oxabicyclo[5.2.0]nonane, 2,8-dioxabicyclo[5.2.0]nonane, 3,8-dioxabicyclo[5.2.0]nonane, 4,8-dioxabicyclo[5.2.0]nonane, 3,9-dioxabicyclo[5.2.0]nonane, 2,5-dioxabicyclo[5.2.0]nonane, 2,6-dioxabicyclo[5.2.0]nonane, 2,5,8-trioxabicyclo[5.2.0]nonane, 3-oxabicyclo[6.2.0]decane, 4-oxabicyclo[6.2.0]decane, 2,9-dioxabicyclo[6.2.0]decane, 3,9-dioxabicyclo[6.2.0]decane, 4,9-dioxabicyclo[6.2.0]decane, 3,10-dioxabicyclo[6.2.0]decane, 4,10-dioxabicyclo[6.2.0]decane, 2,7-dioxabicyclo[6.2.0]decane, 2,6-dioxabicyclo[6.2.0]decane, 2,5-dioxabicyclo[6.2.0]decane, 2,6,9-trioxabicyclo[6.2.0]decane, 2,5,9-trioxabicyclo[6.2.0]decane, 3,6,9-trioxabicyclo[6.2.0]decane, 3,6-dioxabicyclo[6.2.0]decane, hexahydro-1H-cyclopenta[c]furan, hexahydrofuro[3,4-b]furan, hexahydrofuro[3,2-b]furan, hexahydrofuro[3,4-b]furan, tetrahydro-1H,3H-furo[3,4-c]furan, octahydrocyclopenta[c]pyran, octahydrobenzofuran, octahydroisobenzofuran, hexahydro-5H-cyclopenta[b][1,4]dioxine, hexahydro-3H-furo[3,4-c]pyran, hexahydro-2H-furo[3,2-b]pyran, hexahydro-3H-furo[3,4-c]pyran, hexahydro-2H-furo[2,3-c]pyran, hexahydro-2H-furo[3,4-b]pyran, hexahydro-4H-furo[3,2-c]pyran, octahydro-1H-isochromene, octahydrobenzo[b][1,4]dioxine, octahydropyrano[3,2-b]pyran, hexahydro-2H,5H-pyrano[4,3-b]pyran, octahydropyrano[3,4-b]pyran, octahydropyrano[4,3-c]pyran, hexahydro-1H,3H-pyrano[3,4-c]pyran, hexahydro-5H-pyrano[3,4-b][1,4]dioxine, octahydro-1H-cyclohepta[c]furan, octahydro-2H-cyclopenta[b]oxepine, octahydro-1H-cyclopenta[c]oxepine, octahydro-2H-cyclopenta[d]oxepine, octahydrofuro[3,2-b]oxepine, octahydrofuro[3,4-b]oxepine, octahydrofuro[3,2-c]oxepine, hexahydro-1H,3H-furo[3,4-c]oxepine, hexahydro-2H,6H-cyclopenta[b][1,4]dioxepine, octahydrofuro[2,3-d]oxepine, octahydrofuro[3,4-d]oxepine, octahydrocyclopenta[e][1,4]dioxepine, octahydrofuro[2,3-c]oxepine, hexahydro-5H-furo[3,2-e][1,4]dioxepine, hexahydro-5H-furo[3,4-e][1,4]dioxepine, hexahydro-2H-furo[3,4-b][1,4]dioxepine, hexahydrofuro[3,4-b][1,4]dioxine, 2-thiabicyclo[2.1.0]pentane, 2-thiabicyclo[2.2.0]hexane, 3-thiabicyclo[3.1.0]hexane, 6-thiabicyclo[3.2.0]heptane, hexahydro-2H-cyclopenta[b]thiophene, 2-thiabicyclo[4.1.0]heptane, 7-thiabicyclo[4.2.0]octane, octahydrocyclopenta[b]thiopyran, octahydro-2H-thiochromene, 4-thiabicyclo[5.1.0]octane, 8-thiabicyclo[5.2.0]nonane, octahydro-2H-cyclohepta[b]thiophene, 4-thiabicyclo[6.1.0]nonane, 9-thiabicyclo[6.2.0]decane, 2-thiabicyclo[3.1.0]hexane, 3-thiabicyclo[4.1.0]heptane, 3-thiabicyclo[5.1.0]octane, 3-thiabicyclo[6.1.0]nonane, 2-thiabicyclo[5.1.0]octane, 2-thiabicyclo[6.1.0]nonane, 2-oxa-5-thiabicyclo[6.1.0]nonane, 2-oxa-6-thiabicyclo[6.1.0]nonane, 2-oxa-7-thiabicyclo[6.1.0]nonane, 2-oxa-5-thiabicyclo[2.2.0]hexane, 3-thiabicyclo[3.2.0]heptane, 2-thiabicyclo[4.2.0]octane, 2-thiabicyclo[5.2.0]nonane, 2-thiabicyclo[6.2.0]decane, 2-thiabicyclo[3.2.0]heptane, 6-oxa-3-thiabicyclo[3.2.0]heptane, 6-oxa-2-thiabicyclo[3.2.0]heptane, 3-thiabicyclo[4.2.0]octane, 2-oxa-7-thiabicyclo[4.2.0]octane, 3-oxa-7-thiabicyclo[4.2.0]octane, 2-oxa-5-thiabicyclo[4.2.0]octane, 3-thiabicyclo[5.2.0]nonane, 4-thiabicyclo[5.2.0]nonane, 8-thiabicyclo[5.2.0]nonane, 8-oxa-2-thiabicyclo[5.2.0]nonane, 8-oxa-3-thiabicyclo[5.2.0]nonane, 8-oxa-4-thiabicyclo[5.2.0]nonane, 9-oxa-3-thiabicyclo[5.2.0]nonane, 2-oxa-5-thiabicyclo[5.2.0]nonane, 2-oxa-6-thiabicyclo[5.2.0]nonane, 2,8-dioxa-5-thiabicyclo[5.2.0]nonane, 3-thiabicyclo[6.2.0]decane, 4-thiabicyclo[6.2.0]decane, 9-oxa-2-thiabicyclo[6.2.0]decane, 9-oxa-3-thiabicyclo[6.2.0]decane, 9-oxa-4-thiabicyclo[6.2.0]decane, 10-oxa-3-thiabicyclo[6.2.0]decane, 10-oxa-4-thiabicyclo[6.2.0]decane, 2-oxa-7-thiabicyclo[6.2.0]decane, 2-oxa-6-thiabicyclo[6.2.0]decane, 2-oxa-5-thiabicyclo[6.2.0]decane, 2,9-dioxa-6-thiabicyclo[6.2.0]decane, 2,9-dioxa-5-thiabicyclo[6.2.0]decane, 3,9-dioxa-6-thiabicyclo[6.2.0]decane, 3-oxa-6-thiabicyclo[6.2.0]decane, hexahydro-1H-cyclopenta[c]thiophene, hexahydrothieno[2,3-c]furan, hexahydrothieno[3,2-b]furan, hexahydrothieno[3,4-b]furan, tetrahydro-1H,3H-thieno[3,4-c]furan, octahydrocyclopenta[c]thiopyran, octahydrobenzo[b]thiophene, octahydrobenzo[c]thiophene, hexahydro-5H-cyclopenta[b][1,4]oxathiine, hexahydro-3H-thieno[3,4-c]pyran, hexahydro-2H-thieno[3,2-b]pyran, hexahydro-2H-thieno[2,3-c]pyran, hexahydro-2H-thieno[3,4-b]pyran, hexahydro-4H-thieno[3,2-c]pyran, octahydro-1H-isothiochromene, octahydrobenzo[b][1,4]oxathiine, octahydrothiopyrano[3,2-b]pyran, octahydrothiopyrano[2,3-c]pyran, octahydrothiopyrano[4,3-c]pyran, hexahydro-1H,3H-thiopyrano[3,4-c]pyran, hexahydro-5H-thiopyrano[3,4-b][1,4]dioxine, octahydro-1H-cyclohepta[c]thiophene, octahydro-2H-cyclopenta[b]thiepine, octahydro-1H-cyclopenta[c]thiepine, octahydro-2H-cyclopenta[d]thiepine, octahydrothiepino[3,2-b]furan, octahydrothiepino[2,3-c]furan, octahydrothiepino[4,3-b]furan, hexahydro-1H,3H-thiepino[3,4-c]furan, hexahydro-2H,6H-cyclopenta[b][1,4]oxathiepine, octahydrothiepino[4,5-b]furan, octahydrothiepino[4,5-c]furan, octahydrocyclopenta[e][1,4]oxathiepine, octahydrothiepino[3,4-b]furan, hexahydro-5H-furo[2,3-f][1,4]oxathiepine, hexahydro-5H-furo[3,4-f][1,4]oxathiepine, hexahydro-2H-thieno[3,4-b][1,4]dioxepine, hexahydrothieno[3,4-b][1,4]dioxine, 2-azabicyclo[2.1.0]pentane, 2-azabicyclo[2.2.0]hexane, 3-azabicyclo[3.1.0]hexane, 6-azabicyclo[3.2.0]heptane, octahydrocyclopenta[b]pyrrole, 2-azabicyclo[4.1.0]heptane, 7-azabicyclo[4.2.0]octane, octahydro-1H-cyclopenta[b]pyridine, decahydroquinoline, 4-azabicyclo[5.1.0]octane, 8-azabicyclo[5.2.0]nonane, decahydrocyclohepta[b]pyrrole, 4-azabicyclo[6.1.0]nonane, 9-azabicyclo[6.2.0]decane, 2-azabicyclo[3.1.0]hexane, 3-azabicyclo[4.1.0]heptane, 3-azabicyclo[5.1.0]octane, 3-azabicyclo[6.1.0]nonane, 2-azabicyclo[5.1.0]octane, 2-azabicyclo[6.1.0]nonane, 2,5-diazabicyclo[5.1.0]octane, 2,6-diazabicyclo[5.1.0]octane, 2,5-diazabicyclo[6.1.0]nonane, 2,6-diazabicyclo[6.1.0]nonane, 2,7-diazabicyclo[6.1.0]nonane, 2,5-diazabicyclo[2.2.0]hexane, 3-azabicyclo[3.2.0]heptane, 2-azabicyclo[4.2.0]octane, 2-azabicyclo[5.2.0]nonane, 2-azabicyclo[6.2.0]decane, 2-azabicyclo[3.2.0]heptane, 3,6-diazabicyclo[3.2.0]heptane, 2,6-diazabicyclo[3.2.0]heptane, 3-azabicyclo[4.2.0]octane, 2,7-diazabicyclo[4.2.0]octane, 3,7-diazabicyclo[4.2.0]octane, 2,5-diazabicyclo[4.2.0]octane, 3-azabicyclo[5.2.0]nonane, 4-azabicyclo[5.2.0]nonane, 8-azabicyclo[5.2.0]nonane, 2,8-diazabicyclo[5.2.0]nonane, 3,8-diazabicyclo[5.2.0]nonane, 4,8-diazabicyclo[5.2.0]nonane, 3,9-diazabicyclo[5.2.0]nonane, 2,5-diazabicyclo[5.2.0]nonane, 2,6-diazabicyclo[5.2.0]nonane, 2,5,8-triazabicyclo[5.2.0]nonane, 3-azabicyclo[6.2.0]decane, 4-azabicyclo[6.2.0]decane, 2,9-diazabicyclo[6.2.0]decane, 3,9-diazabicyclo[6.2.0]decane, 4,9-diazabicyclo[6.2.0]decane, 3,10-diazabicyclo[6.2.0]decane, 4,10-diazabicyclo[6.2.0]decane, 2,7-diazabicyclo[6.2.0]decane, 2,6-diazabicyclo[6.2.0]decane, 2,5-diazabicyclo[6.2.0]decane, 2,6,9-triazabicyclo[6.2.0]decane, 2,5,9-triazabicyclo[6.2.0]decane, 3,6,9-triazabicyclo[6.2.0]decane, 3,6-diazabicyclo[6.2.0]decane, octahydrocyclopenta[c]pyrrole, octahydropyrrolo[3,4-b]pyrrole, octahydropyrrolo[3,2-b]pyrrole, octahydropyrrolo[3,4-b]pyrrole, octahydropyrrolo[3,4-c]pyrrole, octahydro-1H-cyclopenta[c]pyridine, octahydro-1H-indole, octahydro-1H-isoindole, octahydro-1H-cyclopenta[b]pyrazine, octahydro-1H-pyrrolo[3,4-c]pyridine, octahydro-1H-pyrrolo[3,2-b]pyridine, octahydro-1H-pyrrolo[2,3-c]pyridine, octahydro-1H-pyrrolo[3,4-b]pyridine, octahydro-1H-pyrrolo[3,2-c]pyridine, decahydroisoquinoline, decahydroquinoxaline, decahydro-1,5-naphthyridine, decahydro-1,6-naphthyridine, decahydro-1,7-naphthyridine, decahydro-2,6-naphthyridine, decahydro-2,7-naphthyridine, decahydropyrido[3,4-b]pyrazine, decahydrocyclohepta[c]pyrrole, decahydrocyclopenta[b]azepine, decahydrocyclopenta[c]azepine, decahydrocyclopenta[d]azepine, decahydropyrrolo[3,2-b]azepine, decahydropyrrolo[3,4-b]azepine, decahydropyrrolo[3,2-c]azepine, decahydropyrrolo[3,4-c]azepine, decahydrocyclopenta[b][1,4]diazepine, decahydropyrrolo[2,3-d]azepine, decahydropyrrolo[3,4-d]azepine, decahydrocyclopenta[e][1,4]diazepine, decahydropyrrolo[2,3-c]azepine, decahydropyrrolo[3,2-e][1,4]diazepine, decahydropyrrolo[3,4-e][1,4]diazepine, decahydropyrrolo[3,4-b][1,4]diazepine, octahydro-1H-pyrrolo[3,4-b]pyrazine, 1-azabicyclo[2.2.0]hexane, 1-azabicyclo[3.2.0]heptane, hexahydro-1H-pyrrolizine, 1-azabicyclo[4.2.0]octane, octahydroindolizine, octahydro-2H-quinolizine, 1-azabicyclo[5.2.0]nonane, octahydro-1H-pyrrolo[1,2-a]azepine, 1-azabicyclo[6.2.0]decane, 1,4-diazabicyclo[4.2.0]octane, 1,5-diazabicyclo[5.2.0]nonane, 1,6-diazabicyclo[6.2.0]decane, 1,4-diazabicyclo[5.2.0]nonane, 1,5-diazabicyclo[6.2.0]decane, 1,4-diazabicyclo[6.2.0]decane, octahydropyrrolo[1,2-a]pyrazine, octahydro-2H-pyrido[1,2-a]pyrazine, octahydro-1H-pyrrolo[1,2-d][1,4]diazepine, octahydro-2H-pyrazino[1,2-a]pyrazine, octahydro-1H-pyrrolo[1,2-a][1,4]diazepine, 5-oxa-2-thiabicyclo[5.1.0]octane, 2-oxa-6-thiabicyclo[5.1.0]octane, 5-oxa-2-thiabicyclo[6.1.0]nonane, 6-oxa-2-thiabicyclo[6.1.0]nonane, 3-oxa-6-thiabicyclo[3.2.0]heptane, 2-oxa-6-thiabicyclo[3.2.0]heptane, 7-oxa-2-thiabicyclo[4.2.0]octane, 7-oxa-3-thiabicyclo[4.2.0]octane, 2-oxa-8-thiabicyclo[5.2.0]nonane, 3-oxa-8-thiabicyclo[5.2.0]nonane, 4-oxa-8-thiabicyclo[5.2.0]nonane, 3-oxa-9-thiabicyclo[5.2.0]nonane, 5-oxa-2-thiabicyclo[5.2.0]nonane, 5,8-dioxa-2-thiabicyclo[5.2.0]nonane, 2,5-dioxa-8-thiabicyclo[5.2.0]nonane, 2-oxa-9-thiabicyclo[6.2.0]decane, 3-oxa-9-thiabicyclo[6.2.0]decane, 4-oxa-9-thiabicyclo[6.2.0]decane, 3-oxa-10-thiabicyclo[6.2.0]decane, 4-oxa-10-thiabicyclo[6.2.0]decane, 6-oxa-2-thiabicyclo[6.2.0]decane, 5-oxa-2-thiabicyclo[6.2.0]decane, 6,9-dioxa-3-thiabicyclo[6.2.0]decane, 6,9-dioxa-2-thiabicyclo[6.2.0]decane, 5,9-dioxa-2-thiabicyclo[6.2.0]decane, 2,6-dioxa-9-thiabicyclo[6.2.0]decane, 2,5-dioxa-9-thiabicyclo[6.2.0]decane, 3,6-dioxa-9-thiabicyclo[6.2.0]decane, hexahydrofuro[3,4-b][1,4]oxathiine, hexahydro-3H-thiopyrano[3,4-c]furan, hexahydro-2H-thiopyrano[3,4-b]furan, hexahydro-2H-thiopyrano[3,2-b]furan, hexahydro-2H-thiopyrano[2,3-c]furan, hexahydro-4H-thiopyrano[4,3-b]furan, octahydrobenzo[b][1,4]oxathiine, octahydrothiopyrano[3,4-b]pyran, hexahydro-5H-pyrano[3,4-b][1,4]oxathiine, hexahydro-5H-pyrano[4,3-b][1,4]oxathiine, octahydrothieno[3,2-b]oxepine, octahydrothieno[3,4-b]oxepine, octahydrothieno[3,2-c]oxepine, hexahydro-1H,3H-thieno[3,4-c]oxepine, octahydrothieno[2,3-d]oxepine, octahydrothieno[3,4-d]oxepine, octahydrothieno[2,3-c]oxepine, octahydrocyclopenta[f][1,4]oxathiepine, hexahydro-5H-furo[3,2-e][1,4]oxathiepine, hexahydro-5H-furo[3,4-e][1,4]oxathiepine, hexahydro-2H-furo[3,4-b][1,4]oxathiepine, hexahydro-5H-thieno[3,2-e][1,4]dioxepine, hexahydro-5H-thieno[3,4-e][1,4]dioxepine, 2-oxa-5-azabicyclo[5.1.0]octane, 2-oxa-6-azabicyclo[5.1.0]octane, 2-oxa-5-azabicyclo[6.1.0]nonane, 2-oxa-6-azabicyclo[6.1.0]nonane, 2-oxa-7-azabicyclo[6.1.0]nonane, 2-oxa-5-azabicyclo[2.2.0]hexane, 3-oxa-6-azabicyclo[3.2.0]heptane, 2-oxa-6-azabicyclo[3.2.0]heptane, 2-oxa-7-azabicyclo[4.2.0]octane, 3-oxa-7-azabicyclo[4.2.0]octane, 2-oxa-5-azabicyclo[4.2.0]octane, 8-oxa-2-azabicyclo[5.2.0]nonane, 8-oxa-3-azabicyclo[5.2.0]nonane, 8-oxa-4-azabicyclo[5.2.0]nonane, 9-oxa-3-azabicyclo[5.2.0]nonane, 2-oxa-5-azabicyclo[5.2.0]nonane, 2-oxa-6-azabicyclo[5.2.0]nonane, 2,8-dioxa-5-azabicyclo[5.2.0]nonane, 9-oxa-2-azabicyclo[6.2.0]decane, 9-oxa-3-azabicyclo[6.2.0]decane, 9-oxa-4-azabicyclo[6.2.0]decane, 10-oxa-3-azabicyclo[6.2.0]decane, 10-oxa-4-azabicyclo[6.2.0]decane, 2-oxa-6-azabicyclo[6.2.0]decane, 2-oxa-5-azabicyclo[6.2.0]decane, 9-oxa-2,6-diazabicyclo[6.2.0]decane, 9-oxa-2,5-diazabicyclo[6.2.0]decane, 9-oxa-3,6-diazabicyclo[6.2.0]decane, 3-oxa-6-azabicyclo[6.2.0]decane, hexahydro-1H-furo[3,4-b]pyrrole, hexahydro-2H-furo[3,2-b]pyrrole, hexahydro-2H-furo[2,3-c]pyrrole, hexahydro-1H-furo[3,4-c]pyrrole, octahydrocyclopenta[b][1,4]oxazine, octahydrofuro[3,4-c]pyridine, octahydrofuro[3,2-b]pyridine, octahydrofuro[2,3-c]pyridine, octahydrofuro[3,4-b]pyridine, octahydrofuro[3,2-c]pyridine, octahydro-2H-benzo[b][1,4]oxazine, octahydro-2H-pyrano[3,2-b]pyridine, octahydro-2H-pyrano[3,2-c]pyridine, octahydro-2H-pyrano[2,3-c]pyridine, octahydro-1H-pyrano[4,3-c]pyridine, octahydro-1H-pyrano[3,4-c]pyridine, octahydro-2H-pyrano[3,4-b]pyrazine, octahydro-2H-furo[3,2-b]azepine, octahydro-1H-furo[3,4-b]azepine, octahydro-2H-furo[3,2-c]azepine, octahydro-1H-furo[3,4-c]azepine, octahydro-2H-cyclopenta[b][1,4]oxazepine, octahydro-2H-furo[2,3-d]azepine, octahydro-1H-furo[3,4-d]azepine, octahydro-2H-cyclopenta[f][1,4]oxazepine, octahydro-2H-furo[2,3-c]azepine, octahydro-1H-furo[3,2-e][1,4]diazepine, octahydro-1H-furo[3,4-e][1,4]diazepine, octahydro-1H-furo[3,4-b][1,4]diazepine, octahydropyrrolo[3,4-b][1,4]oxazine, 4-oxa-1-azabicyclo[4.2.0]octane, 5-oxa-1-azabicyclo[6.2.0]decane, 4-oxa-1-azabicyclo[5.2.0]nonane, 5-oxa-1-azabicyclo[6.2.0]decane, 4-oxa-1-azabicyclo[6.2.0]decane, hexahydro-1H-pyrrolo[2,1-c][1,4]oxazine, octahydropyrido[2,1-c][1,4]oxazine, octahydropyrrolo[1,2-d][1,4]oxazepine, octahydropyrazino[2,1-c][1,4]oxazine, hexahydro-1H,3H-pyrrolo[2,1-c][1,4]oxazepine, 5-oxa-2-azabicyclo[5.1.0]octane, 5-oxa-2-azabicyclo[6.1.0]nonane, 6-oxa-2-azabicyclo[6.1.0]nonane, 6-oxa-3-azabicyclo[3.2.0]heptane, 6-oxa-2-azabicyclo[3.2.0]heptane, 7-oxa-2-azabicyclo[4.2.0]octane, 7-oxa-3-azabicyclo[4.2.0]octane, 3-oxa-8-azabicyclo[4.2.0]octane, 8-oxa-3-azabicyclo[4.2.0]octane, 2-oxa-8-azabicyclo[5.2.0]nonane, 3-oxa-8-azabicyclo[5.2.0]nonane, 4-oxa-8-azabicyclo[5.2.0]nonane, 3-oxa-9-azabicyclo[5.2.0]nonane, 5-oxa-2-azabicyclo[5.2.0]nonane, 5,8-dioxa-2-azabicyclo[5.2.0]nonane, 2,5-dioxa-8-azabicyclo[5.2.0]nonane, 2-oxa-9-azabicyclo[6.2.0]decane, 3-oxa-9-azabicyclo[6.2.0]decane, 4-oxa-9-azabicyclo[6.2.0]decane, 3-oxa-10-azabicyclo[6.2.0]decane, 4-oxa-7-azabicyclo[6.2.0]decane, 6-oxa-2-azabicyclo[6.2.0]decane, 5-oxa-2-azabicyclo[6.2.0]decane, 2-oxa-6,9-diazabicyclo[6.2.0]decane, 2-oxa-5,9-diazabicyclo[6.2.0]decane, 3-oxa-6,9-diazabicyclo[6.2.0]decane, 8-oxa-2,5-diazabicyclo[5.2.0]nonane, 5-oxa-2,8-diazabicyclo[5.2.0]nonane, 2-oxa-5,8-diazabicyclo[5.2.0]nonane, 6-oxa-2,9-diazabicyclo[6.2.0]decane, 5-oxa-2,9-diazabicyclo[6.2.0]decane, 6-oxa-3,9-diazabicyclo[6.2.0]decane, 2,9-dioxa-6-azabicyclo[6.2.0]decane, 2,6-dioxa-9-azabicyclo[6.2.0]decane, 6,9-dioxa-2-azabicyclo[6.2.0]decane, 2,9-dioxa-5-azabicyclo[6.2.0]decane, 2,5-dioxa-9-azabicyclo[6.2.0]decane, 5,9-dioxa-2-azabicyclo[6.2.0]decane, 3,9-dioxa-6-azabicyclo[6.2.0]decane, 3,6-dioxa-9-azabicyclo[6.2.0]decane, 6,9-dioxa-3-azabicyclo[6.2.0]decane, octahydropyrano[3,4-c]pyrrole, octahydropyrano[3,4-b]pyrrole, octahydrofuro[3,4-b]pyrazine, hexahydro-5H-[1,4]dioxino[2,3-c]pyrrole, hexahydro-2H-furo[3,4-b][1,4]oxazine, octahydropyrano[3,2-b]pyrrole, octahydropyrano[2,3-c]pyrrole, octahydropyrano[4,3-b]pyrrole, octahydro-2H-pyrano[3,2-b]pyridine, octahydro-2H-pyrano[4,3-b]pyridine, octahydro-1H-pyrano[3,4-b]pyridine, octahydro-2H-pyrido[4,3-b][1,4]oxazine, octahydro-1H-pyrido[3,4-b][1,4]oxazine, octahydropyrano[3,4-b][1,4]oxazine, octahydro-[1,4]dioxino[2,3-c]pyridine, hexahydro-2H,5H-pyrano[4,3-b][1,4]oxazine, octahydro-1H-oxepino[3,2-b]pyrrole, octahydro-2H-oxepino[2,3-c]pyrrole, octahydro-1H-oxepino[4,3-b]pyrrole, octahydro-1H-oxepino[3,4-c]pyrrole, octahydro-1H-oxepino[4,5-b]pyrrole, octahydro-1H-oxepino[4,5-c]pyrrole, octahydro-1H-oxepino[3,4-b]pyrrole, octahydro-1H-cyclopenta[e][1,4]oxazepine, octahydro-2H-pyrrolo[2,3-f][1,4]oxazepine, octahydro-2H-pyrrolo[3,4-f][1,4]oxazepine, octahydrofuro[3,4-b][1,4]oxazepine, octahydro-1H-pyrrolo[3,2-e][1,4]oxazepine, octahydrofuro[2,3-f][1,4]oxazepine, octahydro-[1,4]dioxepino[6,5-b]pyrrole, octahydrofuro[3,2-e][1,4]oxazepine, octahydro-1H-pyrrolo[3,4-e][1,4]oxazepine, octahydrofuro[3,4-f][1,4]oxazepine, octahydro-[1,4]dioxepino[5,6-c]pyrrole, hexahydro-1H,5H-furo[3,4-e][1,4]oxazepine, octahydro-2H-pyrrolo[3,4-b][1,4]oxazepine, hexahydro-1H-[1,4]oxazino[3,4-c][1,4]oxazine, 2-thia-5-azabicyclo[5.1.0]octane, 2-thia-6-azabicyclo[5.1.0]octane, 2-thia-5-azabicyclo[6.1.0]nonane, 2-thia-6-azabicyclo[6.1.0]nonane, 2-thia-7-azabicyclo[6.1.0]nonane, 2-thia-5-azabicyclo[2.2.0]hexane, 3-thia-6-azabicyclo[3.2.0]heptane, 2-thia-6-azabicyclo[3.2.0]heptane, 2-thia-7-azabicyclo[4.2.0]octane, 3-thia-7-azabicyclo[4.2.0]octane, 2-thia-5-azabicyclo[4.2.0]octane, 8-thia-2-azabicyclo[5.2.0]nonane, 8-thia-3-azabicyclo[5.2.0]nonane, 8-thia-4-azabicyclo[5.2.0]nonane, 9-thia-3-azabicyclo[5.2.0]nonane, 2-thia-5-azabicyclo[5.2.0]nonane, 2-thia-6-azabicyclo[5.2.0]nonane, 9-thia-2-azabicyclo[6.2.0]decane, 9-thia-3-azabicyclo[6.2.0]decane, 9-thia-4-azabicyclo[6.2.0]decane, 10-thia-3-azabicyclo[6.2.0]decane, 10-thia-4-azabicyclo[6.2.0]decane, 2-thia-6-azabicyclo[6.2.0]decane, 2-thia-5-azabicyclo[6.2.0]decane, 9-thia-2,6-diazabicyclo[6.2.0]decane, 9-thia-2,5-diazabicyclo[6.2.0]decane, 9-thia-3,6-diazabicyclo[6.2.0]decane, 3-thia-6-azabicyclo[6.2.0]decane, hexahydro-1H-thieno[3,4-b]pyrrole, hexahydro-2H-thieno[3,2-b]pyrrole, hexahydro-2H-thieno[2,3-c]pyrrole, hexahydro-1H-thieno[3,4-c]pyrrole, octahydrocyclopenta[b][1,4]thiazine, octahydrothieno[3,4-c]pyridine, octahydrothieno[3,2-b]pyridine, octahydrothieno[2,3-c]pyridine, octahydrothieno[3,4-b]pyridine, octahydrothieno[3,2-c]pyridine, octahydro-2H-benzo[b][1,4]thiazine, octahydro-2H-thiopyrano[3,2-b]pyridine, octahydro-2H-thiopyrano[3,2-c]pyridine, octahydro-2H-thiopyrano[2,3-c]pyridine, octahydro-1H-thiopyrano[4,3-c]pyridine, octahydro-1H-thiopyrano[3,4-c]pyridine, octahydro-2H-thiopyrano[3,4-b]pyrazine, octahydro-2H-thieno[3,2-b]azepine, octahydro-1H-thieno[3,4-b]azepine, octahydro-2H-thieno[3,2-c]azepine, octahydro-1H-thieno[3,4-c]azepine, octahydro-2H-cyclopenta[b][1,4]thiazepine, octahydro-2H-thieno[2,3-d]azepine, octahydro-1 H-thieno[3,4-d]azepine, octahydro-2H-cyclopenta[f][1,4]thiazepine, octahydro-2H-thieno[2,3-c]azepine, octahydro-1H-thieno[3,2-e][1,4]diazepine, octahydro-1H-thieno[3,4-e][1,4]diazepine, octahydro-1H-thieno[3,4-b][1,4]diazepine, octahydropyrrolo[3,4-b][1,4]thiazine, 4-thia-1-azabicyclo[4.2.0]octane, 5-thia-1-azabicyclo[5.2.0]nonane, 6-thia-1-azabicyclo[6.2.0]decane, 4-thia-1-azabicyclo[5.2.0]nonane, 5-thia-1-azabicyclo[6.2.0]decane, 4-thia-1-azabicyclo[6.2.0]decane, hexahydro-1H-pyrrolo[2,1-c][1,4]thiazine, octahydropyrido[2,1-c][1,4]thiazine, octahydropyrrolo[1,2-d][1,4]thiazepine, octahydropyrazino[2,1-c][1,4]thiazine, hexahydro-1H,3H-pyrrolo[2,1-c][1,4]thiazepine, 5-thia-2-azabicyclo[5.1.0]octane, 5-thia-2-azabicyclo[6.1.0]nonane, 6-thia-2-azabicyclo[6.1.0]nonane, 6-thia-3-azabicyclo[3.2.0]heptane, 6-thia-2-azabicyclo[3.2.0]heptane, 7-thia-2-azabicyclo[4.2.0]octane, 7-thia-3-azabicyclo[4.2.0]octane, 3-thia-8-azabicyclo[4.2.0]octane, 8-thia-3-azabicyclo[4.2.0]octane, 2-thia-8-azabicyclo[5.2.0]nonane, 3-thia-8-azabicyclo[5.2.0]nonane, 4-thia-8-azabicyclo[5.2.0]nonane, 3-thia-9-azabicyclo[5.2.0]nonane, 5-thia-2-azabicyclo[5.2.0]nonane, 2-thia-9-azabicyclo[6.2.0]decane, 3-thia-9-azabicyclo[6.2.0]decane, 4-thia-9-azabicyclo[6.2.0]decane, 3-thia-10-azabicyclo[6.2.0]decane, 4-thia-10-azabicyclo[6.2.0]decane, 2-thia-7-azabicyclo[6.2.0]decane, 6-thia-2-azabicyclo[6.2.0]decane, 5-thia-2-azabicyclo[6.2.0]decane, 2-thia-6,9-diazabicyclo[6.2.0]decane, 2-thia-5,9-diazabicyclo[6.2.0]decane, 3-thia-6,9-diazabicyclo[6.2.0]decane, 8-thia-2,5-diazabicyclo[5.2.0]nonane, 5-thia-2,8-diazabicyclo[5.2.0]nonane, 2-thia-5,8-diazabicyclo[5.2.0]nonane, 6-thia-2,9-diazabicyclo[6.2.0]decane, 5-thia-2,9-diazabicyclo[6.2.0]decane, 6-thia-3,9-diazabicyclo[6.2.0]decane, octahydrothiopyrano[3,4-c]pyrrole, octahydrothiopyrano[3,4-b]pyrrole, octahydrothieno[3,4-b]pyrazine, octahydrothiopyrano[3,2-b]pyrrole, octahydrothiopyrano[2,3-c]pyrrole, octahydrothiopyrano[4,3-b]pyrrole, octahydro-2H-thiopyrano[4,3-b]pyridine, octahydro-1H-thiopyrano[3,4-b]pyridine, octahydro-2H-pyrido[4,3-b][1,4]thiazine, octahydro-1H-pyrido[3,4-b][1,4]thiazine, octahydro-1H-thiepino[3,2-b]pyrrole, octahydro-2H-thiepino[2,3-c]pyrrole, octahydro-1H-thiepino[4,3-b]pyrrole, octahydro-1H-thiepino[3,4-c]pyrrole, octahydro-1H-thiepino[4,5-b]pyrrole, octahydro-1H-thiepino[4,5-c]pyrrole, octahydro-1H-thiepino[3,4-b]pyrrole, octahydro-1H-cyclopenta[e][1,4]thiazepine, octahydro-2H-pyrrolo[2,3-f][1,4]thiazepine, octahydro-2H-pyrrolo[3,4-f][1,4]thiazepine, octahydro-1H-pyrrolo[3,2-e][1,4]thiazepine, octahydro-1H-pyrrolo[3,4-e][1,4]thiazepine, octahydro-2H-pyrrolo[3,4-b][1,4]thiazepine, 8-oxa-2-thia-5-azabicyclo[5.2.0]nonane, 8-oxa-5-thia-2-azabicyclo[5.2.0]nonane, 2-oxa-5-thia-8-azabicyclo[5.2.0]nonane, 2-oxa-9-thia-6-azabicyclo[6.2.0]decane, 6-oxa-2-thia-9-azabicyclo[6.2.0]decane, 2-oxa-9-thia-5-azabicyclo[6.2.0]decane, 5-oxa-2-thia-9-azabicyclo[6.2.0]decane, 3-oxa-9-thia-6-azabicyclo[6.2.0]decane, 6-oxa-3-thia-9-azabicyclo[6.2.0]decane, 6-oxa-9-thia-2-azabicyclo[6.2.0]decane, 5-oxa-9-thia-2-azabicyclo[6.2.0]decane, 6-oxa-9-thia-3-azabicyclo[6.2.0]decane, hexahydro-5H-[1,4]oxathiino[2,3-c]pyrrole, hexahydro-2H-furo[3,4-b][1,4]thiazine, octahydrothiopyrano[3,4-b][1,4]oxazine, octahydro-[1,4]oxathiino[2,3-c]pyridine, hexahydro-2H,5H-thiopyrano[4,3-b][1,4]oxazine, octahydrofuro[2,3-f][1,4]thiazepine, octahydrofuro[3,4-f][1,4]thiazepine, octahydro-[1,4]oxathiepino[6,7-b]pyrrole, octahydro-[1,4]oxathiepino[6,7-c]pyrrole, octahydrofuro[3,4-b][1,4]thiazepine, 2-oxa-8-thia-5-azabicyclo[5.2.0]nonane, 5-oxa-8-thia-2-azabicyclo[5.2.0]nonane, 5-oxa-2-thia-8-azabicyclo[5.2.0]nonane, 9-oxa-2-thia-6-azabicyclo[6.2.0]decane, 2-oxa-6-thia-9-azabicyclo[6.2.0]decane, 9-oxa-2-thia-5-azabicyclo[6.2.0]decane, 2-oxa-5-thia-9-azabicyclo[6.2.0]decane, 9-oxa-3-thia-6-azabicyclo[6.2.0]decane, 3-oxa-6-thia-9-azabicyclo[6.2.0]decane, 9-oxa-6-thia-2-azabicyclo[6.2.0]decane, 9-oxa-5-thia-2-azabicyclo[6.2.0]decane, 9-oxa-6-thia-3-azabicyclo[6.2.0]decane, hexahydro-2H-thieno[3,4-b][1,4]oxazine, octahydrofuro[3,2-e][1,4]thiazepine, hexahydro-1H,5H-furo[3,4-e][1,4]thiazepine, hexahydro-1H-[1,4]thiazino[3,4-c][1,4]oxazine, octahydropyrano[3,4-b][1,4]thiazine, octahydro-[1,4]oxathiino[3,2-c]pyridine, hexahydro-2H,5H-pyrano[4,3-b][1,4]thiazine, octahydrothieno[2,3-f][1,4]oxazepine, octahydrothieno[3,4-f][1,4]oxazepine, octahydro-[1,4]oxathiepino[6,5-b]pyrrole, octahydro-[1,4]oxathiepino[5,6-c]pyrrole, octahydrothieno[3,4-b][1,4]oxazepine, octahydrothieno[3,2-e][1,4]oxazepine, hexahydro-1H,5H-thieno[3,4-e][1,4]oxazepine, 5-oxaspiro[2.3]hexane, 4-oxaspiro[2.4]heptane, 4-oxaspiro[2.5]octane, 4-oxaspiro[2.6]nonane, 4-oxaspiro[2.7]decane, 2-oxaspiro[3.3]heptane, 5-oxaspiro[3.4]octane, 2-oxaspiro[3.5]nonane, 2-oxaspiro[3.6]decane, 2-oxaspiro[3.7]undecane, 1-oxaspiro[4.4]nonane, 2-oxaspiro[4.5]decane, 2-oxaspiro[4.6]undecane, 3-oxaspiro[5.5]undecane, 4-oxaspiro[2.3]hexane, 5-oxaspiro[2.4]heptane, 5-oxaspiro[2.5]octane, 6-oxaspiro[2.5]octane, 4,7-dioxaspiro[2.5]octane, 5-oxaspiro[2.6]nonane, 6-oxaspiro[2.6]nonane, 4,7-dioxaspiro[2.6]nonane, 5,8-dioxaspiro[2.6]nonane, 4,7-dioxaspiro[2.6]nonane, 5,8-dioxaspiro[2.7]decane, 4,7-dioxaspiro[2.7]decane, 5,9-dioxaspiro[2.7]decane, 7-oxaspiro[2.7]decane, 4,8-dioxaspiro[2.7]decane, 4,9-dioxaspiro[2.7]decane, 5-oxaspiro[2.7]decane, 6-oxaspiro[2.7]decane, 1-oxaspiro[3.3]heptane, 2,6-dioxaspiro[3.3]heptane, 1,6-dioxaspiro[3.3]heptane, 6-oxaspiro[3.4]octane, 1-oxaspiro[3.4]octane, 2-oxaspiro[3.4]octane, 2,5-dioxaspiro[3.4]octane, 2,6-dioxaspiro[3.4]octane, 1,6-dioxaspiro[3.4]octane, 2,6-dioxaspiro[3.4]octane, 1-oxaspiro[3.5]nonane, 5-oxaspiro[3.5]nonane, 6-oxaspiro[3.5]nonane, 7-oxaspiro[3.5]nonane, 2,5-dioxaspiro[3.5]nonane, 2,6-dioxaspiro[3.5]nonane, 1,7-dioxaspiro[3.5]nonane, 1,6-dioxaspiro[3.5]nonane, 2,7-dioxaspiro[3.5]nonane, 5,8-dioxaspiro[3.5]nonane, 1-oxaspiro[3.6]decane, 5-oxaspiro[3.6]decane, 6-oxaspiro[3.6]decane, 7-oxaspiro[3.6]decane, 2,6-dioxaspiro[3.6]decane, 2,7-dioxaspiro[3.6]decane, 5,8-dioxaspiro[3.6]decane, 1,6-dioxaspiro[3.6]decane, 1,7-dioxaspiro[3.6]decane, 5,9-dioxaspiro[3.6]decane, 6,9-dioxaspiro[3.6]decane, 2,5-dioxaspiro[3.6]decane, 2,5,8-trioxaspiro[3.6]decane, 1,6,9-trioxaspiro[3.6]decane, 2,6,9-trioxaspiro[3.6]decane, 2,5,9-trioxaspiro[3.6]decane, 1-oxaspiro[3.7]undecane, 5-oxaspiro[3.7]undecane, 6-oxaspiro[3.7]undecane, 7-oxaspiro[3.7]undecane, 2,5-dioxaspiro[3.7]undecane, 2,6-dioxaspiro[3.7]undecane, 2,7-dioxaspiro[3.7]undecane, 1,7-dioxaspiro[3.7]undecane, 5,8-dioxaspiro[3.7]undecane, 1,6-dioxaspiro[3.7]undecane, 1,8-dioxaspiro[3.7]undecane, 5,9-dioxaspiro[3.7]undecane, 2,8-dioxaspiro[3.7]undecane, 6,10-dioxaspiro[3.7]undecane, 5,10-dioxaspiro[3.7]undecane, 6,9-dioxaspiro[3.7]undecane, 2,6,9-trioxaspiro[3.7]undecane, 1,6,9-trioxaspiro[3.7]undecane, 2,5,8-trioxaspiro[3.7]undecane, 2,6,10-trioxaspiro[3.7]undecane, 2,5,9-trioxaspiro[3.7]undecane, 1,6,10-trioxaspiro[3.7]undecane, 2,5,10-trioxaspiro[3.7]undecane, 2-oxaspiro[4.4]nonane, 1,7-dioxaspiro[4.4]nonane, 2,7-dioxaspiro[4.4]nonane, 1-oxaspiro[4.5]decane, 6-oxaspiro[4.5]decane, 7-oxaspiro[4.5]decane, 8-oxaspiro[4.5]decane, 2,6-dioxaspiro[4.5]decane, 1,7-dioxaspiro[4.5]decane, 6,9-dioxaspiro[4.5]decane, 2,7-dioxaspiro[4.5]decane, 2,8-dioxaspiro[4.5]decane, 1,8-dioxaspiro[4.5]decane, 2,6,9-trioxaspiro[4.5]decane, 1-oxaspiro[4.6]undecane, 6-oxaspiro[4.6]undecane, 7-oxaspiro[4.6]undecane, 8-oxaspiro[4.6]undecane, 2,6-dioxaspiro[4.6]undecane, 1,7-dioxaspiro[4.6]undecane, 6,9-dioxaspiro[4.6]undecane, 7,10-dioxaspiro[4.6]undecane, 2,7-dioxaspiro[4.6]undecane, 1,8-dioxaspiro[4.6]undecane, 6,10-dioxaspiro[4.6]undecane, 2,8-dioxaspiro[4.6]undecane, 2,6,9-trioxaspiro[4.6]undecane, 1,7,10-trioxaspiro[4.6]undecane, 2,7,10-trioxaspiro[4.6]undecane, 2,6,10-trioxaspiro[4.6]undecane, 2-oxaspiro[5.5]undecane, 1-oxaspiro[5.5]undecane, 1,4-dioxaspiro[5.5]undecane, 1,9-dioxaspiro[5.5]undecane, 2,8-dioxaspiro[5.5]undecane, 1,8-dioxaspiro[5.5]undecane, 2,9-dioxaspiro[5.5]undecane, 3,9-dioxaspiro[5.5]undecane, 1,4,9-trioxaspiro[5.5]undecane, 1,4,8-trioxaspiro[5.5]undecane, 5-thiaspiro[2.3]hexane, 4-thiaspiro[2.4]heptane, 4-thiaspiro[2.5]octane, 4-thiaspiro[2.6]nonane, 4-thiaspiro[2.7]decane, 2-thiaspiro[3.3]heptane, 5-thiaspiro[3.4]octane, 2-thiaspiro[3.5]nonane, 2-thiaspiro[3.6]decane, 2-thiaspiro[3.7]undecane, 1-thiaspiro[4.4]nonane, 2-thiaspiro[4.5]decane, 2-thiaspiro[4.6]undecane, 3-thiaspiro[5.5]undecane, 4-thiaspiro[2.3]hexane, 5-thiaspiro[2.4]heptane, 5-thiaspiro[2.5]octane, 6-thiaspiro[2.5]octane, 7-oxa-4-thiaspiro[2.5]octane, 5-thiaspiro[2.6]nonane, 6-thiaspiro[2.6]nonane, 7-oxa-4-thiaspiro[2.6]nonane, 5-oxa-8-thiaspiro[2.6]nonane, 4-oxa-7-thiaspiro[2.6]nonane, 5-oxa-8-thiaspiro[2.7]decane, 7-oxa-4-thiaspiro[2.7]decane, 5-oxa-9-thiaspiro[2.7]decane, 7-thiaspiro[2.7]decane, 8-oxa-4-thiaspiro[2.7]decane, 4-oxa-9-thiaspiro[2.7]decane, 5-thiaspiro[2.7]decane, 6-thiaspiro[2.7]decane, 1-thiaspiro[3.3]heptane, 2-oxa-6-thiaspiro[3.3]heptane, 6-oxa-1-thiaspiro[3.3]heptane, 6-thiaspiro[3.4]octane, 1-thiaspiro[3.4]octane, 2-thiaspiro[3.4]octane, 2-oxa-5-thiaspiro[3.4]octane, 2-oxa-6-thiaspiro[3.4]octane, 1-oxa-6-thiaspiro[3.4]octane, 2-oxa-6-thiaspiro[3.4]octane, 1-thiaspiro[3.5]nonane, 5-thiaspiro[3.5]nonane, 6-thiaspiro[3.5]nonane, 7-thiaspiro[3.5]nonane, 2-oxa-5-thiaspiro[3.5]nonane, 2-oxa-6-thiaspiro[3.5]nonane, 1-oxa-7-thiaspiro[3.5]nonane, 1-oxa-6-thiaspiro[3.5]nonane, 2-oxa-7-thiaspiro[3.5]nonane, 8-oxa-5-thiaspiro[3.5]nonane, 1-thiaspiro[3.6]decane, 5-thiaspiro[3.6]decane, 6-thiaspiro[3.6]decane, 7-thiaspiro[3.6]decane, 6-oxa-2-thiaspiro[3.6]decane, 7-oxa-2-thiaspiro[3.6]decane, 8-oxa-5-thiaspiro[3.6]decane, 6-oxa-1-thiaspiro[3.6]decane, 9-oxa-5-thiaspiro[3.6]decane, 6-oxa-9-thiaspiro[3.6]decane, 5-oxa-2-thiaspiro[3.6]decane, 5,8-dioxa-2-thiaspiro[3.6]decane, 6,9-dioxa-1-thiaspiro[3.6]decane, 6,9-dioxa-2-thiaspiro[3.6]decane, 5,9-dioxa-2-thiaspiro[3.6]decane, 1-thiaspiro[3.7]undecane, 5-thiaspiro[3.7]undecane, 6-thiaspiro[3.7]undecane, 7-thiaspiro[3.7]undecane, 2-oxa-5-thiaspiro[3.7]undecane, 2-oxa-6-thiaspiro[3.7]undecane, 2-oxa-7-thiaspiro[3.7]undecane, 1-oxa-7-thiaspiro[3.7]undecane, 8-oxa-5-thiaspiro[3.7]undecane, 1-oxa-6-thiaspiro[3.7]undecane, 1-oxa-8-thiaspiro[3.7]undecane, 5-oxa-9-thiaspiro[3.7]undecane, 2-oxa-8-thiaspiro[3.7]undecane, 6-oxa-10-thiaspiro[3.7]undecane, 5-oxa-10-thiaspiro[3.7]undecane, 6-oxa-9-thiaspiro[3.7]undecane, 6,9-dioxa-2-thiaspiro[3.7]undecane, 6,9-dioxa-1-thiaspiro[3.7]undecane, 5,8-dioxa-2-thiaspiro[3.7]undecane, 6,10-dioxa-2-thiaspiro[3.7]undecane, 5,9-dioxa-2-thiaspiro[3.7]undecane, 6,10-dioxa-1-thiaspiro[3.7]undecane, 5,10-dioxa-2-thiaspiro[3.7]undecane, 2-thiaspiro[4.4]nonane, 7-oxa-1-thiaspiro[4.4]nonane, 2-oxa-7-thiaspiro[4.4]nonane, 1-thiaspiro[4.5]decane, 6-thiaspiro[4.5]decane, 7-thiaspiro[4.5]decane, 8-thiaspiro[4.5]decane, 6-oxa-2-thiaspiro[4.5]decane, 7-oxa-1-thiaspiro[4.5]decane, 9-oxa-6-thiaspiro[4.5]decane, 7-oxa-2-thiaspiro[4.5]decane, 8-oxa-2-thiaspiro[4.5]decane, 8-oxa-1-thiaspiro[4.5]decane, 6,9-dioxa-2-thiaspiro[4.5]decane, 1-thiaspiro[4.6]undecane, 6-thiaspiro[4.6]undecane, 7-thiaspiro[4.6]undecane, 8-thiaspiro[4.6]undecane, 6-oxa-2-thiaspiro[4.6]undecane, 7-oxa-1-thiaspiro[4.6]undecane, 9-oxa-6-thiaspiro[4.6]undecane, 7-oxa-10-thiaspiro[4.6]undecane, 7-oxa-2-thiaspiro[4.6]undecane, 1-oxa-8-thiaspiro[4.6]undecane, 10-oxa-6-thiaspiro[4.6]undecane, 8-oxa-2-thiaspiro[4.6]undecane, 6,9-dioxa-2-thiaspiro[4.6]undecane, 7,10-dioxa-1-thiaspiro[4.6]undecane, 7,10-dioxa-2-thiaspiro[4.6]undecane, 6,10-dioxa-2-thiaspiro[4.6]undecane, 2-thiaspiro[5.5]undecane, 1-thiaspiro[5.5]undecane, 1-oxa-4-thiaspiro[5.5]undecane, 1-oxa-9-thiaspiro[5.5]undecane, 2-oxa-8-thiaspiro[5.5]undecane, 8-oxa-1-thiaspiro[5.5]undecane, 2-oxa-9-thiaspiro[5.5]undecane, 3-oxa-9-thiaspiro[5.5]undecane, 1,4-dioxa-9-thiaspiro[5.5]undecane, 1,4-dioxa-8-thiaspiro[5.5]undecane, 4-oxa-7-thiaspiro[2.5]octane, 4-oxa-7-thiaspiro[2.6]nonane, 7-oxa-4-thiaspiro[2.6]nonane, 8-oxa-5-thiaspiro[2.7]decane, 4-oxa-7-thiaspiro[2.7]decane, 4-oxa-8-thiaspiro[2.7]decane, 9-oxa-4-thiaspiro[2.7]decane, 1-oxa-6-thiaspiro[3.3]heptane, 7-oxa-1-thiaspiro[3.6]decane, 5-oxa-2-thiaspiro[3.4]octane, 6-oxa-2-thiaspiro[3.4]octane, 6-oxa-1-thiaspiro[3.4]octane, 6-oxa-2-thiaspiro[3.4]octane, 5-oxa-2-thiaspiro[3.5]nonane, 6-oxa-2-thiaspiro[3.5]nonane, 7-oxa-1-thiaspiro[3.5]nonane, 6-oxa-1-thiaspiro[3.5]nonane, 7-oxa-2-thiaspiro[3.5]nonane, 5-oxa-8-thiaspiro[3.5]nonane, 2-oxa-6-thiaspiro[3.6]decane, 2-oxa-7-thiaspiro[3.6]decane, 5-oxa-8-thiaspiro[3.6]decane, 1-oxa-6-thiaspiro[3.6]decane, 5-oxa-9-thiaspiro[3.6]decane, 2-oxa-5-thiaspiro[3.6]decane, 1-oxa-7-thiaspiro[3.6]decane, 2,5-dioxa-8-thiaspiro[3.6]decane, 1,6-dioxa-9-thiaspiro[3.6]decane, 2,6-dioxa-9-thiaspiro[3.6]decane, 2,5-dioxa-9-thiaspiro[3.6]decane, 2,8-dioxa-5-thiaspiro[3.6]decane, 1,6-dioxa-9-thiaspiro[3.6]decane, 2,6-dioxa-9-thiaspiro[3.6]decane, 2,9-dioxa-5-thiaspiro[3.6]decane, 5-oxa-2-thiaspiro[3.7]undecane, 6-oxa-2-thiaspiro[3.7]undecane, 7-oxa-2-thiaspiro[3.7]undecane, 7-oxa-1-thiaspiro[3.7]undecane, 6-oxa-1-thiaspiro[3.7]undecane, 8-oxa-1-thiaspiro[3.7]undecane, 8-oxa-2-thiaspiro[3.7]undecane, 5-oxa-8-thiaspiro[3.7]undecane, 9-oxa-5-thiaspiro[3.7]undecane, 10-oxa-5-thiaspiro[3.7]undecane, 9-oxa-6-thiaspiro[3.7]undecane, 2,9-dioxa-6-thiaspiro[3.7]undecane, 1,9-dioxa-6-thiaspiro[3.7]undecane, 2,5-dioxa-8-thiaspiro[3.7]undecane, 2,6-dioxa-10-thiaspiro[3.7]undecane, 2,5-dioxa-9-thiaspiro[3.7]undecane, 1,6-dioxa-10-thiaspiro[3.7]undecane, 2,5-dioxa-10-thiaspiro[3.7]undecane, 2,6-dioxa-9-thiaspiro[3.7]undecane, 1,6-dioxa-9-thiaspiro[3.7]undecane, 2,8-dioxa-5-thiaspiro[3.7]undecane, 2,6-dioxa-10-thiaspiro[3.7]undecane, 2,9-dioxa-5-thiaspiro[3.7]undecane, 1,6-dioxa-10-thiaspiro[3.7]undecane, 5,10-dioxa-2-thiaspiro[3.7]undecane, 1-oxa-7-thiaspiro[4.4]nonane, 2-oxa-7-thiaspiro[4.4]nonane, 2,6-dioxa-9-thiaspiro[4.5]decane, 2,9-dioxa-6-thiaspiro[4.5]decane, 2-oxa-6-thiaspiro[4.5]decane, 1-oxa-7-thiaspiro[4.5]decane, 6-oxa-9-thiaspiro[4.5]decane, 2-oxa-7-thiaspiro[4.5]decane, 2-oxa-8-thiaspiro[4.5]decane, 1-oxa-8-thiaspiro[4.5]decane, 2-oxa-6-thiaspiro[4.6]undecane, 6-oxa-9-thiaspiro[4.6]undecane, 7-oxa-10-thiaspiro[4.6]undecane, 2-oxa-7-thiaspiro[4.6]undecane, 6-oxa-10-thiaspiro[4.6]undecane, 2-oxa-8-thiaspiro[4.6]undecane, 7-oxa-1-thiaspiro[4.6]undecane, 1-oxa-8-thiaspiro[4.6]undecane, 2,6-dioxa-9-thiaspiro[4.6]undecane, 1,7-dioxa-10-thiaspiro[4.6]undecane, 2,7-dioxa-10-thiaspiro[4.6]undecane, 2,6-dioxa-10-thiaspiro[4.6]undecane, 2,9-dioxa-6-thiaspiro[4.6]undecane, 1,7-dioxa-10-thiaspiro[4.6]undecane, 2,7-dioxa-10-thiaspiro[4.6]undecane, 2,10-dioxa-6-thiaspiro[4.6]undecane, 1,9-dioxa-4-thiaspiro[5.5]undecane, 4,8-dioxa-1-thiaspiro[5.5]undecane, 4,9-dioxa-1-thiaspiro[5.5]undecane, 1,8-dioxa-4-thiaspiro[5.5]undecane, 4-oxa-1-thiaspiro[5.5]undecane, 9-oxa-1-thiaspiro[5.5]undecane, 1-oxa-8-thiaspiro[5.5]undecane, 9-oxa-2-thiaspiro[5.5]undecane, 5-azaspiro[2.3]hexane, 4-azaspiro[2.4]heptane, 4-azaspiro[2.5]octane, 4-azaspiro[2.6]nonane, 4-azaspiro[2.7]decane, 2-azaspiro[3.3]heptane, 5-azaspiro[3.4]octane, 2-azaspiro[3.5]nonane, 2-azaspiro[3.6]decane, 2-azaspiro[3.7]undecane, 1-azaspiro[4.4]nonane, 2-azaspiro[4.5]decane, 2-azaspiro[4.6]undecane, 3-azaspiro[5.5]undecane, 4-azaspiro[2.3]hexane, 5-azaspiro[2.4]heptane, 5-azaspiro[2.5]octane, 6-azaspiro[2.5]octane, 4,7-diazaspiro[2.5]octane, 5-azaspiro[2.6]nonane, 6-azaspiro[2.6]nonane, 4,7-diazaspiro[2.6]nonane, 5,8-diazaspiro[2.6]nonane, 4,7-diazaspiro[2.6]nonane, 5,8-diazaspiro[2.7]decane, 4,7-diazaspiro[2.7]decane, 5,9-diazaspiro[2.7]decane, 7-azaspiro[2.7]decane, 4,8-diazaspiro[2.7]decane, 4,9-diazaspiro[2.7]decane, 5-azaspiro[2.7]decane, 6-azaspiro[2.7]decane, 1-azaspiro[3.3]heptane, 2,6-diazaspiro[3.3]heptane, 1,6-diazaspiro[3.3]heptane, 6-azaspiro[3.4]octane, 1-azaspiro[3.4]octane, 2-azaspiro[3.4]octane, 2,5-diazaspiro[3.4]octane, 2,6-diazaspiro[3.4]octane, 1,6-diazaspiro[3.4]octane, 2,6-diazaspiro[3.4]octane, 1-azaspiro[3.5]nonane, 5-azaspiro[3.5]nonane, 6-azaspiro[3.5]nonane, 7-azaspiro[3.5]nonane, 2,5-diazaspiro[3.5]nonane, 2,6-diazaspiro[3.5]nonane, 1,7-diazaspiro[3.5]nonane, 1,6-diazaspiro[3.5]nonane, 2,7-diazaspiro[3.5]nonane, 5,8-diazaspiro[3.5]nonane, 1-azaspiro[3.6]decane, 5-azaspiro[3.6]decane, 6-azaspiro[3.6]decane, 7-azaspiro[3.6]decane, 2,6-diazaspiro[3.6]decane, 2,7-diazaspiro[3.6]decane, 5,8-diazaspiro[3.6]decane, 1,6-diazaspiro[3.6]decane, 1,7-diazaspiro[3.6]decane, 5,9-diazaspiro[3.6]decane, 6,9-diazaspiro[3.6]decane, 2,5-diazaspiro[3.6]decane, 2,5,8-triazaspiro[3.6]decane, 1,6,9-triazaspiro[3.6]decane, 2,6,9-triazaspiro[3.6]decane, 2,5,9-triazaspiro[3.6]decane, 1-azaspiro[3.7]undecane, 5-azaspiro[3.7]undecane, 6-azaspiro[3.7]undecane, 7-azaspiro[3.7]undecane, 2,5-diazaspiro[3.7]undecane, 2,6-diazaspiro[3.7]undecane, 2,7-diazaspiro[3.7]undecane, 1,7-diazaspiro[3.7]undecane, 5,8-diazaspiro[3.7]undecane, 1,6-diazaspiro[3.7]undecane, 1,8-diazaspiro[3.7]undecane, 5,9-diazaspiro[3.7]undecane, 2,8-diazaspiro[3.7]undecane, 6,10-diazaspiro[3.7]undecane, 5,10-diazaspiro[3.7]undecane, 6,9-diazaspiro[3.7]undecane, 2,6,9-triazaspiro[3.7]undecane, 1,6,9-triazaspiro[3.7]undecane, 2,5,8-triazaspiro[3.7]undecane, 2,6,10-triazaspiro[3.7]undecane, 2,5,9-triazaspiro[3.7]undecane, 1,6,10-triazaspiro[3.7]undecane, 2,5,10-triazaspiro[3.7]undecane, 2-azaspiro[4.4]nonane, 1,7-diazaspiro[4.4]nonane, 2,7-diazaspiro[4.4]nonane, 1-azaspiro[4.5]decane, 6-azaspiro[4.5]decane, 7-azaspiro[4.5]decane, 8-azaspiro[4.5]decane, 2,6-diazaspiro[4.5]decane, 1,7-diazaspiro[4.5]decane, 6,9-diazaspiro[4.5]decane, 2,7-diazaspiro[4.5]decane, 2,8-diazaspiro[4.5]decane, 1,8-diazaspiro[4.5]decane, 2,6,9-triazaspiro[4.5]decane, 1-azaspiro[4.6]undecane, 6-azaspiro[4.6]undecane, 7-azaspiro[4.6]undecane, 8-azaspiro[4.6]undecane, 2,6-diazaspiro[4.6]undecane, 1,7-diazaspiro[4.6]undecane, 6,9-diazaspiro[4.6]undecane, 7,10-diazaspiro[4.6]undecane, 2,7-diazaspiro[4.6]undecane, 1,8-diazaspiro[4.6]undecane, 6,10-diazaspiro[4.6]undecane, 2,8-diazaspiro[4.6]undecane, 2,6,9-triazaspiro[4.6]undecane, 1,7,10-triazaspiro[4.6]undecane, 2,7,10-triazaspiro[4.6]undecane, 2,6,10-triazaspiro[4.6]undecane, 2-azaspiro[5.5]undecane, 1-azaspiro[5.5]undecane, 1,4-diazaspiro[5.5]undecane, 1,9-diazaspiro[5.5]undecane, 2,8-diazaspiro[5.5]undecane, 1,8-diazaspiro[5.5]undecane, 2,9-diazaspiro[5.5]undecane, 3,9-diazaspiro[5.5]undecane, 1,4,9-triazaspiro[5.5]undecane, 1,4,8-triazaspiro[5.5]undecane, 7-oxa-4-azaspiro[2.5]octane, 7-oxa-4-azaspiro[2.6]nonane, 5-oxa-8-azaspiro[2.6]nonane, 4-oxa-7-azaspiro[2.6]nonane, 5-oxa-8-azaspiro[2.7]decane, 7-oxa-4-azaspiro[2.7]decane, 5-oxa-9-azaspiro[2.7]decane, 8-oxa-4-azaspiro[2.7]decane, 4-oxa-9-azaspiro[2.7]decane, 2-oxa-6-azaspiro[3.3]heptane, 6-oxa-1-azaspiro[3.3]heptane, 2-oxa-5-azaspiro[3.4]octane, 2-oxa-6-azaspiro[3.4]octane, 1-oxa-6-azaspiro[3.4]octane, 2-oxa-6-azaspiro[3.4]octane, 2-oxa-5-azaspiro[3.5]nonane, 2-oxa-6-azaspiro[3.5]nonane, 1-oxa-7-azaspiro[3.5]nonane, 1-oxa-6-azaspiro[3.5]nonane, 2-oxa-7-azaspiro[3.5]nonane, 8-oxa-5-azaspiro[3.5]nonane, 6-oxa-2-azaspiro[3.6]decane, 7-oxa-2-azaspiro[3.6]decane, 8-oxa-5-azaspiro[3.6]decane, 6-oxa-1-azaspiro[3.6]decane, 9-oxa-5-azaspiro[3.6]decane, 6-oxa-9-azaspiro[3.6]decane, 5-oxa-2-azaspiro[3.6]decane, 5,8-dioxa-2-azaspiro[3.6]decane, 6,9-dioxa-1-azaspiro[3.6]decane, 6,9-dioxa-2-azaspiro[3.6]decane, 5,9-dioxa-2-azaspiro[3.6]decane, 2-oxa-5-azaspiro[3.7]undecane, 2-oxa-6-azaspiro[3.7]undecane, 2-oxa-7-azaspiro[3.7]undecane, 1-oxa-7-azaspiro[3.7]undecane, 8-oxa-5-azaspiro[3.7]undecane, 1-oxa-6-azaspiro[3.7]undecane, 1-oxa-8-azaspiro[3.7]undecane, 5-oxa-9-azaspiro[3.7]undecane, 2-oxa-8-azaspiro[3.7]undecane, 6-oxa-10-azaspiro[3.7]undecane, 5-oxa-10-azaspiro[3.7]undecane, 6-oxa-9-azaspiro[3.7]undecane, 6,9-dioxa-2-azaspiro[3.7]undecane, 6,9-dioxa-1-azaspiro[3.7]undecane, 5,8-dioxa-2-azaspiro[3.7]undecane, 6,10-dioxa-2-azaspiro[3.7]undecane, 5,9-dioxa-2-azaspiro[3.7]undecane, 6,10-dioxa-1-azaspiro[3.7]undecane, 5,10-dioxa-2-azaspiro[3.7]undecane, 7-oxa-1-azaspiro[4.4]nonane, 2-oxa-7-azaspiro[4.4]nonane, 6-oxa-2-azaspiro[4.5]decane, 7-oxa-1-azaspiro[4.5]decane, 9-oxa-6-azaspiro[4.5]decane, 7-oxa-2-azaspiro[4.5]decane, 8-oxa-2-azaspiro[4.5]decane, 8-oxa-1-azaspiro[4.5]decane, 6,9-dioxa-2-azaspiro[4.5]decane, 6-oxa-2-azaspiro[4.6]undecane, 7-oxa-1-azaspiro[4.6]undecane, 9-oxa-6-azaspiro[4.6]undecane, 7-oxa-10-azaspiro[4.6]undecane, 7-oxa-2-azaspiro[4.6]undecane, 1-oxa-8-azaspiro[4.6]undecane, 10-oxa-6-azaspiro[4.6]undecane, 8-oxa-2-azaspiro[4.6]undecane, 6,9-dioxa-2-azaspiro[4.6]undecane, 7,10-dioxa-1-azaspiro[4.6]undecane, 7,10-dioxa-2-azaspiro[4.6]undecane, 6,10-dioxa-2-azaspiro[4.6]undecane, 1-oxa-4-azaspiro[5.5]undecane, 1-oxa-9-azaspiro[5.5]undecane, 2-oxa-8-azaspiro[5.5]undecane, 8-oxa-1-azaspiro[5.5]undecane, 2-oxa-9-azaspiro[5.5]undecane, 3-oxa-9-azaspiro[5.5]undecane, 1,4-dioxa-9-azaspiro[5.5]undecane, 1,4-dioxa-8-azaspiro[5.5]undecane, 4-oxa-7-azaspiro[2.5]octane, 4-oxa-7-azaspiro[2.6]nonane, 7-oxa-4-azaspiro[2.6]nonane, 8-oxa-5-azaspiro[2.7]decane, 4-oxa-7-azaspiro[2.7]decane, 4-oxa-8-azaspiro[2.7]decane, 9-oxa-4-azaspiro[2.7]decane, 1-oxa-6-azaspiro[3.3]heptane, 7-oxa-1-azaspiro[3.6]decane, 5-oxa-2-azaspiro[3.4]octane, 6-oxa-2-azaspiro[3.4]octane, 6-oxa-1-azaspiro[3.4]octane, 6-oxa-2-azaspiro[3.4]octane, 5-oxa-2-azaspiro[3.5]nonane, 6-oxa-2-azaspiro[3.5]nonane, 7-oxa-1-azaspiro[3.5]nonane, 6-oxa-1-azaspiro[3.5]nonane, 7-oxa-2-azaspiro[3.5]nonane, 5-oxa-8-azaspiro[3.5]nonane, 2-oxa-6-azaspiro[3.6]decane, 2-oxa-7-azaspiro[3.6]decane, 5-oxa-8-azaspiro[3.6]decane, 1-oxa-6-azaspiro[3.6]decane, 5-oxa-9-azaspiro[3.6]decane, 2-oxa-5-azaspiro[3.6]decane, 1-oxa-7-azaspiro[3.6]decane, 2,5-dioxa-8-azaspiro[3.6]decane, 1,6-dioxa-9-azaspiro[3.6]decane, 2,6-dioxa-9-azaspiro[3.6]decane, 2,5-dioxa-9-azaspiro[3.6]decane, 2,8-dioxa-5-azaspiro[3.6]decane, 1,6-dioxa-9-azaspiro[3.6]decane, 2,6-dioxa-9-azaspiro[3.6]decane, 2,9-dioxa-5-azaspiro[3.6]decane, 5-oxa-2-azaspiro[3.7]undecane, 6-oxa-2-azaspiro[3.7]undecane, 7-oxa-2-azaspiro[3.7]undecane, 7-oxa-1-azaspiro[3.7]undecane, 6-oxa-1-azaspiro[3.7]undecane, 8-oxa-1-azaspiro[3.7]undecane, 8-oxa-2-azaspiro[3.7]undecane, 5-oxa-8-azaspiro[3.7]undecane, 9-oxa-5-azaspiro[3.7]undecane, 10-oxa-5-azaspiro[3.7]undecane, 9-oxa-6-azaspiro[3.7]undecane, 2,9-dioxa-6-azaspiro[3.7]undecane, 1,9-dioxa-6-azaspiro[3.7]undecane, 2,5-dioxa-8-azaspiro[3.7]undecane, 2,6-dioxa-10-azaspiro[3.7]undecane, 2,5-dioxa-9-azaspiro[3.7]undecane, 1,6-dioxa-10-azaspiro[3.7]undecane, 2,5-dioxa-10-azaspiro[3.7]undecane, 2,6-dioxa-9-azaspiro[3.7]undecane, 1,6-dioxa-9-azaspiro[3.7]undecane, 2,8-dioxa-5-azaspiro[3.7]undecane, 2,6-dioxa-10-azaspiro[3.7]undecane, 2,9-dioxa-5-azaspiro[3.7]undecane, 1,6-dioxa-10-azaspiro[3.7]undecane, 5,10-dioxa-2-azaspiro[3.7]undecane, 1-oxa-7-azaspiro[4.4]nonane, 2-oxa-7-azaspiro[4.4]nonane, 2,6-dioxa-9-azaspiro[4.5]decane, 2,9-dioxa-6-azaspiro[4.5]decane, 2-oxa-6-azaspiro[4.5]decane, 1-oxa-7-azaspiro[4.5]decane, 6-oxa-9-azaspiro[4.5]decane, 2-oxa-7-azaspiro[4.5]decane, 2-oxa-8-azaspiro[4.5]decane, 1-oxa-8-azaspiro[4.5]decane, 2-oxa-6-azaspiro[4.6]undecane, 6-oxa-9-azaspiro[4.6]undecane, 7-oxa-10-azaspiro[4.6]undecane, 2-oxa-7-azaspiro[4.6]undecane, 6-oxa-10-azaspiro[4.6]undecane, 2-oxa-8-azaspiro[4.6]undecane, 7-oxa-1-azaspiro[4.6]undecane, 1-oxa-8-azaspiro[4.6]undecane, 2,6-dioxa-9-azaspiro[4.6]undecane, 1,7-dioxa-10-azaspiro[4.6]undecane, 2,7-dioxa-10-azaspiro[4.6]undecane, 2,6-dioxa-10-azaspiro[4.6]undecane, 2,9-dioxa-6-azaspiro[4.6]undecane, 1,7-dioxa-10-azaspiro[4.6]undecane, 2,7-dioxa-10-azaspiro[4.6]undecane, 2,10-dioxa-6-azaspiro[4.6]undecane, 1,9-dioxa-4-azaspiro[5.5]undecane, 4,8-dioxa-1-azaspiro[5.5]undecane, 4,9-dioxa-1-azaspiro[5.5]undecane, 1,8-dioxa-4-azaspiro[5.5]undecane, 4-oxa-1-azaspiro[5.5]undecane, 9-oxa-1-azaspiro[5.5]undecane, 1-oxa-8-azaspiro[5.5]undecane, 9-oxa-2-azaspiro[5.5]undecane, 2-oxa-5,8-diazaspiro[3.6]decane, 1-oxa-6,9-diazaspiro[3.6]decane, 2-oxa-6,9-diazaspiro[3.6]decane, 2-oxa-5,9-diazaspiro[3.6]decane, 8-oxa-2,5-diazaspiro[3.6]decane, 6-oxa-1,9-diazaspiro[3.6]decane, 6-oxa-2,9-diazaspiro[3.6]decane, 9-oxa-2,5-diazaspiro[3.6]decane, 5-oxa-2,8-diazaspiro[3.6]decane, 6-oxa-1,9-diazaspiro[3.6]decane, 6-oxa-2,9-diazaspiro[3.6]decane, 5-oxa-2,9-diazaspiro[3.6]decane, 2-oxa-6,9-diazaspiro[3.7]undecane, 1-oxa-6,9-diazaspiro[3.7]undecane, 2-oxa-5,8-diazaspiro[3.7]undecane, 2-oxa-6,10-diazaspiro[3.7]undecane, 2-oxa-5,9-diazaspiro[3.7]undecane, 1-oxa-6,10-diazaspiro[3.7]undecane, 2-oxa-5,10-diazaspiro[3.7]undecane, 6-oxa-2,9-diazaspiro[3.7]undecane, 6-oxa-1,9-diazaspiro[3.7]undecane, 8-oxa-2,5-diazaspiro[3.7]undecane, 6-oxa-2,10-diazaspiro[3.7]undecane, 9-oxa-2,5-diazaspiro[3.7]undecane, 6-oxa-1,10-diazaspiro[3.7]undecane, 10-oxa-2,5-diazaspiro[3.7]undecane, 9-oxa-2,6-diazaspiro[3.7]undecane, 9-oxa-1,6-diazaspiro[3.7]undecane, 5-oxa-2,8-diazaspiro[3.7]undecane, 6-oxa-2,10-diazaspiro[3.7]undecane, 5-oxa-2,9-diazaspiro[3.7]undecane, 6-oxa-1,10-diazaspiro[3.7]undecane, 2-oxa-5,10-diazaspiro[3.7]undecane, 2-oxa-6,9-diazaspiro[4.5]decane, 9-oxa-2,6-diazaspiro[4.5]decane, 6-oxa-2,9-diazaspiro[4.5]decane, 2-oxa-6,9-diazaspiro[4.6]undecane, 1-oxa-7,10-diazaspiro[4.6]undecane, 2-oxa-7,10-diazaspiro[4.6]undecane, 2-oxa-6,10-diazaspiro[4.6]undecane, 9-oxa-2,6-diazaspiro[4.6]undecane, 7-oxa-1,10-diazaspiro[4.6]undecane, 7-oxa-2,10-diazaspiro[4.6]undecane, 10-oxa-2,6-diazaspiro[4.6]undecane, 6-oxa-2,9-diazaspiro[4.6]undecane, 7-oxa-1,10-diazaspiro[4.6]undecane, 7-oxa-2,10-diazaspiro[4.6]undecane, 6-oxa-2,10-diazaspiro[4.6]undecane, 9-oxa-1,4-diazaspiro[5.5]undecane, 8-oxa-1,4-diazaspiro[5.5]undecane, 4-oxa-1,9-diazaspiro[5.5]undecane, 1-oxa-4,8-diazaspiro[5.5]undecane, 1-oxa-4,9-diazaspiro[5.5]undecane, 4-oxa-1,8-diazaspiro[5.5]undecane, 7-thia-4-azaspiro[2.5]octane, 7-thia-4-azaspiro[2.6]nonane, 5-thia-8-azaspiro[2.6]nonane, 4-thia-7-azaspiro[2.6]nonane, 5-thia-8-azaspiro[2.7]decane, 7-thia-4-azaspiro[2.7]decane, 5-thia-9-azaspiro[2.7]decane, 8-thia-4-azaspiro[2.7]decane, 4-thia-9-azaspiro[2.7]decane, 2-thia-6-azaspiro[3.3]heptane, 6-thia-1-azaspiro[3.3]heptane, 2-thia-5-azaspiro[3.4]octane, 2-thia-6-azaspiro[3.4]octane, 1-thia-6-azaspiro[3.4]octane, 2-thia-6-azaspiro[3.4]octane, 2-thia-5-azaspiro[3.5]nonane, 2-thia-6-azaspiro[3.5]nonane, 1-thia-7-azaspiro[3.5]nonane, 1-thia-6-azaspiro[3.5]nonane, 2-thia-7-azaspiro[3.5]nonane, 8-thia-5-azaspiro[3.5]nonane, 6-thia-2-azaspiro[3.6]decane, 7-thia-2-azaspiro[3.6]decane, 8-thia-5-azaspiro[3.6]decane, 6-thia-1-azaspiro[3.6]decane, 9-thia-5-azaspiro[3.6]decane, 6-thia-9-azaspiro[3.6]decane, 5-thia-2-azaspiro[3.6]decane, 2-thia-5-azaspiro[3.7]undecane, 2-thia-6-azaspiro[3.7]undecane, 2-thia-7-azaspiro[3.7]undecane, 1-thia-7-azaspiro[3.7]undecane, 8-thia-5-azaspiro[3.7]undecane, 1-thia-6-azaspiro[3.7]undecane, 1-thia-8-azaspiro[3.7]undecane, 5-thia-9-azaspiro[3.7]undecane, 2-thia-8-azaspiro[3.7]undecane, 6-thia-10-azaspiro[3.7]undecane, 5-thia-10-azaspiro[3.7]undecane, 6-thia-9-azaspiro[3.7]undecane, 7-thia-1-azaspiro[4.4]nonane, 2-thia-7-azaspiro[4.4]nonane, 4-thia-7-azaspiro[2.5]octane, 4-thia-7-azaspiro[2.6]nonane, 7-thia-4-azaspiro[2.6]nonane, 8-thia-5-azaspiro[2.7]decane, 4-thia-7-azaspiro[2.7]decane, 4-thia-8-azaspiro[2.7]decane, 9-thia-4-azaspiro[2.7]decane, 1-thia-6-azaspiro[3.3]heptane, 7-thia-1-azaspiro[3.6]decane, 5-thia-2-azaspiro[3.4]octane, 6-thia-2-azaspiro[3.4]octane, 6-thia-1-azaspiro[3.4]octane, 6-thia-2-azaspiro[3.4]octane, 5-thia-2-azaspiro[3.5]nonane, 6-thia-2-azaspiro[3.5]nonane, 7-thia-1-azaspiro[3.5]nonane, 6-thia-1-azaspiro[3.5]nonane, 7-thia-2-azaspiro[3.5]nonane, 5-thia-8-azaspiro[3.5]nonane, 2-thia-6-azaspiro[3.6]decane, 2-thia-7-azaspiro[3.6]decane, 5-thia-8-azaspiro[3.6]decane, 1-thia-6-azaspiro[3.6]decane, 5-thia-9-azaspiro[3.6]decane, 2-thia-5-azaspiro[3.6]decane, 1-thia-7-azaspiro[3.6]decane, 5-thia-2-azaspiro[3.7]undecane, 6-thia-2-azaspiro[3.7]undecane, 7-thia-2-azaspiro[3.7]undecane, 7-thia-1-azaspiro[3.7]undecane, 6-thia-1-azaspiro[3.7]undecane, 8-thia-1-azaspiro[3.7]undecane, 8-thia-2-azaspiro[3.7]undecane, 5-thia-8-azaspiro[3.7]undecane, 9-thia-5-azaspiro[3.7]undecane, 10-thia-5-azaspiro[3.7]undecane, 9-thia-6-azaspiro[3.7]undecane, 1-thia-7-azaspiro[4.4]nonane, 2-thia-7-azaspiro[4.4]nonane, 2-thia-5,8-diazaspiro[3.6]decane, 1-thia-6,9-diazaspiro[3.6]decane, 2-thia-6,9-diazaspiro[3.6]decane, 2-thia-5,9-diazaspiro[3.6]decane, 8-thia-2,5-diazaspiro[3.6]decane, 6-thia-1,9-diazaspiro[3.6]decane, 6-thia-2,9-diazaspiro[3.6]decane, 9-thia-2,5-diazaspiro[3.6]decane, 5-thia-2,8-diazaspiro[3.6]decane, 6-thia-1,9-diazaspiro[3.6]decane, 6-thia-2,9-diazaspiro[3.6]decane, 5-thia-2,9-diazaspiro[3.6]decane, 2-thia-6,9-diazaspiro[4.5]decane, 9-thia-2,6-diazaspiro[4.5]decane, 6-thia-2,9-diazaspiro[4.5]decane, 2-thia-6,9-diazaspiro[4.6]undecane, 1-thia-7,10-diazaspiro[4.6]undecane, 2-thia-7,10-diazaspiro[4.6]undecane, 2-thia-6,10-diazaspiro[4.6]undecane, 9-thia-2,6-diazaspiro[4.6]undecane, 7-thia-1,10-diazaspiro[4.6]undecane, 7-thia-2,10-diazaspiro[4.6]undecane, 10-thia-2,6-diazaspiro[4.6]undecane, 6-thia-2,9-diazaspiro[4.6]undecane, 7-thia-1,10-diazaspiro[4.6]undecane, 7-thia-2,10-diazaspiro[4.6]undecane, 6-thia-2,10-diazaspiro[4.6]undecane, 2-thia-6,9-diazaspiro[3.7]undecane, 1-thia-6,9-diazaspiro[3.7]undecane, 2-thia-5,8-diazaspiro[3.7]undecane, 2-thia-6,10-diazaspiro[3.7]undecane, 2-thia-5,9-diazaspiro[3.7]undecane, 1-thia-6,10-diazaspiro[3.7]undecane, 2-thia-5,10-diazaspiro[3.7]undecane, 6-thia-2,9-diazaspiro[3.7]undecane, 6-thia-1,9-diazaspiro[3.7]undecane, 8-thia-2,5-diazaspiro[3.7]undecane, 6-thia-2,10-diazaspiro[3.7]undecane, 9-thia-2,5-diazaspiro[3.7]undecane, 6-thia-1,10-diazaspiro[3.7]undecane, 10-thia-2,5-diazaspiro[3.7]undecane, 9-thia-2,6-diazaspiro[3.7]undecane, 9-thia-1,6-diazaspiro[3.7]undecane, 5-thia-2,8-diazaspiro[3.7]undecane, 6-thia-2,10-diazaspiro[3.7]undecane, 5-thia-2,9-diazaspiro[3.7]undecane, 6-thia-1,10-diazaspiro[3.7]undecane, 2-thia-5,10-diazaspiro[3.7]undecane, 6-thia-2-azaspiro[4.5]decane, 7-thia-1-azaspiro[4.5]decane, 9-thia-6-azaspiro[4.5]decane, 7-thia-2-azaspiro[4.5]decane, 8-thia-2-azaspiro[4.5]decane, 8-thia-1-azaspiro[4.5]decane, 6-thia-2-azaspiro[4.6]undecane, 7-thia-1-azaspiro[4.6]undecane, 9-thia-6-azaspiro[4.6]undecane, 7-thia-10-azaspiro[4.6]undecane, 7-thia-2-azaspiro[4.6]undecane, 1-thia-8-azaspiro[4.6]undecane, 10-thia-6-azaspiro[4.6]undecane, 8-thia-2-azaspiro[4.6]undecane, 2-thia-6-azaspiro[4.5]decane, 1-thia-7-azaspiro[4.5]decane, 6-thia-9-azaspiro[4.5]decane, 2-thia-7-azaspiro[4.5]decane, 2-thia-8-azaspiro[4.5]decane, 1-thia-8-azaspiro[4.5]decane, 2-thia-6-azaspiro[4.6]undecane, 6-thia-9-azaspiro[4.6]undecane, 7-thia-10-azaspiro[4.6]undecane, 2-thia-7-azaspiro[4.6]undecane, 6-thia-10-azaspiro[4.6]undecane, 2-thia-8-azaspiro[4.6]undecane, 7-thia-1-azaspiro[4.6]undecane, 1-thia-8-azaspiro[4.6]undecane, 1-thia-4-azaspiro[5.5]undecane, 1-thia-9-azaspiro[5.5]undecane, 2-thia-8-azaspiro[5.5]undecane, 8-thia-1-azaspiro[5.5]undecane, 2-thia-9-azaspiro[5.5]undecane, 3-thia-9-azaspiro[5.5]undecane, 4-thia-1-azaspiro[5.5]undecane, 9-thia-1-azaspiro[5.5]undecane, 1-thia-8-azaspiro[5.5]undecane, 9-thia-2-azaspiro[5.5]undecane, 9-thia-1,4-diazaspiro[5.5]undecane, 8-thia-1,4-diazaspiro[5.5]undecane, 4-thia-1,9-diazaspiro[5.5]undecane, 1-thia-4,8-diazaspiro[5.5]undecane, 1-thia-4,9-diazaspiro[5.5]undecane, 4-thia-1,8-diazaspiro[5.5]undecane, 2-oxabicyclo[1.1.1]pentane, 5-oxabicyclo[2.1.1]hexane, 7-oxabicyclo[2.2.1]heptane, 7-oxabicyclo[4.1.1]octane, 8-oxabicyclo[5.1.1]nonane, 9-oxabicyclo[4.2.1]nonane, 3-oxabicyclo[3.3.1]nonane, 2-oxabicyclo[2.2.2]octane, 3-oxabicyclo[3.2.2]nonane, 10-oxabicyclo[5.2.1]decane, 7-oxabicyclo[4.2.2]decane, 2-oxabicyclo[3.3.2]decane, spiro[2.3]hexane, spiro[2.4]heptane, spiro[2.5]octane, spiro[2.6]nonane, spiro[2.7]decane, spiro[2.2]pentane, spiro[3.3]heptane, spiro[3.4]octane, spiro[3.5]nonane, spiro[3.6]decane, spiro[3.7]undecane, spiro[4.4]nonane, spiro[4.5]decane, spiro[4.6]undecane, spiro[4.7]dodecane, spiro[5.5]undecane, spiro[5.6]dodecane, spiro[5.7]tridecane, spiro[6.6]tridecane, spiro[6.7]tetradecane, spiro[7.7]pentadecane, 2-oxabicyclo[2.1.1]hexane, 6-oxabicyclo[3.1.1]heptane, 3-oxabicyclo[3.1.1]heptane, 2-oxabicyclo[3.1.1]heptane, 3,6-dioxabicyclo[3.1.1]heptane, 2-oxabicyclo[2.2.1]heptane, 2,5-dioxabicyclo[2.2.1]heptane, 2,5-dioxabicyclo[2.2.2]octane, 2-oxabicyclo[4.1.1]octane, 3-oxabicyclo[4.1.1]octane, 3,7-dioxabicyclo[4.1.1]octane, 3-oxabicyclo[3.2.1]octane, 2-oxabicyclo[3.2.1]octane, 6-oxabicyclo[3.2.1]octane, 8-oxabicyclo[3.2.1]octane, 3,8-dioxabicyclo[3.2.1]octane, 3,6-dioxabicyclo[3.2.1]octane, 2-oxabicyclo[3.2.2]nonane, 6-oxabicyclo[3.2.2]nonane, 3,6-dioxabicyclo[3.2.2]nonane, 2,6-dioxabicyclo[3.2.2]nonane, 2-oxabicyclo[5.1.1]nonane, 3-oxabicyclo[5.1.1]nonane, 3,8-dioxabicyclo[5.1.1]nonane, 4,8-dioxabicyclo[5.1.1]nonane, 4-oxabicyclo[5.1.1]nonane, 2,6-dioxabicyclo[5.1.1]nonane, 2,5-dioxabicyclo[5.1.1]nonane, 9-oxabicyclo[3.3.1]nonane, 3,9-dioxabicyclo[3.3.1]nonane, 3,7-dioxabicyclo[3.3.1]nonane, 3,7,9-trioxabicyclo[3.3.1]nonane, 2,7-dioxabicyclo[3.3.1]nonane, 2,6-dioxabicyclo[3.3.1]nonane, 7-oxabicyclo[4.2.1]nonane, 2-oxabicyclo[4.2.1]nonane, 3-oxabicyclo[4.2.1]nonane, 3,9-dioxabicyclo[4.2.1]nonane, 3,8-dioxabicyclo[4.2.1]nonane, 2,5-dioxabicyclo[4.2.1]nonane, 3,7-dioxabicyclo[4.2.1]nonane, 2,7-dioxabicyclo[4.2.1]nonane, 8-oxabicyclo[5.2.1]decane, 2-oxabicyclo[5.2.1]decane, 3-oxabicyclo[5.2.1]decane, 4-oxabicyclo[5.2.1]decane, 3,10-dioxabicyclo[5.2.1]decane, 3,9-dioxabicyclo[5.2.1]decane, 2,8-dioxabicyclo[5.2.1]decane, 4,10-dioxabicyclo[5.2.1]decane, 4,8-dioxabicyclo[5.2.1]decane, 2,6-dioxabicyclo[5.2.1]decane, 2,5-dioxabicyclo[5.2.1]decane, 3,8-dioxabicyclo[5.2.1]decane, 2,5,8-trioxabicyclo[5.2.1]decane, 2-oxabicyclo[4.2.2]decane, 3-oxabicyclo[4.2.2]decane, 7,9-dioxabicyclo[4.2.2]decane, 3,8-dioxabicyclo[4.2.2]decane, 2,5-dioxabicyclo[4.2.2]decane, 3,7-dioxabicyclo[4.2.2]decane, 2,7-dioxabicyclo[4.2.2]decane, 3-oxabicyclo[3.3.2]decane, 9-oxabicyclo[3.3.2]decane, 2,10-dioxabicyclo[3.3.2]decane, 3,9-dioxabicyclo[3.3.2]decane, 2,6-dioxabicyclo[3.3.2]decane, 2,7-dioxabicyclo[3.3.2]decane, 3,7-dioxabicyclo[3.3.2]decane, 2-thiabicyclo[1.1.1]pentane, 5-thiabicyclo[2.1.1]hexane, 7-thiabicyclo[2.2.1]heptane, 7-thiabicyclo[4.1.1]octane, 8-thiabicyclo[5.1.1]nonane, 9-thiabicyclo[4.2.1]nonane, 3-thiabicyclo[3.3.1]nonane, 2-thiabicyclo[2.2.2]octane, 3-thiabicyclo[3.2.2]nonane, 10-thiabicyclo[5.2.1]decane, 7-thiabicyclo[4.2.2]decane, 2-thiabicyclo[3.3.2]decane, 2-thiabicyclo[2.1.1]hexane, 6-thiabicyclo[3.1.1]heptane, 3-thiabicyclo[3.1.1]heptane, 2-thiabicyclo[3.1.1]heptane, 6-oxa-3-thiabicyclo[3.1.1]heptane, 2-thiabicyclo[2.2.1]heptane, 2-oxa-5-thiabicyclo[2.2.1]heptane, 2-oxa-5-thiabicyclo[2.2.2]octane, 2-thiabicyclo[4.1.1]octane, 3-thiabicyclo[4.1.1]octane, 3-oxa-7-thiabicyclo[4.1.1]octane, 3-thiabicyclo[3.2.1]octane, 2-thiabicyclo[3.2.1]octane, 6-thiabicyclo[3.2.1]octane, 8-thiabicyclo[3.2.1]octane, 3-oxa-8-thiabicyclo[3.2.1]octane, 3-oxa-6-thiabicyclo[3.2.1]octane, 2-thiabicyclo[3.2.2]nonane, 6-thiabicyclo[3.2.2]nonane, 6-oxa-3-thiabicyclo[3.2.2]nonane, 6-oxa-2-thiabicyclo[3.2.2]nonane, 2-thiabicyclo[5.1.1]nonane, 3-thiabicyclo[5.1.1]nonane, 8-oxa-3-thiabicyclo[5.1.1]nonane, 8-oxa-4-thiabicyclo[5.1.1]nonane, 4-thiabicyclo[5.1.1]nonane, 2-oxa-6-thiabicyclo[5.1.1]nonane, 5-oxa-2-thiabicyclo[5.1.1]nonane, 9-thiabicyclo[3.3.1]nonane, 9-oxa-3-thiabicyclo[3.3.1]nonane, 3,7-dioxabicyclo[3.3.1]nonane, 3,9-dioxa-7-thiabicyclo[3.3.1]nonane, 2-oxa-7-thiabicyclo[3.3.1]nonane, 2-oxa-6-thiabicyclo[3.3.1]nonane, 7-thiabicyclo[4.2.1]nonane, 2-thiabicyclo[4.2.1]nonane, 3-thiabicyclo[4.2.1]nonane, 9-oxa-3-thiabicyclo[4.2.1]nonane, 8-oxa-3-thiabicyclo[4.2.1]nonane, 2-oxa-5-thiabicyclo[4.2.1]nonane, 7-oxa-3-thiabicyclo[4.2.1]nonane, 7-oxa-2-thiabicyclo[4.2.1]nonane, 8-thiabicyclo[5.2.1]decane, 2-thiabicyclo[5.2.1]decane, 3-thiabicyclo[5.2.1]decane, 4-thiabicyclo[5.2.1]decane, 10-oxa-3-thiabicyclo[5.2.1]decane, 9-oxa-3-thiabicyclo[5.2.1]decane, 8-oxa-2-thiabicyclo[5.2.1]decane, 10-oxa-4-thiabicyclo[5.2.1]decane, 8-oxa-4-thiabicyclo[5.2.1]decane, 2-oxa-6-thiabicyclo[5.2.1]decane, 2-oxa-5-thiabicyclo[5.2.1]decane, 8-oxa-3-thiabicyclo[5.2.1]decane, 2,5-dioxa-8-thiabicyclo[5.2.1]decane, 2-thiabicyclo[4.2.2]decane, 3-thiabicyclo[4.2.2]decane, 7-oxa-9-thiabicyclo[4.2.2]decane, 3-oxa-8-thiabicyclo[4.2.2]decane, 2-oxa-5-thiabicyclo[4.2.2]decane, 3-oxa-7-thiabicyclo[4.2.2]decane, 2-oxa-7-thiabicyclo[4.2.2]decane, 3-thiabicyclo[3.3.2]decane, 9-thiabicyclo[3.3.2]decane, 10-oxa-2-thiabicyclo[3.3.2]decane, 9-oxa-3-thiabicyclo[3.3.2]decane, 2-oxa-6-thiabicyclo[3.3.2]decane, 2-oxa-7-thiabicyclo[3.3.2]decane, 3-oxa-7-thiabicyclo[3.3.2]decane, 3-oxa-6-thiabicyclo[3.1.1]heptane, 7-oxa-3-thiabicyclo[4.1.1]octane, 8-oxa-3-thiabicyclo[3.2.1]octane, 6-oxa-3-thiabicyclo[3.2.1]octane, 3-oxa-6-thiabicyclo[3.2.2]nonane, 2-oxa-6-thiabicyclo[3.2.2]nonane, 3-oxa-8-thiabicyclo[5.1.1]nonane, 4-oxa-8-thiabicyclo[5.1.1]nonane, 2-oxa-5-thiabicyclo[5.1.1]nonane, 3-oxa-9-thiabicyclo[3.3.1]nonane, 3,7-dioxa-9-thiabicyclo[3.3.1]nonane, 7-oxa-2-thiabicyclo[3.3.1]nonane, 3-oxa-9-thiabicyclo[4.2.1]nonane, 3-oxa-8-thiabicyclo[4.2.1]nonane, 3-oxa-7-thiabicyclo[4.2.1]nonane, 2-oxa-7-thiabicyclo[4.2.1]nonane, 3-oxa-10-thiabicyclo[5.2.1]decane, 3-oxa-9-thiabicyclo[5.2.1]decane, 2-oxa-8-thiabicyclo[5.2.1]decane, 4-oxa-10-thiabicyclo[5.2.1]decane, 4-oxa-8-thiabicyclo[5.2.1]decane, 3-oxa-8-thiabicyclo[5.2.1]decane, 5-oxa-2-thiabicyclo[5.2.1]decane, 2,8-dioxa-5-thiabicyclo[5.2.1]decane, 5,8-dioxa-2-thiabicyclo[5.2.1]decane, 7-oxa-9-thiabicyclo[4.2.2]decane, 8-oxa-3-thiabicyclo[4.2.2]decane, 7-oxa-3-thiabicyclo[4.2.2]decane, 7-oxa-2-thiabicyclo[4.2.2]decane, 2-oxa-10-thiabicyclo[3.3.2]decane, 3-oxa-9-thiabicyclo[3.3.2]decane, 7-oxa-2-thiabicyclo[3.3.2]decane, 2-azabicyclo[1.1.1]pentane, 5-azabicyclo[2.1.1]hexane, 7-azabicyclo[2.2.1]heptane, 7-azabicyclo[4.1.1]octane, 8-azabicyclo[5.1.1]nonane, 9-azabicyclo[4.2.1]nonane, 3-azabicyclo[3.3.1]nonane, 2-azabicyclo[2.2.2]octane, 3-azabicyclo[3.2.2]nonane, 10-azabicyclo[5.2.1]decane, 7-azabicyclo[4.2.2]decane, 2-azabicyclo[3.3.2]decane, 2-azabicyclo[2.1.1]hexane, 6-azabicyclo[3.1.1]heptane, 3-azabicyclo[3.1.1]heptane, 2-azabicyclo[3.1.1]heptane, 3,6-diazabicyclo[3.1.1]heptane, 2-azabicyclo[2.2.1]heptane, 2,5-diazabicyclo[2.2.1]heptane, 2,5-diazabicyclo[2.2.2]octane, 2-azabicyclo[4.1.1]octane, 3-azabicyclo[4.1.1]octane, 3,7-diazabicyclo[4.1.1]octane, 3-azabicyclo[3.2.1]octane, 2-azabicyclo[3.2.1]octane, 6-azabicyclo[3.2.1]octane, 8-azabicyclo[3.2.1]octane, 3,8-diazabicyclo[3.2.1]octane, 3,6-diazabicyclo[3.2.1]octane, 2-azabicyclo[3.2.2]nonane, 6-azabicyclo[3.2.2]nonane, 3,6-diazabicyclo[3.2.2]nonane, 2,6-diazabicyclo[3.2.2]nonane, 2-azabicyclo[5.1.1]nonane, 3-azabicyclo[5.1.1]nonane, 3,8-diazabicyclo[5.1.1]nonane, 4,8-diazabicyclo[5.1.1]nonane, 4-azabicyclo[5.1.1]nonane, 2,6-diazabicyclo[5.1.1]nonane, 2,5-diazabicyclo[5.1.1]nonane, 9-azabicyclo[3.3.1]nonane, 3,9-diazabicyclo[3.3.1]nonane, 3,7-diazabicyclo[3.3.1]nonane, 3,7,9-triazabicyclo[3.3.1]nonane, 2,7-diazabicyclo[3.3.1]nonane, 2,6-diazabicyclo[3.3.1]nonane, 7-azabicyclo[4.2.1]nonane, 2-azabicyclo[4.2.1]nonane, 3-azabicyclo[4.2.1]nonane, 3,9-diazabicyclo[4.2.1]nonane, 3,8-diazabicyclo[4.2.1]nonane, 2,5-diazabicyclo[4.2.1]nonane, 3,7-diazabicyclo[4.2.1]nonane, 2,7-diazabicyclo[4.2.1]nonane, 8-azabicyclo[5.2.1]decane, 2-azabicyclo[5.2.1]decane, 3-azabicyclo[5.2.1]decane, 4-azabicyclo[5.2.1]decane, 3,10-diazabicyclo[5.2.1]decane, 3,9-diazabicyclo[5.2.1]decane, 2,8-diazabicyclo[5.2.1]decane, 4,10-diazabicyclo[5.2.1]decane, 4,8-diazabicyclo[5.2.1]decane, 2,6-diazabicyclo[5.2.1]decane, 2,5-diazabicyclo[5.2.1]decane, 3,8-diazabicyclo[5.2.1]decane, 2,5,8-triazabicyclo[5.2.1]decane, 2-azabicyclo[4.2.2]decane, 3-azabicyclo[4.2.2]decane, 7,9-diazabicyclo[4.2.2]decane, 3,8-diazabicyclo[4.2.2]decane, 2,5-diazabicyclo[4.2.2]decane, 3,7-diazabicyclo[4.2.2]decane, 2,7-diazabicyclo[4.2.2]decane, 3-azabicyclo[3.3.2]decane, (1s,5s)-9-azabicyclo[3.3.2]decane, (1S,5S)-2,10-diazabicyclo[3.3.2]decane, (1S,5R)-3,9-diazabicyclo[3.3.2]decane, 2,6-diazabicyclo[3.3.2]decane, 2,7-diazabicyclo[3.3.2]decane, 3,7-diazabicyclo[3.3.2]decane, 1-azabicyclo[1.1.1]pentane, 1-azabicyclo[2.1.1]hexane, 1-azabicyclo[2.2.1]heptane, 1-azabicyclo[3.1.1]heptane, 1,4-diazabicyclo[3.1.1]heptane, quinuclidine, 1,4-diazabicyclo[2.2.2]octane, 1-azabicyclo[4.1.1]octane, 1,4-diazabicyclo[4.1.1]octane, 1,5-diazabicyclo[4.1.1]octane, 1-azabicyclo[3.2.1]octane, 1,4-diazabicyclo[3.2.1]octane, 1-azabicyclo[3.2.2]nonane, 1,5-diazabicyclo[3.2.2]nonane, 1,4-diazabicyclo[3.2.2]nonane, 1,4-diazabicyclo[5.1.1]nonane, 1,5-diazabicyclo[5.1.1]nonane, 1,6-diazabicyclo[5.1.1]nonane, 1-azabicyclo[5.1.1]nonane, 1-azabicyclo[3.3.1]nonane, 1,4-diazabicyclo[3.3.1]nonane, 1-azabicyclo[4.2.1]nonane, 1,4-diazabicyclo[4.2.1]nonane, 1,5-diazabicyclo[4.2.1]nonane, 1-azabicyclo[5.2.1]decane, 1,4-diazabicyclo[5.2.1]decane, 1,5-diazabicyclo[5.2.1]decane, 1,6-diazabicyclo[5.2.1]decane, 1-azabicyclo[4.2.2]decane, 1,6-diazabicyclo[4.2.2]decane, 1,4-diazabicyclo[4.2.2]decane, 1,5-diazabicyclo[4.2.2]decane, 1-azabicyclo[3.3.2]decane, 1,4-diazabicyclo[3.3.2]decane, 1,5-diazabicyclo[3.3.2]decane, 1,4-diazabicyclo[3.3.2]decane, 6-oxa-3-azabicyclo[3.1.1]heptane, 2-oxa-5-azabicyclo[2.2.1]heptane, 2-oxa-5-azabicyclo[2.2.2]octane, 7-oxa-3-azabicyclo[4.1.1]octane, 8-oxa-3-azabicyclo[3.2.1]octane, 6-oxa-3-azabicyclo[3.2.1]octane, 3-oxa-6-azabicyclo[3.2.2]nonane, 2-oxa-6-azabicyclo[3.2.2]nonane, 8-oxa-3-azabicyclo[5.1.1]nonane, 8-oxa-4-azabicyclo[5.1.1]nonane, 2-oxa-6-azabicyclo[5.1.1]nonane, 2-oxa-5-azabicyclo[5.1.1]nonane, 3-oxa-9-azabicyclo[3.3.1]nonane, 3-oxa-7-azabicyclo[3.3.1]nonane, 3-oxa-7,9-diazabicyclo[3.3.1]nonane, 7-oxa-2-azabicyclo[3.3.1]nonane, 2-oxa-6-azabicyclo[3.3.1]nonane, 3-oxa-9-azabicyclo[4.2.1]nonane, 3-oxa-8-azabicyclo[4.2.1]nonane, 2-oxa-5-azabicyclo[4.2.1]nonane, 3-oxa-7-azabicyclo[4.2.1]nonane, 2-oxa-7-azabicyclo[4.2.1]nonane, 10-oxa-3-azabicyclo[5.2.1]decane, 3-oxa-9-azabicyclo[5.2.1]decane, 8-oxa-2-azabicyclo[5.2.1]decane, 4-oxa-10-azabicyclo[5.2.1]decane, 8-oxa-4-azabicyclo[5.2.1]decane, 2-oxa-5-azabicyclo[5.2.1]decane, 8-oxa-3-azabicyclo[5.2.1]decane, 2-oxa-5,8-diazabicyclo[5.2.1]decane, 7-oxa-9-azabicyclo[4.2.2]decane, 8-oxa-3-azabicyclo[4.2.2]decane, 2-oxa-5-azabicyclo[4.2.2]decane, 7-oxa-3-azabicyclo[4.2.2]decane, 7-oxa-2-azabicyclo[4.2.2]decane, (1S,5S)-2-oxa-10-azabicyclo[3.3.2]decane, (1S,5R)-9-oxa-3-azabicyclo[3.3.2]decane, 2-oxa-6-azabicyclo[3.3.2]decane, 7-oxa-2-azabicyclo[3.3.2]decane, 3-oxa-7-azabicyclo[3.3.2]decane, 4-oxa-1-azabicyclo[3.1.1]heptane, 4-oxa-1-azabicyclo[4.1.1]octane, 5-oxa-1-azabicyclo[4.1.1]octane, 4-oxa-1-azabicyclo[3.2.1]octane, 4-oxa-1-azabicyclo[3.2.2]nonane, 4-oxa-1-azabicyclo[5.1.1]nonane, 5-oxa-1-azabicyclo[5.1.1]nonane, 6-oxa-1-azabicyclo[5.1.1]nonane, 4-oxa-1-azabicyclo[3.3.1]nonane, 4-oxa-1-azabicyclo[4.2.1]nonane, 5-oxa-1-azabicyclo[4.2.1]nonane, 4-oxa-1-azabicyclo[5.2.1]decane, 5-oxa-1-azabicyclo[5.2.1]decane, 6-oxa-1-azabicyclo[5.2.1]decane, 4-oxa-1-azabicyclo[4.2.2]decane, 5-oxa-1-azabicyclo[4.2.2]decane, 4-oxa-1-azabicyclo[3.3.2]decane, 3-oxa-6-azabicyclo[3.1.1]heptane, 3-oxa-7-azabicyclo[4.1.1]octane, 3-oxa-8-azabicyclo[3.2.1]octane, 3-oxa-6-azabicyclo[3.2.1]octane, 6-oxa-3-azabicyclo[3.2.2]nonane, 6-oxa-2-azabicyclo[3.2.2]nonane, 3-oxa-8-azabicyclo[5.1.1]nonane, 4-oxa-8-azabicyclo[5.1.1]nonane, 5-oxa-2-azabicyclo[5.1.1]nonane, 9-oxa-3-azabicyclo[3.3.1]nonane, 9-oxa-3,7-diazabicyclo[3.3.1]nonane, 2-oxa-7-azabicyclo[3.3.1]nonane, 9-oxa-3-azabicyclo[4.2.1]nonane, 8-oxa-3-azabicyclo[4.2.1]nonane, 7-oxa-3-azabicyclo[4.2.1]nonane, 7-oxa-2-azabicyclo[4.2.1]nonane, 3-oxa-10-azabicyclo[5.2.1]decane, 9-oxa-3-azabicyclo[5.2.1]decane, 10-oxa-4-azabicyclo[5.2.1]decane, 2-oxa-8-azabicyclo[5.2.1]decane, 4-oxa-8-azabicyclo[5.2.1]decane, 2-oxa-6-azabicyclo[5.2.1]decane, 5-oxa-2-azabicyclo[5.2.1]decane, 3-oxa-8-azabicyclo[5.2.1]decane, 8-oxa-2,5-diazabicyclo[5.2.1]decane, 5-oxa-2,8-diazabicyclo[5.2.1]decane, 2,5-dioxa-8-azabicyclo[5.2.1]decane, 2,8-dioxa-5-azabicyclo[5.2.1]decane, 5,8-dioxa-2-azabicyclo[5.2.1]decane, 3-oxa-8-azabicyclo[4.2.2]decane, 3-oxa-7-azabicyclo[4.2.2]decane, 2-oxa-7-azabicyclo[4.2.2]decane, (1S,5S)-10-oxa-2-azabicyclo[3.3.2]decane, (1S,5S)-3-oxa-9-azabicyclo[3.3.2]decane, 2-oxa-7-azabicyclo[3.3.2]decane, 6-thia-3-azabicyclo[3.1.1]heptane, 2-thia-5-azabicyclo[2.2.1]heptane, 2-thia-5-azabicyclo[2.2.2]octane, 7-thia-3-azabicyclo[4.1.1]octane, 8-thia-3-azabicyclo[3.2.1]octane, 6-thia-3-azabicyclo[3.2.1]octane, 3-thia-6-azabicyclo[3.2.2]nonane, 2-thia-6-azabicyclo[3.2.2]nonane, 8-thia-3-azabicyclo[5.1.1]nonane, 8-thia-4-azabicyclo[5.1.1]nonane, 2-thia-6-azabicyclo[5.1.1]nonane, 2-thia-5-azabicyclo[5.1.1]nonane, 3-thia-9-azabicyclo[3.3.1]nonane, 3-thia-7-azabicyclo[3.3.1]nonane, 3-thia-7,9-diazabicyclo[3.3.1]nonane, 7-thia-2-azabicyclo[3.3.1]nonane, 2-thia-6-azabicyclo[3.3.1]nonane, 3-thia-9-azabicyclo[4.2.1]nonane, 3-thia-8-azabicyclo[4.2.1]nonane, 2-thia-5-azabicyclo[4.2.1]nonane, 3-thia-7-azabicyclo[4.2.1]nonane, 2-thia-7-azabicyclo[4.2.1]nonane, 10-thia-3-azabicyclo[5.2.1]decane, 3-thia-9-azabicyclo[5.2.1]decane, 8-thia-2-azabicyclo[5.2.1]decane, 4-thia-10-azabicyclo[5.2.1]decane, 8-thia-4-azabicyclo[5.2.1]decane, 2-thia-5-azabicyclo[5.2.1]decane, 8-thia-3-azabicyclo[5.2.1]decane, 2-thia-5,8-diazabicyclo[5.2.1]decane, 7-thia-9-azabicyclo[4.2.2]decane, 8-thia-3-azabicyclo[4.2.2]decane, 2-thia-5-azabicyclo[4.2.2]decane, 7-thia-3-azabicyclo[4.2.2]decane, 7-thia-2-azabicyclo[4.2.2]decane, (1S,5S)-2-thia-10-azabicyclo[3.3.2]decane, (1S,5R)-9-thia-3-azabicyclo[3.3.2]decane, 2-thia-6-azabicyclo[3.3.2]decane, 7-thia-2-azabicyclo[3.3.2]decane, 3-thia-7-azabicyclo[3.3.2]decane, 4-thia-1-azabicyclo[3.1.1]heptane, 4-thia-1-azabicyclo[4.1.1]octane, 5-thia-1-azabicyclo[4.1.1]octane, 4-thia-1-azabicyclo[3.2.1]octane, 4-thia-1-azabicyclo[3.2.2]nonane, 4-thia-1-azabicyclo[5.1.1]nonane, 5-thia-1-azabicyclo[5.1.1]nonane, 6-thia-1-azabicyclo[5.1.1]nonane, 4-thia-1-azabicyclo[3.3.1]nonane, 4-thia-1-azabicyclo[4.2.1]nonane, 5-thia-1-azabicyclo[4.2.1]nonane, 4-thia-1-azabicyclo[5.2.1]decane, 5-thia-1-azabicyclo[5.2.1]decane, 6-thia-1-azabicyclo[5.2.1]decane, 4-thia-1-azabicyclo[4.2.2]decane, 5-thia-1-azabicyclo[4.2.2]decane, 4-thia-1-azabicyclo[3.3.2]decane, 3-thia-6-azabicyclo[3.1.1]heptane, 3-thia-7-azabicyclo[4.1.1]octane, 3-thia-8-azabicyclo[3.2.1]octane, 3-thia-6-azabicyclo[3.2.1]octane, 6-thia-3-azabicyclo[3.2.2]nonane, 6-thia-2-azabicyclo[3.2.2]nonane, 3-thia-8-azabicyclo[5.1.1]nonane, 4-thia-8-azabicyclo[5.1.1]nonane, 5-thia-2-azabicyclo[5.1.1]nonane, 9-thia-3-azabicyclo[3.3.1]nonane, 9-thia-3,7-diazabicyclo[3.3.1]nonane, 2-thia-7-azabicyclo[3.3.1]nonane, 9-thia-3-azabicyclo[4.2.1]nonane, 8-thia-3-azabicyclo[4.2.1]nonane, 7-thia-3-azabicyclo[4.2.1]nonane, 7-thia-2-azabicyclo[4.2.1]nonane, 3-thia-10-azabicyclo[5.2.1]decane, 9-thia-3-azabicyclo[5.2.1]decane, 10-thia-4-azabicyclo[5.2.1]decane, 2-thia-8-azabicyclo[5.2.1]decane, 4-thia-8-azabicyclo[5.2.1]decane, 2-thia-6-azabicyclo[5.2.1]decane, 5-thia-2-azabicyclo[5.2.1]decane, 3-thia-8-azabicyclo[5.2.1]decane, 8-thia-2,5-diazabicyclo[5.2.1]decane, 5-thia-2,8-diazabicyclo[5.2.1]decane, 5-oxa-2-thia-8-azabicyclo[5.2.1]decane, 2-oxa-8-thia-5-azabicyclo[5.2.1]decane, 8-oxa-5-thia-2-azabicyclo[5.2.1]decane, 3-thia-8-azabicyclo[4.2.2]decane, 3-thia-7-azabicyclo[4.2.2]decane, 2-thia-7-azabicyclo[4.2.2]decane, (1S,5S)-10-thia-2-azabicyclo[3.3.2]decane, (1S,5S)-3-thia-9-azabicyclo[3.3.2]decane, or 2-thia-7-azabicyclo[3.3.2]decane, wherein the heterocyclic ring is optionally substituted with 0, 1, 2, 3, 4, or 5 R⁵ groups as valency permits.

In some embodiments of Formula (I), (I′), (II), or (II′), R¹ or R² is a substituted or unsubstituted 3-10 membered carbocycle that includes, but is not limited to, one of the following: cyclopropane, cyclobutane, cyclopentane, cyclohexane, cycloheptane, cyclooctane, bicyclo[1.1.0]butane, bicyclo[2.1.0]pentane, bicyclo[2.2.0]hexane, bicyclo[3.1.0]hexane, bicyclo[3.2.0]heptane, octahydropentalene, bicyclo[4.1.0]heptane, bicyclo[4.2.0]octane, octahydro-1H-indene, decahydronaphthalene, bicyclo[5.1.0]octane, bicyclo[5.2.0]nonane, decahydroazulene, decahydro-1H-benzo[7]annulene, dodecahydroheptalene, bicyclo[6.1.0]nonane, bicyclo[6.2.0]decane, decahydro-1H-cyclopenta[8]annulene, dodecahydrobenzo[8]annulene, dodecahydro-1H-cyclohepta[8]annulene, tetradecahydrooctalene, bicyclo[1.1.1]pentane, bicyclo[2.1.1]hexane, bicyclo[3.1.1]heptane, bicyclo[2.2.1]heptane, bicyclo[4.1.1]octane, bicyclo[5.1.1]nonane, bicyclo[3.2.1]octane, bicyclo[4.2.1]nonane, bicyclo[3.3.1]nonane, bicyclo[2.2.2]octane, bicyclo[3.2.2]nonane, bicyclo[5.2.1]decane, bicyclo[4.2.2]decane, bicyclo[3.3.2]decane, spiro[2.3]hexane, spiro[2.4]heptane, spiro[2.5]octane, spiro[2.6]nonane, spiro[2.7]decane, spiro[2.2]pentane, spiro[3.3]heptane, spiro[3.4]octane, spiro[3.5]nonane, spiro[3.6]decane, spiro[3.7]undecane, spiro[4.4]nonane, spiro[4.5]decane, spiro[4.6]undecane, spiro[4.7]dodecane, spiro[5.5]undecane, spiro[5.6]dodecane, spiro[5.7]tridecane, spiro[6.6]tridecane, spiro[6.7]tetradecane, or spiro[7.7]pentadecane, wherein the carbocyclic ring is optionally substituted with 0, 1, 2, 3, 4, or 5 R⁵ groups as valency permits.

In some embodiments of Formula (I), (I′), (II), or (II′), at least one R⁵ is halogen. In some embodiments of Formula (I), (I′), (II), or (II′), at least one R⁵ is substituted or unsubstituted C₁₋₆ alkyl. In some embodiments of Formula (I), (I′), (II), or (II′), at least one R⁵ is branched or unbranched C₁₋₆ alkyl. In some embodiments of Formula (I), (I′), (II), or (II′), at least one R⁵ is substituted or unsubstituted methyl. In some embodiments of Formula (I), (I′), (II), or (II′), at least one R⁵ is substituted or unsubstituted ethyl. In some embodiments of Formula (I), (I′), (II), or (II′), at least one R⁵ is substituted or unsubstituted propyl. In some embodiments of Formula (I), (I′), (II), or (II′), at least one R⁵ is substituted or unsubstituted butyl. In some embodiments of Formula (I), (I′), (II), or (II′), at least one R⁵ is substituted or unsubstituted pentyl. In some embodiments of Formula (I), (I′), (II), or (II′), at least one R⁵ is substituted or unsubstituted C₁₋₆ haloalkyl. In some embodiments of Formula (I), (I′), (II), or (II′), at least one R⁵ is substituted or unsubstituted fluoroalkyl. In some embodiments of Formula (I), (I′), (II), or (II′), at least one R⁵ is trifluoromethyl. In some embodiments of Formula (I), (I′), (II), or (II′), at least one R⁵ is substituted or unsubstituted trifluoroethyl. In some embodiments of Formula (I), (I′), (II), or (II′), at least one R⁵ is substituted or unsubstituted C₂₋₆ alkenyl. In some embodiments of Formula (I), (I′), (II), or (II′), at least one R⁵ is substituted or unsubstituted vinyl. In some embodiments of Formula (I), (I′), (II), or (II′), at least one R⁵ is substituted or unsubstituted allyl. In some embodiments of Formula (I), (I′), (II), or (II′), at least one R⁵ is substituted or unsubstituted propenyl. In some embodiments of Formula (I), (I′), (II), or (II′), at least one R⁵ is substituted or unsubstituted butenyl. In some embodiments of Formula (I), (I′), (II), or (II′), at least one R⁵ is substituted or unsubstituted C₂₋₆ alkynyl. In some embodiments of Formula (I), (I′), (II), or (II′), at least one R⁵ is substituted or unsubstituted acetylenyl. In some embodiments of Formula (I), (I′), (II), or (II′), at least one R⁵ is substituted or unsubstituted propargyl. In some embodiments of Formula (I), (I′), (II), or (II′), at least one R⁵ is substituted or unsubstituted butynyl. In some embodiments of Formula (I), (I′), (II), or (II′), at least one R⁵ is substituted or unsubstituted C₁₋₆ alkoxy. In some embodiments of Formula (I), (I′), (II), or (II′), at least one R⁵ is substituted or unsubstituted methoxy. In some embodiments of Formula (I), (I′), (II), or (II′), at least one R⁵ is substituted or unsubstituted ethoxy. In some embodiments of Formula (I), (I′), (II), or (II′), at least one R⁵ is substituted or unsubstituted propoxy. In some embodiments of Formula (I), (I′), (II), or (II′), at least one R⁵ is substituted or unsubstituted butoxy. In some embodiments of Formula (I), (I′), (II), or (II′), at least one R⁵ is substituted or unsubstituted benzyloxy. In some embodiments of Formula (I), (I′), (II), or (II′), at least one R⁵ is trifluoromethoxy. In some embodiments of Formula (I), (I′), (II), or (II′), at least one R⁵ is substituted or unsubstituted C₆₋₁₀ aryl. In some embodiments of Formula (I), (I′), (II), or (II′), at least one R⁵ is substituted or unsubstituted 5-10 membered heteroaryl. In some embodiments of Formula (I), (I′), (II), or (II′), at least one R⁵ is substituted or unsubstituted 4-10 membered heterocyclyl. In some embodiments of Formula (I), (I′), (II), or (II′), at least one R⁵ is substituted or unsubstituted 3-10 membered carbocyclyl. In some embodiments of Formula (I), (I′), (II), or (II′), at least one R⁵ is nitro. In some embodiments of Formula (I), (I′), (II), or (II′), at least one R⁵ is cyano. In some embodiments of Formula (I), (I′), (II), or (II′), at least one R⁵ is acyl. In some embodiments of Formula (I), (I′), (II), or (II′), at least one R⁵ is —NH₂. In some embodiments of Formula (I), (I′), (II), or (II′), at least one R⁵ is —NHR⁷. In some embodiments of Formula (I), (I′), (II), or (II′), at least one R⁵ is —N(R⁷)₂. In some embodiments of Formula (I), (I′), (II), or (II′), at least one R⁵ is —OH. In some embodiments of Formula (I), (I′), (II), or (II′), at least one R⁵ is —SH. In some embodiments of Formula (I), (I′), (II), or (II′), at least one R⁵ is —SO₂R⁷. In some embodiments of Formula (I), (I′), (II), or (II′), at least one R⁵ is —SOR⁷. In some embodiments of Formula (I), (I′), (II), or (II′), at least one R⁵ is —SO₂NR⁷₂. In some embodiments of Formula (I), (I′), (II), or (II′), at least one R⁵ is —SR⁷. In some embodiments of Formula (I), (I′), (II), or (II′), at least one R⁵ is —OR⁷.

In some embodiments of Formula (I), (I′), (II), or (II′), at least one R⁷ is hydrogen. In some embodiments of Formula (I), (I′), (II), or (II′), at least one R⁷ is acyl. In some embodiments of Formula (I), (I′), (II), or (II′), at least one R⁷ is substituted or unsubstituted C₁₋₁₀ alkyl. In some embodiments of Formula (I), (I′), (II), or (II′), at least one R⁷ is substituted or unsubstituted C₆₋₁₀ aryl. In some embodiments of Formula (I), (I′), (II), or (II′), at least one R⁷ is independently substituted or unsubstituted 5-10 membered heteroaryl.

In some embodiments of Formula (I), (I′), (II), or (II′), at least one R⁷ is substituted or unsubstituted 4-10 membered heterocyclyl. In some embodiments of Formula (I), (I′), (II), or (II′), at least one R⁷ is substituted or unsubstituted 3-10 membered carbocyclyl. In some embodiments of Formula (I), (I′), (II), or (II′), two R⁷ groups are taken together with any intervening atoms to form an substituted or unsubstituted heterocyclic ring.

Groups R¹¹, R¹², and R¹³

In some embodiments of Formula (I), (I′), (II), or (II′), R¹¹ is hydrogen. In some embodiments of Formula (I), (I′), (II), or (II′), R¹¹ is substituted or unsubstituted C₁₋₆ alkyl. In some embodiments of Formula (I), (I′), (II), or (II′), R¹¹ is substituted or unsubstituted C₁₋₃ alkyl. In some embodiments of Formula (I), (I′), (II), or (II′), R¹¹ is branched or unbranched substituted or unsubstituted C₁₋₆ alkyl. In some embodiments of Formula (I), (I′), (II), or (II′), R¹¹ is substituted or unsubstituted methyl. In some embodiments of Formula (I), (I′), (II), or (II′), R¹¹ is substituted or unsubstituted ethyl. In some embodiments of Formula (I), (I′), (II), or (II′), R¹¹ is substituted or unsubstituted propyl. In some embodiments of Formula (I), (I′), (II), or (II′), R¹¹ is substituted or unsubstituted butyl. In some embodiments of Formula (I), (I′), (II), or (II′), R¹¹ is substituted or unsubstituted pentyl. In some embodiments of Formula (I), (I′), (II), or (II′), R¹¹ is substituted or unsubstituted C₁₋₆ haloalkyl. In some embodiments of Formula (I), (I′), (II), or (II′), R¹¹ is substituted or unsubstituted fluoroalkyl. In some embodiments of Formula (I), (I′), (II), or (II′), R¹¹ is trifluoromethyl. In some embodiments of Formula (I), (I′), (II), or (II′), R¹¹ is substituted or unsubstituted trifluoroethyl. In some embodiments of Formula (I), (I′), (II), or (II′), R¹¹ is substituted or unsubstituted C₂₋₆ alkenyl. In some embodiments of Formula (I), (I′), (II), or (II′), R¹¹ is substituted or unsubstituted vinyl. In some embodiments of Formula (I), (I′), (II), or (II′), R¹¹ is substituted or unsubstituted allyl. In some embodiments of Formula (I), (I′), (II), or (II′), R¹¹ is substituted or unsubstituted propenyl. In some embodiments of Formula (I), (I′), (II), or (II′), R¹¹ is substituted or unsubstituted butenyl. In some embodiments of Formula (I), (I′), (II), or (II′), R¹¹ is substituted or unsubstituted C₂₋₆ alkynyl. In some embodiments of Formula (I), (I′), (II), or (II′), R¹¹ is substituted or unsubstituted acetylenyl. In some embodiments of Formula (I), (I′), (II), or (II′), R¹¹ is substituted or unsubstituted propargyl. In some embodiments of Formula (I), (I′), (II), or (II′), R¹¹ is substituted or unsubstituted butynyl. In some embodiments of Formula (I), (I′), (II), or (II′), R¹¹ is substituted or unsubstituted C₁₋₆ alkoxy. In some embodiments of Formula (I), (I′), (II), or (II′), R¹¹ is CH₂—OMe. In some embodiments of Formula (I), (I′), (II), or (II′), R¹¹ is CH₂—OEt. In some embodiments of Formula (I), (I′), (II), or (II′), R¹¹CH₂—OPr. In some embodiments of Formula (I), (I′), (II), or (II′), R¹¹ is CH₂—OCF₃. In some embodiments of Formula (I), (I′), (II), or (II′), R¹¹ is substituted or unsubstituted CH₂—OR⁷. In some embodiments of Formula (I), (I′), (II), or (II′), R¹¹ is substituted or unsubstituted C₆₋₁₀ aryl. In some embodiments of Formula (I), (I′), (II), or (II′), R¹¹ is substituted or unsubstituted 5-10 membered heteroaryl. In some embodiments of Formula (I), (I′), (II), or (II′), R¹¹ is substituted or unsubstituted 4-10 membered heterocyclyl. In some embodiments of Formula (I), (I′), (II), or (II′), R¹¹ is substituted or unsubstituted 3-10 membered carbocyclyl. In some embodiments of Formula (I), (I′), (II), or (II′), R¹¹ is acyl. In some embodiments of Formula (I), (I′), (II), or (II′), R¹¹ is a substituted or unsubstituted amide. In some embodiments of Formula (I), (I′), (II), or (II′), R¹ is a substituted or unsubstituted ester. In some embodiments of Formula (I), (I′), (II), or (II′), R¹¹ is —OH. In some embodiments of Formula (I), (I′), (II), or (II′), R¹¹ is —SO₂R⁷. In some embodiments of Formula (I), (I′), (II), or (II′), R¹¹ is —SOR⁷. In some embodiments of Formula (I), (I′), (II), or (II′), R¹ is —SO₂NR⁷₂. In some embodiments of Formula (I), (I′), (II), or (II′), R¹¹ is —OSO₂R⁷. In some embodiments of Formula (I), (I′), (II), or (II′), R¹¹ is —OSOR⁷. In some embodiments of Formula (I), (I′), (II), or (II′), R¹¹ is —OSO₂NR⁷₂. In some embodiments of Formula (I), (I′), (II), or (II′), R¹¹ is —OSO₂OR⁷. In some embodiments of Formula (I), (I′), (II), or (II′), R¹¹ is —O(SO)OR⁷. In some embodiments of Formula (I), (I′), (II), or (II′), R¹¹ is —PO(OR⁷)₂. In some embodiments of Formula (I), (I′), (II), or (II′), R¹¹ is —PO(NR⁷)₂. In some embodiments of Formula (I), (I′), (II), or (II′), R¹¹ is —PO(NR⁷)(OR⁷). In some embodiments of Formula (I), (I′), (II), or (II′), R¹¹ is —OPO(OR⁷)₂. In some embodiments of Formula (I), (I′), (II), or (II′), R¹¹ is —OPO(NR⁷)₂. In some embodiments of Formula (I), (I′), (II), or (II′), R¹¹ is —OPO(NR⁷)(OR⁷). In some embodiments of Formula (I), (I′), (II), or (II′), R¹¹ is a nitrogen protecting group. In some embodiments of Formula (I), (I′), (II), or (II′), R¹¹ is —OR⁷. In some embodiments of Formula (I), (I′), (II), or (II′), R¹¹ is optionally substituted with 0, 1, 2, 3, 4, or 5 occurrences of R⁵. In some embodiments of Formula (I), (I′), (II), or (II′), R¹¹ is optionally substituted with 5 occurrences of R⁵. In some embodiments of Formula (I), (I′), (II), or (II′), R¹¹ is optionally substituted with 4 occurrences of R⁵. In some embodiments of Formula (I), (I′), (II), or (II′), R¹¹ is optionally substituted with 3 occurrences of R⁵. In some embodiments of Formula (I), (I′), (II), or (II′), R¹¹ is optionally substituted with 2 occurrences of R⁵. In some embodiments of Formula (I), (I′), (II), or (II′), R¹¹ is optionally substituted with 1 occurrence of R⁵. In some embodiments of Formula (I), (I′), (II), or (II′), R¹¹ is optionally substituted with 0 occurrences of R⁵.

In some embodiments of Formula (I), (I′), (II), or (II′), R¹² is hydrogen. In some embodiments of Formula (I), (I′), (II), or (II′), R¹² is substituted or unsubstituted C₁₋₆ alkyl. In some embodiments of Formula (I), (I′), (II), or (II′), R¹² is substituted or unsubstituted C₁₋₃ alkyl. In some embodiments of Formula (I), (I′), (II), or (II′), R¹² is branched or unbranched substituted or unsubstituted C₁₋₆ alkyl. In some embodiments of Formula (I), (I′), (II), or (II′), R¹² is substituted or unsubstituted methyl. In some embodiments of Formula (I), (I′), (II), or (II′), R¹² is substituted or unsubstituted ethyl. In some embodiments of Formula (I), (I′), (II), or (II′), R¹² is substituted or unsubstituted propyl. In some embodiments of Formula (I), (I′), (II), or (II′), R¹² is substituted or unsubstituted butyl. In some embodiments of Formula (I), (I′), (II), or (II′), R¹² is substituted or unsubstituted pentyl. In some embodiments of Formula (I), (I′), (II), or (II′), R¹² is substituted or unsubstituted C₁₋₆ haloalkyl. In some embodiments of Formula (I), (I′), (II), or (II′), R¹² is substituted or unsubstituted fluoroalkyl. In some embodiments of Formula (I), (I′), (II), or (II′), R¹² is trifluoromethyl. In some embodiments of Formula (I), (I′), (II), or (II′), R¹² is substituted or unsubstituted trifluoroethyl. In some embodiments of Formula (I), (I′), (II), or (II′), R¹² is substituted or unsubstituted C₂₋₆ alkenyl. In some embodiments of Formula (I), (I′), (II), or (II′), R¹² is substituted or unsubstituted vinyl. In some embodiments of Formula (I), (I′), (II), or (II′), R¹² is substituted or unsubstituted allyl. In some embodiments of Formula (I), (I′), (II), or (II′), R¹² is substituted or unsubstituted propenyl. In some embodiments of Formula (I), (I′), (II), or (II′), R¹² is substituted or unsubstituted butenyl. In some embodiments of Formula (I), (I′), (II), or (II′), R¹² is substituted or unsubstituted C₂₋₆ alkynyl. In some embodiments of Formula (I), (I′), (II), or (II′), R¹² is substituted or unsubstituted acetylenyl. In some embodiments of Formula (I), (I′), (II), or (II′), R¹² is substituted or unsubstituted propargyl. In some embodiments of Formula (I), (I′), (II), or (II′), R¹² is substituted or unsubstituted butynyl. In some embodiments of Formula (I), (I′), (II), or (II′), R¹² is substituted or unsubstituted C₁₋₆ alkoxy. In some embodiments of Formula (I), (I′), (II), or (II′), R¹² is CH₂—OMe. In some embodiments of Formula (I), (I′), (II), or (II′), R¹² is CH₂-OEt. In some embodiments of Formula (I), (I′), (II), or (II′), R¹² is CH₂—OPr. In some embodiments of Formula (I), (I′), (II), or (II′), R¹² is CH₂—OCF₃. In some embodiments of Formula (I), (I′), (II), or (II′), R¹² is substituted or unsubstituted CH₂—OR⁷. In some embodiments of Formula (I), (I′), (II), or (II′), R¹² is substituted or unsubstituted C₆₋₁₀ aryl. In some embodiments of Formula (I), (I′), (II), or (II′), R¹² is substituted or unsubstituted 5-10 membered heteroaryl. In some embodiments of Formula (I), (I′), (II), or (II′), R¹² is substituted or unsubstituted 4-10 membered heterocyclyl. In some embodiments of Formula (I), (I′), (II), or (II′), R¹² is substituted or unsubstituted 3-10 membered carbocyclyl. In some embodiments of Formula (I), (I′), (II), or (II′), R¹² is acyl. In some embodiments of Formula (I), (I′), (II), or (II′), R¹² is a substituted or unsubstituted amide. In some embodiments of Formula (I), (I′), (II), or (II′), R¹² is a substituted or unsubstituted ester. In some embodiments of Formula (I), (I′), (II), or (II′), R¹² is —OH. In some embodiments of Formula (I), (I′), (II), or (II′), R¹² is —SO₂R⁷. In some embodiments of Formula (I), (I′), (II), or (II′), R¹² is —SOR⁷. In some embodiments of Formula (I), (I′), (II), or (II′), R¹² is —SO₂NR⁷₂. In some embodiments of Formula (I), (I′), (II), or (II′), R¹² is —OSO₂R⁷. In some embodiments of Formula (I), (I′), (II), or (II′), R¹² is —OSOR⁷. In some embodiments of Formula (I), (I′), (II), or (II′), R¹² is —OSO₂NR⁷₂. In some embodiments of Formula (I), (I′), (II), or (II′), R¹² is —OSO₂OR⁷. In some embodiments of Formula (I), (I′), (II), or (II′), R¹² is —O(SO)OR⁷. In some embodiments of Formula (I), (I′), (II), or (II′), R¹² is —PO(OR⁷)₂. In some embodiments of Formula (I), (I′), (II), or (II′), R¹² is —PO(NR⁷)₂. In some embodiments of Formula (I), (I′), (II), or (II′), R¹² is —PO(NR⁷)(OR⁷). In some embodiments of Formula (I), (I′), (II), or (II′), R¹² is —OPO(OR⁷)₂. In some embodiments of Formula (I), (I′), (II), or (II′), R¹² is —OPO(NR⁷)₂. In some embodiments of Formula (I), (I′), (II), or (II′), R¹² is —OPO(NR⁷)(OR⁷). In some embodiments of Formula (I), (I′), (II), or (II′), R¹² is a nitrogen protecting group. In some embodiments of Formula (I), (I′), (II), or (II′), R¹² is —OR⁷.

In some embodiments of Formula (I), (I′), (II), or (II′), R¹³ is hydrogen. In some embodiments of Formula (I), (I′), (II), or (II′), R¹³ is substituted or unsubstituted C₁₋₆ alkyl. In some embodiments of Formula (I), (I′), (II), or (II′), R¹³ is substituted or unsubstituted C₁₋₃ alkyl. In some embodiments of Formula (I), (I′), (II), or (II′), R¹³ is branched or unbranched substituted or unsubstituted C₁₋₆ alkyl. In some embodiments of Formula (I), (I′), (II), or (II′), R¹³ is substituted or unsubstituted methyl. In some embodiments of Formula (I), (I′), (II), or (II′), R¹³ is substituted or unsubstituted ethyl. In some embodiments of Formula (I), (I′), (II), or (II′), R¹³ is substituted or unsubstituted propyl. In some embodiments of Formula (I), (I′), (II), or (II′), R¹³ is substituted or unsubstituted butyl. In some embodiments of Formula (I), (I′), (II), or (II′), R¹³ is substituted or unsubstituted pentyl. In some embodiments of Formula (I), (I′), (II), or (II′), R¹³ is substituted or unsubstituted C₁₋₆ haloalkyl. In some embodiments of Formula (I), (I′), (II), or (II′), R¹³ is substituted or unsubstituted fluoroalkyl. In some embodiments of Formula (I), (I′), (II), or (II′), R¹³ is trifluoromethyl. In some embodiments of Formula (I), (I′), (II), or (II′), R¹³ is substituted or unsubstituted trifluoroethyl. In some embodiments of Formula (I), (I′), (II), or (II′), R¹³ is substituted or unsubstituted C₂₋₆ alkenyl. In some embodiments of Formula (I), (I′), (II), or (II′), R¹³ is substituted or unsubstituted vinyl. In some embodiments of Formula (I), (I′), (II), or (II′), R¹³ is substituted or unsubstituted allyl. In some embodiments of Formula (I), (I′), (II), or (II′), R¹³ is substituted or unsubstituted propenyl. In some embodiments of Formula (I), (I′), (II), or (II′), R¹³ is substituted or unsubstituted butenyl. In some embodiments of Formula (I), (I′), (II), or (II′), R¹³ is substituted or unsubstituted C₂₋₆ alkynyl. In some embodiments of Formula (I), (I′), (II), or (II′), R¹³ is substituted or unsubstituted acetylenyl. In some embodiments of Formula (I), (I′), (II), or (II′), R¹³ is substituted or unsubstituted propargyl. In some embodiments of Formula (I), (I′), (II), or (II′), R¹³ is substituted or unsubstituted butynyl. In some embodiments of Formula (I), (I′), (II), or (II′), R¹³ is substituted or unsubstituted C₁₋₆ alkoxy. In some embodiments of Formula (I), (I′), (II), or (II′), R¹³ is CH₂—OMe. In some embodiments of Formula (I), (I′), (II), or (II′), R¹³ is CH₂—OEt. In some embodiments of Formula (I), (I′), (II), or (II′), R¹³ is CH₂—OPr. In some embodiments of Formula (I), (I′), (II), or (II′), R¹³ is CH₂—OCF₃. In some embodiments of Formula (I), (I′), (II), or (II′), R¹³ is substituted or unsubstituted CH₂—OR⁷. In some embodiments of Formula (I), (I′), (II), or (II′), R¹³ is substituted or unsubstituted C₆₋₁₀ aryl. In some embodiments of Formula (I), (I′), (II), or (II′), R¹³ is substituted or unsubstituted 5-10 membered heteroaryl. In some embodiments of Formula (I), (I′), (II), or (II′), R¹³ is substituted or unsubstituted 4-10 membered heterocyclyl. In some embodiments of Formula (I), (I′), (II), or (II′), R¹³ is substituted or unsubstituted 3-10 membered carbocyclyl. In some embodiments of Formula (I), (I′), (II), or (II′), R¹³ is acyl. In some embodiments of Formula (I), (I′), (II), or (II′), R¹³ is a substituted or unsubstituted amide. In some embodiments of Formula (I), (I′), (II), or (II′), R¹³ is a substituted or unsubstituted ester. In some embodiments of Formula (I), (I′), (II), or (II′), R¹³ is —OH. In some embodiments of Formula (I), (I′), (II), or (II′), R¹³ is —SO₂R⁷. In some embodiments of Formula (I), (I′), (II), or (II′), R¹³ is —SOR⁷. In some embodiments of Formula (I), (I′), (II), or (II′), R¹³ is —SO₂NR⁷₂. In some embodiments of Formula (I), (I′), (II), or (II′), R¹³ is —OSO₂R⁷. In some embodiments of Formula (I), (I′), (II), or (II′), R¹³ is —OSOR⁷. In some embodiments of Formula (I), (I′), (II), or (II′), R¹³ is —OSO₂NR⁷₂. In some embodiments of Formula (I), (I′), (II), or (II′), R¹³ is —OSO₂OR⁷. In some embodiments of Formula (I), (I′), (II), or (II′), R¹³ is —O(SO)OR⁷. In some embodiments of Formula (I), (I′), (II), or (II′), R¹³ is —PO(OR⁷)₂. In some embodiments of Formula (I), (I′), (II), or (II′), R¹³ is —PO(NR⁷)₂. In some embodiments of Formula (I), (I′), (II), or (II′), R¹³ is —PO(NR⁷)(OR⁷). In some embodiments of Formula (I), (I′), (II), or (II′), R¹³ is —OPO(OR⁷)₂. In some embodiments of Formula (I), (I′), (II), or (II′), R¹³ is —OPO(NR⁷)₂. In some embodiments of Formula (I), (I′), (II), or (II′), R¹³ is —OPO(NR⁷)(OR⁷). In some embodiments of Formula (I), (I′), (II), or (II′), R¹³ is a nitrogen protecting group. In some embodiments of Formula (I), (I′), (II), or (II′), R¹³ is —OR⁷.

In some embodiments of Formula (I), (I′), (II), or (II′), R¹² is hydrogen; and R¹³ is hydrogen. In some embodiments of Formula (I), (I′), (II), or (II′), R¹² is substituted or unsubstituted C₁₋₆ alkyl; and R¹³ is hydrogen. In some embodiments of Formula (I), (I′), (II), or (II′), R¹² is substituted or unsubstituted C₁₋₃ alkyl; and R¹³ is hydrogen. In some embodiments of Formula (I), (I′), (II), or (II′), R¹² is branched or unbranched substituted or unsubstituted C₁₋₆ alkyl; and R¹³ is hydrogen. In some embodiments of Formula (I), (I′), (II), or (II′), R¹² is substituted or unsubstituted methyl; and R¹³ is hydrogen. In some embodiments of Formula (I), (I′), (II), or (II′), R¹² is substituted or unsubstituted ethyl; and R¹³ is hydrogen. In some embodiments of Formula (I), (I′), (II), or (II′), R¹² is substituted or unsubstituted propyl; and R¹³ is hydrogen. In some embodiments of Formula (I), (I′), (II), or (II′), R¹² is substituted or unsubstituted butyl; and R¹³ is hydrogen. In some embodiments of Formula (I), (I′), (II), or (II′), R¹² is substituted or unsubstituted pentyl; and R¹³ is hydrogen. In some embodiments of Formula (I), (I′), (II), or (II′), R¹² is substituted or unsubstituted C₁₋₆ haloalkyl; and R¹³ is hydrogen. In some embodiments of Formula (I), (I′), (II), or (II′), R¹² is substituted or unsubstituted fluoroalkyl; and R¹³ is hydrogen. In some embodiments of Formula (I), (I′), (II), or (II′), R¹² is trifluoromethyl; and R¹³ is hydrogen. In some embodiments of Formula (I), (I′), (II), or (II′), R¹² is substituted or unsubstituted trifluoroethyl; and R¹³ is hydrogen. In some embodiments of Formula (I), (I′), (II), or (II′), R¹² is substituted or unsubstituted C₂₋₆ alkenyl; and R¹³ is hydrogen. In some embodiments of Formula (I), (I′), (II), or (II′), R¹² is substituted or unsubstituted C₂₋₆ alkenyl; and R¹³ is hydrogen. In some embodiments of Formula (I), (I′), (II), or (II′), R¹² is substituted or unsubstituted vinyl; and R¹³ is hydrogen. In some embodiments of Formula (I), (I′), (II), or (II′), R¹² is substituted or unsubstituted allyl; and R¹³ is hydrogen. In some embodiments of Formula (I), (I′), (II), or (II′), R¹² is substituted or unsubstituted propenyl; and R¹³ is hydrogen. In some embodiments of Formula (I), (I′), (II), or (II′), R¹² is substituted or unsubstituted butenyl; and R¹³ is hydrogen. In some embodiments of Formula (I), (I′), (II), or (II′), R¹² is substituted or unsubstituted C₂₋₆ alkynyl; and R¹³ is hydrogen. In some embodiments of Formula (I), (I′), (II), or (II′), R¹² is substituted or unsubstituted acetylenyl; and R¹³ is hydrogen. In some embodiments of Formula (I), (I′), (II), or (II′), R¹² is substituted or unsubstituted propargyl; and R¹³ is hydrogen. In some embodiments of Formula (I), (I′), (II), or (II′), R¹² is substituted or unsubstituted butynyl; and R¹³ is hydrogen. In some embodiments of Formula (I), (I′), (II), or (II′), R¹² is substituted or unsubstituted C₁₋₆ alkoxy; and R¹³ is hydrogen. In some embodiments of Formula (I), (I′), (II), or (II′), R¹² is CH₂—OMe; and R¹³ is hydrogen. In some embodiments of Formula (I), (I′), (II), or (II′), R¹² is CH₂—OEt; and R¹³ is hydrogen. In some embodiments of Formula (I), (I′), (II), or (II′), (II′), R¹²CH₂—OPr; and R¹³ is hydrogen. In some embodiments of Formula (I), (I′), (II), or (II′), R¹² is CH₂—OCF₃; and R¹³ is hydrogen. In some embodiments of Formula (I), (I′), (II), or (II′), R¹² is substituted or unsubstituted CH₂—OR⁷; and R¹³ is hydrogen. In some embodiments of Formula (I), (I′), (II), or (II′), R¹² is substituted or unsubstituted C₆₋₁₀ aryl; and R¹³ is hydrogen. In some embodiments of Formula (I), (I′), (II), or (II′), R¹² is substituted or unsubstituted 5-10 membered heteroaryl; and R¹³ is hydrogen. In some embodiments of Formula (I), (I′), (II), or (II′), R¹² is substituted or unsubstituted 4-10 membered heterocyclyl; and R¹³ is hydrogen. In some embodiments of Formula (I), (I′), (II), or (II′), R¹² is substituted or unsubstituted 3-10 membered carbocyclyl; and R¹³ is hydrogen. In some embodiments of Formula (I), (I′), (II), or (II′), R¹² is acyl; and R¹³ is hydrogen. In some embodiments of Formula (I), (I′), (II), or (II′), R¹² is a substituted or unsubstituted amide; and R¹³ is hydrogen. In some embodiments of Formula (I), (I′), (II), or (II′), R¹² is a substituted or unsubstituted ester; and R¹³ is hydrogen. In some embodiments of Formula (I), (I′), (II), or (II′), R¹² is —OH; and R¹³ is hydrogen. In some embodiments of Formula (I), (I′), (II), or (II′), R¹² is —SO₂R⁷; and R¹³ is hydrogen. In some embodiments of Formula (I), (I′), (II), or (II′), R¹² is —SOR⁷; and R¹³ is hydrogen. In some embodiments of Formula (I), (I′), (II), or (II′), R¹² is —SO₂NR⁷₂; and R¹³ is hydrogen. In some embodiments of Formula (I), (I′), (II), or (II′), R¹² is —OSO₂R⁷; and R¹³ is hydrogen. In some embodiments of Formula (I), (I′), (II), or (II′), R¹² is —OSOR⁷; and R¹³ is hydrogen. In some embodiments of Formula (I), (I′), (II), or (II′), R¹² is —OSO₂NR⁷₂; and R¹³ is hydrogen. In some embodiments of Formula (I), (I′), (II), or (II′), R¹² is —OSO₂OR⁷; and R¹³ is hydrogen. In some embodiments of Formula (I), (I′), (II), or (II′), R¹² is —O(SO)OR⁷; and R¹³ is hydrogen. In some embodiments of Formula (I), (I′), (II), or (II′), R¹² is —PO(OR⁷)₂; and R¹³ is hydrogen. In some embodiments of Formula (I), (I′), (II), or (II′), R¹² is —PO(NR⁷)₂; and R¹³ is hydrogen. In some embodiments of Formula (I), (I′), (II), or (II′), R¹² is —PO(NR⁷)(OR⁷)₂, and R¹³ is hydrogen. In some embodiments of Formula (I), (I′), (II), or (II′), R¹² is —OPO(OR⁷)₂; and R¹³ is hydrogen. In some embodiments of Formula (I), (I′), (II), or (II′), R¹² is —OPO(NR⁷)₂; and R¹³ is hydrogen. In some embodiments of Formula (I), (I′), (II), or (II′), R¹² is —OPO(NR⁷)(OR⁷); and R¹³ is hydrogen. In some embodiments of Formula (I), (I′), (II), or (II′), R¹² is a nitrogen protecting group; and R¹³ is hydrogen. In some embodiments of Formula (I), (I′), (II), or (II′), R¹² is —OR⁷; and R¹³ is hydrogen.

In some embodiments of Formula (I), (I′), (II), or (II′), R¹² is hydrogen; and R¹³ is substituted or unsubstituted C₁₋₆ alkyl. In some embodiments of Formula (I), (I′), (II), or (II′), R¹² is substituted or unsubstituted C₁₋₆ alkyl; and R¹³ is substituted or unsubstituted C₁₋₆alkyl. In some embodiments of Formula (I), (I′), (II), or (II′), R¹² is substituted or unsubstituted C₁₋₃ alkyl; and R¹³ is substituted or unsubstituted C₁₋₆ alkyl. In some embodiments of Formula (I), (I′), (II), or (II′), R¹² is branched or unbranched substituted or unsubstituted C₁₋₆ alkyl; and R¹³ is substituted or unsubstituted C₁₋₆ alkyl. In some embodiments of Formula (I), (I′), (II), or (II′), R¹² is substituted or unsubstituted methyl; and R¹³ is substituted or unsubstituted C₁₋₆ alkyl. In some embodiments of Formula (I), (I′), (II), or (II′), R¹² is substituted or unsubstituted ethyl; and R¹³ is substituted or unsubstituted C₁₋₆alkyl. In some embodiments of Formula (I), (I′), (II), or (II′), R¹² is substituted or unsubstituted propyl; and R¹³ is substituted or unsubstituted C₁₋₆ alkyl. In some embodiments of Formula (I), (I′), (II), or (II′), R¹² is substituted or unsubstituted butyl; and R¹³ is C₁₋₆ substituted or unsubstituted alkyl. In some embodiments of Formula (I), (I′), (II), or (II′), R¹² is substituted or unsubstituted pentyl; and R¹³ is substituted or unsubstituted C₁₋₆ alkyl. In some embodiments of Formula (I), (I′), (II), or (II′), R¹² is substituted or unsubstituted C₁₋₆haloalkyl; and R¹³ is substituted or unsubstituted C₁₋₆ alkyl. In some embodiments of Formula (I), (I′), (II), or (II′), R¹² is substituted or unsubstituted fluoroalkyl; and R¹³ is substituted or unsubstituted C₁₋₆ alkyl. In some embodiments of Formula (I), (I′), (II), or (II′), R¹² is trifluoromethyl; and R¹³ is substituted or unsubstituted C₁₋₆ alkyl. In some embodiments of Formula (I), (I′), (II), or (II′), R¹² is substituted or unsubstituted trifluoroethyl; and R¹³ is substituted or unsubstituted C₁₋₆ alkyl. In some embodiments of Formula (I), (I′), (II), or (II′), R¹² is substituted or unsubstituted C₂₋₆ alkenyl; and R¹³ is substituted or unsubstituted C₁₋₆ alkyl. In some embodiments of Formula (I), (I′), (II), or (II′), R¹² is substituted or unsubstituted C₂₋₆ alkenyl; and R¹³ is substituted or unsubstituted C₁₋₆ alkyl. In some embodiments of Formula (I), (I′), (II), or (II′), R¹² is substituted or unsubstituted vinyl; and R¹³ is substituted or unsubstituted C₁₋₆ alkyl. In some embodiments of Formula (I), (I′), (II), or (II′), R¹² is substituted or unsubstituted allyl; and R¹³ is substituted or unsubstituted C₁₋₆ alkyl. In some embodiments of Formula (I), (I′), (II), or (II′), R¹² is substituted or unsubstituted propenyl; and R¹³ is substituted or unsubstituted C₁₋₆ alkyl. In some embodiments of Formula (I), (I′), (II), or (II′), R¹² is substituted or unsubstituted butenyl; and R¹³ is substituted or unsubstituted C₁₋₆ alkyl. In some embodiments of Formula (I), (I′), (II), or (II′), R¹² is substituted or unsubstituted C₂₋₆ alkynyl; and R¹³ is substituted or unsubstituted C₁₋₆ alkyl. In some embodiments of Formula (I), (I′), (II), or (II′), R¹² is substituted or unsubstituted acetylenyl; and R¹³ is substituted or unsubstituted C₁₋₆ alkyl. In some embodiments of Formula (I), (I′), (II), or (II′), R¹² is substituted or unsubstituted propargyl; and R¹³ substituted or unsubstituted is C₁₋₆ alkyl. In some embodiments of Formula (I), (I′), (II), or (II′), R¹² is substituted or unsubstituted butynyl; and R¹³ is substituted or unsubstituted C₁₋₆ alkyl. In some embodiments of Formula (I), (I′), (II), or (II′), R¹² is C₁₋₆ substituted or unsubstituted alkoxy; and R¹³ is substituted or unsubstituted C₁₋₆ alkyl. In some embodiments of Formula (I), (I′), (II), or (II′), R¹² is CH₂—OMe; and R¹³ is substituted or unsubstituted C₁₋₆ alkyl. In some embodiments of Formula (I), (I′), (II), or (II′), R¹² is CH₂—OEt; and R¹³ is substituted or unsubstituted C₁₋₆ alkyl. In some embodiments of Formula (I), (I′), (II), or (II′), R¹²CH₂—OPr; and R¹³ is substituted or unsubstituted C₁₋₆ alkyl. In some embodiments of Formula (I), (I′), (II), or (II′), R¹² is CH₂—OCF₃; and R¹³ is substituted or unsubstituted C₁₋₆ alkyl. In some embodiments of Formula (I), (I′), (II), or (II′), R¹² is substituted or unsubstituted CH₂—OR⁷; and R¹³ is substituted or unsubstituted C₁₋₆ alkyl. In some embodiments of Formula (I), (I′), (II), or (II′), R¹² is substituted or unsubstituted C₆₋₁₀ aryl; and R¹³ is substituted or unsubstituted C₁₋₆ alkyl. In some embodiments of Formula (I), (I′), (II), or (II′), R¹² is substituted or unsubstituted 5-10 membered heteroaryl; and R¹³ is substituted or unsubstituted C₁₋₆ alkyl. In some embodiments of Formula (I), (I′), (II), or (II′), R¹² is substituted or unsubstituted 4-10 membered heterocyclyl; and R¹³ is substituted or unsubstituted C₁₋₆ alkyl. In some embodiments of Formula (I), (I′), (II), or (II′), R¹² is substituted or unsubstituted 3-10 membered carbocyclyl; and R¹ is substituted or unsubstituted C₁₋₆ alkyl. In some embodiments of Formula (I), (I′), (II), or (II′), R¹² is acyl; and R¹³ is substituted or unsubstituted C₁₋₆ alkyl. In some embodiments of Formula (I), (I′), (II), or (II′), R¹² is a substituted or unsubstituted amide; and R¹ is substituted or unsubstituted C₁₋₆ alkyl. In some embodiments of Formula (I), (I′), (II), or (II′), R¹² is a substituted or unsubstituted ester; and R¹³ is substituted or unsubstituted C₁₋₆ alkyl. In some embodiments of Formula (I), (I′), (II), or (II′), R¹² is —OH; and R¹³ is substituted or unsubstituted C₁₋₆ alkyl. In some embodiments of Formula (I), (I′), (II), or (II′), R¹² is —SO₂R⁷; and R¹³ is substituted or unsubstituted C₁₋₆alkyl. In some embodiments of Formula (I), (I′), (II), or (II′), R¹² is —SOR⁷; and R¹³ is substituted or unsubstituted C₁₋₆ alkyl. In some embodiments of Formula (I), (I′), (II), or (II′), R¹² is —SO₂NR⁷₂; and R¹³ is substituted or unsubstituted C₁₋₆ alkyl. In some embodiments of Formula (I), (I′), (II), or (II′), R¹² is —OSO₂R⁷; and R¹³ is substituted or unsubstituted C₁₋₆ alkyl. In some embodiments of Formula (I), (I′), (II), or (II′), R¹² is —OSOR⁷; and R¹³ is substituted or unsubstituted C₁₋₆ alkyl. In some embodiments of Formula (I), (I′), (II), or (II′), R¹² is —OSO₂NR⁷₂; and R¹³ is substituted or unsubstituted C₁₋₆ alkyl. In some embodiments of Formula (I), (I′), (II), or (II′), R¹² is —OSO₂OR⁷; and R¹³ is substituted or unsubstituted C₁₋₆ alkyl. In some embodiments of Formula (I), (I′), (II), or (II′), R¹² is —O(SO)OR⁷; and R¹³ is substituted or unsubstituted C₁₋₆ alkyl. In some embodiments of Formula (I), (I′), (II), or (II′), R¹² is —PO(OR⁷)₂; and R¹³ is substituted or unsubstituted C₁₋₆ alkyl. In some embodiments of Formula (I), (I′), (II), or (II′), R¹² is —PO(NR⁷)₂; and R¹³ is substituted or unsubstituted C₁₋₆ alkyl. In some embodiments of Formula (I), (I′), (II), or (II′), R¹² is —PO(NR⁷)(OR⁷); and R¹³ is substituted or unsubstituted C₁₋₆ alkyl. In some embodiments of Formula (I), (I′), (II), or (II′), R¹² is —OPO(OR⁷)₂; and R¹³ is substituted or unsubstituted C₁₋₆ alkyl. In some embodiments of Formula (I), (I′), (II), or (II′), R¹² is —OPO(NR⁷)₂; and R¹³ is substituted or unsubstituted C₁₋₆ alkyl. In some embodiments of Formula (I), (I′), (II), or (II′), R¹² is —OPO(NR⁷)(OR⁷); and R¹³ is substituted or unsubstituted C₁₋₆ alkyl. In some embodiments of Formula (I), (I′), (II), or (II′), R¹² is a nitrogen protecting group; and R¹ is substituted or unsubstituted C₁₋₆ alkyl. In some embodiments of Formula (I), (I′), (II), or (II′), R¹² is —OR⁷; and R¹³ is substituted or unsubstituted C₁₋₆ alkyl.

In some embodiments of Formula (I), (I′), (II), or (II′), R¹² is hydrogen; and R¹³ is substituted or unsubstituted C₁₋₃ alkyl. In some embodiments of Formula (I), (I′), (II), or (II′), R¹² is substituted or unsubstituted C₁₋₆ alkyl; and R¹³ is C₁₋₃ alkyl. In some embodiments of Formula (I), (I′), (II), or (II′), R¹² is substituted or unsubstituted C₁₋₃ alkyl; and R¹³ is substituted or unsubstituted C₁₋₃ alkyl. In some embodiments of Formula (I), (I′), (II), or (II′), R¹² is branched or unbranched substituted or unsubstituted C₁₋₆ alkyl; and R¹³ is substituted or unsubstituted C₁₋₃ alkyl. In some embodiments of Formula (I), (I′), (II), or (II′), R¹² is substituted or unsubstituted methyl; and R¹³ is substituted or unsubstituted C₁₋₃alkyl. In some embodiments of Formula (I), (I′), (II), or (II′), R¹² is substituted or unsubstituted ethyl; and R¹³ is substituted or unsubstituted C₁₋₃ alkyl. In some embodiments of Formula (I), (I′), (II), or (II′), R¹² is substituted or unsubstituted propyl; and R¹³ is substituted or unsubstituted C₁₋₃ alkyl. In some embodiments of Formula (I), (I′), (II), or (II′), R¹² is substituted or unsubstituted butyl; and R¹³ is substituted or unsubstituted C₁₋₃alkyl. In some embodiments of Formula (I), (I′), (II), or (II′), R¹² is substituted or unsubstituted pentyl; and R¹³ is substituted or unsubstituted C₁₋₃ alkyl. In some embodiments of Formula (I), (I′), (II), or (II′), R¹² is substituted or unsubstituted C₁₋₆ haloalkyl; and R¹³ is substituted or unsubstituted C₁₋₃ alkyl. In some embodiments of Formula (I), (I′), (II), or (II′), R¹² is substituted or unsubstituted fluoroalkyl; and R¹³ is substituted or unsubstituted C₁₋₃ alkyl. In some embodiments of Formula (I), (I′), (II), or (II′), R¹² is trifluoromethyl; and R¹³ is substituted or unsubstituted C₁₋₃ alkyl. In some embodiments of Formula (I), (I′), (II), or (II′), R¹² is substituted or unsubstituted trifluoroethyl; and R¹³ is substituted or unsubstituted C₁₋₃ alkyl. In some embodiments of Formula (I), (I′), (II), or (II′), R¹² is substituted or unsubstituted C₂₋₆ alkenyl; and R¹³ is substituted or unsubstituted C₁₋₃ alkyl. In some embodiments of Formula (I), (I′), (II), or (II′), R¹² is substituted or unsubstituted C₂₋₆ alkenyl; and R¹³ is substituted or unsubstituted C₁₋₃ alkyl. In some embodiments of Formula (I), (I′), (II), or (II′), R¹² is substituted or unsubstituted vinyl; and R¹³ is substituted or unsubstituted C₁₋₃ alkyl. In some embodiments of Formula (I), (I′), (II), or (II′), R¹² is substituted or unsubstituted allyl; and R¹³ is substituted or unsubstituted C₁₋₃ alkyl. In some embodiments of Formula (I), (I′), (II), or (II′), R¹² is substituted or unsubstituted propenyl; and R¹³ is substituted or unsubstituted C₁₋₃ alkyl. In some embodiments of Formula (I), (I′), (II), or (II′), R¹² is substituted or unsubstituted butenyl; and R¹³ is substituted or unsubstituted C₁₋₃ alkyl. In some embodiments of Formula (I), (I′), (II), or (II′), R¹² is substituted or unsubstituted C₂₋₆ alkynyl; and R¹³ is substituted or unsubstituted C₁₋₃ alkyl. In some embodiments of Formula (I), (I′), (II), or (II′), R¹² is substituted or unsubstituted acetylenyl; and R¹³ is substituted or unsubstituted C₁₋₃ alkyl. In some embodiments of Formula (I), (I′), (II), or (II′), R¹² is substituted or unsubstituted propargyl; and R¹³ is substituted or unsubstituted C₁₋₃ alkyl. In some embodiments of Formula (I), (I′), (II), or (II′), R¹² is substituted or unsubstituted butynyl; and R¹³ is substituted or unsubstituted C₁₋₃alkyl. In some embodiments of Formula (I), (I′), (II), or (II′), R¹² is substituted or unsubstituted C₁₋₆ alkoxy; and R¹³ is substituted or unsubstituted C₁₋₃ alkyl. In some embodiments of Formula (I), (I′), (II), or (II′), R¹² is CH₂—OMe; and R¹³ is substituted or unsubstituted C₁₋₃ alkyl. In some embodiments of Formula (I), (I′), (II), or (II′), R¹² is CH₂-OEt; and R¹³ is substituted or unsubstituted C₁₋₃ alkyl. In some embodiments of Formula (I), (I′), (II), or (II′), R¹²CH₂—OPr; and R¹³ is substituted or unsubstituted C₁₋₃ alkyl. In some embodiments of Formula (I), (I′), (II), or (II′), R¹² is CH₂—OCF₃; and R¹³ is substituted or unsubstituted C₁₋₃ alkyl. In some embodiments of Formula (I), (I′), (II), or (II′), R¹² is substituted or unsubstituted CH₂—OR⁷; and R¹³ is substituted or unsubstituted C₁₋₃ alkyl. In some embodiments of Formula (I), (I′), (II), or (II′), R¹² is substituted or unsubstituted C₆₋₁₀ aryl; and R¹³ is substituted or unsubstituted C₁₋₃ alkyl. In some embodiments of Formula (I), (I′), (II), or (II′), R¹² is substituted or unsubstituted 5-10 membered heteroaryl; and R¹³ is substituted or unsubstituted C₁₋₃ alkyl. In some embodiments of Formula (I), (I′), (II), or (II′), R¹² is substituted or unsubstituted 4-10 membered heterocyclyl; and R¹³ is substituted or unsubstituted C₁₋₃ alkyl. In some embodiments of Formula (I), (I′), (II), or (II′), R¹² is substituted or unsubstituted 3-10 membered carbocyclyl; and R¹³ is substituted or unsubstituted C₁₋₃ alkyl. In some embodiments of Formula (I), (I′), (II), or (II′), R¹² is acyl; and R¹³ is substituted or unsubstituted C₁₋₃ alkyl. In some embodiments of Formula (I), (I′), (II), or (II′), R¹² is a substituted or unsubstituted amide; and R¹³ is substituted or unsubstituted C₁₋₃ alkyl. In some embodiments of Formula (I), (I′), (II), or (II′), R¹² is a substituted or unsubstituted ester; and R¹³ is substituted or unsubstituted C₁₋₃ alkyl. In some embodiments of Formula (I), (I′), (II), or (II′), R¹² is —OH; and R¹³ is substituted or unsubstituted C₁₋₃ alkyl. In some embodiments of Formula (I), (I′), (II), or (II′), R¹² is —SO₂R⁷; and R¹³ is substituted or unsubstituted C₁₋₃ alkyl. In some embodiments of Formula (I), (I′), (II), or (II′), R¹² is —SOR⁷; and R¹³ is substituted or unsubstituted C₁₋₃ alkyl. In some embodiments of Formula (I), (I′), (II), or (II′), R¹² is —SO₂NR⁷₂; and R¹³ is substituted or unsubstituted C₁₋₃ alkyl. In some embodiments of Formula (I), (I′), (II), or (II′), R¹² is —OSO₂R⁷; and R¹³ is substituted or unsubstituted C₁₋₃ alkyl. In some embodiments of Formula (I), (I′), (II), or (II′), R¹² is —OSOR⁷; and R¹³ is substituted or unsubstituted C₁₋₃ alkyl. In some embodiments of Formula (I), (I′), (II), or (II′), R¹² is —OSO₂NR⁷₂; and R¹³ is substituted or unsubstituted C₁₋₃ alkyl. In some embodiments of Formula (I), (I′), (II), or (II′), R¹² is —OSO₂OR⁷; and R¹³ is substituted or unsubstituted C₁₋₃ alkyl. In some embodiments of Formula (I), (I′), (II), or (II′), R¹² is —O(SO)OR⁷; and R¹³ is substituted or unsubstituted C₁₋₃ alkyl. In some embodiments of Formula (I), (I′), (II), or (II′), R¹² is —PO(OR⁷)₂; and R¹³ is substituted or unsubstituted C₁₋₃ alkyl. In some embodiments of Formula (I), (I′), (II), or (II′), R¹² is —PO(NR⁷)₂; and R¹³ is substituted or unsubstituted C₁₋₃alkyl. In some embodiments of Formula (I), (I′), (II), or (II′), R¹² is —PO(NR⁷)(OR⁷); and R¹³ is substituted or unsubstituted C₁₋₃ alkyl. In some embodiments of Formula (I), (I′), (II), or (II′), R¹² is —OPO(OR⁷)₂; and R¹³ is substituted or unsubstituted C₁₋₃ alkyl. In some embodiments of Formula (I), (I′), (II), or (II′), R¹² is —OPO(NR⁷)₂; and R¹³ is substituted or unsubstituted C₁₋₃ alkyl. In some embodiments of Formula (I), (I′), (II), or (II′), R¹² is —OPO(NR⁷)(OR⁷); and R¹³ is substituted or unsubstituted C₁₋₃ alkyl. In some embodiments of Formula (I), (I′), (II), or (II′), R¹² is a nitrogen protecting group; and R¹³ is substituted or unsubstituted C₁₋₃ alkyl. In some embodiments of Formula (I), (I′), (II), or (II′), R¹² is —OR⁷; and R¹³ is substituted or unsubstituted C₁₋₃ alkyl.

In some embodiments of Formula (I), (I′), (II), or (II′), R¹² is hydrogen; and R¹³ is substituted or unsubstituted methyl. In some embodiments of Formula (I), (I′), (II), or (II′), R¹² is substituted or unsubstituted C₁₋₆ alkyl; and R¹³ is substituted or unsubstituted methyl. In some embodiments of Formula (I), (I′), (II), or (II′), R¹² is substituted or unsubstituted C₁₋₃ alkyl; and R¹³ is substituted or unsubstituted methyl. In some embodiments of Formula (I), (I′), (II), or (II′), R¹² is branched or unbranched substituted or unsubstituted C₁₋₆ alkyl; and R¹³ is substituted or unsubstituted methyl. In some embodiments of Formula (I), (I′), (II), or (II′), R¹² is substituted or unsubstituted methyl; and R¹³ is substituted or unsubstituted methyl. In some embodiments of Formula (I), (I′), (II), or (II′), R¹² is substituted or unsubstituted ethyl; and R¹³ is substituted or unsubstituted methyl. In some embodiments of Formula (I), (I′), (II), or (II′), R¹² is substituted or unsubstituted propyl; and R¹³ is substituted or unsubstituted methyl. In some embodiments of Formula (I), (I′), (II), or (II′), R¹² is substituted or unsubstituted butyl; and R¹³ is substituted or unsubstituted methyl. In some embodiments of Formula (I), (I′), (II), or (II′), R¹² is substituted or unsubstituted pentyl; and R¹³ is substituted or unsubstituted methyl. In some embodiments of Formula (I), (I′), (II), or (II′), R¹² is substituted or unsubstituted C₁₋₆ haloalkyl; and R¹³ is substituted or unsubstituted methyl. In some embodiments of Formula (I), (I′), (II), or (II′), R¹² is substituted or unsubstituted fluoroalkyl; and R¹³ is substituted or unsubstituted methyl. In some embodiments of Formula (I), (I′), (II), or (II′), R¹² is trifluoromethyl; and R¹³ is substituted or unsubstituted methyl. In some embodiments of Formula (I), (I′), (II), or (II′), R¹² is substituted or unsubstituted trifluoroethyl; and R¹³ is substituted or unsubstituted methyl. In some embodiments of Formula (I), (I′), (II), or (II′), R¹² is substituted or unsubstituted C₂₋₆ alkenyl; and R¹³ is substituted or unsubstituted methyl. In some embodiments of Formula (I), (I′), (II), or (II′), R¹² is substituted or unsubstituted C₂₋₆ alkenyl; and R¹³ is substituted or unsubstituted methyl. In some embodiments of Formula (I), (I′), (II), or (II′), R¹² is substituted or unsubstituted vinyl; and R¹³ is substituted or unsubstituted methyl. In some embodiments of Formula (I), (I′), (II), or (II′), R¹² is substituted or unsubstituted allyl; and R¹³ is substituted or unsubstituted methyl. In some embodiments of Formula (I), (I′), (II), or (II′), R¹² is substituted or unsubstituted propenyl; and R¹³ is substituted or unsubstituted methyl. In some embodiments of Formula (I), (I′), (II), or (II′), R¹² is substituted or unsubstituted butenyl; and R¹³ is substituted or unsubstituted methyl. In some embodiments of Formula (I), (I′), (II), or (II′), R¹² is substituted or unsubstituted C₂₋₆ alkynyl; and R¹³ is substituted or unsubstituted methyl. In some embodiments of Formula (I), (I′), (II), or (II′), R¹² is substituted or unsubstituted acetylenyl; and R¹³ is substituted or unsubstituted methyl. In some embodiments of Formula (I), (I′), (II), or (II′), R¹² is substituted or unsubstituted propargyl; and R¹³ is substituted or unsubstituted methyl. In some embodiments of Formula (I), (I′), (II), or (II′), R¹² is substituted or unsubstituted butynyl; and R¹³ is substituted or unsubstituted methyl. In some embodiments of Formula (I), (I′), (II), or (II′), R¹² is substituted or unsubstituted C₁₋₆ alkoxy; and R¹³ is substituted or unsubstituted methyl. In some embodiments of Formula (I), (I′), (II), or (II′), R¹² is CH₂—OMe; and R¹³ is substituted or unsubstituted methyl. In some embodiments of Formula (I), (I′), (II), or (II′), R¹² is CH₂-OEt; and R¹³ is substituted or unsubstituted methyl. In some embodiments of Formula (I), (I′), (II), or (II′), R¹²CH₂—OPr; and R¹³ is substituted or unsubstituted methyl. In some embodiments of Formula (I), (I′), (II), or (II′), R¹² is CH₂—OCF₃; and R¹³ is substituted or unsubstituted methyl. In some embodiments of Formula (I), (I′), (II), or (II′), R¹² is substituted or unsubstituted CH₂—OR⁷; and R¹³ is substituted or unsubstituted methyl. In some embodiments of Formula (I), (I′), (II), or (II′), R¹² is substituted or unsubstituted C₆₋₁₀ aryl; and R¹³ is substituted or unsubstituted methyl. In some embodiments of Formula (I), (I′), (II), or (II′), R¹² is substituted or unsubstituted 5-10 membered heteroaryl; and R¹³ is substituted or unsubstituted methyl. In some embodiments of Formula (I), (I′), (II), or (II′), R¹² is substituted or unsubstituted 4-10 membered heterocyclyl; and R¹³ substituted or unsubstituted methyl. In some embodiments of Formula (I), (I′), (II), or (II′), R¹² is substituted or unsubstituted 3-10 membered carbocyclyl; and R¹³ is substituted or unsubstituted methyl. In some embodiments of Formula (I), (I′), (II), or (II′), R¹² is acyl; and R¹³ is substituted or unsubstituted methyl. In some embodiments of Formula (I), (I′), (II), or (II′), R¹² is a substituted or unsubstituted amide; and R¹³ is substituted or unsubstituted methyl. In some embodiments of Formula (I), (I′), (II), or (II′), R¹² is a substituted or unsubstituted ester; and R¹³ is substituted or unsubstituted methyl. In some embodiments of Formula (I), (I′), (II), or (II′), R¹² is —OH; and R¹³ is substituted or unsubstituted methyl. In some embodiments of Formula (I), (I′), (II), or (II′), R¹² is —SO₂R⁷; and R¹³ is substituted or unsubstituted methyl. In some embodiments of Formula (I), (I′), (II), or (II′), R¹² is —SOR⁷; and R¹³ is substituted or unsubstituted methyl. In some embodiments of Formula (I), (I′), (II), or (II′), R¹² is —SO₂NR⁷₂; and R¹³ is substituted or unsubstituted methyl. In some embodiments of Formula (I), (I′), (II), or (II′), R¹² is —OSO₂R⁷; and R¹³ is substituted or unsubstituted methyl. In some embodiments of Formula (I), (I′), (II), or (II′), R¹² is —OSOR⁷; and R¹³ is substituted or unsubstituted methyl. In some embodiments of Formula (I), (I′), (II), or (II′), R¹² is —OSO₂NR⁷₂; and R¹³ is substituted or unsubstituted methyl. In some embodiments of Formula (I), (I′), (II), or (II′), R¹² is —OSO₂OR⁷; and R¹³ is substituted or unsubstituted methyl. In some embodiments of Formula (I), (I′), (II), or (II′), R¹² is —O(SO)OR⁷; and R¹³ is substituted or unsubstituted methyl. In some embodiments of Formula (I), (I′), (II), or (II′), R¹² is —PO(OR⁷)₂; and R¹³ is substituted or unsubstituted methyl. In some embodiments of Formula (I), (I′), (II), or (II′), R¹² is —PO(NR⁷)₂; and R¹³ is substituted or unsubstituted methyl. In some embodiments of Formula (I), (I′), (II), or (II′), R¹² is —PO(NR⁷)(OR⁷); and R¹³ is substituted or unsubstituted methyl. In some embodiments of Formula (I), (I′), (II), or (II′), R¹² is —OPO(OR⁷)₂; and R¹³ is substituted or unsubstituted methyl. In some embodiments of Formula (I), (I′), (II), or (II′), R¹² is —OPO(NR⁷)₂; and R¹³ is substituted or unsubstituted methyl. In some embodiments of Formula (I), (I′), (II), or (II′), R¹² is —OPO(NR⁷)(OR⁷); and R¹³ is substituted or unsubstituted methyl. In some embodiments of Formula (I), (I′), (II), or (II′), R¹² is a nitrogen protecting group; and R¹³ is substituted or unsubstituted methyl. In some embodiments of Formula (I), (I′), (II), or (II′), R¹² is —OR⁷; and R¹³ is substituted or unsubstituted methyl.

In some embodiments of Formula (I), (I′), (II), or (II′), R¹² is hydrogen; and R¹³ is substituted or unsubstituted ethyl. In some embodiments of Formula (I), (I′), (II), or (II′), R¹² is substituted or unsubstituted C₁₋₆ alkyl; and R¹³ is substituted or unsubstituted ethyl. In some embodiments of Formula (I), (I′), (II), or (II′), R¹² is substituted or unsubstituted C₁₋₃alkyl; and R¹³ is substituted or unsubstituted ethyl. In some embodiments of Formula (I), (I′), (II), or (II′), R¹² is branched or unbranched substituted or unsubstituted C₁₋₆ alkyl; and R¹³ is substituted or unsubstituted ethyl. In some embodiments of Formula (I), (I′), (II), or (II′), R¹² is substituted or unsubstituted methyl; and R¹³ is substituted or unsubstituted ethyl. In some embodiments of Formula (I), (I′), (II), or (II′), R¹² is substituted or unsubstituted ethyl; and R¹³ is substituted or unsubstituted ethyl. In some embodiments of Formula (I), (I′), (II), or (II′), R¹² is substituted or unsubstituted propyl; and R¹³ is substituted or unsubstituted ethyl. In some embodiments of Formula (I), (I′), (II), or (II′), R¹² is substituted or unsubstituted butyl; and R¹³ is substituted or unsubstituted ethyl. In some embodiments of Formula (I), (I′), (II), or (II′), R¹² is substituted or unsubstituted pentyl; and R¹³ is substituted or unsubstituted ethyl. In some embodiments of Formula (I), (I′), (II), or (II′), R¹² is substituted or unsubstituted C₁₋₆ haloalkyl; and R¹³ is substituted or unsubstituted ethyl. In some embodiments of Formula (I), (I′), (II), or (II′), R¹² is substituted or unsubstituted fluoroalkyl; and R¹³ is substituted or unsubstituted ethyl. In some embodiments of Formula (I), (I′), (II), or (II′), R¹² is trifluoromethyl; and R¹³ is substituted or unsubstituted ethyl. In some embodiments of Formula (I), (I′), (II), or (II′), R¹² is substituted or unsubstituted trifluoroethyl; and R¹³ is substituted or unsubstituted ethyl. In some embodiments of Formula (I), (I′), (II), or (II′), R¹² is substituted or unsubstituted C₂₋₆ alkenyl; and R¹³ is substituted or unsubstituted ethyl. In some embodiments of Formula (I), (I′), (II), or (II′), R¹² is substituted or unsubstituted C₂₋₆ alkenyl; and R¹³ is substituted or unsubstituted ethyl. In some embodiments of Formula (I), (I′), (II), or (II′), R¹² is substituted or unsubstituted vinyl; and R¹³ is substituted or unsubstituted ethyl. In some embodiments of Formula (I), (I′), (II), or (II′), R¹² is substituted or unsubstituted allyl; and R¹³ is substituted or unsubstituted ethyl. In some embodiments of Formula (I), (I′), (II), or (II′), R¹² is substituted or unsubstituted propenyl; and R¹³ is substituted or unsubstituted ethyl. In some embodiments of Formula (I), (I′), (II), or (II′), R¹² is substituted or unsubstituted butenyl; and R¹³ is substituted or unsubstituted ethyl. In some embodiments of Formula (I), (I′), (II), or (II′), R¹² is substituted or unsubstituted C₂₋₆ alkynyl; and R¹³ is substituted or unsubstituted ethyl. In some embodiments of Formula (I), (I′), (II), or (II′), R¹² is substituted or unsubstituted acetylenyl; and R¹³ is substituted or unsubstituted ethyl. In some embodiments of Formula (I), (I′), (II), or (II′), R¹² is substituted or unsubstituted propargyl; and R¹³ is substituted or unsubstituted ethyl. In some embodiments of Formula (I), (I′), (II), or (II′), R¹² is substituted or unsubstituted butynyl; and R¹³ is substituted or unsubstituted ethyl. In some embodiments of Formula (I), (I′), (II), or (II′), R¹² is substituted or unsubstituted C₁₋₆ alkoxy; and R¹³ is substituted or unsubstituted ethyl. In some embodiments of Formula (I), (I′), (II), or (II′), R¹² is CH₂—OMe; and R¹³ is substituted or unsubstituted ethyl. In some embodiments of Formula (I), (I′), (II), or (II′), R¹² is CH₂—OEt; and R¹³ is substituted or unsubstituted ethyl. In some embodiments of Formula (I), (I′), (II), or (II′), R¹²CH₂—OPr; and R¹³ is substituted or unsubstituted ethyl. In some embodiments of Formula (I), (I′), (II), or (II′), R¹² is CH₂—OCF₃; and R¹³ is substituted or unsubstituted ethyl. In some embodiments of Formula (I), (I′), (II), or (II′), R¹² is substituted or unsubstituted CH₂—OR⁷; and R¹³ is substituted or unsubstituted ethyl. In some embodiments of Formula (I), (I′), (II), or (II′), R¹² is substituted or unsubstituted C₆₋₁₀ aryl; and R¹³ is substituted or unsubstituted ethyl. In some embodiments of Formula (I), (I′), (II), or (II′), R¹² is substituted or unsubstituted 5-10 membered heteroaryl; and R¹³ is substituted or unsubstituted ethyl. In some embodiments of Formula (I), (I′), (II), or (II′), R¹² is substituted or unsubstituted 4-10 membered heterocyclyl; and R¹³ is substituted or unsubstituted ethyl. In some embodiments of Formula (I), (I′), (II), or (II′), R¹² is substituted or unsubstituted 3-10 membered carbocyclyl; and R¹³ is substituted or unsubstituted ethyl. In some embodiments of Formula (I), (I′), (II), or (II′), R¹² is acyl; and R¹³ is substituted or unsubstituted ethyl. In some embodiments of Formula (I), (I′), (II), or (II′), R¹² is a substituted or unsubstituted amide; and R¹³ is substituted or unsubstituted ethyl. In some embodiments of Formula (I), (I′), (II), or (II′), R¹² is a substituted or unsubstituted ester; and R¹³ is substituted or unsubstituted ethyl. In some embodiments of Formula (I), (I′), (II), or (II′), R¹² is —OH; and R¹³ is substituted or unsubstituted ethyl. In some embodiments of Formula (I), (I′), (II), or (II′), R¹² is —SO₂R⁷; and R¹³ is substituted or unsubstituted ethyl. In some embodiments of Formula (I), (I′), (II), or (II′), R¹² is —SOR⁷; and R¹³ is substituted or unsubstituted ethyl. In some embodiments of Formula (I), (I′), (II), or (II′), R¹² is —SO₂NR⁷₂; and R¹³ is substituted or unsubstituted ethyl. In some embodiments of Formula (I), (I′), (II), or (II′), R¹² is —OSO₂R⁷; and R¹³ is substituted or unsubstituted ethyl. In some embodiments of Formula (I), (I′), (II), or (II′), R¹² is —OSOR⁷; and R¹³ is substituted or unsubstituted ethyl. In some embodiments of Formula (I), (I′), (II), or (II′), R¹² is —OSO₂NR⁷₂; and R¹³ is substituted or unsubstituted ethyl. In some embodiments of Formula (I), (I′), (II), or (II′), R¹² is —OSO₂OR⁷; and R¹³ is substituted or unsubstituted ethyl. In some embodiments of Formula (I), (I′), (II), or (II′), R¹² is —O(SO)OR⁷; and R¹³ is substituted or unsubstituted ethyl. In some embodiments of Formula (I), (I′), (II), or (II′), R¹² is —PO(OR⁷)₂; and R¹³ is substituted or unsubstituted ethyl. In some embodiments of Formula (I), (I′), (II), or (II′), R¹² is —PO(NR⁷)₂; and R¹³ is substituted or unsubstituted ethyl. In some embodiments of Formula (I), (I′), (II), or (II′), R¹² is —PO(NR⁷)(OR⁷); and R¹³ is substituted or unsubstituted ethyl. In some embodiments of Formula (I), (I′), (II), or (II′), R¹² is —OPO(OR⁷)₂; and R¹³ is substituted or unsubstituted ethyl. In some embodiments of Formula (I), (I′), (II), or (II′), R¹² is —OPO(NR⁷)₂; and R¹³ is substituted or unsubstituted ethyl. In some embodiments of Formula (I), (I′), (II), or (II′), R¹² is —OPO(NR⁷)(OR⁷); and R¹³ is substituted or unsubstituted ethyl. In some embodiments of Formula (I), (I′), (II), or (II′), R¹² is a nitrogen protecting group; and R¹³ is substituted or unsubstituted ethyl. In some embodiments of Formula (I), (I′), (II), or (II′), R¹² is —OR⁷; and R¹³ is substituted or unsubstituted ethyl.

In some embodiments of Formula (I), (I′), (II), or (II′), R¹² is hydrogen; and R¹³ is substituted or unsubstituted propyl. In some embodiments of Formula (I), (I′), (II), or (II′), R¹² is substituted or unsubstituted C₁₋₆ alkyl; and R¹³ is substituted or unsubstituted propyl. In some embodiments of Formula (I), (I′), (II), or (II′), R¹² is substituted or unsubstituted C₁₋₃ alkyl; and R¹³ is substituted or unsubstituted propyl. In some embodiments of Formula (I), (I′), (II), or (II′), R¹² is branched or unbranched substituted or unsubstituted C₁₋₆ alkyl; and R¹³ is substituted or unsubstituted propyl. In some embodiments of Formula (I), (I′), (II), or (II′), R¹² is substituted or unsubstituted methyl; and R¹³ is substituted or unsubstituted propyl. In some embodiments of Formula (I), (I′), (II), or (II′), R¹² is substituted or unsubstituted ethyl; and R¹³ is substituted or unsubstituted propyl. In some embodiments of Formula (I), (I′), (II), or (II′), R¹² is substituted or unsubstituted propyl; and R¹³ is substituted or unsubstituted propyl. In some embodiments of Formula (I), (I′), (II), or (II′), R¹² is substituted or unsubstituted butyl; and R¹³ is substituted or unsubstituted propyl. In some embodiments of Formula (I), (I′), (II), or (II′), R¹² is substituted or unsubstituted pentyl; and R¹³ is substituted or unsubstituted propyl. In some embodiments of Formula (I), (I′), (II), or (II′), R¹² is substituted or unsubstituted C₁₋₆ haloalkyl; and R¹³ is substituted or unsubstituted propyl. In some embodiments of Formula (I), (I′), (II), or (II′), R¹² is substituted or unsubstituted fluoroalkyl; and R¹³ is substituted or unsubstituted propyl. In some embodiments of Formula (I), (I′), (II), or (II′), R¹² is trifluoromethyl; and R¹³ is substituted or unsubstituted propyl. In some embodiments of Formula (I), (I′), (II), or (II′), R¹² is substituted or unsubstituted trifluoroethyl; and R¹³ is substituted or unsubstituted propyl. In some embodiments of Formula (I), (I′), (II), or (II′), R¹² is substituted or unsubstituted C₂₋₆ alkenyl; and R¹³ is substituted or unsubstituted propyl. In some embodiments of Formula (I), (I′), (II), or (II′), R¹² is substituted or unsubstituted C₂₋₆ alkenyl; and R¹³ is substituted or unsubstituted propyl. In some embodiments of Formula (I), (I′), (II), or (II′), R¹² is substituted or unsubstituted vinyl; and R¹³ is substituted or unsubstituted propyl. In some embodiments of Formula (I), (I′), (II), or (II′), R¹² is substituted or unsubstituted allyl; and R¹³ is substituted or unsubstituted propyl. In some embodiments of Formula (I), (I′), (II), or (II′), R¹² is substituted or unsubstituted propenyl; and R¹³ is substituted or unsubstituted propyl. In some embodiments of Formula (I), (I′), (II), or (II′), R¹² is substituted or unsubstituted butenyl; and R¹³ is substituted or unsubstituted propyl. In some embodiments of Formula (I), (I′), (II), or (II′), R¹² is substituted or unsubstituted C₂₋₆ alkynyl; and R¹³ is substituted or unsubstituted propyl. In some embodiments of Formula (I), (I′), (II), or (II′), R¹² is substituted or unsubstituted acetylenyl; and R¹³ is substituted or unsubstituted propyl. In some embodiments of Formula (I), (I′), (II), or (II′), R¹² is substituted or unsubstituted propargyl; and R¹³ is substituted or unsubstituted propyl. In some embodiments of Formula (I), (I′), (II), or (II′), R¹² is substituted or unsubstituted butynyl; and R¹³ is substituted or unsubstituted propyl. In some embodiments of Formula (I), (I′), (II), or (II′), R¹² is substituted or unsubstituted C₁₋₆ alkoxy; and R¹³ is substituted or unsubstituted propyl. In some embodiments of Formula (I), (I′), (II), or (II′), R¹² is CH₂—OMe; and R¹³ is substituted or unsubstituted propyl. In some embodiments of Formula (I), (I′), (II), or (II′), R¹² is CH₂-OEt; and R¹³ is substituted or unsubstituted propyl. In some embodiments of Formula (I), (I′), (II), or (II′), R¹²CH₂—OPr; and R¹³ is substituted or unsubstituted propyl. In some embodiments of Formula (I), (I′), (II), or (II′), R¹² is CH₂—OCF₃; and R¹³ is substituted or unsubstituted propyl. In some embodiments of Formula (I), (I′), (II), or (II′), R¹² is substituted or unsubstituted CH₂—OR⁷; and R¹³ is substituted or unsubstituted propyl. In some embodiments of Formula (I), (I′), (II), or (II′), R¹² is substituted or unsubstituted C₆₋₁₀ aryl; and R¹³ is substituted or unsubstituted propyl. In some embodiments of Formula (I), (I′), (II), or (II′), R¹² is substituted or unsubstituted 5-10 membered heteroaryl; and R¹³ is substituted or unsubstituted propyl. In some embodiments of Formula (I), (I′), (II), or (II′), R¹² is substituted or unsubstituted 4-10 membered heterocyclyl; and R¹³ is substituted or unsubstituted propyl. In some embodiments of Formula (I), (I′), (II), or (II′), R¹² is substituted or unsubstituted 3-10 membered carbocyclyl; and R¹³ is substituted or unsubstituted propyl. In some embodiments of Formula (I), (I′), (II), or (II′), R¹² is acyl; and R¹³ is substituted or unsubstituted propyl. In some embodiments of Formula (I), (I′), (II), or (II′), R¹² is a substituted or unsubstituted amide; and R¹³ is substituted or unsubstituted propyl. In some embodiments of Formula (I), (I′), (II), or (II′), R¹² is a substituted or unsubstituted ester; and R¹³ is substituted or unsubstituted propyl. In some embodiments of Formula (I), (I′), (II), or (II′), R¹² is —OH; and R¹³ is substituted or unsubstituted propyl. In some embodiments of Formula (I), (I′), (II), or (II′), R¹² is —SO₂R⁷; and R¹³ is substituted or unsubstituted propyl. In some embodiments of Formula (I), (I′), (II), or (II′), R¹² is —SOR⁷; and R¹³ is substituted or unsubstituted propyl. In some embodiments of Formula (I), (I′), (II), or (II′), R¹² is —SO₂NR⁷₂; and R¹³ is substituted or unsubstituted propyl. In some embodiments of Formula (I), (I′), (II), or (II′), R¹² is —OSO₂R⁷; and R¹³ is substituted or unsubstituted propyl. In some embodiments of Formula (I), (I′), (II), or (II′), R¹² is —OSOR⁷; and R¹³ is substituted or unsubstituted propyl. In some embodiments of Formula (I), (I′), (II), or (II′), R¹² is —OSO₂NR⁷₂; and R¹³ is substituted or unsubstituted propyl. In some embodiments of Formula (I), (I′), (II), or (II′), R¹² is —OSO₂OR⁷; and R¹³ is substituted or unsubstituted propyl. In some embodiments of Formula (I), (I′), (II), or (II′), R¹² is —O(SO)OR⁷; and R¹³ is substituted or unsubstituted propyl. In some embodiments of Formula (I), (I′), (II), or (II′), R¹² is —PO(OR⁷)₂; and R¹³ is substituted or unsubstituted propyl. In some embodiments of Formula (I), (I′), (II), or (II′), R¹² is —PO(NR⁷)₂; and R¹³ is substituted or unsubstituted propyl. In some embodiments of Formula (I), (I′), (II), or (II′), R¹² is —PO(NR⁷)(OR⁷); and R¹³ is substituted or unsubstituted propyl. In some embodiments of Formula (I), (I′), (II), or (II′), R¹² is —OPO(OR⁷)₂; and R¹³ is substituted or unsubstituted propyl. In some embodiments of Formula (I), (I′), (II), or (II′), R¹² is —OPO(NR⁷)₂; and R¹³ is substituted or unsubstituted propyl. In some embodiments of Formula (I), (I′), (II), or (II′), R¹² is —OPO(NR⁷)(OR⁷); and R¹³ is substituted or unsubstituted propyl. In some embodiments of Formula (I), (I′), (II), or (II′), R¹² is a nitrogen protecting group; and R¹³ is substituted or unsubstituted propyl. In some embodiments of Formula (I), (I′), (II), or (II′), R¹² is —OR⁷; and R¹³ is substituted or unsubstituted propyl.

In some embodiments of Formula (I), (I′), (II), or (II′), R¹² is hydrogen; and R¹³ is substituted or unsubstituted butyl. In some embodiments of Formula (I), (I′), (II), or (II′), R¹² is substituted or unsubstituted C₁₋₆ alkyl; and R¹³ is substituted or unsubstituted butyl. In some embodiments of Formula (I), (I′), (II), or (II′), R¹² is substituted or unsubstituted C₁₋₃alkyl; and R¹³ is substituted or unsubstituted butyl. In some embodiments of Formula (I), (I′), (II), or (II′), R¹² is branched or unbranched substituted or unsubstituted C₁₋₆ alkyl; and R¹³ is substituted or unsubstituted butyl. In some embodiments of Formula (I), (I′), (II), or (II′), R¹² is substituted or unsubstituted methyl; and R¹³ is substituted or unsubstituted butyl.

In some embodiments of Formula (I), (I′), (II), or (II′), R¹² is substituted or unsubstituted ethyl; and R¹³ is substituted or unsubstituted butyl. In some embodiments of Formula (I), (I′), (II), or (II′), R¹² is substituted or unsubstituted propyl; and R¹³ is substituted or unsubstituted butyl. In some embodiments of Formula (I), (I′), (II), or (II′), R¹² is substituted or unsubstituted butyl; and R¹³ is substituted or unsubstituted butyl. In some embodiments of Formula (I), (I′), (II), or (II′), R¹² is substituted or unsubstituted pentyl; and R¹³ is substituted or unsubstituted butyl. In some embodiments of Formula (I), (I′), (II), or (II′), R¹² is substituted or unsubstituted C₁₋₆ haloalkyl; and R¹³ is substituted or unsubstituted butyl. In some embodiments of Formula (I), (I′), (II), or (II′), R¹² is substituted or unsubstituted fluoroalkyl; and R¹³ is substituted or unsubstituted butyl. In some embodiments of Formula (I), (I′), (II), or (II′), R¹² is trifluoromethyl; and R¹³ is substituted or unsubstituted butyl. In some embodiments of Formula (I), (I′), (II), or (II′), R¹² is substituted or unsubstituted trifluoroethyl; and R¹³ is substituted or unsubstituted butyl. In some embodiments of Formula (I), (I′), (II), or (II′), R¹² is substituted or unsubstituted C₂₋₆ alkenyl; and R¹³ is substituted or unsubstituted butyl. In some embodiments of Formula (I), (I′), (II), or (II′), R¹² is substituted or unsubstituted C₂₋₆ alkenyl; and R¹³ is substituted or unsubstituted butyl. In some embodiments of Formula (I), (I′), (II), or (II′), R¹² is substituted or unsubstituted vinyl; and R¹³ is substituted or unsubstituted butyl. In some embodiments of Formula (I), (I′), (II), or (II′), R¹² is substituted or unsubstituted allyl; and R¹³ is substituted or unsubstituted butyl. In some embodiments of Formula (I), (I′), (II), or (II′), R¹² is substituted or unsubstituted propenyl; and R¹³ is substituted or unsubstituted butyl. In some embodiments of Formula (I), (I′), (II), or (II′), R¹² is substituted or unsubstituted butenyl; and R¹³ is substituted or unsubstituted butyl. In some embodiments of Formula (I), (I′), (II), or (II′), R¹² is substituted or unsubstituted C₂₋₆ alkynyl; and R¹³ is substituted or unsubstituted butyl. In some embodiments of Formula (I), (I′), (II), or (II′), R¹² is substituted or unsubstituted acetylenyl; and R¹³ is substituted or unsubstituted butyl. In some embodiments of Formula (I), (I′), (II), or (II′), R¹² is substituted or unsubstituted propargyl; and R¹³ is substituted or unsubstituted butyl. In some embodiments of Formula (I), (I′), (II), or (II′), R¹² is substituted or unsubstituted butynyl; and R¹³ is substituted or unsubstituted butyl. In some embodiments of Formula (I), (I′), (II), or (II′), R¹² is substituted or unsubstituted C₁₋₆alkoxy; and R¹³ is substituted or unsubstituted butyl. In some embodiments of Formula (I), (I′), (II), or (II′), R¹² is CH₂—OMe; and R¹³ is substituted or unsubstituted butyl. In some embodiments of Formula (I), (I′), (II), or (II′), R¹² is CH₂-OEt; and R¹³ is substituted or unsubstituted butyl. In some embodiments of Formula (I), (I′), (II), or (II′), R¹²CH₂—OPr; and R¹³ is substituted or unsubstituted butyl. In some embodiments of Formula (I), (I′), (II), or (II′), R¹² is CH₂—OCF₃; and R¹³ is substituted or unsubstituted butyl. In some embodiments of Formula (I), (I′), (II), or (II′), R¹² is substituted or unsubstituted CH₂—OR⁷; and R¹³ is substituted or unsubstituted butyl. In some embodiments of Formula (I), (I′), (II), or (II′), R¹² is substituted or unsubstituted C₆₋₁₀ aryl; and R¹³ is substituted or unsubstituted butyl. In some embodiments of Formula (I), (I′), (II), or (II′), R¹² is substituted or unsubstituted 5-10 membered heteroaryl; and R¹³ is substituted or unsubstituted butyl. In some embodiments of Formula (I), (I′), (II), or (II′), R¹² is substituted or unsubstituted 4-10 membered heterocyclyl; and R¹³ is substituted or unsubstituted butyl. In some embodiments of Formula (I), (I′), (II), or (II′), R¹² is substituted or unsubstituted 3-10 membered carbocyclyl; and R¹³ is substituted or unsubstituted butyl. In some embodiments of Formula (I), (I′), (II), or (II′), R¹² is acyl; and R¹³ is substituted or unsubstituted butyl. In some embodiments of Formula (I), (I′), (II), or (II′), R¹² is a substituted or unsubstituted amide; and R¹³ is substituted or unsubstituted butyl. In some embodiments of Formula (I), (I′), (II), or (II′), R¹² is a substituted or unsubstituted ester; and R¹³ is substituted or unsubstituted butyl. In some embodiments of Formula (I), (I′), (II), or (II′), R¹² is —OH; and R¹³ is substituted or unsubstituted butyl. In some embodiments of Formula (I), (I′), (II), or (II′), R¹² is —SO₂R⁷; and R¹³ is substituted or unsubstituted butyl. In some embodiments of Formula (I), (I′), (II), or (II′), R¹² is —SOR⁷; and R¹³ is substituted or unsubstituted butyl. In some embodiments of Formula (I), (I′), (II), or (II′), R¹² is —SO₂NR⁷₂; and R¹³ is substituted or unsubstituted butyl. In some embodiments of Formula (I), (I′), (II), or (II′), R¹² is —OSO₂R⁷; and R¹³ is substituted or unsubstituted butyl. In some embodiments of Formula (I), (I′), (II), or (II′), R¹² is —OSOR⁷; and R¹³ is substituted or unsubstituted butyl. In some embodiments of Formula (I), (I′), (II), or (II′), R¹² is —OSO₂NR⁷₂; and R¹³ is substituted or unsubstituted butyl. In some embodiments of Formula (I), (I′), (II), or (II′), R¹² is —OSO₂OR⁷; and R¹³ is substituted or unsubstituted butyl. In some embodiments of Formula (I), (I′), (II), or (II′), R¹² is —O(SO)OR⁷; and R¹³ is substituted or unsubstituted butyl. In some embodiments of Formula (I), (I′), (II), or (II′), R¹² is —PO(OR⁷)₂; and R¹³ is substituted or unsubstituted butyl. In some embodiments of Formula (I), (I′), (II), or (II′), R¹² is —PO(NR⁷)₂; and R¹³ is substituted or unsubstituted butyl. In some embodiments of Formula (I), (I′), (II), or (II′), R¹² is —PO(NR⁷)(OR⁷); and R¹³ is substituted or unsubstituted butyl. In some embodiments of Formula (I), (I′), (II), or (II′), R¹² is —OPO(OR⁷)₂; and R¹³ is substituted or unsubstituted butyl. In some embodiments of Formula (I), (I′), (II), or (II′), R¹² is —OPO(NR⁷)₂; and R¹³ is substituted or unsubstituted butyl. In some embodiments of Formula (I), (I′), (II), or (II′), R¹² is —OPO(NR⁷)(OR⁷); and R¹³ is substituted or unsubstituted butyl. In some embodiments of Formula (I), (I′), (II), or (II′), R¹² is a nitrogen protecting group; and R¹³ is substituted or unsubstituted butyl. In some embodiments of Formula (I), (I′), (II), or (II′), R¹² is —OR⁷; and R¹³ is substituted or unsubstituted butyl.

In some embodiments of Formula (I), (I′), (II), or (II′), R¹² is hydrogen; and R¹³ is substituted or unsubstituted pentyl. In some embodiments of Formula (I), (I′), (II), or (II′), R¹² is substituted or unsubstituted C₁₋₆ alkyl; and R¹³ is substituted or unsubstituted pentyl. In some embodiments of Formula (I), (I′), (II), or (II′), R¹² is substituted or unsubstituted C₁₋₃ alkyl; and R¹³ is substituted or unsubstituted pentyl. In some embodiments of Formula (I), (I′), (II), or (II′), R¹² is branched or unbranched substituted or unsubstituted C₁₋₆ alkyl; and R¹³ is substituted or unsubstituted pentyl. In some embodiments of Formula (I), (I′), (II), or (II′), R¹² is substituted or unsubstituted methyl; and R¹³ is substituted or unsubstituted pentyl. In some embodiments of Formula (I), (I′), (II), or (II′), R¹² is substituted or unsubstituted ethyl; and R¹³ is substituted or unsubstituted pentyl. In some embodiments of Formula (I), (I′), (II), or (II′), R¹² is substituted or unsubstituted propyl; and R¹³ is substituted or unsubstituted pentyl. In some embodiments of Formula (I), (I′), (II), or (II′), R¹² is substituted or unsubstituted butyl; and R¹³ is substituted or unsubstituted pentyl. In some embodiments of Formula (I), (I′), (II), or (II′), R¹² is substituted or unsubstituted pentyl; and R¹³ is substituted or unsubstituted pentyl. In some embodiments of Formula (I), (I′), (II), or (II′), R¹² is substituted or unsubstituted C₁₋₆ haloalkyl; and R¹³ is substituted or unsubstituted pentyl. In some embodiments of Formula (I), (I′), (II), or (II′), R¹² is substituted or unsubstituted fluoroalkyl; and R¹³ is substituted or unsubstituted pentyl. In some embodiments of Formula (I), (I′), (II), or (II′), R¹² is trifluoromethyl; and R¹³ is substituted or unsubstituted pentyl. In some embodiments of Formula (I), (I′), (II), or (II′), R¹² is substituted or unsubstituted trifluoroethyl; and R¹³ is substituted or unsubstituted pentyl. In some embodiments of Formula (I), (I′), (II), or (II′), R¹² is substituted or unsubstituted C₂₋₆ alkenyl; and R¹³ is substituted or unsubstituted pentyl. In some embodiments of Formula (I), (I′), (II), or (II′), R¹² is substituted or unsubstituted C₂₋₆ alkenyl; and R¹³ is substituted or unsubstituted pentyl. In some embodiments of Formula (I), (I′), (II), or (II′), R¹² is substituted or unsubstituted vinyl; and R¹³ is substituted or unsubstituted pentyl. In some embodiments of Formula (I), (I′), (II), or (II′), R¹² is substituted or unsubstituted allyl; and R¹³ is substituted or unsubstituted pentyl. In some embodiments of Formula (I), (I′), (II), or (II′), R¹² is substituted or unsubstituted propenyl; and R¹³ is substituted or unsubstituted pentyl. In some embodiments of Formula (I), (I′), (II), or (II′), R¹² is substituted or unsubstituted butenyl; and R¹³ is substituted or unsubstituted pentyl. In some embodiments of Formula (I), (I′), (II), or (II′), R¹² is substituted or unsubstituted C₂₋₆ alkynyl; and R¹³ is substituted or unsubstituted pentyl. In some embodiments of Formula (I), (I′), (II), or (II′), R¹² is substituted or unsubstituted acetylenyl; and R¹³ is substituted or unsubstituted pentyl. In some embodiments of Formula (I), (I′), (II), or (II′), R¹² is substituted or unsubstituted propargyl; and R¹³ is substituted or unsubstituted pentyl. In some embodiments of Formula (I), (I′), (II), or (II′), R¹² is substituted or unsubstituted butynyl; and R¹³ is substituted or unsubstituted pentyl. In some embodiments of Formula (I), (I′), (II), or (II′), R¹² is substituted or unsubstituted C₁₋₆ alkoxy; and R¹³ is substituted or unsubstituted pentyl. In some embodiments of Formula (I), (I′), (II), or (II′), R¹² is CH₂—OMe; and R¹³ is substituted or unsubstituted pentyl. In some embodiments of Formula (I), (I′), (II), or (II′), R¹² is CH₂-OEt; and R¹³ is substituted or unsubstituted pentyl. In some embodiments of Formula (I), (I′), (II), or (II′), R¹²CH₂—OPr; and R¹³ is substituted or unsubstituted pentyl. In some embodiments of Formula (I), (I′), (II), or (II′), R¹² is CH₂—OCF₃; and R¹³ is substituted or unsubstituted pentyl. In some embodiments of Formula (I), (I′), (II), or (II′), R¹² is substituted or unsubstituted CH₂—OR⁷; and R¹³ is substituted or unsubstituted pentyl. In some embodiments of Formula (I), (I′), (II), or (II′), R¹² is substituted or unsubstituted C₆₋₁₀ aryl; and R¹³ is substituted or unsubstituted pentyl. In some embodiments of Formula (I), (I′), (II), or (II′), R¹² is substituted or unsubstituted 5-10 membered heteroaryl; and R¹³ is substituted or unsubstituted pentyl. In some embodiments of Formula (I), (I′), (II), or (II′), R¹² is substituted or unsubstituted 4-10 membered heterocyclyl; and R¹³ is substituted or unsubstituted pentyl. In some embodiments of Formula (I), (I′), (II), or (II′), R¹² is substituted or unsubstituted 3-10 membered carbocyclyl; and R¹³ is substituted or unsubstituted pentyl. In some embodiments of Formula (I), (I′), (II), or (II′), R¹² is acyl; and R¹³ is substituted or unsubstituted pentyl. In some embodiments of Formula (I), (I′), (II), or (II′), R¹² is a substituted or unsubstituted amide; and R¹³ is substituted or unsubstituted pentyl. In some embodiments of Formula (I), (I′), (II), or (II′), R¹² is a substituted or unsubstituted ester; and R¹³ is substituted or unsubstituted pentyl. In some embodiments of Formula (I), (I′), (II), or (II′), R¹² is —OH; and R¹³ is substituted or unsubstituted pentyl. In some embodiments of Formula (I), (I′), (II), or (II′), R¹² is —SO₂R⁷; and R¹³ is substituted or unsubstituted pentyl. In some embodiments of Formula (I), (I′), (II), or (II′), R¹² is —SOR⁷; and R¹³ is substituted or unsubstituted pentyl. In some embodiments of Formula (I), (I′), (II), or (II′), R¹² is —SO₂NR⁷₂; and R¹³ is substituted or unsubstituted pentyl. In some embodiments of Formula (I), (I′), (II), or (II′), R¹² is —OSO₂R⁷; and R¹³ is substituted or unsubstituted pentyl. In some embodiments of Formula (I), (I′), (II), or (II′), R¹² is —OSOR⁷; and R¹³ is substituted or unsubstituted pentyl. In some embodiments of Formula (I), (I′), (II), or (II′), R¹² is —OSO₂NR⁷₂; and R¹³ is substituted or unsubstituted pentyl. In some embodiments of Formula (I), (I′), (II), or (II′), R¹² is —OSO₂OR⁷; and R¹³ is substituted or unsubstituted pentyl. In some embodiments of Formula (I), (I′), (II), or (II′), R¹² is —O(SO)OR⁷; and R¹³ is substituted or unsubstituted pentyl. In some embodiments of Formula (I), (I′), (II), or (II′), R¹² is —PO(OR⁷)₂; and R¹³ is substituted or unsubstituted pentyl. In some embodiments of Formula (I), (I′), (II), or (II′), R¹² is —PO(NR⁷)₂; and R¹³ is substituted or unsubstituted pentyl. In some embodiments of Formula (I), (I′), (II), or (II′), R¹² is —PO(NR⁷)(OR⁷); and R¹³ is substituted or unsubstituted pentyl. In some embodiments of Formula (I), (I′), (II), or (II′), R¹² is —OPO(OR⁷)₂; and R¹³ is substituted or unsubstituted pentyl. In some embodiments of Formula (I), (I′), (II), or (II′), R¹² is —OPO(NR⁷)₂; and R¹³ is substituted or unsubstituted pentyl. In some embodiments of Formula (I), (I′), (II), or (II′), R¹² is —OPO(NR⁷)(OR⁷); and R¹³ is substituted or unsubstituted pentyl. In some embodiments of Formula (I), (I′), (II), or (II′), R¹² is a nitrogen protecting group; and R¹³ is substituted or unsubstituted pentyl. In some embodiments of Formula (I), (I′), (II), or (II′), R¹² is —OR⁷; and R¹³ is substituted or unsubstituted pentyl.

In some embodiments of Formula (I), (I′), (II), or (II′), R¹² is hydrogen; and R¹³ is substituted or unsubstituted C₁₋₆ haloalkyl. In some embodiments of Formula (I), (I′), (II), or (II′), R¹² is substituted or unsubstituted C₁₋₆ alkyl; and R¹³ is substituted or unsubstituted C₁₋₆haloalkyl. In some embodiments of Formula (I), (I′), (II), or (II′), R¹² is substituted or unsubstituted C₁₋₃ alkyl; and R¹³ is substituted or unsubstituted C₁₋₆ haloalkyl. In some embodiments of Formula (I), (I′), (II), or (II′), R¹² is branched or unbranched substituted or unsubstituted C₁₋₆ alkyl; and R¹³ is substituted or unsubstituted C₁₋₆ haloalkyl. In some embodiments of Formula (I), (I′), (II), or (II′), R¹² is substituted or unsubstituted methyl; and R¹³ is substituted or unsubstituted C₁₋₆ haloalkyl. In some embodiments of Formula (I), (I′), (II), or (II′), R¹² is substituted or unsubstituted ethyl; and R¹³ is substituted or unsubstituted C₁₋₆ haloalkyl. In some embodiments of Formula (I), (I′), (II), or (II′), R¹² is substituted or unsubstituted propyl; and R¹³ is substituted or unsubstituted C₁₋₆ haloalkyl. In some embodiments of Formula (I), (I′), (II), or (II′), R¹² is substituted or unsubstituted butyl; and R¹³ is substituted or unsubstituted C₁₋₆ haloalkyl. In some embodiments of Formula (I), (I′), (II), or (II′), R¹² is substituted or unsubstituted pentyl; and R¹³ is substituted or unsubstituted C₁₋₆ haloalkyl. In some embodiments of Formula (I), (I′), (II), or (II′), R¹² is substituted or unsubstituted C₁₋₆ haloalkyl; and R¹³ is substituted or unsubstituted C₁₋₆ haloalkyl. In some embodiments of Formula (I), (I′), (II), or (II′), R¹² is substituted or unsubstituted fluoroalkyl; and R¹³ is substituted or unsubstituted C₁₋₆ haloalkyl. In some embodiments of Formula (I), (I′), (II), or (II′), R¹² is trifluoromethyl; and R¹³ is substituted or unsubstituted C₁₋₆ haloalkyl.

In some embodiments of Formula (I), (I′), (II), or (II′), R¹² is substituted or unsubstituted trifluoroethyl; and R¹³ is substituted or unsubstituted C₁₋₆ haloalkyl. In some embodiments of Formula (I), (I′), (II), or (II′), R¹² is substituted or unsubstituted C₂₋₆ alkenyl; and R¹³ is substituted or unsubstituted C₁₋₆ haloalkyl. In some embodiments of Formula (I), (I′), (II), or (II′), R¹² is substituted or unsubstituted C₂₋₆ alkenyl; and R¹³ is substituted or unsubstituted C₁₋₆ haloalkyl. In some embodiments of Formula (I), (I′), (II), or (II′), R¹² is substituted or unsubstituted vinyl; and R¹³ is substituted or unsubstituted C₁₋₆ haloalkyl. In some embodiments of Formula (I), (I′), (II), or (II′), R¹² is substituted or unsubstituted allyl; and R¹³ is substituted or unsubstituted C₁₋₆ haloalkyl. In some embodiments of Formula (I), (I′), (II), or (II′), R¹² is substituted or unsubstituted propenyl; and R¹³ is substituted or unsubstituted C₁₋₆ haloalkyl. In some embodiments of Formula (I), (I′), (II), or (II′), R¹² is substituted or unsubstituted butenyl; and R¹³ is substituted or unsubstituted C₁₋₆ haloalkyl. In some embodiments of Formula (I), (I′), (II), or (II′), R¹² is substituted or unsubstituted C₂₋₆ alkynyl; and R¹³ is substituted or unsubstituted C₁₋₆ haloalkyl. In some embodiments of Formula (I), (I′), (II), or (II′), R¹² is substituted or unsubstituted acetylenyl; and R¹³ is substituted or unsubstituted C₁₋₆ haloalkyl. In some embodiments of Formula (I), (I′), (II), or (II′), R¹² is substituted or unsubstituted propargyl; and R¹³ is substituted or unsubstituted C₁₋₆ haloalkyl. In some embodiments of Formula (I), (I′), (II), or (II′), R¹² is substituted or unsubstituted butynyl; and R¹³ is substituted or unsubstituted C₁₋₆ haloalkyl. In some embodiments of Formula (I), (I′), (II), or (II′), R¹² is substituted or unsubstituted C₁₋₆ alkoxy; and R¹³ is substituted or unsubstituted C₁₋₆ haloalkyl. In some embodiments of Formula (I), (I′), (II), or (II′), R¹² is CH₂—OMe; and R¹³ is substituted or unsubstituted C₁₋₆ haloalkyl. In some embodiments of Formula (I), (I′), (II), or (II′), R¹² is CH₂—OEt; and R¹³ is substituted or unsubstituted C₁₋₆ haloalkyl. In some embodiments of Formula (I), (I′), (II), or (II′), R¹²CH₂—OPr; and R¹³ is substituted or unsubstituted C₁₋₆ haloalkyl. In some embodiments of Formula (I), (I′), (II), or (II′), R¹² is CH₂—OCF₃; and R¹³ is substituted or unsubstituted C₁₋₆haloalkyl. In some embodiments of Formula (I), (I′), (II), or (II′), R¹² is substituted or unsubstituted CH₂—OR⁷; and R¹³ is substituted or unsubstituted C₁₋₆ haloalkyl. In some embodiments of Formula (I), (I′), (II), or (II′), R¹² is substituted or unsubstituted C₆₋₁₀ aryl; and R¹³ is substituted or unsubstituted C₁₋₆ haloalkyl. In some embodiments of Formula (I), (I′), (II), or (II′), R¹² is substituted or unsubstituted 5-10 membered heteroaryl; and R¹³ is substituted or unsubstituted C₁₋₆ haloalkyl. In some embodiments of Formula (I), (I′), (II), or (II′), R¹² is substituted or unsubstituted 4-10 membered heterocyclyl; and R¹³ is substituted or unsubstituted C₁₋₆ haloalkyl. In some embodiments of Formula (I), (I′), (II), or (II′), R¹² is substituted or unsubstituted 3-10 membered carbocyclyl; and R¹³ is substituted or unsubstituted C₁₋₆ haloalkyl. In some embodiments of Formula (I), (I′), (II), or (II′), R¹² is acyl; and R¹³ is substituted or unsubstituted C₁₋₆ haloalkyl. In some embodiments of Formula (I), (I′), (II), or (II′), R¹² is a substituted or unsubstituted amide; and R¹³ is substituted or unsubstituted C₁₋₆ haloalkyl. In some embodiments of Formula (I), (I′), (II), or (II′), R¹² is a substituted or unsubstituted ester; and R¹³ is substituted or unsubstituted C₁₋₆ haloalkyl. In some embodiments of Formula (I), (I′), (II), or (II′), R¹² is —OH; and R¹³ is substituted or unsubstituted C₁₋₆ haloalkyl. In some embodiments of Formula (I), (I′), (II), or (II′), R¹² is —SO₂R⁷; and R¹³ is substituted or unsubstituted C₁₋₆ haloalkyl. In some embodiments of Formula (I), (I′), (II), or (II′), R¹² is —SOR⁷; and R¹³ is substituted or unsubstituted C₁₋₆haloalkyl. In some embodiments of Formula (I), (I′), (II), or (II′), R¹² is —SO₂NR⁷₂; and R¹³ is substituted or unsubstituted C₁₋₆ haloalkyl. In some embodiments of Formula (I), (I′), (II), or (II′), R¹² is —OSO₂R⁷; and R¹³ is substituted or unsubstituted C₁₋₆ haloalkyl. In some embodiments of Formula (I), (I′), (II), or (II′), R¹² is —OSOR⁷; and R¹³ is substituted or unsubstituted C₁₋₆ haloalkyl. In some embodiments of Formula (I), (I′), (II), or (II′), R¹² is —OSO₂NR⁷₂; and R¹³ is substituted or unsubstituted C₁₋₆ haloalkyl. In some embodiments of Formula (I), (I′), (II), or (II′), R¹² is —OSO₂OR⁷; and R¹³ is substituted or unsubstituted C₁₋₆ haloalkyl. In some embodiments of Formula (I), (I′), (II), or (II′), R¹² is —O(SO)OR⁷; and R¹³ is substituted or unsubstituted C₁₋₆ haloalkyl. In some embodiments of Formula (I), (I′), (II), or (II′), R¹² is —PO(OR⁷)₂; and R¹³ is substituted or unsubstituted C₁₋₆ haloalkyl. In some embodiments of Formula (I), (I′), (II), or (II′), R¹² is —PO(NR⁷)₂; and R¹³ is substituted or unsubstituted C₁₋₆ haloalkyl. In some embodiments of Formula (I), (I′), (II), or (II′), R¹² is —PO(NR⁷)(OR⁷); and R¹³ is substituted or unsubstituted C₁₋₆ haloalkyl. In some embodiments of Formula (I), (I′), (II), or (II′), R¹² is —OPO(OR⁷)₂; and R¹³ is substituted or unsubstituted C₁₋₆ haloalkyl. In some embodiments of Formula (I), (I′), (II), or (II′), R¹² is —OPO(NR⁷)₂; and R¹³ is substituted or unsubstituted C₁₋₆ haloalkyl. In some embodiments of Formula (I), (I′), (II), or (II′), R¹² is —OPO(NR⁷)(OR⁷); and R¹³ is substituted or unsubstituted C₁₋₆ haloalkyl. In some embodiments of Formula (I), (I′), (II), or (II′), R¹² is a nitrogen protecting group; and R³ is substituted or unsubstituted C₁₋₆ haloalkyl. In some embodiments of Formula (I), (I′), (II), or (II′), R¹² is —OR⁷; and R¹³ is substituted or unsubstituted C₁₋₆ haloalkyl.

In some embodiments of Formula (I), (I′), (II), or (II′), R¹² is hydrogen; and R¹³ is substituted or unsubstituted fluoroalkyl. In some embodiments of Formula (I), (I′), (II), or (II′), R¹² is substituted or unsubstituted C₁₋₆ alkyl; and R¹³ is substituted or unsubstituted fluoroalkyl. In some embodiments of Formula (I), (I′), (II), or (II′), R¹² is substituted or unsubstituted C₁₋₃ alkyl; and R¹³ is substituted or unsubstituted fluoroalkyl. In some embodiments of Formula (I), (I′), (II), or (II′), R¹² is branched or unbranched substituted or unsubstituted C₁₋₆ alkyl; and R¹³ is substituted or unsubstituted fluoroalkyl. In some embodiments of Formula (I), (I′), (II), or (II′), R¹² is substituted or unsubstituted methyl; and R¹³ is substituted or unsubstituted fluoroalkyl. In some embodiments of Formula (I), (I′), (II), or (II′), R¹² is substituted or unsubstituted ethyl; and R¹³ is substituted or unsubstituted fluoroalkyl. In some embodiments of Formula (I), (I′), (II), or (II′), R¹² is substituted or unsubstituted propyl; and R¹³ is substituted or unsubstituted fluoroalkyl. In some embodiments of Formula (I), (I′), (II), or (II′), R¹² is substituted or unsubstituted butyl; and R¹³ is substituted or unsubstituted fluoroalkyl. In some embodiments of Formula (I), (I′), (II), or (II′), R¹² is substituted or unsubstituted pentyl; and R¹³ is substituted or unsubstituted fluoroalkyl. In some embodiments of Formula (I), (I′), (II), or (II′), R¹² is substituted or unsubstituted C₁₋₆ haloalkyl; and R¹³ is substituted or unsubstituted fluoroalkyl. In some embodiments of Formula (I), (I′), (II), or (II′), R¹² is substituted or unsubstituted fluoroalkyl; and R¹³ is substituted or unsubstituted fluoroalkyl. In some embodiments of Formula (I), (I′), (II), or (II′), R¹² is trifluoromethyl; and R¹³ is substituted or unsubstituted fluoroalkyl. In some embodiments of Formula (I), (I′), (II), or (II′), R¹² is substituted or unsubstituted trifluoroethyl; and R¹³ is substituted or unsubstituted fluoroalkyl. In some embodiments of Formula (I), (I′), (II), or (II′), R¹² is substituted or unsubstituted C₂₋₆ alkenyl; and R¹³ is substituted or unsubstituted fluoroalkyl. In some embodiments of Formula (I), (I′), (II), or (II′), R¹² is substituted or unsubstituted C₂₋₆ alkenyl; and R¹³ is substituted or unsubstituted fluoroalkyl. In some embodiments of Formula (I), (I′), (II), or (II′), R¹² is substituted or unsubstituted vinyl; and R¹³ is substituted or unsubstituted fluoroalkyl. In some embodiments of Formula (I), (I′), (II), or (II′), R¹² is substituted or unsubstituted allyl; and R¹³ is substituted or unsubstituted fluoroalkyl. In some embodiments of Formula (I), (I′), (II), or (II′), R¹² is substituted or unsubstituted propenyl; and R¹³ is substituted or unsubstituted fluoroalkyl. In some embodiments of Formula (I), (I′), (II), or (II′), R¹² is substituted or unsubstituted butenyl; and R¹³ is substituted or unsubstituted fluoroalkyl. In some embodiments of Formula (I), (I′), (II), or (II′), R¹² is substituted or unsubstituted C₂₋₆ alkynyl; and R¹³ is substituted or unsubstituted fluoroalkyl. In some embodiments of Formula (I), (I′), (II), or (II′), R¹² is substituted or unsubstituted acetylenyl; and R¹³ is substituted or unsubstituted fluoroalkyl. In some embodiments of Formula (I), (I′), (II), or (II′), R¹² is substituted or unsubstituted propargyl; and R¹³ is substituted or unsubstituted fluoroalkyl. In some embodiments of Formula (I), (I′), (II), or (II′), R¹² is substituted or unsubstituted butynyl; and R¹³ is substituted or unsubstituted fluoroalkyl. In some embodiments of Formula (I), (I′), (II), or (II′), R¹² is substituted or unsubstituted C₁₋₆ alkoxy; and R¹³ is substituted or unsubstituted fluoroalkyl. In some embodiments of Formula (I), (I′), (II), or (II′), R¹² is CH₂—OMe; and R¹³ is substituted or unsubstituted fluoroalkyl. In some embodiments of Formula (I), (I′), (II), or (II′), R¹² is CH₂—OEt; and R¹³ is substituted or unsubstituted fluoroalkyl. In some embodiments of Formula (I), (I′), (II), or (II′), R¹²CH₂—OPr; and R¹³ is substituted or unsubstituted fluoroalkyl. In some embodiments of Formula (I), (I′), (II), or (II′), R¹² is CH₂—OCF₃; and R¹³ is substituted or unsubstituted fluoroalkyl. In some embodiments of Formula (I), (I′), (II), or (II′), R¹² is substituted or unsubstituted CH₂—OR⁷; and R¹³ is substituted or unsubstituted fluoroalkyl. In some embodiments of Formula (I), (I′), (II), or (II′), R¹² is substituted or unsubstituted C₆₋₁₀ aryl; and R¹³ is substituted or unsubstituted fluoroalkyl. In some embodiments of Formula (I), (I′), (II), or (II′), R¹² is substituted or unsubstituted 5-10 membered heteroaryl; and R¹³ is substituted or unsubstituted fluoroalkyl. In some embodiments of Formula (I), (I′), (II), or (II′), R¹² is substituted or unsubstituted 4-10 membered heterocyclyl; and R¹³ is substituted or unsubstituted fluoroalkyl. In some embodiments of Formula (I), (I′), (II), or (II′), R¹² is substituted or unsubstituted 3-10 membered carbocyclyl; and R¹³ is substituted or unsubstituted fluoroalkyl. In some embodiments of Formula (I), (I′), (II), or (II′), R¹² is acyl; and R¹³ is substituted or unsubstituted fluoroalkyl. In some embodiments of Formula (I), (I′), (II), or (II′), R¹² is a substituted or unsubstituted amide; and R¹³ is substituted or unsubstituted fluoroalkyl. In some embodiments of Formula (I), (I′), (II), or (II′), R¹² is a substituted or unsubstituted ester; and R¹³ is substituted or unsubstituted fluoroalkyl. In some embodiments of Formula (I), (I′), (II), or (II′), R¹² is —OH; and R¹³ is substituted or unsubstituted fluoroalkyl. In some embodiments of Formula (I), (I′), (II), or (II′), R¹² is —SO₂R⁷; and R¹³ is substituted or unsubstituted fluoroalkyl. In some embodiments of Formula (I), (I′), (II), or (II′), R¹² is —SOR⁷; and R¹³ is substituted or unsubstituted fluoroalkyl. In some embodiments of Formula (I), (I′), (II), or (II′), R¹² is —SO₂NR⁷₂; and R¹³ is substituted or unsubstituted fluoroalkyl. In some embodiments of Formula (I), (I′), (II), or (II′), R¹² is —OSO₂R⁷; and R¹³ is substituted or unsubstituted fluoroalkyl. In some embodiments of Formula (I), (I′), (II), or (II′), R¹² is —OSOR⁷; and R¹³ is substituted or unsubstituted fluoroalkyl. In some embodiments of Formula (I), (I′), (II), or (II′), R¹² is —OSO₂NR⁷₂; and R¹³ is substituted or unsubstituted fluoroalkyl. In some embodiments of Formula (I), (I′), (II), or (II′), R¹² is —OSO₂OR⁷; and R¹³ is substituted or unsubstituted fluoroalkyl. In some embodiments of Formula (I), (I′), (II), or (II′), R¹² is —O(SO)OR⁷; and R¹³ is substituted or unsubstituted fluoroalkyl. In some embodiments of Formula (I), (I′), (II), or (II′), R¹² is —PO(OR⁷)₂; and R¹³ is substituted or unsubstituted fluoroalkyl. In some embodiments of Formula (I), (I′), (II), or (II′), R¹² is —PO(NR⁷)₂; and R¹³ is substituted or unsubstituted fluoroalkyl. In some embodiments of Formula (I), (I′), (II), or (II′), R¹² is —PO(NR⁷)(OR⁷); and R¹³ is substituted or unsubstituted fluoroalkyl. In some embodiments of Formula (I), (I′), (II), or (II′), R¹² is —OPO(OR⁷)₂; and R¹³ is substituted or unsubstituted fluoroalkyl. In some embodiments of Formula (I), (I′), (II), or (II′), R¹² is —OPO(NR⁷)₂; and R¹³ is substituted or unsubstituted fluoroalkyl. In some embodiments of Formula (I), (I′), (II), or (II′), R¹² is —OPO(NR⁷)(OR⁷); and R¹³ is substituted or unsubstituted fluoroalkyl. In some embodiments of Formula (I), (I′), (II), or (II′), R¹² is a nitrogen protecting group; and R¹³ is substituted or unsubstituted fluoroalkyl. In some embodiments of Formula (I), (I′), (II), or (II′), R¹² is —OR⁷; and R¹³ is substituted or unsubstituted fluoroalkyl.

In some embodiments of Formula (I), (I′), (II), or (II′), R¹² is hydrogen; and R¹³ is trifluoromethyl. In some embodiments of Formula (I), (I′), (II), or (II′), R¹² is substituted or unsubstituted C₁₋₆ alkyl; and R¹³ is trifluoromethyl. In some embodiments of Formula (I), (I′), (II), or (II′), R¹² is substituted or unsubstituted C₁₋₃ alkyl; and R¹³ is trifluoromethyl. In some embodiments of Formula (I), (I′), (II), or (II′), R¹² is branched or unbranched substituted or unsubstituted C₁₋₆ alkyl; and R¹³ is trifluoromethyl. In some embodiments of Formula (I), (I′), (II), or (II′), R¹² is substituted or unsubstituted methyl; and R¹³ is trifluoromethyl. In some embodiments of Formula (I), (I′), (II), or (II′), R¹² is substituted or unsubstituted ethyl; and R¹³ is trifluoromethyl. In some embodiments of Formula (I), (I′), (II), or (II′), R¹² is substituted or unsubstituted propyl; and R¹³ is trifluoromethyl. In some embodiments of Formula (I), (I′), (II), or (II′), R¹² is substituted or unsubstituted butyl; and R¹³ is trifluoromethyl. In some embodiments of Formula (I), (I′), (II), or (II′), R¹² is substituted or unsubstituted pentyl; and R¹³ is trifluoromethyl. In some embodiments of Formula (I), (I′), (II), or (II′), R¹² is substituted or unsubstituted C₁₋₆ haloalkyl; and R¹³ is trifluoromethyl. In some embodiments of Formula (I), (I′), (II), or (II′), R¹² is substituted or unsubstituted fluoroalkyl; and R¹³ is trifluoromethyl. In some embodiments of Formula (I), (I′), (II), or (II′), R¹² is trifluoromethyl; and R¹³ is trifluoromethyl. In some embodiments of Formula (I), (I′), (II), or (II′), R¹² is substituted or unsubstituted trifluoroethyl; and R¹³ is trifluoromethyl. In some embodiments of Formula (I), (I′), (II), or (II′), R¹² is substituted or unsubstituted C₂₋₆ alkenyl; and R¹³ is trifluoromethyl. In some embodiments of Formula (I), (I′), (II), or (II′), R¹² is substituted or unsubstituted C₂₋₆ alkenyl; and R¹³ is trifluoromethyl. In some embodiments of Formula (I), (I′), (II), or (II′), R¹² is substituted or unsubstituted vinyl; and R¹³ is trifluoromethyl. In some embodiments of Formula (I), (I′), (II), or (II′), R¹² is substituted or unsubstituted allyl; and R¹³ is trifluoromethyl. In some embodiments of Formula (I), (I′), (II), or (II′), R¹² is substituted or unsubstituted propenyl; and R¹³ is trifluoromethyl. In some embodiments of Formula (I), (I′), (II), or (II′), R¹² is substituted or unsubstituted butenyl; and R¹³ is trifluoromethyl. In some embodiments of Formula (I), (I′), (II), or (II′), R¹² is substituted or unsubstituted C₂₋₆ alkynyl; and R¹³ is trifluoromethyl. In some embodiments of Formula (I), (I′), (II), or (II′), R¹² is substituted or unsubstituted acetylenyl; and R¹³ is trifluoromethyl. In some embodiments of Formula (I), (I′), (II), or (II′), R¹² is substituted or unsubstituted propargyl; and R¹³ is trifluoromethyl. In some embodiments of Formula (I), (I′), (II), or (II′), R¹² is substituted or unsubstituted butynyl; and R¹³ is trifluoromethyl. In some embodiments of Formula (I), (I′), (II), or (II′), R¹² is substituted or unsubstituted C₁₋₆ alkoxy; and R¹³ is trifluoromethyl. In some embodiments of Formula (I), (I′), (II), or (II′), R¹² is CH₂—OMe; and R¹³ is trifluoromethyl. In some embodiments of Formula (I), (I′), (II), or (II′), R¹² is CH₂-OEt; and R¹³ is trifluoromethyl. In some embodiments of Formula (I), (I′), (II), or (II′), R¹²CH—OPr; and R¹³ is trifluoromethyl. In some embodiments of Formula (I), (I′), (II), or (II′), R¹² is CH₂—OCF₃; and R¹³ is trifluoromethyl. In some embodiments of Formula (I), (I′), (II), or (II′), R¹² is substituted or unsubstituted CH₂—OR⁷; and R¹³ is trifluoromethyl. In some embodiments of Formula (I), (I′), (II), or (II′), R¹² is substituted or unsubstituted C₆₋₁₀ aryl; and R¹³ is trifluoromethyl. In some embodiments of Formula (I), (I′), (II), or (II′), R¹² is substituted or unsubstituted 5-10 membered heteroaryl; and R¹³ is trifluoromethyl. In some embodiments of Formula (I), (I′), (II), or (II′), R¹² is substituted or unsubstituted 4-10 membered heterocyclyl; and R¹³ is trifluoromethyl. In some embodiments of Formula (I), (I′), (II), or (II′), R¹² is substituted or unsubstituted 3-10 membered carbocyclyl; and R¹³ is trifluoromethyl. In some embodiments of Formula (I), (I′), (II), or (II′), R¹² is acyl; and R¹³ is trifluoromethyl. In some embodiments of Formula (I), (I′), (II), or (II′), R¹² is a substituted or unsubstituted amide; and R¹³ is trifluoromethyl. In some embodiments of Formula (I), (I′), (II), or (II′), R¹² is a substituted or unsubstituted ester; and R¹³ is trifluoromethyl. In some embodiments of Formula (I), (I′), (II), or (II′), R¹² is —OH; and R¹³ is trifluoromethyl. In some embodiments of Formula (I), (I′), (II), or (II′), R¹² is —SO₂R⁷; and R¹³ is trifluoromethyl. In some embodiments of Formula (I), (I′), (II), or (II′), R¹² is —SOR⁷; and R¹³ is trifluoromethyl. In some embodiments of Formula (I), (I′), (II), or (II′), R¹² is —SO₂NR⁷₂; and R¹³ is trifluoromethyl. In some embodiments of Formula (I), (I′), (II), or (II′), R¹² is —OSO₂R⁷; and R¹³ is trifluoromethyl. In some embodiments of Formula (I), (I′), (II), or (II′), R¹² is —OSOR⁷; and R¹³ is trifluoromethyl. In some embodiments of Formula (I), (I′), (II), or (II′), R¹² is —OSO₂NR⁷₂; and R¹¹ is trifluoromethyl. In some embodiments of Formula (I), (I′), (II), or (II′), R¹² is —OSO₂OR⁷; and R³ is trifluoromethyl. In some embodiments of Formula (I), (I′), (II), or (II′), R¹² is —O(SO)OR⁷; and R¹³ is trifluoromethyl.

In some embodiments of Formula (I), (I′), (II), or (II′), R¹² is —PO(OR⁷)₂; and R¹³ is trifluoromethyl. In some embodiments of Formula (I), (I′), (II), or (II′), R¹² is —PO(NR⁷)₂; and R¹³ is trifluoromethyl. In some embodiments of Formula (I), (I′), (II), or (II′), R¹² is —PO(NR⁷)(OR⁷); and R¹³ is trifluoromethyl. In some embodiments of Formula (I), (I′), (II), or (II′), R¹² is —OPO(OR⁷)₂; and R¹³ is trifluoromethyl. In some embodiments of Formula (I), (I′), (II), or (II′), R¹² is —OPO(NR⁷)₂; and R¹³ is trifluoromethyl. In some embodiments of Formula (I), (I′), (II), or (II′), R¹² is —OPO(NR⁷)(OR⁷); and R¹³ is trifluoromethyl. In some embodiments of Formula (I), (I′), (II), or (II′), R¹² is a nitrogen protecting group; and R¹³ is trifluoromethyl. In some embodiments of Formula (I), (I′), (II), or (II′), R¹² is —OR⁷; and R¹³ is trifluoromethyl.

In some embodiments of Formula (I), (I′), (II), or (II′), R¹² is hydrogen; and R¹³ is substituted or unsubstituted trifluoroethyl. In some embodiments of Formula (I), (I′), (II), or (II′), R¹² is substituted or unsubstituted C₁₋₆ alkyl; and R¹³ is substituted or unsubstituted trifluoroethyl. In some embodiments of Formula (I), (I′), (II), or (II′), R¹² is substituted or unsubstituted C₁₋₃ alkyl; and R¹³ is substituted or unsubstituted trifluoroethyl. In some embodiments of Formula (I), (I′), (II), or (II′), R¹² is branched or unbranched substituted or unsubstituted C₁₋₆ alkyl; and R¹³ is substituted or unsubstituted trifluoroethyl. In some embodiments of Formula (I), (I′), (II), or (II′), R¹² is substituted or unsubstituted methyl; and R¹³ is substituted or unsubstituted trifluoroethyl. In some embodiments of Formula (I), (I′), (II), or (II′), R¹² is substituted or unsubstituted ethyl; and R¹³ is substituted or unsubstituted trifluoroethyl. In some embodiments of Formula (I), (I′), (II), or (II′), R¹² is substituted or unsubstituted propyl; and R¹³ is substituted or unsubstituted trifluoroethyl. In some embodiments of Formula (I), (I′), (II), or (II′), R¹² is substituted or unsubstituted butyl; and R¹³ is substituted or unsubstituted trifluoroethyl. In some embodiments of Formula (I), (I′), (II), or (II′), R¹² is substituted or unsubstituted pentyl; and R¹³ is substituted or unsubstituted trifluoroethyl. In some embodiments of Formula (I), (I′), (II), or (II′), R¹² is substituted or unsubstituted C₁₋₆ haloalkyl; and R¹³ is substituted or unsubstituted trifluoroethyl. In some embodiments of Formula (I), (I′), (II), or (II′), R¹² is substituted or unsubstituted fluoroalkyl; and R¹³ is substituted or unsubstituted trifluoroethyl. In some embodiments of Formula (I), (I′), (II), or (II′), R¹² is trifluoromethyl; and R¹³ is substituted or unsubstituted trifluoroethyl. In some embodiments of Formula (I), (I′), (II), or (II′), R¹² is substituted or unsubstituted trifluoroethyl; and R¹³ is substituted or unsubstituted trifluoroethyl. In some embodiments of Formula (I), (I′), (II), or (II′), R¹² is substituted or unsubstituted C₂₋₆ alkenyl; and R¹³ is substituted or unsubstituted trifluoroethyl. In some embodiments of Formula (I), (I′), (II), or (II′), R¹² is substituted or unsubstituted C₂₋₆ alkenyl; and R¹³ is substituted or unsubstituted trifluoroethyl. In some embodiments of Formula (I), (I′), (II), or (II′), R¹² is substituted or unsubstituted vinyl; and R¹³ is substituted or unsubstituted trifluoroethyl. In some embodiments of Formula (I), (I′), (II), or (II′), R¹² is substituted or unsubstituted allyl; and R¹³ is substituted or unsubstituted trifluoroethyl. In some embodiments of Formula (I), (I′), (II), or (II′), R¹² is substituted or unsubstituted propenyl; and R¹³ is substituted or unsubstituted trifluoroethyl. In some embodiments of Formula (I), (I′), (II), or (II′), R¹² is substituted or unsubstituted butenyl; and R¹³ is substituted or unsubstituted trifluoroethyl. In some embodiments of Formula (I), (I′), (II), or (II′), R¹² is substituted or unsubstituted C₂₋₆ alkynyl; and R¹³ is substituted or unsubstituted trifluoroethyl. In some embodiments of Formula (I), (I′), (II), or (II′), R¹² is substituted or unsubstituted acetylenyl; and R¹³ is substituted or unsubstituted trifluoroethyl. In some embodiments of Formula (I), (I′), (II), or (II′), R¹² is substituted or unsubstituted propargyl; and R¹³ is substituted or unsubstituted trifluoroethyl. In some embodiments of Formula (I), (I′), (II), or (II′), R¹² is substituted or unsubstituted butynyl; and R¹³ is substituted or unsubstituted trifluoroethyl. In some embodiments of Formula (I), (I′), (II), or (II′), R¹² is substituted or unsubstituted C₁₋₆ alkoxy; and R¹³ is substituted or unsubstituted trifluoroethyl. In some embodiments of Formula (I), (I′), (II), or (II′), R¹² is CH₂—OMe; and R¹³ is substituted or unsubstituted trifluoroethyl. In some embodiments of Formula (I), (I′), (II), or (II′), R¹² is CH₂—OEt; and R¹³ is substituted or unsubstituted trifluoroethyl. In some embodiments of Formula (I), (I′), (II), or (II′), R¹²CH₂—OPr; and R¹³ is substituted or unsubstituted trifluoroethyl. In some embodiments of Formula (I), (I′), (II), or (II′), R¹² is CH₂—OCF₃; and R¹³ is substituted or unsubstituted trifluoroethyl. In some embodiments of Formula (I), (I′), (II), or (II′), R¹² is substituted or unsubstituted CH₂—OR⁷; and R¹³ is substituted or unsubstituted trifluoroethyl. In some embodiments of Formula (I), (I′), (II), or (II′), R¹² is substituted or unsubstituted C₆₋₁₀ aryl; and R¹³ is substituted or unsubstituted trifluoroethyl. In some embodiments of Formula (I), (I′), (II), or (II′), R¹² is substituted or unsubstituted 5-10 membered heteroaryl; and R¹³ is substituted or unsubstituted trifluoroethyl. In some embodiments of Formula (I), (I′), (II), or (II′), R¹² is substituted or unsubstituted 4-10 membered heterocyclyl; and R¹³ is substituted or unsubstituted trifluoroethyl. In some embodiments of Formula (I), (I′), (II), or (II′), R¹² is substituted or unsubstituted 3-10 membered carbocyclyl; and R¹³ is substituted or unsubstituted trifluoroethyl. In some embodiments of Formula (I), (I′), (II), or (II′), R¹² is acyl; and R¹³ is substituted or unsubstituted trifluoroethyl. In some embodiments of Formula (I), (I′), (II), or (II′), R¹² is a substituted or unsubstituted amide; and R¹³ is substituted or unsubstituted trifluoroethyl. In some embodiments of Formula (I), (I′), (II), or (II′), R¹² is a substituted or unsubstituted ester; and R¹³ is substituted or unsubstituted trifluoroethyl. In some embodiments of Formula (I), (I′), (II), or (II′), R¹² is —OH; and R¹³ is substituted or unsubstituted trifluoroethyl. In some embodiments of Formula (I), (I′), (II), or (II′), R¹² is —SO₂R⁷; and R¹³ is substituted or unsubstituted trifluoroethyl. In some embodiments of Formula (I), (I′), (II), or (II′), R¹² is —SOR⁷; and R¹³ is substituted or unsubstituted trifluoroethyl. In some embodiments of Formula (I), (I′), (II), or (II′), R¹² is —SO₂NR⁷₂; and R¹³ is substituted or unsubstituted trifluoroethyl. In some embodiments of Formula (I), (I′), (II), or (II′), R¹² is —OSO₂R⁷; and R¹³ is substituted or unsubstituted trifluoroethyl. In some embodiments of Formula (I), (I′), (II), or (II′), R¹² is —OSOR⁷; and R¹³ is substituted or unsubstituted trifluoroethyl. In some embodiments of Formula (I), (I′), (II), or (II′), R¹² is —OSO₂NR⁷₂; and R¹³ is substituted or unsubstituted trifluoroethyl. In some embodiments of Formula (I), (I′), (II), or (II′), R¹² is —OSO₂OR⁷; and R¹³ is substituted or unsubstituted trifluoroethyl. In some embodiments of Formula (I), (I′), (II), or (II′), R¹² is —O(SO)OR⁷; and R¹³ is substituted or unsubstituted trifluoroethyl. In some embodiments of Formula (I), (I′), (II), or (II′), R¹² is —PO(OR⁷)₂; and R¹³ is substituted or unsubstituted trifluoroethyl. In some embodiments of Formula (I), (I′), (II), or (II′), R¹² is —PO(NR⁷)₂; and R¹³ is substituted or unsubstituted trifluoroethyl. In some embodiments of Formula (I), (I′), (II), or (II′), R¹² is —PO(NR⁷)(OR⁷); and R¹³ is substituted or unsubstituted trifluoroethyl. In some embodiments of Formula (I), (I′), (II), or (II′), R¹² is —OPO(OR⁷)₂; and R¹³ is substituted or unsubstituted trifluoroethyl. In some embodiments of Formula (I), (I′), (II), or (II′), R¹² is —OPO(NR⁷)₂; and R¹³ is substituted or unsubstituted trifluoroethyl. In some embodiments of Formula (I), (I′), (II), or (II′), R¹² is —OPO(NR⁷)(OR⁷); and R¹³ is substituted or unsubstituted trifluoroethyl. In some embodiments of Formula (I), (I′), (II), or (II′), R¹² is a nitrogen protecting group; and R¹³ is substituted or unsubstituted trifluoroethyl. In some embodiments of Formula (I), (I′), (II), or (II′), R¹² is —OR⁷; and R¹³ is substituted or unsubstituted trifluoroethyl.

In some embodiments of Formula (I), (I′), (II), or (II′), R¹² is hydrogen; and R¹³ is substituted or unsubstituted C₂₋₆ alkenyl. In some embodiments of Formula (I), (I′), (II), or (II′), R¹² is substituted or unsubstituted C₁₋₆ alkyl; and R¹³ is substituted or unsubstituted C₂₋₆ alkenyl. In some embodiments of Formula (I), (I′), (II), or (II′), R¹² is substituted or unsubstituted C₁₋₃ alkyl; and R¹³ is substituted or unsubstituted C₂₋₆ alkenyl. In some embodiments of Formula (I), (I′), (II), or (II′), R¹² is branched or unbranched substituted or unsubstituted C₁₋₆ alkyl; and R¹³ is substituted or unsubstituted C₂₋₆ alkenyl. In some embodiments of Formula (I), (I′), (II), or (II′), R¹² is substituted or unsubstituted methyl; and R¹³ is substituted or unsubstituted C₂₋₆ alkenyl. In some embodiments of Formula (I), (I′), (II), or (II′), R¹² is substituted or unsubstituted ethyl; and R¹³ is substituted or unsubstituted C₂₋₆ alkenyl. In some embodiments of Formula (I), (I′), (II), or (II′), R¹² is substituted or unsubstituted propyl; and R¹³ is substituted or unsubstituted C₂₋₆ alkenyl. In some embodiments of Formula (I), (I′), (II), or (II′), R¹² is substituted or unsubstituted butyl; and R¹³ is substituted or unsubstituted C₂₋₆ alkenyl. In some embodiments of Formula (I), (I′), (II), or (II′), R¹² is substituted or unsubstituted pentyl; and R¹³ is substituted or unsubstituted C₂₋₆ alkenyl. In some embodiments of Formula (I), (I′), (II), or (II′), R¹² is substituted or unsubstituted C₁₋₆ haloalkyl; and R¹³ is substituted or unsubstituted C₂₋₆ alkenyl. In some embodiments of Formula (I), (I′), (II), or (II′), R¹² is substituted or unsubstituted fluoroalkyl; and R¹³ is substituted or unsubstituted C₂₋₆ alkenyl. In some embodiments of Formula (I), (I′), (II), or (II′), R¹² is trifluoromethyl; and R¹³ is substituted or unsubstituted C₂₋₆ alkenyl. In some embodiments of Formula (I), (I′), (II), or (II′), R¹² is substituted or unsubstituted trifluoroethyl; and R¹³ is substituted or unsubstituted C₂₋₆ alkenyl. In some embodiments of Formula (I), (I′), (II), or (II′), R¹² is substituted or unsubstituted C₂₋₆ alkenyl; and R¹³ is substituted or unsubstituted C₂₋₆ alkenyl. In some embodiments of Formula (I), (I′), (II), or (II′), R¹² is substituted or unsubstituted C₂₋₆ alkenyl; and R¹³ is substituted or unsubstituted C₂₋₆ alkenyl. In some embodiments of Formula (I), (I′), (II), or (II′), R¹² is substituted or unsubstituted vinyl; and R¹³ is substituted or unsubstituted C₂₋₆ alkenyl. In some embodiments of Formula (I), (I′), (II), or (II′), R¹² is substituted or unsubstituted allyl; and R¹³ is substituted or unsubstituted C₂₋₆ alkenyl. In some embodiments of Formula (I), (I′), (II), or (II′), R¹² is substituted or unsubstituted propenyl; and R¹³ is substituted or unsubstituted C₂₋₆ alkenyl. In some embodiments of Formula (I), (I′), (II), or (II′), R¹² is substituted or unsubstituted butenyl; and R¹³ is substituted or unsubstituted C₂₋₆ alkenyl. In some embodiments of Formula (I), (I′), (II), or (II′), R¹² is substituted or unsubstituted C₂₋₆ alkynyl; and R¹³ is substituted or unsubstituted C₂₋₆ alkenyl. In some embodiments of Formula (I), (I′), (II), or (II′), R¹² is substituted or unsubstituted acetylenyl; and R¹³ is substituted or unsubstituted C₂₋₆ alkenyl. In some embodiments of Formula (I), (I′), (II), or (II′), R¹² is substituted or unsubstituted propargyl; and R¹³ is substituted or unsubstituted C₂₋₆ alkenyl. In some embodiments of Formula (I), (I′), (II), or (II′), R¹² is substituted or unsubstituted butynyl; and R¹³ is substituted or unsubstituted C₂₋₆ alkenyl. In some embodiments of Formula (I), (I′), (II), or (II′), R¹² is substituted or unsubstituted C₁₋₆ alkoxy; and R¹³ is substituted or unsubstituted C₂₋₆ alkenyl. In some embodiments of Formula (I), (I′), (II), or (II′), R¹² is CH₂—OMe; and R¹³ is substituted or unsubstituted C₂₋₆ alkenyl. In some embodiments of Formula (I), (I′), (II), or (II′), R¹² is CH₂—OEt; and R¹³ is substituted or unsubstituted C₂₋₆ alkenyl. In some embodiments of Formula (I), (I′), (II), or (II′), R¹²CH₂—OPr; and R¹³ is substituted or unsubstituted C₂₋₆ alkenyl. In some embodiments of Formula (I), (I′), (II), or (II′), R¹² is CH₂—OCF₃; and R¹³ is substituted or unsubstituted C₂₋₆ alkenyl. In some embodiments of Formula (I), (I′), (II), or (II′), R¹² is substituted or unsubstituted CH₂—OR⁷; and R¹³ is substituted or unsubstituted C₂₋₆ alkenyl. In some embodiments of Formula (I), (I′), (II), or (II′), R¹² is substituted or unsubstituted C₆₋₁₀ aryl; and R¹³ is substituted or unsubstituted C₂₋₆ alkenyl. In some embodiments of Formula (I), (I′), (II), or (II′), R¹² is substituted or unsubstituted 5-10 membered heteroaryl; and R¹³ is substituted or unsubstituted C₂₋₆ alkenyl. In some embodiments of Formula (I), (I′), (II), or (II′), R¹² is substituted or unsubstituted 4-10 membered heterocyclyl; and R¹³ is substituted or unsubstituted C₂₋₆ alkenyl. In some embodiments of Formula (I), (I′), (II), or (II′), R¹² is substituted or unsubstituted 3-10 membered carbocyclyl; and R¹³ is substituted or unsubstituted C₂₋₆ alkenyl. In some embodiments of Formula (I), (I′), (II), or (II′), R¹² is acyl; and R¹³ is substituted or unsubstituted C₂₋₆ alkenyl. In some embodiments of Formula (I), (I′), (II), or (II′), R¹² is a substituted or unsubstituted amide; and R¹³ is substituted or unsubstituted C₂₋₆ alkenyl. In some embodiments of Formula (I), (I′), (II), or (II′), R¹² is a substituted or unsubstituted ester; and R¹³ is substituted or unsubstituted C₂₋₆ alkenyl. In some embodiments of Formula (I), (I′), (II), or (II′), R¹² is —OH; and R¹³ is substituted or unsubstituted C₂₋₆ alkenyl. In some embodiments of Formula (I), (I′), (II), or (II′), R¹² is —SO₂R⁷; and R¹³ is substituted or unsubstituted C₂₋₆ alkenyl. In some embodiments of Formula (I), (I′), (II), or (II′), R¹² is —SOR⁷; and R¹³ is substituted or unsubstituted C₂₋₆ alkenyl. In some embodiments of Formula (I), (I′), (II), or (II′), R¹² is —SO₂NR⁷₂; and R¹³ is substituted or unsubstituted C₂₋₆ alkenyl. In some embodiments of Formula (I), (I′), (II), or (II′), R¹² is —OSO₂R⁷; and R¹³ is substituted or unsubstituted C₂₋₆ alkenyl. In some embodiments of Formula (I), (I′), (II), or (II′), R¹² is —OSOR⁷; and R¹³ is substituted or unsubstituted C₂₋₆ alkenyl. In some embodiments of Formula (I), (I′), (II), or (II′), R¹² is —OSO₂NR⁷₂; and R¹³ is substituted or unsubstituted C₂₋₆ alkenyl. In some embodiments of Formula (I), (I′), (II), or (II′), R¹² is —OSO₂OR⁷; and R¹³ is substituted or unsubstituted C₂₋₆ alkenyl. In some embodiments of Formula (I), (I′), (II), or (II′), R¹² is —O(SO)OR⁷; and R¹³ is substituted or unsubstituted C₂₋₆ alkenyl. In some embodiments of Formula (I), (I′), (II), or (II′), R¹² is —PO(OR⁷)₂; and R¹³ is substituted or unsubstituted C₂₋₆ alkenyl. In some embodiments of Formula (I), (I′), (II), or (II′), R¹² is —PO(NR⁷)₂; and R¹³ is substituted or unsubstituted C₂₋₆ alkenyl. In some embodiments of Formula (I), (I′), (II), or (II′), R¹² is —PO(NR⁷)(OR⁷); and R¹³ is substituted or unsubstituted C₂₋₆ alkenyl. In some embodiments of Formula (I), (I′), (II), or (II′), R¹² is —OPO(OR⁷)₂; and R¹³ is substituted or unsubstituted C₂₋₆ alkenyl. In some embodiments of Formula (I), (I′), (II), or (II′), R¹² is —OPO(NR⁷)₂; and R¹³ is substituted or unsubstituted C₂₋₆ alkenyl. In some embodiments of Formula (I), (I′), (II), or (II′), R¹² is —OPO(NR⁷)(OR⁷); and R¹³ is substituted or unsubstituted C₂₋₆ alkenyl. In some embodiments of Formula (I), (I′), (II), or (II′), R¹² is a nitrogen protecting group; and R¹³ is substituted or unsubstituted C₂₋₆ alkenyl. In some embodiments of Formula (I), (I′), (II), or (II′), R¹² is —OR⁷; and R¹³ is substituted or unsubstituted C₂₋₆ alkenyl.

In some embodiments of Formula (I), (I′), (II), or (II′), R¹² is hydrogen; and R¹³ is substituted or unsubstituted vinyl. In some embodiments of Formula (I), (I′), (II), or (II′), R¹² is substituted or unsubstituted C₁₋₆ alkyl; and R¹³ is substituted or unsubstituted vinyl. In some embodiments of Formula (I), (I′), (II), or (II′), R¹² is substituted or unsubstituted C₁₋₃alkyl; and R¹³ is substituted or unsubstituted vinyl. In some embodiments of Formula (I), (I′), (II), or (II′), R¹² is branched or unbranched substituted or unsubstituted C₁₋₆ alkyl; and R¹³ is substituted or unsubstituted vinyl. In some embodiments of Formula (I), (I′), (II), or (II′), R¹² is substituted or unsubstituted methyl; and R¹³ is substituted or unsubstituted vinyl. In some embodiments of Formula (I), (I′), (II), or (II′), R¹² is substituted or unsubstituted ethyl; and R¹³ is substituted or unsubstituted vinyl. In some embodiments of Formula (I), (I′), (II), or (II′), R¹² is substituted or unsubstituted propyl; and R¹³ is substituted or unsubstituted vinyl. In some embodiments of Formula (I), (I′), (II), or (II′), R¹² is substituted or unsubstituted butyl; and R¹³ is substituted or unsubstituted vinyl. In some embodiments of Formula (I), (I′), (II), or (II′), R¹² is substituted or unsubstituted pentyl; and R¹³ is substituted or unsubstituted vinyl. In some embodiments of Formula (I), (I′), (II), or (II′), R¹² is substituted or unsubstituted C₁₋₆ haloalkyl; and R¹³ is substituted or unsubstituted vinyl. In some embodiments of Formula (I), (I′), (II), or (II′), R¹² is substituted or unsubstituted fluoroalkyl; and R¹³ is substituted or unsubstituted vinyl. In some embodiments of Formula (I), (I′), (II), or (II′), R¹² is trifluoromethyl; and R¹³ is substituted or unsubstituted vinyl. In some embodiments of Formula (I), (I′), (II), or (II′), R¹² is substituted or unsubstituted trifluoroethyl; and R¹³ is substituted or unsubstituted vinyl. In some embodiments of Formula (I), (I′), (II), or (II′), R¹² is substituted or unsubstituted C₂₋₆ alkenyl; and R¹³ is substituted or unsubstituted vinyl. In some embodiments of Formula (I), (I′), (II), or (II′), R¹² is substituted or unsubstituted C₂₋₆ alkenyl; and R¹³ is substituted or unsubstituted vinyl. In some embodiments of Formula (I), (I′), (II), or (II′), R¹² is substituted or unsubstituted vinyl; and R¹³ is substituted or unsubstituted vinyl. In some embodiments of Formula (I), (I′), (II), or (II′), R¹² is substituted or unsubstituted allyl; and R¹³ is substituted or unsubstituted vinyl. In some embodiments of Formula (I), (I′), (II), or (II′), R¹² is substituted or unsubstituted propenyl; and R¹³ is substituted or unsubstituted vinyl. In some embodiments of Formula (I), (I′), (II), or (II′), R¹² is substituted or unsubstituted butenyl; and R¹³ is substituted or unsubstituted vinyl. In some embodiments of Formula (I), (I′), (II), or (II′), R¹² is substituted or unsubstituted C₂₋₆ alkynyl; and R¹³ is substituted or unsubstituted vinyl. In some embodiments of Formula (I), (I′), (II), or (II′), R¹² is substituted or unsubstituted acetylenyl; and R¹³ is substituted or unsubstituted vinyl. In some embodiments of Formula (I), (I′), (II), or (II′), R¹² is substituted or unsubstituted propargyl; and R¹³ is substituted or unsubstituted vinyl. In some embodiments of Formula (I), (I′), (II), or (II′), R¹² is substituted or unsubstituted butynyl; and R¹³ is substituted or unsubstituted vinyl. In some embodiments of Formula (I), (I′), (II), or (II′), R¹² is substituted or unsubstituted C₁₋₆alkoxy; and R¹³ is substituted or unsubstituted vinyl. In some embodiments of Formula (I), (I′), (II), or (II′), R¹² is CH₂—OMe; and R¹³ is substituted or unsubstituted vinyl. In some embodiments of Formula (I), (I′), (II), or (II′), R¹² is CH₂-OEt; and R¹³ is substituted or unsubstituted vinyl. In some embodiments of Formula (I), (I′), (II), or (II′), R¹²CH₂—OPr; and R¹³ is substituted or unsubstituted vinyl. In some embodiments of Formula (I), (I′), (II), or (II′), R¹² is CH₂—OCF₃; and R¹³ is substituted or unsubstituted vinyl. In some embodiments of Formula (I), (I′), (II), or (II′), R¹² is substituted or unsubstituted CH₂—OR⁷; and R¹³ is substituted or unsubstituted vinyl. In some embodiments of Formula (I), (I′), (II), or (II′), R¹² is substituted or unsubstituted C₆₋₁₀ aryl; and R¹³ is substituted or unsubstituted vinyl. In some embodiments of Formula (I), (I′), (II), or (II′), R¹² is substituted or unsubstituted 5-10 membered heteroaryl; and R¹³ is substituted or unsubstituted vinyl. In some embodiments of Formula (I), (I′), (II), or (II′), R¹² is substituted or unsubstituted 4-10 membered heterocyclyl; and R¹³ is substituted or unsubstituted vinyl. In some embodiments of Formula (I), (I′), (II), or (II′), R¹² is substituted or unsubstituted 3-10 membered carbocyclyl; and R¹³ is substituted or unsubstituted vinyl. In some embodiments of Formula (I), (I′), (II), or (II′), R¹² is acyl; and R¹³ is substituted or unsubstituted vinyl. In some embodiments of Formula (I), (I′), (II), or (II′), R¹² is a substituted or unsubstituted amide; and R¹³ is substituted or unsubstituted vinyl. In some embodiments of Formula (I), (I′), (II), or (II′), R¹² is a substituted or unsubstituted ester; and R¹³ is substituted or unsubstituted vinyl. In some embodiments of Formula (I), (I′), (II), or (II′), R¹² is —OH; and R¹³ is substituted or unsubstituted vinyl. In some embodiments of Formula (I), (I′), (II), or (II′), R¹² is —SO₂R⁷; and R¹³ is substituted or unsubstituted vinyl. In some embodiments of Formula (I), (I′), (II), or (II′), R¹² is —SOR⁷; and R¹³ is substituted or unsubstituted vinyl. In some embodiments of Formula (I), (I′), (II), or (II′), R¹² is —SO₂NR⁷₂; and R¹³ is substituted or unsubstituted vinyl. In some embodiments of Formula (I), (I′), (II), or (II′), R¹² is —OSO₂R⁷; and R¹³ is substituted or unsubstituted vinyl. In some embodiments of Formula (I), (I′), (II), or (II′), R¹² is —OSOR⁷; and R¹³ is substituted or unsubstituted vinyl. In some embodiments of Formula (I), (I′), (II), or (II′), R¹² is —OSO₂NR⁷₂; and R¹³ is substituted or unsubstituted vinyl. In some embodiments of Formula (I), (I′), (II), or (II′), R¹² is —OSO₂OR⁷; and R¹³ is substituted or unsubstituted vinyl. In some embodiments of Formula (I), (I′), (II), or (II′), R¹² is —O(SO)OR⁷; and R¹³ is substituted or unsubstituted vinyl. In some embodiments of Formula (I), (I′), (II), or (II′), R¹² is —PO(OR⁷)₂; and R¹³ is substituted or unsubstituted vinyl. In some embodiments of Formula (I), (I′), (II), or (II′), R¹² is —PO(NR⁷)₂; and R¹³ is substituted or unsubstituted vinyl. In some embodiments of Formula (I), (I′), (II), or (II′), R¹² is —PO(NR⁷)(OR⁷); and R¹³ is substituted or unsubstituted vinyl. In some embodiments of Formula (I), (I′), (II), or (II′), R¹² is —OPO(OR⁷)₂; and R¹³ is substituted or unsubstituted vinyl. In some embodiments of Formula (I), (I′), (II), or (II′), R¹² is —OPO(NR⁷)₂; and R¹³ is substituted or unsubstituted vinyl. In some embodiments of Formula (I), (I′), (II), or (II′), R¹² is —OPO(NR⁷)(OR⁷); and R¹³ is substituted or unsubstituted vinyl. In some embodiments of Formula (I), (I′), (II), or (II′), R¹² is a nitrogen protecting group; and R¹³ is substituted or unsubstituted vinyl. In some embodiments of Formula (I), (I′), (II), or (II′), R¹² is —OR⁷; and R¹³ is substituted or unsubstituted vinyl.

In some embodiments of Formula (I), (I′), (II), or (II′), R¹² is hydrogen; and R¹³ is substituted or unsubstituted allyl. In some embodiments of Formula (I), (I′), (II), or (II′), R¹² is substituted or unsubstituted C₁₋₆ alkyl; and R¹³ is substituted or unsubstituted allyl. In some embodiments of Formula (I), (I′), (II), or (II′), R¹² is substituted or unsubstituted C₁₋₃ alkyl; and R¹³ is substituted or unsubstituted allyl. In some embodiments of Formula (I), (I′), (II), or (II′), R¹² is branched or unbranched substituted or unsubstituted C₁₋₆ alkyl; and R¹³ is substituted or unsubstituted allyl. In some embodiments of Formula (I), (I′), (II), or (II′), R¹² is substituted or unsubstituted methyl; and R¹³ is substituted or unsubstituted allyl. In some embodiments of Formula (I), (I′), (II), or (II′), R¹² is substituted or unsubstituted ethyl; and R¹³ is substituted or unsubstituted allyl. In some embodiments of Formula (I), (I′), (II), or (II′), R¹² is substituted or unsubstituted propyl; and R¹³ is substituted or unsubstituted allyl. In some embodiments of Formula (I), (I′), (II), or (II′), R¹² is substituted or unsubstituted butyl; and R¹³ is substituted or unsubstituted allyl. In some embodiments of Formula (I), (I′), (II), or (II′), R¹² is substituted or unsubstituted pentyl; and R¹³ is substituted or unsubstituted allyl. In some embodiments of Formula (I), (I′), (II), or (II′), R¹² is substituted or unsubstituted C₁₋₆ haloalkyl; and R¹³ is substituted or unsubstituted allyl. In some embodiments of Formula (I), (I′), (II), or (II′), R¹² is substituted or unsubstituted fluoroalkyl; and R¹³ is substituted or unsubstituted allyl. In some embodiments of Formula (I), (I′), (II), or (II′), R¹² is trifluoromethyl; and R¹³ is substituted or unsubstituted allyl. In some embodiments of Formula (I), (I′), (II), or (II′), R¹² is substituted or unsubstituted trifluoroethyl; and R¹³ is substituted or unsubstituted allyl. In some embodiments of Formula (I), (I′), (II), or (II′), R¹² is substituted or unsubstituted C₂₋₆ alkenyl; and R¹³ is substituted or unsubstituted allyl. In some embodiments of Formula (I), (I′), (II), or (II′), R¹² is substituted or unsubstituted C₂₋₆ alkenyl; and R¹³ is substituted or unsubstituted allyl. In some embodiments of Formula (I), (I′), (II), or (II′), R¹² is substituted or unsubstituted vinyl; and R¹³ is substituted or unsubstituted allyl. In some embodiments of Formula (I), (I′), (II), or (II′), R¹² is substituted or unsubstituted allyl; and R¹³ is substituted or unsubstituted allyl. In some embodiments of Formula (I), (I′), (II), or (II′), R¹² is substituted or unsubstituted propenyl; and R¹³ is substituted or unsubstituted allyl. In some embodiments of Formula (I), (I′), (II), or (II′), R¹² is substituted or unsubstituted butenyl; and R¹³ is substituted or unsubstituted allyl. In some embodiments of Formula (I), (I′), (II), or (II′), R¹² is substituted or unsubstituted C₂₋₆ alkynyl; and R¹³ is substituted or unsubstituted allyl. In some embodiments of Formula (I), (I′), (II), or (II′), R¹² is substituted or unsubstituted acetylenyl; and R¹³ is substituted or unsubstituted allyl. In some embodiments of Formula (I), (I′), (II), or (II′), R¹² is substituted or unsubstituted propargyl; and R¹³ is substituted or unsubstituted allyl. In some embodiments of Formula (I), (I′), (II), or (II′), R¹² is substituted or unsubstituted butynyl; and R¹³ is substituted or unsubstituted allyl. In some embodiments of Formula (I), (I′), (II), or (II′), R¹² is substituted or unsubstituted C₁₋₆ alkoxy; and R¹³ is substituted or unsubstituted allyl. In some embodiments of Formula (I), (I′), (II), or (II′), R¹² is CH₂—OMe; and R¹³ is substituted or unsubstituted allyl. In some embodiments of Formula (I), (I′), (II), or (II′), R¹² is CH₂—OEt; and R¹³ is substituted or unsubstituted allyl. In some embodiments of Formula (I), (I′), (II), or (II′), R¹²CH₂—OPr; and R¹³ is substituted or unsubstituted allyl. In some embodiments of Formula (I), (I′), (II), or (II′), R¹² is CH₂—OCF₃; and R¹³ is substituted or unsubstituted allyl. In some embodiments of Formula (I), (I′), (II), or (II′), R¹² is substituted or unsubstituted CH₂—OR⁷; and R¹³ is substituted or unsubstituted allyl. In some embodiments of Formula (I), (I′), (II), or (II′), R¹² is substituted or unsubstituted C₆₋₁₀ aryl; and R¹³ is substituted or unsubstituted allyl. In some embodiments of Formula (I), (I′), (II), or (II′), R¹² is substituted or unsubstituted 5-10 membered heteroaryl; and R¹³ is substituted or unsubstituted allyl. In some embodiments of Formula (I), (I′), (II), or (II′), R¹² is substituted or unsubstituted 4-10 membered heterocyclyl; and R¹³ is substituted or unsubstituted allyl. In some embodiments of Formula (I), (I′), (II), or (II′), R¹² is substituted or unsubstituted 3-10 membered carbocyclyl; and R¹³ is substituted or unsubstituted allyl. In some embodiments of Formula (I), (I′), (II), or (II′), R¹² is acyl; and R¹³ is substituted or unsubstituted allyl. In some embodiments of Formula (I), (I′), (II), or (II′), R¹² is a substituted or unsubstituted amide; and R¹³ is substituted or unsubstituted allyl. In some embodiments of Formula (I), (I′), (II), or (II′), R¹² is a substituted or unsubstituted ester; and R¹³ is substituted or unsubstituted allyl. In some embodiments of Formula (I), (I′), (II), or (II′), R¹² is —OH; and R¹³ is substituted or unsubstituted allyl. In some embodiments of Formula (I), (I′), (II), or (II′), R¹² is —SO₂R⁷; and R¹³ is substituted or unsubstituted allyl. In some embodiments of Formula (I), (I′), (II), or (II′), R¹² is —SOR⁷; and R¹³ is substituted or unsubstituted allyl. In some embodiments of Formula (I), (I′), (II), or (II′), R¹² is —SO₂NR⁷₂; and R¹³ is substituted or unsubstituted allyl. In some embodiments of Formula (I), (I′), (II), or (II′), R¹² is —OSO₂R⁷; and R¹³ is substituted or unsubstituted allyl. In some embodiments of Formula (I), (I′), (II), or (II′), R¹² is —OSOR⁷; and R¹³ is substituted or unsubstituted allyl. In some embodiments of Formula (I), (I′), (II), or (II′), R¹² is —OSO₂NR⁷₂; and R¹³ is substituted or unsubstituted allyl. In some embodiments of Formula (I), (I′), (II), or (II′), R¹² is —OSO₂OR⁷; and R¹³ is substituted or unsubstituted allyl. In some embodiments of Formula (I), (I′), (II), or (II′), R¹² is —O(SO)OR⁷; and R¹³ is substituted or unsubstituted allyl. In some embodiments of Formula (I), (I′), (II), or (II′), R¹² is —PO(OR⁷)₂; and R¹³ is substituted or unsubstituted allyl. In some embodiments of Formula (I), (I′), (II), or (II′), R¹² is —PO(NR⁷)₂; and R¹³ is substituted or unsubstituted allyl. In some embodiments of Formula (I), (I′), (II), or (II′), R¹² is —PO(NR⁷)(OR⁷); and R¹³ is substituted or unsubstituted allyl. In some embodiments of Formula (I), (I′), (II), or (II′), R¹² is —OPO(OR⁷)₂; and R¹³ is substituted or unsubstituted allyl. In some embodiments of Formula (I), (I′), (II), or (II′), R¹² is —OPO(NR⁷)₂; and R¹³ is substituted or unsubstituted allyl. In some embodiments of Formula (I), (I′), (II), or (II′), R¹² is —OPO(NR⁷)(OR⁷); and R¹³ is substituted or unsubstituted allyl. In some embodiments of Formula (I), (I′), (II), or (II′), R¹² is a nitrogen protecting group; and R¹³ is substituted or unsubstituted allyl. In some embodiments of Formula (I), (I′), (II), or (II′), R¹² is —OR⁷; and R¹³ is substituted or unsubstituted allyl.

In some embodiments of Formula (I), (I′), (II), or (II′), R¹² is hydrogen; and R¹³ is substituted or unsubstituted propenyl. In some embodiments of Formula (I), (I′), (II), or (II′), R¹² is substituted or unsubstituted C₁₋₆ alkyl; and R¹³ is substituted or unsubstituted propenyl. In some embodiments of Formula (I), (I′), (II), or (II′), R¹² is substituted or unsubstituted C₁₋₃ alkyl; and R¹³ is substituted or unsubstituted propenyl. In some embodiments of Formula (I), (I′), (II), or (II′), R¹² is branched or unbranched substituted or unsubstituted C₁₋₆ alkyl; and R¹³ is substituted or unsubstituted propenyl. In some embodiments of Formula (I), (I′), (II), or (II′), R¹² is substituted or unsubstituted methyl; and R¹³ is substituted or unsubstituted propenyl. In some embodiments of Formula (I), (I′), (II), or (II′), R¹² is substituted or unsubstituted ethyl; and R¹³ is substituted or unsubstituted propenyl. In some embodiments of Formula (I), (I′), (II), or (II′), R¹² is substituted or unsubstituted propyl; and R¹³ is substituted or unsubstituted propenyl. In some embodiments of Formula (I), (I′), (II), or (II′), R¹² is substituted or unsubstituted butyl; and R¹³ is substituted or unsubstituted propenyl. In some embodiments of Formula (I), (I′), (II), or (II′), R¹² is substituted or unsubstituted pentyl; and R¹³ is substituted or unsubstituted propenyl. In some embodiments of Formula (I), (I′), (II), or (II′), R¹² is substituted or unsubstituted C₁₋₆ haloalkyl; and R¹³ is substituted or unsubstituted propenyl. In some embodiments of Formula (I), (I′), (II), or (II′), R¹² is substituted or unsubstituted fluoroalkyl; and R¹³ is substituted or unsubstituted propenyl. In some embodiments of Formula (I), (I′), (II), or (II′), R¹² is trifluoromethyl; and R¹³ is substituted or unsubstituted propenyl. In some embodiments of Formula (I), (I′), (II), or (II′), R¹² is substituted or unsubstituted trifluoroethyl; and R¹³ is substituted or unsubstituted propenyl. In some embodiments of Formula (I), (I′), (II), or (II′), R¹² is substituted or unsubstituted C₂₋₆ alkenyl; and R¹³ is substituted or unsubstituted propenyl. In some embodiments of Formula (I), (I′), (II), or (II′), R¹² is substituted or unsubstituted C₂₋₆ alkenyl; and R¹³ is substituted or unsubstituted propenyl. In some embodiments of Formula (I), (I′), (II), or (II′), R¹² is substituted or unsubstituted vinyl; and R¹³ is substituted or unsubstituted propenyl. In some embodiments of Formula (I), (I′), (II), or (II′), R¹² is substituted or unsubstituted allyl; and R¹³ is substituted or unsubstituted propenyl. In some embodiments of Formula (I), (I′), (II), or (II′), R¹² is substituted or unsubstituted propenyl; and R¹³ is substituted or unsubstituted propenyl. In some embodiments of Formula (I), (I′), (II), or (II′), R¹² is substituted or unsubstituted butenyl; and R¹³ is substituted or unsubstituted propenyl. In some embodiments of Formula (I), (I′), (II), or (II′), R¹² is substituted or unsubstituted C₂₋₆ alkynyl; and R¹³ is substituted or unsubstituted propenyl. In some embodiments of Formula (I), (I′), (II), or (II′), R¹² is substituted or unsubstituted acetylenyl; and R¹³ is substituted or unsubstituted propenyl. In some embodiments of Formula (I), (I′), (II), or (II′), R¹² is substituted or unsubstituted propargyl; and R¹³ is substituted or unsubstituted propenyl. In some embodiments of Formula (I), (I′), (II), or (II′), R¹² is substituted or unsubstituted butynyl; and R¹³ is substituted or unsubstituted propenyl. In some embodiments of Formula (I), (I′), (II), or (II′), R¹² is substituted or unsubstituted C₁₋₆ alkoxy; and R¹³ is substituted or unsubstituted propenyl. In some embodiments of Formula (I), (I′), (II), or (II′), R¹² is CH₂—OMe; and R¹³ is substituted or unsubstituted propenyl. In some embodiments of Formula (I), (I′), (II), or (II′), R¹² is CH₂—OEt; and R¹³ is substituted or unsubstituted propenyl. In some embodiments of Formula (I), (I′), (II), or (II′), R¹²CH₂—OPr; and R¹³ is substituted or unsubstituted propenyl. In some embodiments of Formula (I), (I′), (II), or (II′), R¹² is CH₂—OCF₃; and R¹³ is substituted or unsubstituted propenyl. In some embodiments of Formula (I), (I′), (II), or (II′), R¹² is substituted or unsubstituted CH₂—OR⁷; and R¹³ is substituted or unsubstituted propenyl. In some embodiments of Formula (I), (I′), (II), or (II′), R¹² is substituted or unsubstituted C₆₋₁₀ aryl; and R¹³ is substituted or unsubstituted propenyl. In some embodiments of Formula (I), (I′), (II), or (II′), R¹² is substituted or unsubstituted 5-10 membered heteroaryl; and R¹³ is substituted or unsubstituted propenyl. In some embodiments of Formula (I), (I′), (II), or (II′), R¹² is substituted or unsubstituted 4-10 membered heterocyclyl; and R¹³ is substituted or unsubstituted propenyl. In some embodiments of Formula (I), (I′), (II), or (II′), R¹² is substituted or unsubstituted 3-10 membered carbocyclyl; and R¹³ is substituted or unsubstituted propenyl. In some embodiments of Formula (I), (I′), (II), or (II′), R¹² is acyl; and R¹³ is substituted or unsubstituted propenyl. In some embodiments of Formula (I), (I′), (II), or (II′), R¹² is a substituted or unsubstituted amide; and R¹³ is substituted or unsubstituted propenyl. In some embodiments of Formula (I), (I′), (II), or (II′), R¹² is a substituted or unsubstituted ester; and R¹³ is substituted or unsubstituted propenyl. In some embodiments of Formula (I), (I′), (II), or (II′), R¹² is —OH; and R¹³ is substituted or unsubstituted propenyl. In some embodiments of Formula (I), (I′), (II), or (II′), R¹² is —SO₂R⁷; and R¹³ is substituted or unsubstituted propenyl. In some embodiments of Formula (I), (I′), (II), or (II′), R¹² is —SOR⁷; and R¹³ is substituted or unsubstituted propenyl. In some embodiments of Formula (I), (I′), (II), or (II′), R¹² is —SO₂NR⁷₂; and R¹³ is substituted or unsubstituted propenyl. In some embodiments of Formula (I), (I′), (II), or (II′), R¹² is —OSO₂R⁷; and R¹³ is substituted or unsubstituted propenyl. In some embodiments of Formula (I), (I′), (II), or (II′), R¹² is —OSOR⁷; and R¹³ is substituted or unsubstituted propenyl. In some embodiments of Formula (I), (I′), (II), or (II′), R¹² is —OSO₂NR⁷₂; and R¹³ is substituted or unsubstituted propenyl. In some embodiments of Formula (I), (I′), (II), or (II′), R¹² is —OSO₂OR⁷; and R¹³ is substituted or unsubstituted propenyl. In some embodiments of Formula (I), (I′), (II), or (II′), R¹² is —O(SO)OR⁷; and R¹³ is substituted or unsubstituted propenyl. In some embodiments of Formula (I), (I′), (II), or (II′), R¹² is —PO(OR⁷)₂; and R¹³ is substituted or unsubstituted propenyl. In some embodiments of Formula (I), (I′), (II), or (II′), R¹² is —PO(NR⁷)₂; and R¹³ is substituted or unsubstituted propenyl. In some embodiments of Formula (I), (I′), (II), or (II′), R¹² is —PO(NR⁷)(OR⁷); and R¹³ is substituted or unsubstituted propenyl. In some embodiments of Formula (I), (I′), (II), or (II′), R¹² is —OPO(OR⁷)₂; and R¹³ is substituted or unsubstituted propenyl. In some embodiments of Formula (I), (I′), (II), or (II′), R¹² is —OPO(NR⁷)₂; and R¹³ is substituted or unsubstituted propenyl. In some embodiments of Formula (I), (I′), (II), or (II′), R¹² is —OPO(NR⁷)(OR⁷); and R¹³ is substituted or unsubstituted propenyl. In some embodiments of Formula (I), (I′), (II), or (II′), R¹² is a nitrogen protecting group; and R¹³ is substituted or unsubstituted propenyl. In some embodiments of Formula (I), (I′), (II), or (II′), R¹² is —OR⁷; and R¹³ is substituted or unsubstituted propenyl.

In some embodiments of Formula (I), (I′), (II), or (II′), R¹² is hydrogen; and R¹³ is substituted or unsubstituted butenyl. In some embodiments of Formula (I), (I′), (II), or (II′), R¹² is substituted or unsubstituted C₁₋₆ alkyl; and R¹³ is substituted or unsubstituted butenyl. In some embodiments of Formula (I), (I′), (II), or (II′), R¹² is substituted or unsubstituted C₁₋₃ alkyl; and R¹³ is substituted or unsubstituted butenyl. In some embodiments of Formula (I), (I′), (II), or (II′), R¹² is branched or unbranched substituted or unsubstituted C₁₋₆ alkyl; and R¹³ is substituted or unsubstituted butenyl. In some embodiments of Formula (I), (I′), (II), or (II′), R¹² is substituted or unsubstituted methyl; and R¹³ is substituted or unsubstituted butenyl. In some embodiments of Formula (I), (I′), (II), or (II′), R¹² is substituted or unsubstituted ethyl; and R¹³ is substituted or unsubstituted butenyl. In some embodiments of Formula (I), (I′), (II), or (II′), R¹² is substituted or unsubstituted propyl; and R¹³ is substituted or unsubstituted butenyl. In some embodiments of Formula (I), (I′), (II), or (II′), R¹² is substituted or unsubstituted butyl; and R¹³ is substituted or unsubstituted butenyl. In some embodiments of Formula (I), (I′), (II), or (II′), R¹² is substituted or unsubstituted pentyl; and R¹³ is substituted or unsubstituted butenyl. In some embodiments of Formula (I), (I′), (II), or (II′), R¹² is substituted or unsubstituted C₁₋₆ haloalkyl; and R¹³ is substituted or unsubstituted butenyl. In some embodiments of Formula (I), (I′), (II), or (II′), R¹² is substituted or unsubstituted fluoroalkyl; and R¹³ is substituted or unsubstituted butenyl. In some embodiments of Formula (I), (I′), (II), or (II′), R¹² is trifluoromethyl; and R¹³ is substituted or unsubstituted butenyl. In some embodiments of Formula (I), (I′), (II), or (II′), R¹² is substituted or unsubstituted trifluoroethyl; and R¹³ is substituted or unsubstituted butenyl. In some embodiments of Formula (I), (I′), (II), or (II′), R¹² is substituted or unsubstituted C₂₋₆ alkenyl; and R¹³ is substituted or unsubstituted butenyl. In some embodiments of Formula (I), (I′), (II), or (II′), R¹² is substituted or unsubstituted C₂₋₆ alkenyl; and R¹³ is substituted or unsubstituted butenyl. In some embodiments of Formula (I), (I′), (II), or (II′), R¹² is substituted or unsubstituted vinyl; and R¹³ is substituted or unsubstituted butenyl. In some embodiments of Formula (I), (I′), (II), or (II′), R¹² is substituted or unsubstituted allyl; and R¹³ is substituted or unsubstituted butenyl. In some embodiments of Formula (I), (I′), (II), or (II′), R¹² is substituted or unsubstituted propenyl; and R¹³ is substituted or unsubstituted butenyl. In some embodiments of Formula (I), (I′), (II), or (II′), R¹² is substituted or unsubstituted butenyl; and R¹³ is substituted or unsubstituted butenyl. In some embodiments of Formula (I), (I′), (II), or (II′), R¹² is substituted or unsubstituted C₂₋₆ alkynyl; and R¹³ is substituted or unsubstituted butenyl. In some embodiments of Formula (I), (I′), (II), or (II′), R¹² is substituted or unsubstituted acetylenyl; and R¹³ is substituted or unsubstituted butenyl. In some embodiments of Formula (I), (I′), (II), or (II′), R¹² is substituted or unsubstituted propargyl; and R¹³ is substituted or unsubstituted butenyl. In some embodiments of Formula (I), (I′), (II), or (II′), R¹² is substituted or unsubstituted butynyl; and R¹³ is substituted or unsubstituted butenyl. In some embodiments of Formula (I), (I′), (II), or (II′), R¹² is substituted or unsubstituted C₁₋₆ alkoxy; and R¹³ is substituted or unsubstituted butenyl. In some embodiments of Formula (I), (I′), (II), or (II′), R¹² is CH₂—OMe; and R¹³ is substituted or unsubstituted butenyl. In some embodiments of Formula (I), (I′), (II), or (II′), R¹² is CH₂-OEt; and R¹³ is substituted or unsubstituted butenyl. In some embodiments of Formula (I), (I′), (II), or (II′), R¹²CH₂—OPr; and R¹³ is substituted or unsubstituted butenyl. In some embodiments of Formula (I), (I′), (II), or (II′), R¹² is CH₂—OCF₃; and R¹³ is substituted or unsubstituted butenyl. In some embodiments of Formula (I), (I′), (II), or (II′), R¹² is substituted or unsubstituted CH₂—OR⁷; and R¹³ is substituted or unsubstituted butenyl. In some embodiments of Formula (I), (I′), (II), or (II′), R¹² is substituted or unsubstituted C₆₋₁₀ aryl; and R¹³ is substituted or unsubstituted butenyl. In some embodiments of Formula (I), (I′), (II), or (II′), R¹² is substituted or unsubstituted 5-10 membered heteroaryl; and R¹³ is substituted or unsubstituted butenyl. In some embodiments of Formula (I), (I′), (II), or (II′), R¹² is substituted or unsubstituted 4-10 membered heterocyclyl; and R¹³ is substituted or unsubstituted butenyl. In some embodiments of Formula (I), (I′), (II), or (II′), R¹² is substituted or unsubstituted 3-10 membered carbocyclyl; and R¹³ is substituted or unsubstituted butenyl. In some embodiments of Formula (I), (I′), (II), or (II′), R¹² is acyl; and R¹³ is substituted or unsubstituted butenyl. In some embodiments of Formula (I), (I′), (II), or (II′), R¹² is a substituted or unsubstituted amide; and R¹³ is substituted or unsubstituted butenyl. In some embodiments of Formula (I), (I′), (II), or (II′), R¹² is a substituted or unsubstituted ester; and R¹³ is substituted or unsubstituted butenyl. In some embodiments of Formula (I), (I′), (II), or (II′), R¹² is —OH; and R¹³ is substituted or unsubstituted butenyl. In some embodiments of Formula (I), (I′), (II), or (II′), R¹² is —SO₂R⁷; and R¹³ is substituted or unsubstituted butenyl. In some embodiments of Formula (I), (I′), (II), or (II′), R¹² is —SOR⁷; and R¹³ is substituted or unsubstituted butenyl. In some embodiments of Formula (I), (I′), (II), or (II′), R¹² is —SO₂NR⁷₂; and R¹³ is substituted or unsubstituted butenyl. In some embodiments of Formula (I), (I′), (II), or (II′), R¹² is —OSO₂R⁷; and R¹³ is substituted or unsubstituted butenyl. In some embodiments of Formula (I), (I′), (II), or (II′), R¹² is —OSOR⁷; and R¹³ is substituted or unsubstituted butenyl. In some embodiments of Formula (I), (I′), (II), or (II′), R¹² is —OSO₂NR⁷₂; and R¹³ is substituted or unsubstituted butenyl. In some embodiments of Formula (I), (I′), (II), or (II′), R¹² is —OSO₂OR⁷; and R¹³ is substituted or unsubstituted butenyl. In some embodiments of Formula (I), (I′), (II), or (II′), R¹² is —O(SO)OR⁷; and R¹³ is substituted or unsubstituted butenyl. In some embodiments of Formula (I), (I′), (II), or (II′), R¹² is —PO(OR⁷)₂; and R¹³ is substituted or unsubstituted butenyl. In some embodiments of Formula (I), (I′), (II), or (II′), R¹² is —PO(NR⁷)₂; and R¹³ is substituted or unsubstituted butenyl. In some embodiments of Formula (I), (I′), (II), or (II′), R¹² is —PO(NR⁷)(OR⁷); and R¹³ is substituted or unsubstituted butenyl. In some embodiments of Formula (I), (I′), (II), or (II′), R¹² is —OPO(OR⁷)₂; and R¹³ is substituted or unsubstituted butenyl. In some embodiments of Formula (I), (I′), (II), or (II′), R¹² is —OPO(NR⁷)₂; and R¹³ is substituted or unsubstituted butenyl. In some embodiments of Formula (I), (I′), (II), or (II′), R¹² is —OPO(NR⁷)(OR⁷); and R¹³ is substituted or unsubstituted butenyl. In some embodiments of Formula (I), (I′), (II), or (II′), R¹² is a nitrogen protecting group; and R¹³ is substituted or unsubstituted butenyl. In some embodiments of Formula (I), (I′), (II), or (II′), R¹² is —OR⁷; and R¹³ is substituted or unsubstituted butenyl.

In some embodiments of Formula (I), (I′), (II), or (II′), R¹² is hydrogen; and R¹³ is substituted or unsubstituted C₂₋₆ alkynyl. In some embodiments of Formula (I), (I′), (II), or (II′), R¹² is substituted or unsubstituted C₁₋₆ alkyl; and R¹³ is substituted or unsubstituted C₂₋₆ alkynyl. In some embodiments of Formula (I), (I′), (II), or (II′), R¹² is substituted or unsubstituted C₁₋₃ alkyl; and R¹³ is substituted or unsubstituted C₂₋₆ alkynyl. In some embodiments of Formula (I), (I′), (II), or (II′), R¹² is branched or unbranched substituted or unsubstituted C₁₋₆ alkyl; and R¹³ is substituted or unsubstituted C₂₋₆ alkynyl. In some embodiments of Formula (I), (I′), (II), or (II′), R¹² is substituted or unsubstituted methyl; and R¹³ is substituted or unsubstituted C₂₋₆ alkynyl. In some embodiments of Formula (I), (I′), (II), or (II′), R¹² is substituted or unsubstituted ethyl; and R¹³ is substituted or unsubstituted C₂₋₆ alkynyl. In some embodiments of Formula (I), (I′), (II), or (II′), R¹² is substituted or unsubstituted propyl; and R¹³ is substituted or unsubstituted C₂₋₆ alkynyl. In some embodiments of Formula (I), (I′), (II), or (II′), R¹² is substituted or unsubstituted butyl; and R¹³ is substituted or unsubstituted C₂₋₆ alkynyl. In some embodiments of Formula (I), (I′), (II), or (II′), R¹² is substituted or unsubstituted pentyl; and R¹³ is substituted or unsubstituted C₂₋₆ alkynyl. In some embodiments of Formula (I), (I′), (II), or (II′), R¹² is substituted or unsubstituted C₁₋₆ haloalkyl; and R¹³ is substituted or unsubstituted C₂₋₆ alkynyl. In some embodiments of Formula (I), (I′), (II), or (II′), R¹² is substituted or unsubstituted fluoroalkyl; and R¹³ is substituted or unsubstituted C₂₋₆ alkynyl. In some embodiments of Formula (I), (I′), (II), or (II′), R¹² is trifluoromethyl; and R¹³ is substituted or unsubstituted C₂₋₆ alkynyl. In some embodiments of Formula (I), (I′), (II), or (II′), R¹² is substituted or unsubstituted trifluoroethyl; and R¹³ is substituted or unsubstituted C₂₋₆ alkynyl. In some embodiments of Formula (I), (I′), (II), or (II′), R¹² is substituted or unsubstituted C₂₋₆ alkenyl; and R¹³ is substituted or unsubstituted C₂₋₆ alkynyl. In some embodiments of Formula (I), (I′), (II), or (II′), R¹² is substituted or unsubstituted C₂₋₆ alkenyl; and R¹³ is substituted or unsubstituted C₂₋₆ alkynyl. In some embodiments of Formula (I), (I′), (II), or (II′), R¹² is substituted or unsubstituted vinyl; and R¹³ is substituted or unsubstituted C₂₋₆ alkynyl. In some embodiments of Formula (I), (I′), (II), or (II′), R¹² is substituted or unsubstituted allyl; and R¹³ is substituted or unsubstituted C₂₋₆ alkynyl. In some embodiments of Formula (I), (I′), (II), or (II′), R¹² is substituted or unsubstituted propenyl; and R¹³ is substituted or unsubstituted C₂₋₆ alkynyl. In some embodiments of Formula (I), (I′), (II), or (II′), R¹² is substituted or unsubstituted butenyl; and R¹³ is substituted or unsubstituted C₂₋₆ alkynyl. In some embodiments of Formula (I), (I′), (II), or (II′), R¹² is substituted or unsubstituted C₂₋₆ alkynyl; and R¹³ is substituted or unsubstituted C₂₋₆ alkynyl. In some embodiments of Formula (I), (I′), (II), or (II′), R¹² is substituted or unsubstituted acetylenyl; and R¹³ is substituted or unsubstituted C₂₋₆ alkynyl. In some embodiments of Formula (I), (I′), (II), or (II′), R¹² is substituted or unsubstituted propargyl; and R¹³ is substituted or unsubstituted C₂₋₆ alkynyl. In some embodiments of Formula (I), (I′), (II), or (II′), R¹² is substituted or unsubstituted butynyl; and R¹³ is substituted or unsubstituted C₂₋₆ alkynyl. In some embodiments of Formula (I), (I′), (II), or (II′), R¹² is substituted or unsubstituted C₁₋₆ alkoxy; and R¹³ is substituted or unsubstituted C₂₋₆ alkynyl. In some embodiments of Formula (I), (I′), (II), or (II′), R¹² is CH₂—OMe; and R¹³ is substituted or unsubstituted C₂₋₆ alkynyl. In some embodiments of Formula (I), (I′), (II), or (II′), R¹² is CH₂—OEt; and R¹³ is substituted or unsubstituted C₂₋₆ alkynyl. In some embodiments of Formula (I), (I′), (II), or (II′), R¹²CH₂—OPr; and R¹³ is substituted or unsubstituted C₂₋₆ alkynyl. In some embodiments of Formula (I), (I′), (II), or (II′), R¹² is CH₂—OCF₃; and R¹³ is substituted or unsubstituted C₂₋₆ alkynyl. In some embodiments of Formula (I), (I′), (II), or (II′), R¹² is substituted or unsubstituted CH₂—OR⁷; and R¹³ is substituted or unsubstituted C₂₋₆ alkynyl. In some embodiments of Formula (I), (I′), (II), or (II′), R¹² is substituted or unsubstituted C₆₋₁₀ aryl; and R¹³ is substituted or unsubstituted C₂₋₆ alkynyl. In some embodiments of Formula (I), (I′), (II), or (II′), R¹² is substituted or unsubstituted 5-10 membered heteroaryl; and R¹³ is substituted or unsubstituted C₂₋₆ alkynyl. In some embodiments of Formula (I), (I′), (II), or (II′), R¹² is substituted or unsubstituted 4-10 membered heterocyclyl; and R¹³ is substituted or unsubstituted C₂₋₆ alkynyl. In some embodiments of Formula (I), (I′), (II), or (II′), R¹² is substituted or unsubstituted 3-10 membered carbocyclyl; and R¹³ is substituted or unsubstituted C₂₋₆ alkynyl. In some embodiments of Formula (I), (I′), (II), or (II′), R¹² is acyl; and R¹³ is substituted or unsubstituted C₂₋₆ alkynyl. In some embodiments of Formula (I), (I′), (II), or (II′), R¹² is a substituted or unsubstituted amide; and R¹³ is substituted or unsubstituted C₂₋₆ alkynyl. In some embodiments of Formula (I), (I′), (II), or (II′), R¹² is a substituted or unsubstituted ester; and R¹³ is substituted or unsubstituted C₂₋₆ alkynyl. In some embodiments of Formula (I), (I′), (II), or (II′), R¹² is —OH; and R¹³ is substituted or unsubstituted C₂₋₆ alkynyl. In some embodiments of Formula (I), (I′), (II), or (II′), R¹² is —SO₂R⁷; and R¹³ is substituted or unsubstituted C₂₋₆ alkynyl. In some embodiments of Formula (I), (I′), (II), or (II′), R¹² is —SOR⁷; and R¹³ is substituted or unsubstituted C₂₋₆ alkynyl. In some embodiments of Formula (I), (I′), (II), or (II′), R¹² is —SO₂NR⁷₂; and R¹³ is substituted or unsubstituted C₂₋₆ alkynyl. In some embodiments of Formula (I), (I′), (II), or (II′), R¹² is —OSO₂R⁷; and R¹³ is substituted or unsubstituted C₂₋₆ alkynyl. In some embodiments of Formula (I), (I′), (II), or (II′), R¹² is —OSOR⁷; and R¹³ is substituted or unsubstituted C₂₋₆ alkynyl. In some embodiments of Formula (I), (I′), (II), or (II′), R¹² is —OSO₂NR⁷₂; and R¹³ is substituted or unsubstituted C₂₋₆ alkynyl. In some embodiments of Formula (I), (I′), (II), or (II′), R¹² is —OSO₂OR⁷; and R¹³ is substituted or unsubstituted C₂₋₆ alkynyl. In some embodiments of Formula (I), (I′), (II), or (II′), R¹² is —O(SO)OR⁷; and R¹³ is substituted or unsubstituted C₂₋₆ alkynyl. In some embodiments of Formula (I), (I′), (II), or (II′), R¹² is —PO(OR⁷)₂; and R¹³ is substituted or unsubstituted C₂₋₆ alkynyl. In some embodiments of Formula (I), (I′), (II), or (II′), R¹² is —PO(NR⁷)₂; and R¹³ is substituted or unsubstituted C₂₋₆ alkynyl. In some embodiments of Formula (I), (I′), (II), or (II′), R¹² is —PO(NR⁷)(OR⁷); and R¹³ is substituted or unsubstituted C₂₋₆ alkynyl. In some embodiments of Formula (I), (I′), (II), or (II′), R¹² is —OPO(OR⁷)₂; and R¹³ is substituted or unsubstituted C₂₋₆ alkynyl. In some embodiments of Formula (I), (I′), (II), or (II′), R¹² is —OPO(NR⁷)₂; and R¹³ is substituted or unsubstituted C₂₋₆ alkynyl. In some embodiments of Formula (I), (I′), (II), or (II′), R¹² is —OPO(NR⁷)(OR⁷); and R¹³ is substituted or unsubstituted C₂₋₆ alkynyl. In some embodiments of Formula (I), (I′), (II), or (II′), R¹² is a nitrogen protecting group; and R¹³ is substituted or unsubstituted C₂₋₆ alkynyl. In some embodiments of Formula (I), (I′), (II), or (II′), R¹² is —OR⁷; and R¹³ is substituted or unsubstituted C₂₋₆ alkynyl.

In some embodiments of Formula (I), (I′), (II), or (II′), R¹² is hydrogen; and R¹³ is substituted or unsubstituted acetylenyl. In some embodiments of Formula (I), (I′), (II), or (II′), R¹² is substituted or unsubstituted C₁₋₆ alkyl; and R¹³ is substituted or unsubstituted acetylenyl. In some embodiments of Formula (I), (I′), (II), or (II′), R¹² is substituted or unsubstituted C₁₋₃ alkyl; and R¹³ is substituted or unsubstituted acetylenyl. In some embodiments of Formula (I), (I′), (II), or (II′), R¹² is branched or unbranched substituted or unsubstituted C₁₋₆ alkyl; and R¹³ is substituted or unsubstituted acetylenyl. In some embodiments of Formula (I), (I′), (II), or (II′), R¹² is substituted or unsubstituted methyl; and R¹³ is substituted or unsubstituted acetylenyl. In some embodiments of Formula (I), (I′), (II), or (II′), R¹² is substituted or unsubstituted ethyl; and R¹³ is substituted or unsubstituted acetylenyl. In some embodiments of Formula (I), (I′), (II), or (II′), R¹² is substituted or unsubstituted propyl; and R¹³ is substituted or unsubstituted acetylenyl. In some embodiments of Formula (I), (I′), (II), or (II′), R¹² is substituted or unsubstituted butyl; and R¹³ is substituted or unsubstituted acetylenyl. In some embodiments of Formula (I), (I′), (II), or (II′), R¹² is substituted or unsubstituted pentyl; and R¹³ is substituted or unsubstituted acetylenyl. In some embodiments of Formula (I), (I′), (II), or (II′), R¹² is substituted or unsubstituted C₁₋₆ haloalkyl; and R¹³ is substituted or unsubstituted acetylenyl. In some embodiments of Formula (I), (I′), (II), or (II′), R¹² is substituted or unsubstituted fluoroalkyl; and R¹³ is substituted or unsubstituted acetylenyl. In some embodiments of Formula (I), (I′), (II), or (II′), R¹² is trifluoromethyl; and R¹³ is substituted or unsubstituted acetylenyl. In some embodiments of Formula (I), (I′), (II), or (II′), R¹² is substituted or unsubstituted trifluoroethyl; and R¹³ is substituted or unsubstituted acetylenyl. In some embodiments of Formula (I), (I′), (II), or (II′), R¹² is substituted or unsubstituted C₂₋₆ alkenyl; and R¹³ is substituted or unsubstituted acetylenyl. In some embodiments of Formula (I), (I′), (II), or (II′), R¹² is substituted or unsubstituted C₂₋₆ alkenyl; and R¹³ is substituted or unsubstituted acetylenyl. In some embodiments of Formula (I), (I′), (II), or (II′), R¹² is substituted or unsubstituted vinyl; and R¹³ is substituted or unsubstituted acetylenyl. In some embodiments of Formula (I), (I′), (II), or (II′), R¹² is substituted or unsubstituted allyl; and R¹³ is substituted or unsubstituted acetylenyl. In some embodiments of Formula (I), (I′), (II), or (II′), R¹² is substituted or unsubstituted propenyl; and R¹³ is substituted or unsubstituted acetylenyl. In some embodiments of Formula (I), (I′), (II), or (II′), R¹² is substituted or unsubstituted butenyl; and R¹³ is substituted or unsubstituted acetylenyl. In some embodiments of Formula (I), (I′), (II), or (II′), R¹² is substituted or unsubstituted C₂₋₆ alkynyl; and R¹³ is substituted or unsubstituted acetylenyl. In some embodiments of Formula (I), (I′), (II), or (II′), R¹² is substituted or unsubstituted acetylenyl; and R¹³ is substituted or unsubstituted acetylenyl. In some embodiments of Formula (I), (I′), (II), or (II′), R¹² is substituted or unsubstituted propargyl; and R¹³ is substituted or unsubstituted acetylenyl. In some embodiments of Formula (I), (I′), (II), or (II′), R¹² is substituted or unsubstituted butynyl; and R¹³ is substituted or unsubstituted acetylenyl. In some embodiments of Formula (I), (I′), (II), or (II′), R¹² is substituted or unsubstituted C₁₋₆ alkoxy; and R¹³ is substituted or unsubstituted acetylenyl. In some embodiments of Formula (I), (I′), (II), or (II′), R¹² is CH₂—OMe; and R¹³ is substituted or unsubstituted acetylenyl. In some embodiments of Formula (I), (I′), (II), or (II′), R¹² is CH₂—OEt; and R¹³ is substituted or unsubstituted acetylenyl. In some embodiments of Formula (I), (I′), (II), or (II′), R¹²CH₂—OPr; and R¹³ is substituted or unsubstituted acetylenyl. In some embodiments of Formula (I), (I′), (II), or (II′), R¹² is CH₂—OCF₃; and R¹³ is substituted or unsubstituted acetylenyl. In some embodiments of Formula (I), (I′), (II), or (II′), R¹² is substituted or unsubstituted CH₂—OR⁷; and R¹³ is substituted or unsubstituted acetylenyl. In some embodiments of Formula (I), (I′), (II), or (II′), R¹² is substituted or unsubstituted C₆₋₁₀ aryl; and R¹³ is substituted or unsubstituted acetylenyl. In some embodiments of Formula (I), (I′), (II), or (II′), R¹² is substituted or unsubstituted 5-10 membered heteroaryl; and R¹³ is substituted or unsubstituted acetylenyl. In some embodiments of Formula (I), (I′), (II), or (II′), R¹² is substituted or unsubstituted 4-10 membered heterocyclyl; and R¹³ is substituted or unsubstituted acetylenyl. In some embodiments of Formula (I), (I′), (II), or (II′), R¹² is substituted or unsubstituted 3-10 membered carbocyclyl; and R¹³ is substituted or unsubstituted acetylenyl. In some embodiments of Formula (I), (I′), (II), or (II′), R¹² is acyl; and R¹³ is substituted or unsubstituted acetylenyl. In some embodiments of Formula (I), (I′), (II), or (II′), R¹² is a substituted or unsubstituted amide; and R¹³ is substituted or unsubstituted acetylenyl. In some embodiments of Formula (I), (I′), (II), or (II′), R¹² is a substituted or unsubstituted ester; and R¹³ is substituted or unsubstituted acetylenyl. In some embodiments of Formula (I), (I′), (II), or (II′), R¹² is —OH; and R¹³ is substituted or unsubstituted acetylenyl. In some embodiments of Formula (I), (I′), (II), or (II′), R¹² is —SO₂R⁷; and R¹³ is substituted or unsubstituted acetylenyl. In some embodiments of Formula (I), (I′), (II), or (II′), R¹² is —SOR⁷; and R¹³ is substituted or unsubstituted acetylenyl. In some embodiments of Formula (I), (I′), (II), or (II′), R¹² is —SO₂NR⁷₂; and R¹³ is substituted or unsubstituted acetylenyl. In some embodiments of Formula (I), (I′), (II), or (II′), R¹² is —OSO₂R⁷; and R¹³ is substituted or unsubstituted acetylenyl. In some embodiments of Formula (I), (I′), (II), or (II′), R¹² is —OSOR⁷; and R¹³ is substituted or unsubstituted acetylenyl. In some embodiments of Formula (I), (I′), (II), or (II′), R¹² is —OSO₂NR⁷₂; and R¹³ is substituted or unsubstituted acetylenyl. In some embodiments of Formula (I), (I′), (II), or (II′), R¹² is —OSO₂OR⁷; and R¹³ is substituted or unsubstituted acetylenyl. In some embodiments of Formula (I), (I′), (II), or (II′), R¹² is —O(SO)OR⁷; and R¹³ is substituted or unsubstituted acetylenyl. In some embodiments of Formula (I), (I′), (II), or (II′), R¹² is —PO(OR⁷)₂; and R¹³ is substituted or unsubstituted acetylenyl. In some embodiments of Formula (I), (I′), (II), or (II′), R¹² is —PO(NR⁷)₂; and R¹³ is substituted or unsubstituted acetylenyl. In some embodiments of Formula (I), (I′), (II), or (II′), R¹² is —PO(NR⁷)(OR⁷); and R¹³ is substituted or unsubstituted acetylenyl. In some embodiments of Formula (I), (I′), (II), or (II′), R¹² is —OPO(OR⁷)₂; and R¹³ is substituted or unsubstituted acetylenyl. In some embodiments of Formula (I), (I′), (II), or (II′), R¹² is —OPO(NR⁷)₂; and R¹³ is substituted or unsubstituted acetylenyl. In some embodiments of Formula (I), (I′), (II), or (II′), R¹² is —OPO(NR⁷)(OR⁷); and R¹³ is substituted or unsubstituted acetylenyl. In some embodiments of Formula (I), (I′), (II), or (II′), R¹² is a nitrogen protecting group; and R¹³ is substituted or unsubstituted acetylenyl. In some embodiments of Formula (I), (I′), (II), or (II′), R¹² is —OR⁷; and R¹³ is substituted or unsubstituted acetylenyl.

In some embodiments of Formula (I), (I′), (II), or (II′), R¹² is hydrogen; and R¹³ is substituted or unsubstituted propargyl. In some embodiments of Formula (I), (I′), (II), or (II′), R¹² is substituted or unsubstituted C₁₋₆ alkyl; and R¹³ is substituted or unsubstituted propargyl. In some embodiments of Formula (I), (I′), (II), or (II′), R¹² is substituted or unsubstituted C₁₋₃ alkyl; and R¹³ is substituted or unsubstituted propargyl. In some embodiments of Formula (I), (I′), (II), or (II′), R¹² is branched or unbranched substituted or unsubstituted C₁₋₆ alkyl; and R¹³ is substituted or unsubstituted propargyl. In some embodiments of Formula (I), (I′), (II), or (II′), R¹² is substituted or unsubstituted methyl; and R¹³ is substituted or unsubstituted propargyl. In some embodiments of Formula (I), (I′), (II), or (II′), R¹² is substituted or unsubstituted ethyl; and R¹³ is substituted or unsubstituted propargyl. In some embodiments of Formula (I), (I′), (II), or (II′), R¹² is substituted or unsubstituted propyl; and R¹³ is substituted or unsubstituted propargyl. In some embodiments of Formula (I), (I′), (II), or (II′), R¹² is substituted or unsubstituted butyl; and R¹³ is substituted or unsubstituted propargyl. In some embodiments of Formula (I), (I′), (II), or (II′), R¹² is substituted or unsubstituted pentyl; and R¹³ is substituted or unsubstituted propargyl. In some embodiments of Formula (I), (I′), (II), or (II′), R¹² is substituted or unsubstituted C₁₋₆ haloalkyl; and R¹³ is substituted or unsubstituted propargyl. In some embodiments of Formula (I), (I′), (II), or (II′), R¹² is substituted or unsubstituted fluoroalkyl; and R¹³ is substituted or unsubstituted propargyl. In some embodiments of Formula (I), (I′), (II), or (II′), R¹² is trifluoromethyl; and R¹³ is substituted or unsubstituted propargyl. In some embodiments of Formula (I), (I′), (II), or (II′), R¹² is substituted or unsubstituted trifluoroethyl; and R¹³ is substituted or unsubstituted propargyl. In some embodiments of Formula (I), (I′), (II), or (II′), R¹² is substituted or unsubstituted C₂₋₆ alkenyl; and R¹³ is substituted or unsubstituted propargyl. In some embodiments of Formula (I), (I′), (II), or (II′), R¹² is substituted or unsubstituted C₂₋₆ alkenyl; and R¹³ is substituted or unsubstituted propargyl. In some embodiments of Formula (I), (I′), (II), or (II′), R¹² is substituted or unsubstituted vinyl; and R¹³ is substituted or unsubstituted propargyl. In some embodiments of Formula (I), (I′), (II), or (II′), R¹² is substituted or unsubstituted allyl; and R¹³ is substituted or unsubstituted propargyl. In some embodiments of Formula (I), (I′), (II), or (II′), R¹² is substituted or unsubstituted propenyl; and R¹³ is substituted or unsubstituted propargyl. In some embodiments of Formula (I), (I′), (II), or (II′), R¹² is substituted or unsubstituted butenyl; and R¹³ is substituted or unsubstituted propargyl. In some embodiments of Formula (I), (I′), (II), or (II′), R¹² is substituted or unsubstituted C₂₋₆ alkynyl; and R¹³ is substituted or unsubstituted propargyl. In some embodiments of Formula (I), (I′), (II), or (II′), R¹² is substituted or unsubstituted acetylenyl; and R¹³ is substituted or unsubstituted propargyl. In some embodiments of Formula (I), (I′), (II), or (II′), R¹² is substituted or unsubstituted propargyl; and R¹³ is substituted or unsubstituted propargyl. In some embodiments of Formula (I), (I′), (II), or (II′), R¹² is substituted or unsubstituted butynyl; and R¹³ is substituted or unsubstituted propargyl. In some embodiments of Formula (I), (I′), (II), or (II′), R¹² is substituted or unsubstituted C₁₋₆ alkoxy; and R¹³ is substituted or unsubstituted propargyl. In some embodiments of Formula (I), (I′), (II), or (II′), R¹² is CH₂—OMe; and R¹³ is substituted or unsubstituted propargyl. In some embodiments of Formula (I), (I′), (II), or (II′), R¹² is CH₂—OEt; and R¹³ is substituted or unsubstituted propargyl. In some embodiments of Formula (I), (I′), (II), or (II′), R¹²CH₂—OPr; and R¹³ is substituted or unsubstituted propargyl. In some embodiments of Formula (I), (I′), (II), or (II′), R¹² is CH₂—OCF₃; and R¹³ is substituted or unsubstituted propargyl. In some embodiments of Formula (I), (I′), (II), or (II′), R¹² is substituted or unsubstituted CH₂—OR⁷; and R¹³ is substituted or unsubstituted propargyl. In some embodiments of Formula (I), (I′), (II), or (II′), R¹² is substituted or unsubstituted C₆₋₁₀ aryl; and R¹³ is substituted or unsubstituted propargyl. In some embodiments of Formula (I), (I′), (II), or (II′), R¹² is substituted or unsubstituted 5-10 membered heteroaryl; and R¹³ is substituted or unsubstituted propargyl. In some embodiments of Formula (I), (I′), (II), or (II′), R¹² is substituted or unsubstituted 4-10 membered heterocyclyl; and R¹³ is substituted or unsubstituted propargyl. In some embodiments of Formula (I), (I′), (II), or (II′), R¹² is substituted or unsubstituted 3-10 membered carbocyclyl; and R¹³ is substituted or unsubstituted propargyl. In some embodiments of Formula (I), (I′), (II), or (II′), R¹² is acyl; and R¹³ is substituted or unsubstituted propargyl. In some embodiments of Formula (I), (I′), (II), or (II′), R¹² is a substituted or unsubstituted amide; and R¹³ is substituted or unsubstituted propargyl. In some embodiments of Formula (I), (I′), (II), or (II′), R¹² is a substituted or unsubstituted ester; and R¹³ is substituted or unsubstituted propargyl. In some embodiments of Formula (I), (I′), (II), or (II′), R¹² is —OH; and R¹³ is substituted or unsubstituted propargyl. In some embodiments of Formula (I), (I′), (II), or (II′), R¹² is —SO₂R⁷; and R¹³ is substituted or unsubstituted propargyl. In some embodiments of Formula (I), (I′), (II), or (II′), R¹² is —SOR⁷; and R¹³ is substituted or unsubstituted propargyl. In some embodiments of Formula (I), (I′), (II), or (II′), R¹² is —SO₂NR⁷₂; and R¹³ is substituted or unsubstituted propargyl. In some embodiments of Formula (I), (I′), (II), or (II′), R¹² is —OSO₂R⁷; and R¹³ is substituted or unsubstituted propargyl. In some embodiments of Formula (I), (I′), (II), or (II′), R¹² is —OSOR⁷; and R¹³ is substituted or unsubstituted propargyl. In some embodiments of Formula (I), (I′), (II), or (II′), R¹² is —OSO₂NR⁷₂; and R¹³ is substituted or unsubstituted propargyl. In some embodiments of Formula (I), (I′), (II), or (II′), R¹² is —OSO₂OR⁷; and R¹³ is substituted or unsubstituted propargyl. In some embodiments of Formula (I), (I′), (II), or (II′), R¹² is —O(SO)OR⁷; and R¹³ is substituted or unsubstituted propargyl. In some embodiments of Formula (I), (I′), (II), or (II′), R¹² is —PO(OR⁷)₂; and R¹³ is substituted or unsubstituted propargyl. In some embodiments of Formula (I), (I′), (II), or (II′), R¹² is —PO(NR⁷)₂; and R¹³ is substituted or unsubstituted propargyl. In some embodiments of Formula (I), (I′), (II), or (II′), R¹² is —PO(NR⁷)(OR⁷); and R¹³ is substituted or unsubstituted propargyl. In some embodiments of Formula (I), (I′), (II), or (II′), R¹² is —OPO(OR⁷)₂; and R¹³ is substituted or unsubstituted propargyl. In some embodiments of Formula (I), (I′), (II), or (II′), R¹² is —OPO(NR⁷)₂; and R¹³ is substituted or unsubstituted propargyl. In some embodiments of Formula (I), (I′), (II), or (II′), R¹² is —OPO(NR⁷)(OR⁷); and R¹³ is substituted or unsubstituted propargyl. In some embodiments of Formula (I), (I′), (II), or (II′), R¹² is a nitrogen protecting group; and R¹³ is substituted or unsubstituted propargyl. In some embodiments of Formula (I), (I′), (II), or (II′), R¹² is —OR⁷; and R¹³ is substituted or unsubstituted propargyl.

In some embodiments of Formula (I), (I′), (II), or (II′), R¹² is hydrogen; and R¹³ is substituted or unsubstituted butynyl. In some embodiments of Formula (I), (I′), (II), or (II′), R¹² is substituted or unsubstituted C₁₋₆ alkyl; and R¹³ is substituted or unsubstituted butynyl. In some embodiments of Formula (I), (I′), (II), or (II′), R¹² is substituted or unsubstituted C₁₋₃ alkyl; and R¹³ is substituted or unsubstituted butynyl. In some embodiments of Formula (I), (I′), (II), or (II′), R¹² is branched or unbranched substituted or unsubstituted C₁₋₆ alkyl; and R¹³ is substituted or unsubstituted butynyl. In some embodiments of Formula (I), (I′), (II), or (II′), R¹² is substituted or unsubstituted methyl; and R¹³ is substituted or unsubstituted butynyl. In some embodiments of Formula (I), (I′), (II), or (II′), R¹² is substituted or unsubstituted ethyl; and R¹³ is substituted or unsubstituted butynyl. In some embodiments of Formula (I), (I′), (II), or (II′), R¹² is substituted or unsubstituted propyl; and R¹³ is substituted or unsubstituted butynyl. In some embodiments of Formula (I), (I′), (II), or (II′), R¹² is substituted or unsubstituted butyl; and R¹³ is substituted or unsubstituted butynyl. In some embodiments of Formula (I), (I′), (II), or (II′), R¹² is substituted or unsubstituted pentyl; and R¹³ is substituted or unsubstituted butynyl. In some embodiments of Formula (I), (I′), (II), or (II′), R¹² is substituted or unsubstituted C₁₋₆ haloalkyl; and R¹³ is substituted or unsubstituted butynyl. In some embodiments of Formula (I), (I′), (II), or (II′), R¹² is substituted or unsubstituted fluoroalkyl; and R¹³ is substituted or unsubstituted butynyl. In some embodiments of Formula (I), (I′), (II), or (II′), R¹² is trifluoromethyl; and R¹³ is substituted or unsubstituted butynyl. In some embodiments of Formula (I), (I′), (II), or (II′), R¹² is substituted or unsubstituted trifluoroethyl; and R¹³ is substituted or unsubstituted butynyl. In some embodiments of Formula (I), (I′), (II), or (II′), R¹² is substituted or unsubstituted C₂₋₆ alkenyl; and R¹³ is substituted or unsubstituted butynyl. In some embodiments of Formula (I), (I′), (II), or (II′), R¹² is substituted or unsubstituted C₂₋₆ alkenyl; and R¹³ is substituted or unsubstituted butynyl. In some embodiments of Formula (I), (I′), (II), or (II′), R¹² is substituted or unsubstituted vinyl; and R¹³ is substituted or unsubstituted butynyl. In some embodiments of Formula (I), (I′), (II), or (II′), R¹² is substituted or unsubstituted allyl; and R¹³ is substituted or unsubstituted butynyl. In some embodiments of Formula (I), (I′), (II), or (II′), R¹² is substituted or unsubstituted propenyl; and R¹³ is substituted or unsubstituted butynyl. In some embodiments of Formula (I), (I′), (II), or (II′), R¹² is substituted or unsubstituted butenyl; and R¹³ is substituted or unsubstituted butynyl. In some embodiments of Formula (I), (I′), (II), or (II′), R¹² is substituted or unsubstituted C₂₋₆ alkynyl; and R¹³ is substituted or unsubstituted butynyl. In some embodiments of Formula (I), (I′), (II), or (II′), R¹² is substituted or unsubstituted acetylenyl; and R¹³ is substituted or unsubstituted butynyl. In some embodiments of Formula (I), (I′), (II), or (II′), R¹² is substituted or unsubstituted propargyl; and R¹³ is substituted or unsubstituted butynyl. In some embodiments of Formula (I), (I′), (II), or (II′), R¹² is substituted or unsubstituted butynyl; and R¹³ is substituted or unsubstituted butynyl. In some embodiments of Formula (I), (I′), (II), or (II′), R¹² is substituted or unsubstituted C₁₋₆ alkoxy; and R¹³ is substituted or unsubstituted butynyl. In some embodiments of Formula (I), (I′), (II), or (II′), R¹² is CH₂—OMe; and R¹³ is substituted or unsubstituted butynyl. In some embodiments of Formula (I), (I′), (II), or (II′), R¹² is CH₂—OEt; and R¹³ is substituted or unsubstituted butynyl. In some embodiments of Formula (I), (I′), (II), or (II′), R¹²CH₂—OPr; and R¹³ is substituted or unsubstituted butynyl. In some embodiments of Formula (I), (I′), (II), or (II′), R¹² is CH₂—OCF₃; and R¹³ is substituted or unsubstituted butynyl. In some embodiments of Formula (I), (I′), (II), or (II′), R¹² is substituted or unsubstituted CH₂—OR⁷; and R¹³ is substituted or unsubstituted butynyl. In some embodiments of Formula (I), (I′), (II), or (II′), R¹² is substituted or unsubstituted C₆₋₁₀ aryl; and R¹³ is substituted or unsubstituted butynyl. In some embodiments of Formula (I), (I′), (II), or (II′), R¹² is substituted or unsubstituted 5-10 membered heteroaryl; and R¹³ is substituted or unsubstituted butynyl. In some embodiments of Formula (I), (I′), (II), or (II′), R¹² is substituted or unsubstituted 4-10 membered heterocyclyl; and R¹³ is substituted or unsubstituted butynyl. In some embodiments of Formula (I), (I′), (II), or (II′), R¹² is substituted or unsubstituted 3-10 membered carbocyclyl; and R¹³ is substituted or unsubstituted butynyl. In some embodiments of Formula (I), (I′), (II), or (II′), R¹² is acyl; and R¹³ is substituted or unsubstituted butynyl. In some embodiments of Formula (I), (I′), (II), or (II′), R¹² is a substituted or unsubstituted amide; and R¹³ is substituted or unsubstituted butynyl. In some embodiments of Formula (I), (I′), (II), or (II′), R¹² is a substituted or unsubstituted ester; and R¹³ is substituted or unsubstituted butynyl. In some embodiments of Formula (I), (I′), (II), or (II′), R¹² is —OH; and R¹³ is substituted or unsubstituted butynyl. In some embodiments of Formula (I), (I′), (II), or (II′), R¹² is —SO₂R⁷; and R¹³ is substituted or unsubstituted butynyl. In some embodiments of Formula (I), (I′), (II), or (II′), R¹² is —SOR⁷; and R¹³ is substituted or unsubstituted butynyl. In some embodiments of Formula (I), (I′), (II), or (II′), R¹² is —SO₂NR⁷₂; and R¹³ is substituted or unsubstituted butynyl. In some embodiments of Formula (I), (I′), (II), or (II′), R¹² is —OSO₂R⁷; and R¹³ is substituted or unsubstituted butynyl. In some embodiments of Formula (I), (I′), (II), or (II′), R¹² is —OSOR⁷; and R¹³ is substituted or unsubstituted butynyl. In some embodiments of Formula (I), (I′), (II), or (II′), R¹² is —OSO₂NR⁷₂; and R¹³ is substituted or unsubstituted butynyl. In some embodiments of Formula (I), (I′), (II), or (II′), R¹² is —OSO₂OR⁷; and R¹³ is substituted or unsubstituted butynyl. In some embodiments of Formula (I), (I′), (II), or (II′), R¹² is —O(SO)OR⁷; and R¹³ is substituted or unsubstituted butynyl. In some embodiments of Formula (I), (I′), (II), or (II′), R¹² is —PO(OR⁷)₂; and R¹³ is substituted or unsubstituted butynyl. In some embodiments of Formula (I), (I′), (II), or (II′), R¹² is —PO(NR⁷)₂; and R¹³ is substituted or unsubstituted butynyl. In some embodiments of Formula (I), (I′), (II), or (II′), R¹² is —PO(NR⁷)(OR⁷); and R¹³ is substituted or unsubstituted butynyl. In some embodiments of Formula (I), (I′), (II), or (II′), R¹² is —OPO(OR⁷)₂; and R¹³ is substituted or unsubstituted butynyl. In some embodiments of Formula (I), (I′), (II), or (II′), R¹² is —OPO(NR⁷)₂; and R¹³ is substituted or unsubstituted butynyl. In some embodiments of Formula (I), (I′), (II), or (II′), R¹² is —OPO(NR⁷)(OR⁷); and R¹³ is substituted or unsubstituted butynyl. In some embodiments of Formula (I), (I′), (II), or (II′), R¹² is a nitrogen protecting group; and R¹³ is substituted or unsubstituted butynyl. In some embodiments of Formula (I), (I′), (II), or (II′), R¹² is —OR⁷; and R¹³ is substituted or unsubstituted butynyl.

In some embodiments of Formula (I), (I′), (II), or (II′), R¹² is hydrogen; and R¹³ is substituted or unsubstituted C₁₋₆ alkoxy. In some embodiments of Formula (I), (I′), (II), or (II′), R¹² is substituted or unsubstituted C₁₋₆ alkyl; and R¹³ is substituted or unsubstituted C₁₋₆ alkoxy. In some embodiments of Formula (I), (I′), (II), or (II′), R¹² is substituted or unsubstituted C₁₋₃ alkyl; and R¹³ is substituted or unsubstituted C₁₋₆ alkoxy. In some embodiments of Formula (I), (I′), (II), or (II′), R¹² is branched or unbranched substituted or unsubstituted C₁₋₆ alkyl; and R¹³ is substituted or unsubstituted C₁₋₆ alkoxy. In some embodiments of Formula (I), (I′), (II), or (II′), R¹² is substituted or unsubstituted methyl; and R¹³ is substituted or unsubstituted C₁₋₆ alkoxy. In some embodiments of Formula (I), (I′), (II), or (II′), R¹² is substituted or unsubstituted ethyl; and R¹³ is substituted or unsubstituted C₁₋₆ alkoxy. In some embodiments of Formula (I), (I′), (II), or (II′), R¹² is substituted or unsubstituted propyl; and R¹³ is substituted or unsubstituted C₁₋₆ alkoxy. In some embodiments of Formula (I), (I′), (II), or (II′), R¹² is substituted or unsubstituted butyl; and R¹³ is substituted or unsubstituted C₁₋₆ alkoxy. In some embodiments of Formula (I), (I′), (II), or (II′), R¹² is substituted or unsubstituted pentyl; and R¹³ is substituted or unsubstituted C₁₋₆ alkoxy. In some embodiments of Formula (I), (I′), (II), or (II′), R¹² is substituted or unsubstituted C₁₋₆ haloalkyl; and R¹³ is substituted or unsubstituted C₁₋₆ alkoxy. In some embodiments of Formula (I), (I′), (II), or (II′), R¹² is substituted or unsubstituted fluoroalkyl; and R¹³ is substituted or unsubstituted C₁₋₆ alkoxy. In some embodiments of Formula (I), (I′), (II), or (II′), R¹² is trifluoromethyl; and R¹³ is substituted or unsubstituted C₁₋₆ alkoxy. In some embodiments of Formula (I), (I′), (II), or (II′), R¹² is substituted or unsubstituted trifluoroethyl; and R¹³ is substituted or unsubstituted C₁₋₆ alkoxy. In some embodiments of Formula (I), (I′), (II), or (II′), R¹² is substituted or unsubstituted C₂₋₆ alkenyl; and R¹³ is substituted or unsubstituted C₁₋₆ alkoxy. In some embodiments of Formula (I), (I′), (II), or (II′), R¹² is substituted or unsubstituted C₂₋₆ alkenyl; and R¹³ is substituted or unsubstituted C₁₋₆ alkoxy. In some embodiments of Formula (I), (I′), (II), or (II′), R¹² is substituted or unsubstituted vinyl; and R¹³ is substituted or unsubstituted C₁₋₆ alkoxy. In some embodiments of Formula (I), (I′), (II), or (II′), R¹² is substituted or unsubstituted allyl; and R¹³ is substituted or unsubstituted C₁₋₆ alkoxy. In some embodiments of Formula (I), (I′), (II), or (II′), R¹² is substituted or unsubstituted propenyl; and R³ is substituted or unsubstituted C₁₋₆ alkoxy. In some embodiments of Formula (I), (I′), (II), or (II′), R¹² is substituted or unsubstituted butenyl; and R¹³ is substituted or unsubstituted C₁₋₆ alkoxy. In some embodiments of Formula (I), (I′), (II), or (II′), R¹² is substituted or unsubstituted C₂₋₆ alkynyl; and R¹³ is substituted or unsubstituted C₁₋₆ alkoxy. In some embodiments of Formula (I), (I′), (II), or (II′), R¹² is substituted or unsubstituted acetylenyl; and R¹³ is substituted or unsubstituted C₁₋₆ alkoxy. In some embodiments of Formula (I), (I′), (II), or (II′), R¹² is substituted or unsubstituted propargyl; and R³ is substituted or unsubstituted C₁₋₆ alkoxy. In some embodiments of Formula (I), (I′), (II), or (II′), R¹² is substituted or unsubstituted butynyl; and R³ is substituted or unsubstituted C₁₋₆ alkoxy. In some embodiments of Formula (I), (I′), (II), or (II′), R¹² is substituted or unsubstituted C₁₋₆ alkoxy; and R¹³ is substituted or unsubstituted C₁₋₆ alkoxy. In some embodiments of Formula (I), (I′), (II), or (II′), R¹² is CH₂—OMe; and R¹³ is substituted or unsubstituted C₁₋₆ alkoxy. In some embodiments of Formula (I), (I′), (II), or (II′), R¹² is CH₂—OEt; and R¹³ is substituted or unsubstituted C₁₋₆ alkoxy. In some embodiments of Formula (I), (I′), (II), or (II′), R¹²CH₂—OPr; and R¹³ is substituted or unsubstituted C₁₋₆ alkoxy. In some embodiments of Formula (I), (I′), (II), or (II′), R¹² is CH₂—OCF₃; and R¹³ is substituted or unsubstituted C₁₋₆alkoxy. In some embodiments of Formula (I), (I′), (II), or (II′), R¹² is substituted or unsubstituted CH₂—OR⁷; and R¹³ is substituted or unsubstituted C₁₋₆ alkoxy. In some embodiments of Formula (I), (I′), (II), or (II′), R¹² is substituted or unsubstituted C₆₋₁₀ aryl; and R¹³ is substituted or unsubstituted C₁₋₆ alkoxy. In some embodiments of Formula (I), (I′), (II), or (II′), R¹² is substituted or unsubstituted 5-10 membered heteroaryl; and R¹³ is substituted or unsubstituted C₁₋₆ alkoxy. In some embodiments of Formula (I), (I′), (II), or (II′), R¹² is substituted or unsubstituted 4-10 membered heterocyclyl; and R¹³ is substituted or unsubstituted C₁₋₆ alkoxy. In some embodiments of Formula (I), (I′), (II), or (II′), R¹² is substituted or unsubstituted 3-10 membered carbocyclyl; and R¹³ is substituted or unsubstituted C₁₋₆ alkoxy. In some embodiments of Formula (I), (I′), (II), or (II′), R¹² is acyl; and R¹³ is substituted or unsubstituted C₁₋₆ alkoxy. In some embodiments of Formula (I), (I′), (II), or (II′), R¹² is a substituted or unsubstituted amide; and R¹³ is substituted or unsubstituted C₁₋₆ alkoxy. In some embodiments of Formula (I), (I′), (II), or (II′), R¹² is a substituted or unsubstituted ester; and R¹³ is substituted or unsubstituted C₁₋₆ alkoxy. In some embodiments of Formula (I), (I′), (II), or (II′), R¹² is —OH; and R¹³ is substituted or unsubstituted C₁₋₆ alkoxy. In some embodiments of Formula (I), (I′), (II), or (II′), R¹² is —SO₂R⁷; and R¹³ is substituted or unsubstituted C₁₋₆ alkoxy. In some embodiments of Formula (I), (I′), (II), or (II′), R¹² is —SOR⁷; and R¹³ is substituted or unsubstituted C₁₋₆ alkoxy. In some embodiments of Formula (I), (I′), (II), or (II′), R¹² is —SO₂NR⁷₂; and R¹³ is substituted or unsubstituted C₁₋₆ alkoxy. In some embodiments of Formula (I), (I′), (II), or (II′), R¹² is —OSO₂R⁷; and R¹³ is substituted or unsubstituted C₁₋₆ alkoxy. In some embodiments of Formula (I), (I′), (II), or (II′), R¹² is —OSOR⁷; and R¹³ is substituted or unsubstituted C₁₋₆alkoxy. In some embodiments of Formula (I), (I′), (II), or (II′), R¹² is —OSO₂NR⁷₂; and R¹³ is substituted or unsubstituted C₁₋₆ alkoxy. In some embodiments of Formula (I), (I′), (II), or (II′), R¹² is —OSO₂OR⁷; and R¹³ is substituted or unsubstituted C₁₋₆ alkoxy. In some embodiments of Formula (I), (I′), (II), or (II′), R¹² is —O(SO)OR⁷; and R¹³ is substituted or unsubstituted C₁₋₆ alkoxy. In some embodiments of Formula (I), (I′), (II), or (II′), R¹² is —PO(OR⁷)₂; and R¹³ is substituted or unsubstituted C₁₋₆ alkoxy. In some embodiments of Formula (I), (I′), (II), or (II′), R¹² is —PO(NR⁷)₂; and R¹³ is substituted or unsubstituted C₁₋₆alkoxy. In some embodiments of Formula (I), (I′), (II), or (II′), R¹² is —PO(NR⁷)(OR⁷); and R¹³ is substituted or unsubstituted C₁₋₆ alkoxy. In some embodiments of Formula (I), (I′), (II), or (II′), R¹² is —OPO(OR⁷)₂; and R¹³ is substituted or unsubstituted C₁₋₆ alkoxy. In some embodiments of Formula (I), (I′), (II), or (II′), R¹² is —OPO(NR⁷)₂; and R¹³ is substituted or unsubstituted C₁₋₆ alkoxy. In some embodiments of Formula (I), (I′), (II), or (II′), R¹² is —OPO(NR⁷)(OR⁷); and R¹³ is substituted or unsubstituted C₁₋₆ alkoxy. In some embodiments of Formula (I), (I′), (II), or (II′), R¹² is a nitrogen protecting group; and R¹³ is substituted or unsubstituted C₁₋₆ alkoxy. In some embodiments of Formula (I), (I′), (II), or (II′), R¹² is —OR⁷; and R¹³ is substituted or unsubstituted C₁₋₆ alkoxy.

In some embodiments of Formula (I), (I′), (II), or (II′), R¹² is hydrogen; and R¹³ is CH₂—OMe. In some embodiments of Formula (I), (I′), (II), or (II′), R¹² is substituted or unsubstituted C₁₋₆ alkyl; and R¹³ is CH₂—OMe. In some embodiments of Formula (I), (I′), (II), or (II′), R¹² is substituted or unsubstituted C₁₋₃ alkyl; and R¹³ is CH₂—OMe. In some embodiments of Formula (I), (I′), (II), or (II′), R¹² is branched or unbranched substituted or unsubstituted C₁₋₆ alkyl; and R¹³ is CH₂—OMe. In some embodiments of Formula (I), (I′), (II), or (II′), R¹² is substituted or unsubstituted methyl; and R¹³ is CH₂—OMe. In some embodiments of Formula (I), (I′), (II), or (II′), R¹² is substituted or unsubstituted ethyl; and R¹³ is CH₂—OMe. In some embodiments of Formula (I), (I′), (II), or (II′), R¹² is substituted or unsubstituted propyl; and R¹³ is CH₂—OMe. In some embodiments of Formula (I), (I′), (II), or (II′), R¹² is substituted or unsubstituted butyl; and R¹³ is CH₂—OMe. In some embodiments of Formula (I), (I′), (II), or (II′), R¹² is substituted or unsubstituted pentyl; and R¹³ is CH₂—OMe. In some embodiments of Formula (I), (I′), (II), or (II′), R¹² is substituted or unsubstituted C₁₋₆ haloalkyl; and R¹³ is CH₂—OMe. In some embodiments of Formula (I), (I′), (II), or (II′), R¹² is substituted or unsubstituted fluoroalkyl; and R¹³ is CH₂—OMe. In some embodiments of Formula (I), (I′), (II), or (II′), R¹² is trifluoromethyl; and R¹³ is CH₂—OMe. In some embodiments of Formula (I), (I′), (II), or (II′), R¹² is substituted or unsubstituted trifluoroethyl; and R¹³ is CH₂—OMe. In some embodiments of Formula (I), (I′), (II), or (II′), R¹² is substituted or unsubstituted C₂₋₆ alkenyl; and R¹³ is CH₂—OMe. In some embodiments of Formula (I), (I′), (II), or (II′), R¹² is substituted or unsubstituted C₂₋₆ alkenyl; and R¹³ is CH₂—OMe. In some embodiments of Formula (I), (I′), (II), or (II′), R¹² is substituted or unsubstituted vinyl; and R¹³ is CH₂—OMe. In some embodiments of Formula (I), (I′), (II), or (II′), R¹² is substituted or unsubstituted allyl; and R¹³ is CH₂—OMe. In some embodiments of Formula (I), (I′), (II), or (II′), R¹² is substituted or unsubstituted propenyl; and R¹³ is CH₂—OMe. In some embodiments of Formula (I), (I′), (II), or (II′), R¹² is substituted or unsubstituted butenyl; and R¹³ is CH₂—OMe. In some embodiments of Formula (I), (I′), (II), or (II′), R¹² is substituted or unsubstituted C₂₋₆ alkynyl; and R¹³ is CH₂—OMe. In some embodiments of Formula (I), (I′), (II), or (II′), R¹² is substituted or unsubstituted acetylenyl; and R¹³ is CH₂—OMe. In some embodiments of Formula (I), (I′), (II), or (II′), R¹² is substituted or unsubstituted propargyl; and R¹³ is CH₂—OMe. In some embodiments of Formula (I), (I′), (II), or (II′), R¹² is substituted or unsubstituted butynyl; and R¹³ is CH₂—OMe. In some embodiments of Formula (I), (I′), (II), or (II′), R¹² is substituted or unsubstituted C₁₋₆ alkoxy; and R¹³ is CH₂—OMe. In some embodiments of Formula (I), (I′), (II), or (II′), R¹² is CH₂—OMe; and R¹³ is CH₂—OMe. In some embodiments of Formula (I), (I′), (II), or (II′), R¹² is CH₂—OEt; and R¹³ is CH₂—OMe. In some embodiments of Formula (I), (I′), (II), or (II′), R¹²CH₂—OPr; and R¹³ is CH₂—OMe. In some embodiments of Formula (I), (I′), (II), or (II′), R¹² is CH₂—OCF₃; and R¹³ is CH₂—OMe. In some embodiments of Formula (I), (I′), (II), or (II′), R¹² is substituted or unsubstituted CH₂—OR⁷; and R¹³ is CH₂—OMe. In some embodiments of Formula (I), (I′), (II), or (II′), R¹² is substituted or unsubstituted C₆₋₁₀ aryl; and R¹³ is CH₂—OMe. In some embodiments of Formula (I), (I′), (II), or (II′), R¹² is substituted or unsubstituted 5-10 membered heteroaryl; and R¹³ is CH₂—OMe. In some embodiments of Formula (I), (I′), (II), or (II′), R¹² is substituted or unsubstituted 4-10 membered heterocyclyl; and R¹³ is CH₂—OMe. In some embodiments of Formula (I), (I′), (II), or (II′), R¹² is substituted or unsubstituted 3-10 membered carbocyclyl; and R¹³ is CH₂—OMe. In some embodiments of Formula (I), (I′), (II), or (II′), R¹² is acyl; and R¹³ is CH₂—OMe. In some embodiments of Formula (I), (I′), (II), or (II′), R¹² is a substituted or unsubstituted amide; and R¹³ is CH₂—OMe. In some embodiments of Formula (I), (I′), (II), or (II′), R¹² is a substituted or unsubstituted ester; and R¹³ is CH₂—OMe. In some embodiments of Formula (I), (I′), (II), or (II′), R¹² is —OH; and R¹³ is CH₂—OMe. In some embodiments of Formula (I), (I′), (II), or (II′), R¹² is —SO₂R⁷; and R¹³ is CH₂—OMe. In some embodiments of Formula (I), (I′), (II), or (II′), R¹² is —SOR⁷; and R¹³ is CH₂—OMe. In some embodiments of Formula (I), (I′), (II), or (II′), R¹² is —SO₂NR⁷₂; and R¹³ is CH₂—OMe. In some embodiments of Formula (I), (I′), (II), or (II′), R¹² is —OSO₂R⁷; and R¹³ is CH₂—OMe. In some embodiments of Formula (I), (I′), (II), or (II′), R¹² is —OSOR⁷; and R¹³ is CH₂—OMe. In some embodiments of Formula (I), (I′), (II), or (II′), R¹² is —OSO₂NR⁷₂; and R¹³ is CH₂—OMe. In some embodiments of Formula (I), (I′), (II), or (II′), R¹² is —OSO₂OR⁷; and R¹³ is CH₂—OMe. In some embodiments of Formula (I), (I′), (II), or (II′), R¹² is —O(SO)OR⁷; and R¹³ is CH₂—OMe. In some embodiments of Formula (I), (I′), (II), or (II′), R¹² is —PO(OR⁷)₂; and R¹³ is CH₂—OMe. In some embodiments of Formula (I), (I′), (II), or (II′), R¹² is —PO(NR⁷)₂; and R¹³ is CH₂—OMe. In some embodiments of Formula (I), (I′), (II), or (II′), R¹² is —PO(NR⁷)(OR⁷); and R¹³ is CH₂—OMe. In some embodiments of Formula (I), (I′), (II), or (II′), R¹² is —OPO(OR⁷)₂; and R¹³ is CH₂—OMe. In some embodiments of Formula (I), (I′), (II), or (II′), R¹² is —OPO(NR⁷)₂; and R¹³ is CH₂—OMe. In some embodiments of Formula (I), (I′), (II), or (II′), R¹² is —OPO(NR⁷)(OR⁷); and R¹³ is CH₂—OMe. In some embodiments of Formula (I), (I′), (II), or (II′), R¹² is a nitrogen protecting group; and R¹³ is CH₂—OMe. In some embodiments of Formula (I), (I′), (II), or (II′), R¹² is —OR⁷; and R¹³ is CH₂—OMe.

In some embodiments of Formula (I), (I′), (II), or (II′), R¹² is hydrogen; and R¹³ is CH₂—OEt. In some embodiments of Formula (I), (I′), (II), or (II′), R¹² is substituted or unsubstituted C₁₋₆ alkyl; and R¹³ is CH₂—OEt. In some embodiments of Formula (I), (I′), (II), or (II′), R¹² is substituted or unsubstituted C₁₋₃ alkyl; and R¹³ is CH₂—OEt. In some embodiments of Formula (I), (I′), (II), or (II′), R¹² is branched or unbranched substituted or unsubstituted C₁₋₆ alkyl; and R¹³ is CH₂—OEt. In some embodiments of Formula (I), (I′), (II), or (II′), R¹² is substituted or unsubstituted methyl; and R¹³ is CH₂—OEt. In some embodiments of Formula (I), (I′), (II), or (II′), R¹² is substituted or unsubstituted ethyl; and R¹³ is CH₂—OEt. In some embodiments of Formula (I), (I′), (II), or (II′), R¹² is substituted or unsubstituted propyl; and R¹³ is CH₂—OEt. In some embodiments of Formula (I), (I′), (II), or (II′), R¹² is substituted or unsubstituted butyl; and R¹³ is CH₂—OEt. In some embodiments of Formula (I), (I′), (II), or (II′), R¹² is substituted or unsubstituted pentyl; and R¹³ is CH₂—OEt. In some embodiments of Formula (I), (I′), (II), or (II′), R¹² is substituted or unsubstituted C₁₋₆ haloalkyl; and R¹³ is CH₂—OEt. In some embodiments of Formula (I), (I′), (II), or (II′), R¹² is substituted or unsubstituted fluoroalkyl; and R¹³ is CH₂—OEt. In some embodiments of Formula (I), (I′), (II), or (II′), R¹² is trifluoromethyl; and R¹³ is CH₂—OEt. In some embodiments of Formula (I), (I′), (II), or (II′), R¹² is substituted or unsubstituted trifluoroethyl; and R¹³ is CH₂—OEt. In some embodiments of Formula (I), (I′), (II), or (II′), R¹² is substituted or unsubstituted C₂₋₆ alkenyl; and R¹³ is CH₂—OEt. In some embodiments of Formula (I), (I′), (II), or (II′), R¹² is substituted or unsubstituted C₂₋₆ alkenyl; and R¹³ is CH₂—OEt. In some embodiments of Formula (I), (I′), (II), or (II′), R¹² is substituted or unsubstituted vinyl; and R¹³ is CH₂—OEt. In some embodiments of Formula (I), (I′), (II), or (II′), R¹² is substituted or unsubstituted allyl; and R¹³ is CH₂—OEt. In some embodiments of Formula (I), (I′), (II), or (II′), R¹² is substituted or unsubstituted propenyl; and R¹³ is CH₂-OEt. In some embodiments of Formula (I), (I′), (II), or (II′), R¹² is substituted or unsubstituted butenyl; and R¹³ is CH₂—OEt. In some embodiments of Formula (I), (I′), (II), or (II′), R¹² is substituted or unsubstituted C₂₋₆ alkynyl; and R¹³ is CH₂—OEt. In some embodiments of Formula (I), (I′), (II), or (II′), R¹² is substituted or unsubstituted acetylenyl; and R¹³ is CH₂—OEt. In some embodiments of Formula (I), (I′), (II), or (II′), R¹² is substituted or unsubstituted propargyl; and R¹³ is CH₂—OEt. In some embodiments of Formula (I), (I′), (II), or (II′), R¹² is substituted or unsubstituted butynyl; and R¹³ is CH₂-OEt. In some embodiments of Formula (I), (I′), (II), or (II′), R¹² is substituted or unsubstituted C₁₋₆ alkoxy; and R¹³ is CH₂—OEt. In some embodiments of Formula (I), (I′), (II), or (II′), R¹² is CH₂—OMe; and R¹³ is CH₂-OEt. In some embodiments of Formula (I), (I′), (II), or (II′), R¹² is CH₂—OEt; and R¹³ is CH₂—OEt. In some embodiments of Formula (I), (I′), (II), or (II′), (II′), R¹²CH₂—OPr; and R¹³ is CH₂—OEt. In some embodiments of Formula (I), (I′), (II), or (II′), R¹² is CH₂—OCF₃; and R¹³ is CH₂—OEt. In some embodiments of Formula (I), (I′), (II), or (II′), R¹² is substituted or unsubstituted CH₂—OR⁷; and R¹³ is CH₂-OEt. In some embodiments of Formula (I), (I′), (II), or (II′), R¹² is substituted or unsubstituted C₆₋₁₀ aryl; and R¹³ is CH₂—OEt. In some embodiments of Formula (I), (I′), (II), or (II′), R¹² is substituted or unsubstituted 5-10 membered heteroaryl; and R¹³ is CH₂—OEt. In some embodiments of Formula (I), (I′), (II), or (II′), R¹² is substituted or unsubstituted 4-10 membered heterocyclyl; and R¹³ is CH₂—OEt. In some embodiments of Formula (I), (I′), (II), or (II′), R¹² is substituted or unsubstituted 3-10 membered carbocyclyl; and R¹³ is CH₂-OEt. In some embodiments of Formula (I), (I′), (II), or (II′), R¹² is acyl; and R¹³ is CH₂-OEt. In some embodiments of Formula (I), (I′), (II), or (II′), R¹² is a substituted or unsubstituted amide; and R¹³ is CH₂—OEt. In some embodiments of Formula (I), (I′), (II), or (II′), R¹² is a substituted or unsubstituted ester; and R¹³ is CH₂—OEt. In some embodiments of Formula (I), (I′), (II), or (II′), R¹² is —OH; and R¹³ is CH₂—OEt. In some embodiments of Formula (I), (I′), (II), or (II′), R¹² is —SO₂R⁷; and R¹³ is CH₂—OEt. In some embodiments of Formula (I), (I′), (II), or (II′), R¹² is —SOR⁷; and R¹³ is CH₂—OEt. In some embodiments of Formula (I), (I′), (II), or (II′), R¹² is —SO₂NR⁷₂; and R¹³ is CH₂—OEt. In some embodiments of Formula (I), (I′), (II), or (II′), R¹² is —OSO₂R⁷; and R¹³ is CH₂—OEt. In some embodiments of Formula (I), (I′), (II), or (II′), R¹² is —OSOR⁷; and R¹³ is CH₂—OEt. In some embodiments of Formula (I), (I′), (II), or (II′), R¹² is —OSO₂NR⁷₂; and R¹³ is CH₂—OEt. In some embodiments of Formula (I), (I′), (II), or (II′), R¹² is —OSO₂R⁷; and R¹³ is CH₂—OEt. In some embodiments of Formula (I), (I′), (II), or (II′), R¹² is —O(SO)OR⁷; and R¹³ is CH₂-OEt. In some embodiments of Formula (I), (I′), (II), or (II′), R¹² is —PO(OR⁷)₂; and R¹³ is CH₂—OEt. In some embodiments of Formula (I), (I′), (II), or (II′), R¹² is —PO(NR⁷)₂; and R¹³ is CH₂—OEt. In some embodiments of Formula (I), (I′), (II), or (II′), R¹² is —PO(NR⁷)(OR⁷); and R¹¹ is CH₂—OEt. In some embodiments of Formula (I), (I′), (II), or (II′), R¹² is —OPO(OR⁷)₂; and R¹³ is CH₂—OEt. In some embodiments of Formula (I), (I′), (II), or (II′), R¹² is —OPO(NR⁷)₂; and R¹³ is CH₂—OEt. In some embodiments of Formula (I), (I′), (II), or (II′), R¹² is —OPO(NR⁷)(OR⁷); and R¹³ is CH₂—OEt. In some embodiments of Formula (I), (I′), (II), or (II′), R¹² is a nitrogen protecting group; and R¹³ is CH₂—OEt. In some embodiments of Formula (I), (I′), (II), or (II′), R¹² is —OR⁷; and R¹³ is CH₂—OEt.

In some embodiments of Formula (I), (I′), (II), or (II′), R¹² is hydrogen; and R¹³ is CH₂—OPr. In some embodiments of Formula (I), (I′), (II), or (II′), R¹² is substituted or unsubstituted C₁₋₆ alkyl; and R¹³ is CH₂—OPr. In some embodiments of Formula (I), (I′), (II), or (II′), R¹² is substituted or unsubstituted C₁₋₃ alkyl; and R¹³ is CH₂—OPr. In some embodiments of Formula (I), (I′), (II), or (II′), R¹² is branched or unbranched substituted or unsubstituted C₁₋₆ alkyl; and R¹³ is CH₂—OPr. In some embodiments of Formula (I), (I′), (II), or (II′), R¹² is substituted or unsubstituted methyl; and R¹³ is CH₂—OPr. In some embodiments of Formula (I), (I′), (II), or (II′), R¹² is substituted or unsubstituted ethyl; and R¹³ is CH₂—OPr. In some embodiments of Formula (I), (I′), (II), or (II′), R¹² is substituted or unsubstituted propyl; and R¹³ is CH₂—OPr. In some embodiments of Formula (I), (I′), (II), or (II′), R¹² is substituted or unsubstituted butyl; and R¹³ is CH₂—OPr. In some embodiments of Formula (I), (I′), (II), or (II′), R¹² is substituted or unsubstituted pentyl; and R¹³ is CH₂—OPr. In some embodiments of Formula (I), (I′), (II), or (II′), R¹² is substituted or unsubstituted C₁₋₆ haloalkyl; and R¹³ is CH₂—OPr. In some embodiments of Formula (I), (I′), (II), or (II′), R¹² is substituted or unsubstituted fluoroalkyl; and R¹³ is CH₂—OPr. In some embodiments of Formula (I), (I′), (II), or (II′), R¹² is trifluoromethyl; and R¹³ is CH₂—OPr. In some embodiments of Formula (I), (I′), (II), or (II′), R¹² is substituted or unsubstituted trifluoroethyl; and R¹³ is CH₂—OPr. In some embodiments of Formula (I), (I′), (II), or (II′), R¹² is substituted or unsubstituted C₂₋₆ alkenyl; and R¹³ is CH₂—OPr. In some embodiments of Formula (I), (I′), (II), or (II′), R¹² is substituted or unsubstituted C₂₋₆ alkenyl; and R¹³ is CH₂—OPr. In some embodiments of Formula (I), (I′), (II), or (II′), R¹² is substituted or unsubstituted vinyl; and R¹³ is CH₂—OPr. In some embodiments of Formula (I), (I′), (II), or (II′), R¹² is substituted or unsubstituted allyl; and R¹³ is CH₂—OPr. In some embodiments of Formula (I), (I′), (II), or (II′), R¹² is substituted or unsubstituted propenyl; and R¹³ is CH₂—OPr. In some embodiments of Formula (I), (I′), (II), or (II′), R¹² is substituted or unsubstituted butenyl; and R¹³ is CH₂—OPr. In some embodiments of Formula (I), (I′), (II), or (II′), R¹² is substituted or unsubstituted C₂₋₆ alkynyl; and R¹³ is CH₂—OPr. In some embodiments of Formula (I), (I′), (II), or (II′), R¹² is substituted or unsubstituted acetylenyl; and R¹³ is CH₂—OPr. In some embodiments of Formula (I), (I′), (II), or (II′), R¹² is substituted or unsubstituted propargyl; and R¹³ is CH₂—OPr. In some embodiments of Formula (I), (I′), (II), or (II′), R¹² is substituted or unsubstituted butynyl; and R¹³ is CH₂—OPr. In some embodiments of Formula (I), (I′), (II), or (II′), R¹² is substituted or unsubstituted C₁₋₆ alkoxy; and R¹³ is CH₂—OPr. In some embodiments of Formula (I), (I′), (II), or (II′), R¹² is CH₂—OMe; and R¹¹ is CH₂—OPr. In some embodiments of Formula (I), (I′), (II), or (II′), R¹² is CH₂—OEt; and R¹³ is CH₂—OPr. In some embodiments of Formula (I), (I′), (II), or (II′), (II′), R¹²CH₂—OPr; and R¹³ is CH₂—OPr. In some embodiments of Formula (I), (I′), (II), or (II′), R¹² is CH₂—OCF₃; and R¹³ is CH₂—OPr. In some embodiments of Formula (I), (I′), (II), or (II′), R¹² is substituted or unsubstituted CH₂—OR⁷; and R¹³ is CH₂—OPr. In some embodiments of Formula (I), (I′), (II), or (II′), R¹² is substituted or unsubstituted C₆₋₁₀ aryl; and R¹³ is CH₂—OPr. In some embodiments of Formula (I), (I′), (II), or (II′), R¹² is substituted or unsubstituted 5-10 membered heteroaryl; and R¹³ is CH₂—OPr. In some embodiments of Formula (I), (I′), (II), or (II′), R¹² is substituted or unsubstituted 4-10 membered heterocyclyl; and R¹³ is CH₂—OPr. In some embodiments of Formula (I), (I′), (II), or (II′), R¹² is substituted or unsubstituted 3-10 membered carbocyclyl; and R¹³ is CH₂—OPr. In some embodiments of Formula (I), (I′), (II), or (II′), R¹² is acyl; and R¹³ is CH₂—OPr. In some embodiments of Formula (I), (I′), (II), or (II′), R¹² is a substituted or unsubstituted amide; and R¹³ is CH₂—OPr. In some embodiments of Formula (I), (I′), (II), or (II′), R¹² is a substituted or unsubstituted ester; and R¹³ is CH₂—OPr. In some embodiments of Formula (I), (I′), (II), or (II′), R¹² is —OH; and R¹³ is CH₂—OPr. In some embodiments of Formula (I), (I′), (II), or (II′), R¹² is —SO₂R⁷; and R¹³ is CH₂—OPr. In some embodiments of Formula (I), (I′), (II), or (II′), R¹² is —SOR⁷; and R¹³ is CH₂—OPr. In some embodiments of Formula (I), (I′), (II), or (II′), R¹² is —SO₂NR⁷₂; and R¹³ is CH₂—OPr. In some embodiments of Formula (I), (I′), (II), or (II′), R¹² is —OSO₂R⁷; and R¹³ is CH₂—OPr. In some embodiments of Formula (I), (I′), (II), or (II′), R¹² is —OSOR⁷; and R¹³ is CH₂—OPr. In some embodiments of Formula (I), (I′), (II), or (II′), R¹² is —OSO₂NR⁷₂; and R¹³ is CH₂—OPr. In some embodiments of Formula (I), (I′), (II), or (II′), R¹² is —OSO₂OR⁷; and R¹³ is CH₂—OPr. In some embodiments of Formula (I), (I′), (II), or (II′), R¹² is —O(SO)OR⁷; and R¹³ is CH₂—OPr. In some embodiments of Formula (I), (I′), (II), or (II′), R¹² is —PO(OR⁷)₂; and R¹³ is CH₂—OPr. In some embodiments of Formula (I), (I′), (II), or (II′), R¹² is —PO(NR⁷)₂; and R¹³ is CH₂—OPr. In some embodiments of Formula (I), (I′), (II), or (II′), R¹² is —PO(NR⁷)(OR⁷); and R¹³ is CH₂—OPr. In some embodiments of Formula (I), (I′), (II), or (II′), R¹² is —OPO(OR⁷)₂; and R¹³ is CH₂—OPr. In some embodiments of Formula (I), (I′), (II), or (II′), R¹² is —OPO(NR⁷)₂; and R¹³ is CH₂—OPr. In some embodiments of Formula (I), (I′), (II), or (II′), R¹² is —OPO(NR⁷)(OR⁷); and R¹³ is CH₂—OPr. In some embodiments of Formula (I), (I′), (II), or (II′), R¹² is a nitrogen protecting group; and R¹³ is CH₂—OPr. In some embodiments of Formula (I), (I′), (II), or (II′), R¹² is —OR⁷; and R¹³ is CH₂—OPr.

In some embodiments of Formula (I), (I′), (II), or (II′), R¹² is hydrogen; and R¹³ is CH₂—OCF₃. In some embodiments of Formula (I), (I′), (II), or (II′), R¹² is substituted or unsubstituted C₁₋₆ alkyl; and R¹³ is CH₂—OCF₃. In some embodiments of Formula (I), (I′), (II), or (II′), R¹² is substituted or unsubstituted C₁₋₃ alkyl; and R¹¹ is CH₂—OCF₃. In some embodiments of Formula (I), (I′), (II), or (II′), R¹² is branched or unbranched substituted or unsubstituted C₁₋₆ alkyl; and R¹³ is CH₂—OCF₃. In some embodiments of Formula (I), (I′), (II), or (II′), R¹² is substituted or unsubstituted methyl; and R¹³ is CH₂—OCF₃. In some embodiments of Formula (I), (I′), (II), or (II′), R¹² is substituted or unsubstituted ethyl; and R¹³ is CH—OCF₃. In some embodiments of Formula (I), (I′), (II), or (II′), R¹² is substituted or unsubstituted propyl; and R¹³ is CH₂—OCF₃. In some embodiments of Formula (I), (I′), (II), or (II′), R¹² is substituted or unsubstituted butyl; and R³ is CH₂—OCF₃. In some embodiments of Formula (I), (I′), (II), or (II′), R¹² is substituted or unsubstituted pentyl; and R¹³ is CH₂—OCF₃. In some embodiments of Formula (I), (I′), (II), or (II′), R¹² is substituted or unsubstituted C₁₋₆ haloalkyl; and R¹³ is CH₂—OCF₃. In some embodiments of Formula (I), (I′), (II), or (II′), R¹² is substituted or unsubstituted fluoroalkyl; and R¹³ is CH₂—OCF₃. In some embodiments of Formula (I), (I′), (II), or (II′), R¹² is trifluoromethyl; and R¹³ is CH₂—OCF₃. In some embodiments of Formula (I), (I′), (II), or (II′), R¹² is substituted or unsubstituted trifluoroethyl; and R¹³ is CH₂—OCF₃. In some embodiments of Formula (I), (I′), (II), or (II′), R¹² is substituted or unsubstituted C₂₋₆ alkenyl; and R¹³ is CH₂—OCF₃. In some embodiments of Formula (I), (I′), (II), or (II′), R¹² is substituted or unsubstituted C₂₋₆ alkenyl; and R¹³ is CH₂—OCF₃. In some embodiments of Formula (I), (I′), (II), or (II′), R¹² is substituted or unsubstituted vinyl; and R¹³ is CH₂—OCF₃. In some embodiments of Formula (I), (I′), (II), or (II′), R¹² is substituted or unsubstituted allyl; and R¹³ is CH₂—OCF₃. In some embodiments of Formula (I), (I′), (II), or (II′), R¹² is substituted or unsubstituted propenyl; and R¹³ is CH₂—OCF₃. In some embodiments of Formula (I), (I′), (II), or (II′), R¹² is substituted or unsubstituted butenyl; and R¹³ is CH₂—OCF₃. In some embodiments of Formula (I), (I′), (II), or (II′), R¹² is substituted or unsubstituted C₂₋₆ alkynyl; and R¹³ is CH₂—OCF₃. In some embodiments of Formula (I), (I′), (II), or (II′), R¹² is substituted or unsubstituted acetylenyl; and R¹³ is CH₂—OCF₃. In some embodiments of Formula (I), (I′), (II), or (II′), R¹² is substituted or unsubstituted propargyl; and R¹³ is CH₂—OCF₃. In some embodiments of Formula (I), (I′), (II), or (II′), R¹² is substituted or unsubstituted butynyl; and R¹³ is CH₂—OCF₃. In some embodiments of Formula (I), (I′), (II), or (II′), R¹² is substituted or unsubstituted C₁₋₆ alkoxy; and R¹³ is CH₂—OCF₃. In some embodiments of Formula (I), (I′), (II), or (II′), R¹² is CH₂—OMe; and R¹³ is CH₂—OCF₃. In some embodiments of Formula (I), (I′), (II), or (II′), R¹² is CH₂-OEt; and R¹³ is CH₂—OCF₃. In some embodiments of Formula (I), (I′), (II), or (II′), R¹²CH—OPr; and R¹³ is CH₂—OCF₃. In some embodiments of Formula (I), (I′), (II), or (II′), R¹² is CH₂—OCF₃; and R¹³ is CH₂—OCF₃. In some embodiments of Formula (I), (I′), (II), or (II′), R¹² is substituted or unsubstituted CH₂—OR⁷; and R¹³ is CH₂—OCF₃. In some embodiments of Formula (I), (I′), (II), or (II′), R¹² is substituted or unsubstituted C₆₋₁₀ aryl; and R¹³ is CH₂—OCF₃. In some embodiments of Formula (I), (I′), (II), or (II′), R¹² is substituted or unsubstituted 5-10 membered heteroaryl; and R¹³ is CH₂—OCF₃. In some embodiments of Formula (I), (I′), (II), or (II′), R¹² is substituted or unsubstituted 4-10 membered heterocyclyl; and R¹³ is CH₂—OCF₃. In some embodiments of Formula (I), (I′), (II), or (II′), R¹² is substituted or unsubstituted 3-10 membered carbocyclyl; and R¹³ is CH₂—OCF₃. In some embodiments of Formula (I), (I′), (II), or (II′), R¹² is acyl; and R¹³ is CH₂—OCF₃. In some embodiments of Formula (I), (I′), (II), or (II′), R¹² is a substituted or unsubstituted amide; and R¹³ is CH₂—OCF₃. In some embodiments of Formula (I), (I′), (II), or (II′), R¹² is a substituted or unsubstituted ester; and R¹³ is CH₂—OCF₃. In some embodiments of Formula (I), (I′), (II), or (II′), R¹² is —OH; and R¹³ is CH₂—OCF₃. In some embodiments of Formula (I), (I′), (II), or (II′), R¹² is —SO₂R⁷; and R¹³ is CH₂—OCF₃. In some embodiments of Formula (I), (I′), (II), or (II′), R¹² is —SOR⁷; and R¹³ is CH₂—OCF₃. In some embodiments of Formula (I), (I′), (II), or (II′), R¹² is —SO₂NR⁷₂; and R¹¹ is CH₂—OCF₃. In some embodiments of Formula (I), (I′), (II), or (II′), R¹² is —OSO₂R⁷; and R¹³ is CH₂—OCF₃. In some embodiments of Formula (I), (I′), (II), or (II′), R¹² is —OSOR⁷; and R¹³ is CH₂—OCF₃. In some embodiments of Formula (I), (I′), (II), or (II′), R¹² is —OSO₂NR⁷₂; and R¹³ is CH₂—OCF₃. In some embodiments of Formula (I), (I′), (II), or (II′), R¹² is —CO₂OR⁷; and R³ is CH₂—OCF₃. In some embodiments of Formula (I), (I′), (II), or (II′), R¹² is —O(SO)OR⁷; and R¹³ is CH₂—OCF₃. In some embodiments of Formula (I), (I′), (II), or (II′), R¹² is —PO(OR⁷)₂; and R¹³ is CH₂—OCF₃. In some embodiments of Formula (I), (I′), (II), or (II′), R¹² is —PO(NR⁷)₂; and R¹³ is CH₂—OCF₃. In some embodiments of Formula (I), (I′), (II), or (II′), R¹² is —PO(NR⁷)(OR⁷); and R¹³ is CH₂—OCF₃. In some embodiments of Formula (I), (I′), (II), or (II′), R¹² is —OPO(OR⁷)₂; and R¹³ is CH₂—OCF₃. In some embodiments of Formula (I), (I′), (II), or (II′), R¹² is —OPO(NR⁷)₂; and R¹³ is CH₂—OCF₃. In some embodiments of Formula (I), (I′), (II), or (II′), R¹² is —OPO(NR⁷)(OR⁷); and R¹³ is CH₂—OCF₃. In some embodiments of Formula (I), (I′), (II), or (II′), R¹² is a nitrogen protecting group; and R¹³ is CH₂—OCF₃. In some embodiments of Formula (I), (I′), (II), or (II′), R¹² is —OR⁷; and R¹³ is CH₂—OCF₃.

In some embodiments of Formula (I), (I′), (II), or (II′), R¹² is hydrogen; and R¹³ is substituted or unsubstituted CH₂—OR⁷. In some embodiments of Formula (I), (I′), (II), or (II′), R¹² is substituted or unsubstituted C₁₋₆ alkyl; and R¹¹ is substituted or unsubstituted CH₂—OR⁷. In some embodiments of Formula (I), (I′), (II), or (II′), R¹² is substituted or unsubstituted C₁₋₃ alkyl; and R¹³ is substituted or unsubstituted CH₂—OR⁷. In some embodiments of Formula (I), (I′), (II), or (II′), R¹² is branched or unbranched substituted or unsubstituted C₁₋₆ alkyl; and R¹³ is substituted or unsubstituted CH₂—OR⁷. In some embodiments of Formula (I), (I′), (II), or (II′), R¹² is substituted or unsubstituted methyl; and R¹³ is substituted or unsubstituted CH₂—OR⁷. In some embodiments of Formula (I), (I′), (II), or (II′), R¹² is substituted or unsubstituted ethyl; and R¹³ is substituted or unsubstituted CH₂—OR⁷. In some embodiments of Formula (I), (I′), (II), or (II′), R¹² is substituted or unsubstituted propyl; and R¹³ is substituted or unsubstituted CH₂—OR⁷. In some embodiments of Formula (I), (I′), (II), or (II′), R¹² is substituted or unsubstituted butyl; and R¹³ is substituted or unsubstituted CH₂—OR⁷. In some embodiments of Formula (I), (I′), (II), or (II′), R¹² is substituted or unsubstituted pentyl; and R¹³ is substituted or unsubstituted CH₂—OR⁷. In some embodiments of Formula (I), (I′), (II), or (II′), R¹² is substituted or unsubstituted C₁₋₆ haloalkyl; and R¹³ is substituted or unsubstituted CH₂—OR⁷. In some embodiments of Formula (I), (I′), (II), or (II′), R¹² is substituted or unsubstituted fluoroalkyl; and R¹³ is substituted or unsubstituted CH₂—OR⁷. In some embodiments of Formula (I), (I′), (II), or (II′), R¹² is trifluoromethyl; and R¹³ is substituted or unsubstituted CH₂—OR⁷. In some embodiments of Formula (I), (I′), (II), or (II′), R¹² is substituted or unsubstituted trifluoroethyl; and R¹³ is substituted or unsubstituted CH₂—OR⁷. In some embodiments of Formula (I), (I′), (II), or (II′), R¹² is substituted or unsubstituted C₂₋₆ alkenyl; and R¹³ is substituted or unsubstituted CH₂—OR⁷. In some embodiments of Formula (I), (I′), (II), or (II′), R¹² is substituted or unsubstituted C₂₋₆ alkenyl; and R¹³ is substituted or unsubstituted CH₂—OR⁷. In some embodiments of Formula (I), (I′), (II), or (II′), R¹² is substituted or unsubstituted vinyl; and R¹³ is substituted or unsubstituted CH₂—OR⁷. In some embodiments of Formula (I), (I′), (II), or (II′), R¹² is substituted or unsubstituted allyl; and R¹³ is substituted or unsubstituted CH₂—OR⁷. In some embodiments of Formula (I), (I′), (II), or (II′), R¹² is substituted or unsubstituted propenyl; and R¹³ is substituted or unsubstituted CH₂—OR⁷. In some embodiments of Formula (I), (I′), (II), or (II′), R¹² is substituted or unsubstituted butenyl; and R¹³ is substituted or unsubstituted CH₂—OR⁷. In some embodiments of Formula (I), (I′), (II), or (II′), R¹² is substituted or unsubstituted C₂₋₆ alkynyl; and R¹³ is substituted or unsubstituted CH₂—OR⁷. In some embodiments of Formula (I), (I′), (II), or (II′), R¹² is substituted or unsubstituted acetylenyl; and R¹³ is substituted or unsubstituted CH₂—OR⁷. In some embodiments of Formula (I), (I′), (II), or (II′), R¹² is substituted or unsubstituted propargyl; and R¹³ is substituted or unsubstituted CH₂—OR⁷. In some embodiments of Formula (I), (I′), (II), or (II′), R¹² is substituted or unsubstituted butynyl; and R¹³ is substituted or unsubstituted CH₂—OR⁷. In some embodiments of Formula (I), (I′), (II), or (II′), R¹² is substituted or unsubstituted C₁₋₆ alkoxy; and R¹³ is substituted or unsubstituted CH₂—OR⁷. In some embodiments of Formula (I), (I′), (II), or (II′), R¹² is CH₂—OMe; and R¹³ is substituted or unsubstituted CH₂—OR⁷. In some embodiments of Formula (I), (I′), (II), or (II′), R¹² is CH₂—OEt; and R¹³ is substituted or unsubstituted CH₂—OR⁷. In some embodiments of Formula (I), (I′), (II), or (II′), R¹²CH₂—OPr; and R¹³ is substituted or unsubstituted CH₂—OR⁷. In some embodiments of Formula (I), (I′), (II), or (II′), R¹² is CH₂—OCF₃; and R¹³ is substituted or unsubstituted CH₂—OR⁷. In some embodiments of Formula (I), (I′), (II), or (II′), R¹² is substituted or unsubstituted CH₂—OR⁷; and R¹³ is substituted or unsubstituted CH₂—OR⁷. In some embodiments of Formula (I), (I′), (II), or (II′), R¹² is substituted or unsubstituted C₆₋₁₀ aryl; and R¹³ is substituted or unsubstituted CH₂—OR⁷. In some embodiments of Formula (I), (I′), (II), or (II′), R¹² is substituted or unsubstituted 5-10 membered heteroaryl; and R¹³ is substituted or unsubstituted CH₂—OR⁷. In some embodiments of Formula (I), (I′), (II), or (II′), R¹² is substituted or unsubstituted 4-10 membered heterocyclyl; and R¹³ is substituted or unsubstituted CH₂—OR⁷. In some embodiments of Formula (I), (I′), (II), or (II′), R¹² is substituted or unsubstituted 3-10 membered carbocyclyl; and R¹³ is substituted or unsubstituted CH₂—OR⁷. In some embodiments of Formula (I), (I′), (II), or (II′), R¹² is acyl; and R¹³ is substituted or unsubstituted CH₂—OR⁷. In some embodiments of Formula (I), (I′), (II), or (II′), R¹² is a substituted or unsubstituted amide; and R¹³ is substituted or unsubstituted CH₂—OR⁷. In some embodiments of Formula (I), (I′), (II), or (II′), R¹² is a substituted or unsubstituted ester; and R¹³ is substituted or unsubstituted CH₂—OR⁷. In some embodiments of Formula (I), (I′), (II), or (II′), R¹² is —OH; and R¹³ is substituted or unsubstituted CH₂—OR⁷. In some embodiments of Formula (I), (I′), (II), or (II′), R¹² is —SO₂R⁷; and R¹³ is substituted or unsubstituted CH₂—OR⁷. In some embodiments of Formula (I), (I′), (II), or (II′), R¹² is —SOR⁷; and R¹³ is substituted or unsubstituted CH₂—OR⁷. In some embodiments of Formula (I), (I′), (II), or (II′), R¹² is —SO₂NR⁷₂; and R¹³ is substituted or unsubstituted CH₂—OR⁷. In some embodiments of Formula (I), (I′), (II), or (II′), R¹² is —OSO₂R⁷; and R¹³ is substituted or unsubstituted CH₂—OR⁷. In some embodiments of Formula (I), (I′), (II), or (II′), R¹² is —OSOR⁷; and R¹³ is substituted or unsubstituted CH₂—OR⁷. In some embodiments of Formula (I), (I′), (II), or (II′), R¹² is —OSO₂NR⁷₂; and R¹³ is substituted or unsubstituted CH₂—OR⁷. In some embodiments of Formula (I), (I′), (II), or (II′), R¹² is —OSO₂OR⁷; and R¹³ is substituted or unsubstituted CH₂—OR⁷. In some embodiments of Formula (I), (I′), (II), or (II′), R¹² is —O(SO)OR⁷; and R¹³ is substituted or unsubstituted CH₂—OR⁷. In some embodiments of Formula (I), (I′), (II), or (II′), R¹² is —PO(OR⁷)₂; and R¹³ is substituted or unsubstituted CH₂—OR⁷. In some embodiments of Formula (I), (I′), (II), or (II′), R¹² is —PO(NR⁷)₂; and R¹³ is substituted or unsubstituted CH₂—OR⁷. In some embodiments of Formula (I), (I′), (II), or (II′), R¹² is —PO(NR⁷)(OR⁷); and R¹³ is substituted or unsubstituted CH₂—OR⁷. In some embodiments of Formula (I), (I′), (II), or (II′), R¹² is —OPO(OR⁷)₂; and R¹³ is substituted or unsubstituted CH₂—OR⁷. In some embodiments of Formula (I), (I′), (II), or (II′), R¹² is —OPO(NR⁷)₂; and R¹³ is substituted or unsubstituted CH₂—OR⁷. In some embodiments of Formula (I), (I′), (II), or (II′), R¹² is —OPO(NR⁷)(OR⁷); and R¹³ is substituted or unsubstituted CH₂—OR⁷. In some embodiments of Formula (I), (I′), (II), or (II′), R¹² is a nitrogen protecting group; and R¹³ is substituted or unsubstituted CH₂—OR⁷. In some embodiments of Formula (I), (I′), (II), or (II′), R¹² is —OR⁷; and R¹³ is substituted or unsubstituted CH₂—OR⁷.

In some embodiments of Formula (I), (I′), (II), or (II′), R¹² is hydrogen; and R¹³ is substituted or unsubstituted C₆₋₁₀ aryl. In some embodiments of Formula (I), (I′), (II), or (II′), R¹² is substituted or unsubstituted C₁₋₆ alkyl; and R¹³ is substituted or unsubstituted C₆₋₁₀ aryl. In some embodiments of Formula (I), (I′), (II), or (II′), R¹² is substituted or unsubstituted C₁₋₃ alkyl; and R¹³ is substituted or unsubstituted C₆₋₁₀ aryl. In some embodiments of Formula (I), (I′), (II), or (II′), R¹² is branched or unbranched substituted or unsubstituted C₁₋₆ alkyl; and R¹³ is substituted or unsubstituted C₆₋₁₀ aryl. In some embodiments of Formula (I), (I′), (II), or (II′), R¹² is substituted or unsubstituted methyl; and R¹³ is substituted or unsubstituted C₆₋₁₀ aryl. In some embodiments of Formula (I), (I′), (II), or (II′), R¹² is substituted or unsubstituted ethyl; and R¹³ is substituted or unsubstituted C₆₋₁₀ aryl. In some embodiments of Formula (I), (I′), (II), or (II′), R¹² is substituted or unsubstituted propyl; and R¹³ is substituted or unsubstituted C₆₋₁₀ aryl. In some embodiments of Formula (I), (I′), (II), or (II′), R¹² is substituted or unsubstituted butyl; and R¹³ is substituted or unsubstituted C₆₋₁₀ aryl. In some embodiments of Formula (I), (I′), (II), or (II′), R¹² is substituted or unsubstituted pentyl; and R¹³ is substituted or unsubstituted C₆₋₁₀ aryl. In some embodiments of Formula (I), (I′), (II), or (II′), R¹² is substituted or unsubstituted C₁₋₆ haloalkyl; and R¹³ is substituted or unsubstituted C₆₋₁₀ aryl. In some embodiments of Formula (I), (I′), (II), or (II′), R¹² is substituted or unsubstituted fluoroalkyl; and R¹³ is substituted or unsubstituted C₆₋₁₀ aryl. In some embodiments of Formula (I), (I′), (II), or (II′), R¹² is trifluoromethyl; and R¹³ is substituted or unsubstituted C₆₋₁₀ aryl. In some embodiments of Formula (I), (I′), (II), or (II′), R¹² is substituted or unsubstituted trifluoroethyl; and R¹³ is substituted or unsubstituted C₆₋₁₀ aryl. In some embodiments of Formula (I), (I′), (II), or (II′), R¹² is substituted or unsubstituted C₂₋₆ alkenyl; and R¹³ is substituted or unsubstituted C₆₋₁₀ aryl. In some embodiments of Formula (I), (I′), (II), or (II′), R¹² is substituted or unsubstituted C₂₋₆ alkenyl; and R¹³ is substituted or unsubstituted C₆₋₁₀ aryl. In some embodiments of Formula (I), (I′), (II), or (II′), R¹² is substituted or unsubstituted vinyl; and R¹³ is substituted or unsubstituted C₆₋₁₀ aryl. In some embodiments of Formula (I), (I′), (II), or (II′), R¹² is substituted or unsubstituted allyl; and R¹³ is substituted or unsubstituted C₆₋₁₀ aryl. In some embodiments of Formula (I), (I′), (II), or (II′), R¹² is substituted or unsubstituted propenyl; and R¹³ is substituted or unsubstituted C₆₋₁₀ aryl. In some embodiments of Formula (I), (I′), (II), or (II′), R¹² is substituted or unsubstituted butenyl; and R¹³ is substituted or unsubstituted C₆₋₁₀ aryl. In some embodiments of Formula (I), (I′), (II), or (II′), R¹² is substituted or unsubstituted C₂₋₆ alkynyl; and R¹³ is substituted or unsubstituted C₆₋₁₀ aryl. In some embodiments of Formula (I), (I′), (II), or (II′), R¹² is substituted or unsubstituted acetylenyl; and R¹³ is substituted or unsubstituted C₆₋₁₀ aryl. In some embodiments of Formula (I), (I′), (II), or (II′), R¹² is substituted or unsubstituted propargyl; and R¹³ is substituted or unsubstituted C₆₋₁₀ aryl. In some embodiments of Formula (I), (I′), (II), or (II′), R¹² is substituted or unsubstituted butynyl; and R¹³ is substituted or unsubstituted C₆₋₁₀ aryl. In some embodiments of Formula (I), (I′), (II), or (II′), R¹² is substituted or unsubstituted C₁₋₆ alkoxy; and R¹³ is substituted or unsubstituted C₆₋₁₀ aryl. In some embodiments of Formula (I), (I′), (II), or (II′), R¹² is CH₂—OMe; and R¹³ is substituted or unsubstituted C₆₋₁₀ aryl. In some embodiments of Formula (I), (I′), (II), or (II′), R¹² is CH₂-OEt; and R¹³ is substituted or unsubstituted C₆₋₁₀ aryl. In some embodiments of Formula (I), (I′), (II), or (II′), R¹²CH₂—OPr; and R¹³ is substituted or unsubstituted C₆₋₁₀ aryl. In some embodiments of Formula (I), (I′), (II), or (II′), R¹² is CH₂—OCF₃; and R¹³ is substituted or unsubstituted C₆₋₁₀ aryl. In some embodiments of Formula (I), (I′), (II), or (II′), R¹² is substituted or unsubstituted CH₂—OR⁷; and R¹³ is substituted or unsubstituted C₆₋₁₀ aryl. In some embodiments of Formula (I), (I′), (II), or (II′), R¹² is substituted or unsubstituted C₆₋₁₀ aryl; and R¹³ is substituted or unsubstituted C₆₋₁₀ aryl. In some embodiments of Formula (I), (I′), (II), or (II′), R¹² is substituted or unsubstituted 5-10 membered heteroaryl; and R¹³ is substituted or unsubstituted C₆₋₁₀ aryl. In some embodiments of Formula (I), (I′), (II), or (II′), R¹² is substituted or unsubstituted 4-10 membered heterocyclyl; and R¹³ is substituted or unsubstituted C₆₋₁₀ aryl. In some embodiments of Formula (I), (I′), (II), or (II′), R¹² is substituted or unsubstituted 3-10 membered carbocyclyl; and R¹³ is substituted or unsubstituted C₆₋₁₀ aryl. In some embodiments of Formula (I), (I′), (II), or (II′), R¹² is acyl; and R¹³ is substituted or unsubstituted C₆₋₁₀ aryl. In some embodiments of Formula (I), (I′), (II), or (II′), R¹² is a substituted or unsubstituted amide; and R¹³ is substituted or unsubstituted C₆₋₁₀ aryl. In some embodiments of Formula (I), (I′), (II), or (II′), R¹² is a substituted or unsubstituted ester; and R¹³ is substituted or unsubstituted C₆₋₁₀ aryl. In some embodiments of Formula (I), (I′), (II), or (II′), R¹² is —OH; and R¹³ is substituted or unsubstituted C₆₋₁₀ aryl. In some embodiments of Formula (I), (I′), (II), or (II′), R¹² is —SO₂R⁷; and R¹³ is substituted or unsubstituted C₆₋₁₀ aryl. In some embodiments of Formula (I), (I′), (II), or (II′), R¹² is —SOR⁷; and R¹³ is substituted or unsubstituted C₆₋₁₀ aryl. In some embodiments of Formula (I), (I′), (II), or (II′), R¹² is —SO₂NR⁷₂; and R¹³ is substituted or unsubstituted C₆₋₁₀ aryl. In some embodiments of Formula (I), (I′), (II), or (II′), R¹² is —OSO₂R⁷; and R¹³ is substituted or unsubstituted C₆₋₁₀ aryl. In some embodiments of Formula (I), (I′), (II), or (II′), R¹² is —OSOR⁷; and R¹³ is substituted or unsubstituted C₆₋₁₀ aryl. In some embodiments of Formula (I), (I′), (II), or (II′), R¹² is —OSO₂NR⁷₂; and R¹³ is substituted or unsubstituted C₆₋₁₀ aryl. In some embodiments of Formula (I), (I′), (II), or (II′), R¹² is —OSO₂OR⁷; and R¹³ is substituted or unsubstituted C₆₋₁₀ aryl. In some embodiments of Formula (I), (I′), (II), or (II′), R¹² is —O(SO)OR⁷; and R¹³ is substituted or unsubstituted C₆₋₁₀ aryl. In some embodiments of Formula (I), (I′), (II), or (II′), R¹² is —PO(OR⁷)₂; and R¹³ is substituted or unsubstituted C₆₋₁₀ aryl. In some embodiments of Formula (I), (I′), (II), or (II′), R¹² is —PO(NR⁷)₂; and R¹³ is substituted or unsubstituted C₆₋₁₀ aryl. In some embodiments of Formula (I), (I′), (II), or (II′), R¹² is —PO(NR⁷)(OR⁷); and R¹³ is substituted or unsubstituted C₆₋₁₀ aryl. In some embodiments of Formula (I), (I′), (II), or (II′), R¹² is —OPO(OR⁷)₂; and R¹³ is substituted or unsubstituted C₆₋₁₀ aryl. In some embodiments of Formula (I), (I′), (II), or (II′), R¹² is —OPO(NR⁷)₂; and R¹³ is substituted or unsubstituted C₆₋₁₀ aryl. In some embodiments of Formula (I), (I′), (II), or (II′), R¹² is —OPO(NR⁷)(OR⁷); and R¹³ is substituted or unsubstituted C₆₋₁₀ aryl. In some embodiments of Formula (I), (I′), (II), or (II′), R¹² is a nitrogen protecting group; and R³ is substituted or unsubstituted C₆₋₁₀ aryl. In some embodiments of Formula (I), (I′), (II), or (II′), R¹² is —OR⁷; and R¹³ is substituted or unsubstituted C₆₋₁₀ aryl.

In some embodiments of Formula (I), (I′), (II), or (II′), R¹² is hydrogen; and R¹³ is substituted or unsubstituted 5-10 membered heteroaryl. In some embodiments of Formula (I), (I′), (II), or (II′), R¹² is substituted or unsubstituted C₁₋₆ alkyl; and R³ is substituted or unsubstituted 5-10 membered heteroaryl. In some embodiments of Formula (I), (I′), (II), or (II′), R¹² is substituted or unsubstituted C₁₋₃ alkyl; and R¹³ is substituted or unsubstituted 5-10 membered heteroaryl. In some embodiments of Formula (I), (I′), (II), or (II′), R¹² is branched or unbranched substituted or unsubstituted C₁₋₆ alkyl; and R¹³ is substituted or unsubstituted 5-10 membered heteroaryl. In some embodiments of Formula (I), (I′), (II), or (II′), R¹² is substituted or unsubstituted methyl; and R¹³ is substituted or unsubstituted 5-10 membered heteroaryl. In some embodiments of Formula (I), (I′), (II), or (II′), R¹² is substituted or unsubstituted ethyl; and R¹³ is substituted or unsubstituted 5-10 membered heteroaryl. In some embodiments of Formula (I), (I′), (II), or (II′), R¹² is substituted or unsubstituted propyl; and R¹³ is substituted or unsubstituted 5-10 membered heteroaryl. In some embodiments of Formula (I), (I′), (II), or (II′), R¹² is substituted or unsubstituted butyl; and R¹³ is substituted or unsubstituted 5-10 membered heteroaryl. In some embodiments of Formula (I), (I′), (II), or (II′), R¹² is substituted or unsubstituted pentyl; and R¹³ is substituted or unsubstituted 5-10 membered heteroaryl. In some embodiments of Formula (I), (I′), (II), or (II′), R¹² is substituted or unsubstituted C₁₋₆ haloalkyl; and R¹³ is substituted or unsubstituted 5-10 membered heteroaryl. In some embodiments of Formula (I), (I′), (II), or (II′), R¹² is substituted or unsubstituted fluoroalkyl; and R¹³ is substituted or unsubstituted 5-10 membered heteroaryl. In some embodiments of Formula (I), (I′), (II), or (II′), R¹² is trifluoromethyl; and R¹³ is substituted or unsubstituted 5-10 membered heteroaryl. In some embodiments of Formula (I), (I′), (II), or (II′), R¹² is substituted or unsubstituted trifluoroethyl; and R¹³ is substituted or unsubstituted 5-10 membered heteroaryl. In some embodiments of Formula (I), (I′), (II), or (II′), R¹² is substituted or unsubstituted C₂₋₆ alkenyl; and R¹³ is substituted or unsubstituted 5-10 membered heteroaryl. In some embodiments of Formula (I), (I′), (II), or (II′), R¹² is substituted or unsubstituted C₂₋₆ alkenyl; and R¹³ is substituted or unsubstituted 5-10 membered heteroaryl. In some embodiments of Formula (I), (I′), (II), or (II′), R¹² is substituted or unsubstituted vinyl; and R¹³ is substituted or unsubstituted 5-10 membered heteroaryl. In some embodiments of Formula (I), (I′), (II), or (II′), R¹² is substituted or unsubstituted allyl; and R¹³ is substituted or unsubstituted 5-10 membered heteroaryl. In some embodiments of Formula (I), (I′), (II), or (II′), R¹² is substituted or unsubstituted propenyl; and R¹³ is substituted or unsubstituted 5-10 membered heteroaryl. In some embodiments of Formula (I), (I′), (II), or (II′), R¹² is substituted or unsubstituted butenyl; and R¹³ is substituted or unsubstituted 5-10 membered heteroaryl. In some embodiments of Formula (I), (I′), (II), or (II′), R¹² is substituted or unsubstituted C₂₋₆ alkynyl; and R¹³ is substituted or unsubstituted 5-10 membered heteroaryl. In some embodiments of Formula (I), (I′), (II), or (II′), R¹² is substituted or unsubstituted acetylenyl; and R¹³ is substituted or unsubstituted 5-10 membered heteroaryl. In some embodiments of Formula (I), (I′), (II), or (II′), R¹² is substituted or unsubstituted propargyl; and R¹³ is substituted or unsubstituted 5-10 membered heteroaryl. In some embodiments of Formula (I), (I′), (II), or (II′), R¹² is substituted or unsubstituted butynyl; and R¹³ is substituted or unsubstituted 5-10 membered heteroaryl. In some embodiments of Formula (I), (I′), (II), or (II′), R¹² is substituted or unsubstituted C₁₋₆ alkoxy; and R¹³ is substituted or unsubstituted 5-10 membered heteroaryl. In some embodiments of Formula (I), (I′), (II), or (II′), R¹² is CH₂—OMe; and R¹³ is substituted or unsubstituted 5-10 membered heteroaryl. In some embodiments of Formula (I), (I′), (II), or (II′), R¹² is CH₂—OEt; and R¹³ is substituted or unsubstituted 5-10 membered heteroaryl. In some embodiments of Formula (I), (I′), (II), or (II′), R¹²CH₂—OPr; and R¹³ is substituted or unsubstituted 5-10 membered heteroaryl. In some embodiments of Formula (I), (I′), (II), or (II′), R¹² is CH₂—OCF₃; and R¹³ is substituted or unsubstituted 5-10 membered heteroaryl. In some embodiments of Formula (I), (I′), (II), or (II′), R¹² is substituted or unsubstituted CH₂—OR⁷; and R¹³ is substituted or unsubstituted 5-10 membered heteroaryl. In some embodiments of Formula (I), (I′), (II), or (II′), R¹² is substituted or unsubstituted C₆₋₁₀ aryl; and R¹³ is substituted or unsubstituted 5-10 membered heteroaryl. In some embodiments of Formula (I), (I′), (II), or (II′), R¹² is substituted or unsubstituted 5-10 membered heteroaryl; and R¹³ is substituted or unsubstituted 5-10 membered heteroaryl. In some embodiments of Formula (I), (I′), (II), or (II′), R¹² is substituted or unsubstituted 4-10 membered heterocyclyl; and R¹³ is substituted or unsubstituted 5-10 membered heteroaryl. In some embodiments of Formula (I), (I′), (II), or (II′), R¹² is substituted or unsubstituted 3-10 membered carbocyclyl; and R¹³ is substituted or unsubstituted 5-10 membered heteroaryl. In some embodiments of Formula (I), (I′), (II), or (II′), R¹² is acyl; and R¹³ is substituted or unsubstituted 5-10 membered heteroaryl. In some embodiments of Formula (I), (I′), (II), or (II′), R¹² is a substituted or unsubstituted amide; and R¹³ is substituted or unsubstituted 5-10 membered heteroaryl. In some embodiments of Formula (I), (I′), (II), or (II′), R¹² is a substituted or unsubstituted ester; and R¹³ is substituted or unsubstituted 5-10 membered heteroaryl. In some embodiments of Formula (I), (I′), (II), or (II′), R¹² is —OH; and R¹³ is substituted or unsubstituted 5-10 membered heteroaryl. In some embodiments of Formula (I), (I′), (II), or (II′), R¹² is —SO₂R⁷; and R¹³ is substituted or unsubstituted 5-10 membered heteroaryl. In some embodiments of Formula (I), (I′), (II), or (II′), R¹² is —SOR⁷; and R¹³ is substituted or unsubstituted 5-10 membered heteroaryl. In some embodiments of Formula (I), (I′), (II), or (II′), R¹² is —SO₂NR⁷₂; and R¹³ is substituted or unsubstituted 5-10 membered heteroaryl. In some embodiments of Formula (I), (I′), (II), or (II′), R¹² is —OSO₂R⁷; and R¹³ is substituted or unsubstituted 5-10 membered heteroaryl. In some embodiments of Formula (I), (I′), (II), or (II′), R¹² is —OSOR⁷; and R¹³ is substituted or unsubstituted 5-10 membered heteroaryl. In some embodiments of Formula (I), (I′), (II), or (II′), R¹² is —OSO₂NR⁷₂; and R¹³ is substituted or unsubstituted 5-10 membered heteroaryl. In some embodiments of Formula (I), (I′), (II), or (II′), R¹² is —OSO₂OR⁷; and R¹³ is substituted or unsubstituted 5-10 membered heteroaryl. In some embodiments of Formula (I), (I′), (II), or (II′), R¹² is —O(SO)OR⁷; and R¹³ is substituted or unsubstituted 5-10 membered heteroaryl. In some embodiments of Formula (I), (I′), (II), or (II′), R¹² is —PO(OR⁷)₂; and R¹³ is substituted or unsubstituted 5-10 membered heteroaryl. In some embodiments of Formula (I), (I′), (II), or (II′), R¹² is —PO(NR⁷)₂; and R¹³ is substituted or unsubstituted 5-10 membered heteroaryl. In some embodiments of Formula (I), (I′), (II), or (II′), R¹² is —PO(NR⁷)(OR⁷); and R¹³ is substituted or unsubstituted 5-10 membered heteroaryl. In some embodiments of Formula (I), (I′), (II), or (II′), R¹² is —OPO(OR⁷)₂; and R¹³ is substituted or unsubstituted 5-10 membered heteroaryl. In some embodiments of Formula (I), (I′), (II), or (II′), R¹² is —OPO(NR⁷)₂; and R¹³ is substituted or unsubstituted 5-10 membered heteroaryl. In some embodiments of Formula (I), (I′), (II), or (II′), R¹² is —OPO(NR⁷)(OR⁷); and R¹³ is substituted or unsubstituted 5-10 membered heteroaryl. In some embodiments of Formula (I), (I′), (II), or (II′), R¹² is a nitrogen protecting group; and R¹³ is substituted or unsubstituted 5-10 membered heteroaryl. In some embodiments of Formula (I), (I′), (II), or (II′), R¹² is —OR⁷; and R¹³ is substituted or unsubstituted 5-10 membered heteroaryl.

In some embodiments of Formula (I), (I′), (II), or (II′), R¹² is hydrogen; and R¹³ is substituted or unsubstituted 4-10 membered heterocyclyl. In some embodiments of Formula (I), (I′), (II), or (II′), R¹² is substituted or unsubstituted C₁₋₆ alkyl; and R¹³ is substituted or unsubstituted 4-10 membered heterocyclyl. In some embodiments of Formula (I), (I′), (II), or (II′), R¹² is substituted or unsubstituted C₁₋₃ alkyl; and R¹³ is substituted or unsubstituted 4-10 membered heterocyclyl. In some embodiments of Formula (I), (I′), (II), or (II′), R¹² is branched or unbranched substituted or unsubstituted C₁₋₆ alkyl; and R¹³ is substituted or unsubstituted 4-10 membered heterocyclyl. In some embodiments of Formula (I), (I′), (II), or (II′), R¹² is substituted or unsubstituted methyl; and R¹³ is substituted or unsubstituted 4-10 membered heterocyclyl. In some embodiments of Formula (I), (I′), (II), or (II′), R¹² is substituted or unsubstituted ethyl; and R¹³ is substituted or unsubstituted 4-10 membered heterocyclyl. In some embodiments of Formula (I), (I′), (II), or (II′), R¹² is substituted or unsubstituted propyl; and R¹³ is substituted or unsubstituted 4-10 membered heterocyclyl. In some embodiments of Formula (I), (I′), (II), or (II′), R¹² is substituted or unsubstituted butyl; and R¹³ is substituted or unsubstituted 4-10 membered heterocyclyl. In some embodiments of Formula (I), (I′), (II), or (II′), R¹² is substituted or unsubstituted pentyl; and R¹³ is substituted or unsubstituted 4-10 membered heterocyclyl. In some embodiments of Formula (I), (I′), (II), or (II′), R¹² is substituted or unsubstituted C₁₋₆ haloalkyl; and R¹³ is substituted or unsubstituted 4-10 membered heterocyclyl. In some embodiments of Formula (I), (I′), (II), or (II′), R¹² is substituted or unsubstituted fluoroalkyl; and R¹³ is substituted or unsubstituted 4-10 membered heterocyclyl. In some embodiments of Formula (I), (I′), (II), or (II′), R¹² is trifluoromethyl; and R¹³ is substituted or unsubstituted 4-10 membered heterocyclyl. In some embodiments of Formula (I), (I′), (II), or (II′), R¹² is substituted or unsubstituted trifluoroethyl; and R¹³ is substituted or unsubstituted 4-10 membered heterocyclyl. In some embodiments of Formula (I), (I′), (II), or (II′), R¹² is substituted or unsubstituted C₂₋₆ alkenyl; and R¹³ is substituted or unsubstituted 4-10 membered heterocyclyl. In some embodiments of Formula (I), (I′), (II), or (II′), R¹² is substituted or unsubstituted C₂₋₆ alkenyl; and R¹³ is substituted or unsubstituted 4-10 membered heterocyclyl. In some embodiments of Formula (I), (I′), (II), or (II′), R¹² is substituted or unsubstituted vinyl; and R¹³ is substituted or unsubstituted 4-10 membered heterocyclyl. In some embodiments of Formula (I), (I′), (II), or (II′), R¹² is substituted or unsubstituted allyl; and R¹³ is substituted or unsubstituted 4-10 membered heterocyclyl. In some embodiments of Formula (I), (I′), (II), or (II′), R¹² is substituted or unsubstituted propenyl; and R¹³ is substituted or unsubstituted 4-10 membered heterocyclyl. In some embodiments of Formula (I), (I′), (II), or (II′), R¹² is substituted or unsubstituted butenyl; and R¹³ is substituted or unsubstituted 4-10 membered heterocyclyl. In some embodiments of Formula (I), (I′), (II), or (II′), R¹² is substituted or unsubstituted C₂₋₆ alkynyl; and R¹³ is substituted or unsubstituted 4-10 membered heterocyclyl. In some embodiments of Formula (I), (I′), (II), or (II′), R¹² is substituted or unsubstituted acetylenyl; and R¹³ is substituted or unsubstituted 4-10 membered heterocyclyl. In some embodiments of Formula (I), (I′), (II), or (II′), R¹² is substituted or unsubstituted propargyl; and R¹³ is substituted or unsubstituted 4-10 membered heterocyclyl. In some embodiments of Formula (I), (I′), (II), or (II′), R¹² is substituted or unsubstituted butynyl; and R¹³ is substituted or unsubstituted 4-10 membered heterocyclyl.

In some embodiments of Formula (I), (I′), (II), or (II′), R¹² is substituted or unsubstituted C₁₋₆ alkoxy; and R¹³ is substituted or unsubstituted 4-10 membered heterocyclyl. In some embodiments of Formula (I), (I′), (II), or (II′), R¹² is CH₂—OMe; and R¹³ is substituted or unsubstituted 4-10 membered heterocyclyl. In some embodiments of Formula (I), (I′), (II), or (II′), R¹² is CH₂—OEt; and R¹³ is substituted or unsubstituted 4-10 membered heterocyclyl. In some embodiments of Formula (I), (I′), (II), or (II′), R¹²CH₂—OPr; and R¹³ is substituted or unsubstituted 4-10 membered heterocyclyl. In some embodiments of Formula (I), (I′), (II), or (II′), R¹² is CH₂—OCF₃; and R¹³ is substituted or unsubstituted 4-10 membered heterocyclyl. In some embodiments of Formula (I), (I′), (II), or (II′), R¹² is substituted or unsubstituted CH₂—OR⁷; and R¹³ is substituted or unsubstituted 4-10 membered heterocyclyl. In some embodiments of Formula (I), (I′), (II), or (II′), R¹² is substituted or unsubstituted C₆₋₁₀ aryl; and R¹³ is substituted or unsubstituted 4-10 membered heterocyclyl. In some embodiments of Formula (I), (I′), (II), or (II′), R¹² is substituted or unsubstituted 5-10 membered heteroaryl; and R¹³ is substituted or unsubstituted 4-10 membered heterocyclyl. In some embodiments of Formula (I), (I′), (II), or (II′), R¹² is substituted or unsubstituted 4-10 membered heterocyclyl; and R¹³ is substituted or unsubstituted 4-10 membered heterocyclyl. In some embodiments of Formula (I), (I′), (II), or (II′), R¹² is substituted or unsubstituted 3-10 membered carbocyclyl; and R¹³ is substituted or unsubstituted 4-10 membered heterocyclyl. In some embodiments of Formula (I), (I′), (II), or (II′), R¹² is acyl; and R¹³ is substituted or unsubstituted 4-10 membered heterocyclyl. In some embodiments of Formula (I), (I′), (II), or (II′), R¹² is a substituted or unsubstituted amide; and R¹³ is substituted or unsubstituted 4-10 membered heterocyclyl. In some embodiments of Formula (I), (I′), (II), or (II′), R¹² is a substituted or unsubstituted ester; and R¹³ is substituted or unsubstituted 4-10 membered heterocyclyl. In some embodiments of Formula (I), (I′), (II), or (II′), R¹² is —OH; and R¹³ is substituted or unsubstituted 4-10 membered heterocyclyl. In some embodiments of Formula (I), (I′), (II), or (II′), R¹² is —SO₂R⁷; and R¹³ is substituted or unsubstituted 4-10 membered heterocyclyl. In some embodiments of Formula (I), (I′), (II), or (II′), R¹² is —SOR⁷; and R¹³ is substituted or unsubstituted 4-10 membered heterocyclyl. In some embodiments of Formula (I), (I′), (II), or (II′), R¹² is —SO₂NR⁷₂; and R¹³ is substituted or unsubstituted 4-10 membered heterocyclyl. In some embodiments of Formula (I), (I′), (II), or (II′), R¹² is —OSO₂R⁷; and R¹³ is substituted or unsubstituted 4-10 membered heterocyclyl. In some embodiments of Formula (I), (I′), (II), or (II′), R¹² is —OSOR⁷; and R¹³ is substituted or unsubstituted 4-10 membered heterocyclyl. In some embodiments of Formula (I), (I′), (II), or (II′), R¹² is —OSO₂NR⁷₂; and R¹³ is substituted or unsubstituted 4-10 membered heterocyclyl. In some embodiments of Formula (I), (I′), (II), or (II′), R¹² is —OSO₂OR⁷; and R¹³ is substituted or unsubstituted 4-10 membered heterocyclyl. In some embodiments of Formula (I), (I′), (II), or (II′), R¹² is —O(SO)OR⁷; and R¹³ is substituted or unsubstituted 4-10 membered heterocyclyl. In some embodiments of Formula (I), (I′), (II), or (II′), R¹² is —PO(OR⁷)₂; and R¹³ is substituted or unsubstituted 4-10 membered heterocyclyl. In some embodiments of Formula (I), (I′), (II), or (II′), R¹² is —PO(NR⁷)₂; and R¹³ is substituted or unsubstituted 4-10 membered heterocyclyl. In some embodiments of Formula (I), (I′), (II), or (II′), R¹² is —PO(NR⁷)(OR⁷); and R¹³ is substituted or unsubstituted 4-10 membered heterocyclyl. In some embodiments of Formula (I), (I′), (II), or (II′), R¹² is —OPO(OR⁷)₂; and R¹³ is substituted or unsubstituted 4-10 membered heterocyclyl. In some embodiments of Formula (I), (I′), (II), or (II′), R¹² is —OPO(NR⁷)₂; and R¹³ is substituted or unsubstituted 4-10 membered heterocyclyl. In some embodiments of Formula (I), (I′), (II), or (II′), R¹² is —OPO(NR⁷)(OR⁷); and R¹³ is substituted or unsubstituted 4-10 membered heterocyclyl. In some embodiments of Formula (I), (I′), (II), or (II′), R¹² is a nitrogen protecting group; and R¹³ is substituted or unsubstituted 4-10 membered heterocyclyl. In some embodiments of Formula (I), (I′), (II), or (II′), R¹² is —OR⁷; and R¹³ is substituted or unsubstituted 4-10 membered heterocyclyl.

In some embodiments of Formula (I), (I′), (II), or (II′), R¹² is hydrogen; and R¹³ is substituted or unsubstituted 3-10 membered carbocyclyl. In some embodiments of Formula (I), (I′), (II), or (II′), R¹² is substituted or unsubstituted C₁₋₆ alkyl; and R¹³ is substituted or unsubstituted 3-10 membered carbocyclyl. In some embodiments of Formula (I), (I′), (II), or (II′), R¹² is substituted or unsubstituted C₁₋₃ alkyl; and R¹³ is substituted or unsubstituted 3-10 membered carbocyclyl. In some embodiments of Formula (I), (I′), (II), or (II′), R¹² is branched or unbranched substituted or unsubstituted C₁₋₆ alkyl; and R¹³ is substituted or unsubstituted 3-10 membered carbocyclyl. In some embodiments of Formula (I), (I′), (II), or (II′), R¹² is substituted or unsubstituted methyl; and R¹³ is substituted or unsubstituted 3-10 membered carbocyclyl. In some embodiments of Formula (I), (I′), (II), or (II′), R¹² is substituted or unsubstituted ethyl; and R¹³ is substituted or unsubstituted 3-10 membered carbocyclyl. In some embodiments of Formula (I), (I′), (II), or (II′), R¹² is substituted or unsubstituted propyl; and R¹³ is substituted or unsubstituted 3-10 membered carbocyclyl. In some embodiments of Formula (I), (I′), (II), or (II′), R¹² is substituted or unsubstituted butyl; and R¹³ is substituted or unsubstituted 3-10 membered carbocyclyl. In some embodiments of Formula (I), (I′), (II), or (II′), R¹² is substituted or unsubstituted pentyl; and R¹³ is substituted or unsubstituted 3-10 membered carbocyclyl. In some embodiments of Formula (I), (I′), (II), or (II′), R¹² is substituted or unsubstituted C₁₋₆ haloalkyl; and R¹³ is substituted or unsubstituted 3-10 membered carbocyclyl. In some embodiments of Formula (I), (I′), (II), or (II′), R¹² is substituted or unsubstituted fluoroalkyl; and R¹³ is substituted or unsubstituted 3-10 membered carbocyclyl. In some embodiments of Formula (I), (I′), (II), or (II′), R¹² is trifluoromethyl; and R¹³ is substituted or unsubstituted 3-10 membered carbocyclyl. In some embodiments of Formula (I), (I′), (II), or (II′), R¹² is substituted or unsubstituted trifluoroethyl; and R¹³ is substituted or unsubstituted 3-10 membered carbocyclyl. In some embodiments of Formula (I), (I′), (II), or (II′), R¹² is substituted or unsubstituted C₂₋₆ alkenyl; and R¹³ is substituted or unsubstituted 3-10 membered carbocyclyl. In some embodiments of Formula (I), (I′), (II), or (II′), R¹² is substituted or unsubstituted C₂₋₆ alkenyl; and R¹³ is substituted or unsubstituted 3-10 membered carbocyclyl. In some embodiments of Formula (I), (I′), (II), or (II′), R¹² is substituted or unsubstituted vinyl; and R¹³ is substituted or unsubstituted 3-10 membered carbocyclyl. In some embodiments of Formula (I), (I′), (II), or (II′), R¹² is substituted or unsubstituted allyl; and R¹³ is substituted or unsubstituted 3-10 membered carbocyclyl. In some embodiments of Formula (I), (I′), (II), or (II′), R¹² is substituted or unsubstituted propenyl; and R¹³ is substituted or unsubstituted 3-10 membered carbocyclyl. In some embodiments of Formula (I), (I′), (II), or (II′), R¹² is substituted or unsubstituted butenyl; and R¹³ is substituted or unsubstituted 3-10 membered carbocyclyl. In some embodiments of Formula (I), (I′), (II), or (II′), R¹² is substituted or unsubstituted C₂₋₆ alkynyl; and R¹³ is substituted or unsubstituted 3-10 membered carbocyclyl. In some embodiments of Formula (I), (I′), (II), or (II′), R¹² is substituted or unsubstituted acetylenyl; and R¹³ is substituted or unsubstituted 3-10 membered carbocyclyl. In some embodiments of Formula (I), (I′), (II), or (II′), R¹² is substituted or unsubstituted propargyl; and R¹³ is substituted or unsubstituted 3-10 membered carbocyclyl. In some embodiments of Formula (I), (I′), (II), or (II′), R¹² is substituted or unsubstituted butynyl; and R¹³ is substituted or unsubstituted 3-10 membered carbocyclyl. In some embodiments of Formula (I), (I′), (II), or (II′), R¹² is substituted or unsubstituted C₁₋₆ alkoxy; and R¹³ is substituted or unsubstituted 3-10 membered carbocyclyl. In some embodiments of Formula (I), (I′), (II), or (II′), R¹² is CH₂—OMe; and R¹³ is substituted or unsubstituted 3-10 membered carbocyclyl. In some embodiments of Formula (I), (I′), (II), or (II′), R¹² is CH₂-OEt; and R¹³ is substituted or unsubstituted 3-10 membered carbocyclyl. In some embodiments of Formula (I), (I′), (II), or (II′), R¹²CH₂—OPr; and R¹³ is substituted or unsubstituted 3-10 membered carbocyclyl. In some embodiments of Formula (I), (I′), (II), or (II′), R¹² is CH₂—OCF₃; and R¹³ is substituted or unsubstituted 3-10 membered carbocyclyl. In some embodiments of Formula (I), (I′), (II), or (II′), R¹² is substituted or unsubstituted CH₂—OR⁷; and R¹³ is substituted or unsubstituted 3-10 membered carbocyclyl. In some embodiments of Formula (I), (I′), (II), or (II′), R¹² is substituted or unsubstituted C₆₋₁₀ aryl; and R¹³ is substituted or unsubstituted 3-10 membered carbocyclyl. In some embodiments of Formula (I), (I′), (II), or (II′), R¹² is substituted or unsubstituted 5-10 membered heteroaryl; and R¹³ is substituted or unsubstituted 3-10 membered carbocyclyl. In some embodiments of Formula (I), (I′), (II), or (II′), R¹² is substituted or unsubstituted 4-10 membered heterocyclyl; and R¹³ is substituted or unsubstituted 3-10 membered carbocyclyl. In some embodiments of Formula (I), (I′), (II), or (II′), R¹² is substituted or unsubstituted 3-10 membered carbocyclyl; and R¹³ is substituted or unsubstituted 3-10 membered carbocyclyl. In some embodiments of Formula (I), (I′), (II), or (II′), R¹² is acyl; and R¹³ is substituted or unsubstituted 3-10 membered carbocyclyl. In some embodiments of Formula (I), (I′), (II), or (II′), R¹² is a substituted or unsubstituted amide; and R¹³ is substituted or unsubstituted 3-10 membered carbocyclyl. In some embodiments of Formula (I), (I′), (II), or (II′), R¹² is a substituted or unsubstituted ester; and R¹³ is substituted or unsubstituted 3-10 membered carbocyclyl. In some embodiments of Formula (I), (I′), (II), or (II′), R¹² is —OH; and R¹³ is substituted or unsubstituted 3-10 membered carbocyclyl. In some embodiments of Formula (I), (I′), (II), or (II′), R¹² is —SO₂R⁷; and R¹³ is substituted or unsubstituted 3-10 membered carbocyclyl. In some embodiments of Formula (I), (I′), (II), or (II′), R¹² is —SOR⁷; and R¹³ is substituted or unsubstituted 3-10 membered carbocyclyl. In some embodiments of Formula (I), (I′), (II), or (II′), R¹² is —SO₂NR⁷₂; and R¹³ is substituted or unsubstituted 3-10 membered carbocyclyl. In some embodiments of Formula (I), (I′), (II), or (II′), R¹² is —OSO₂R⁷; and R¹³ is substituted or unsubstituted 3-10 membered carbocyclyl. In some embodiments of Formula (I), (I′), (II), or (II′), R¹² is —OSOR⁷; and R¹³ is substituted or unsubstituted 3-10 membered carbocyclyl. In some embodiments of Formula (I), (I′), (II), or (II′), R¹² is —OSO₂NR⁷₂; and R¹³ is substituted or unsubstituted 3-10 membered carbocyclyl. In some embodiments of Formula (I), (I′), (II), or (II′), R¹² is —OSO₂OR⁷; and R¹³ is substituted or unsubstituted 3-10 membered carbocyclyl. In some embodiments of Formula (I), (I′), (II), or (II′), R¹² is —O(SO)OR⁷; and R¹³ is substituted or unsubstituted 3-10 membered carbocyclyl. In some embodiments of Formula (I), (I′), (II), or (II′), R¹² is —PO(OR⁷)₂; and R¹³ is substituted or unsubstituted 3-10 membered carbocyclyl. In some embodiments of Formula (I), (I′), (II), or (II′), R¹² is —PO(NR⁷)₂; and R¹³ is substituted or unsubstituted 3-10 membered carbocyclyl. In some embodiments of Formula (I), (I′), (II), or (II′), R¹² is —PO(NR⁷)(OR⁷); and R¹³ is substituted or unsubstituted 3-10 membered carbocyclyl. In some embodiments of Formula (I), (I′), (II), or (II′), R¹² is —OPO(OR⁷)₂; and R¹³ is substituted or unsubstituted 3-10 membered carbocyclyl. In some embodiments of Formula (I), (I′), (II), or (II′), R¹² is —OPO(NR⁷)₂; and R¹³ is substituted or unsubstituted 3-10 membered carbocyclyl. In some embodiments of Formula (I), (I′), (II), or (II′), R¹² is —OPO(NR⁷)(OR⁷); and R¹³ is substituted or unsubstituted 3-10 membered carbocyclyl. In some embodiments of Formula (I), (I′), (II), or (II′), R¹² is a nitrogen protecting group; and R¹³ is substituted or unsubstituted 3-10 membered carbocyclyl. In some embodiments of Formula (I), (I′), (II), or (II′), R¹² is —OR⁷; and R¹³ is substituted or unsubstituted 3-10 membered carbocyclyl.

In some embodiments of Formula (I), (I′), (II), or (II′), R¹² is hydrogen; and R¹³ is acyl. In some embodiments of Formula (I), (I′), (II), or (II′), R¹² is substituted or unsubstituted C₁₋₆ alkyl; and R¹³ is acyl. In some embodiments of Formula (I), (I′), (II), or (II′), R¹² is substituted or unsubstituted C₁₋₃ alkyl; and R¹³ is acyl. In some embodiments of Formula (I), (I′), (II), or (II′), R¹² is branched or unbranched substituted or unsubstituted C₁₋₆ alkyl; and R¹³ is acyl. In some embodiments of Formula (I), (I′), (II), or (II′), R¹² is substituted or unsubstituted methyl; and R¹³ is acyl. In some embodiments of Formula (I), (I′), (II), or (II′), R¹² is substituted or unsubstituted ethyl; and R¹³ is acyl. In some embodiments of Formula (I), (I′), (II), or (II′), R¹² is substituted or unsubstituted propyl; and R¹³ is acyl. In some embodiments of Formula (I), (I′), (II), or (II′), R¹² is substituted or unsubstituted butyl; and R¹³ is acyl. In some embodiments of Formula (I), (I′), (II), or (II′), R¹² is substituted or unsubstituted pentyl; and R¹³ is acyl. In some embodiments of Formula (I), (I′), (II), or (II′), R¹² is substituted or unsubstituted C₁₋₆ haloalkyl; and R¹³ is acyl. In some embodiments of Formula (I), (I′), (II), or (II′), R¹² is substituted or unsubstituted fluoroalkyl; and R¹³ is acyl. In some embodiments of Formula (I), (I′), (II), or (II′), R¹² is trifluoromethyl; and R¹³ is acyl. In some embodiments of Formula (I), (I′), (II), or (II′), R¹² is substituted or unsubstituted trifluoroethyl; and R¹³ is acyl. In some embodiments of Formula (I), (I′), (II), or (II′), R¹² is substituted or unsubstituted C₂₋₆ alkenyl; and R¹³ is acyl. In some embodiments of Formula (I), (I′), (II), or (II′), R¹² is substituted or unsubstituted C₂₋₆ alkenyl; and R¹³ is acyl. In some embodiments of Formula (I), (I′), (II), or (II′), R¹² is substituted or unsubstituted vinyl; and R¹³ is acyl. In some embodiments of Formula (I), (I′), (II), or (II′), R¹² is substituted or unsubstituted allyl; and R¹³ is acyl. In some embodiments of Formula (I), (I′), (II), or (II′), R¹² is substituted or unsubstituted propenyl; and R¹³ is acyl. In some embodiments of Formula (I), (I′), (II), or (II′), R¹² is substituted or unsubstituted butenyl; and R³ is acyl. In some embodiments of Formula (I), (I′), (II), or (II′), R¹² is substituted or unsubstituted C₂₋₆ alkynyl; and R¹³ is acyl. In some embodiments of Formula (I), (I′), (II), or (II′), R¹² is substituted or unsubstituted acetylenyl; and R¹³ is acyl. In some embodiments of Formula (I), (I′), (II), or (II′), R¹² is substituted or unsubstituted propargyl; and R¹³ is acyl. In some embodiments of Formula (I), (I′), (II), or (II′), R¹² is substituted or unsubstituted butynyl; and R¹³ is acyl. In some embodiments of Formula (I), (I′), (II), or (II′), R¹² is substituted or unsubstituted C₁₋₆ alkoxy; and R¹³ is acyl. In some embodiments of Formula (I), (I′), (II), or (II′), R¹² is CH₂—OMe; and R¹³ is acyl. In some embodiments of Formula (I), (I′), (II), or (II′), R¹² is CH₂—OEt; and R¹³ is acyl. In some embodiments of Formula (I), (I′), (II), or (II′), R¹²CH₂—OPr; and R¹³ is acyl. In some embodiments of Formula (I), (I′), (II), or (II′), R¹² is CH₂—OCF₃; and R¹³ is acyl. In some embodiments of Formula (I), (I′), (II), or (II′), R¹² is substituted or unsubstituted CH₂—OR⁷; and R¹³ is acyl. In some embodiments of Formula (I), (I′), (II), or (II′), R¹² is substituted or unsubstituted C₆₋₁₀ aryl; and R¹³ is acyl. In some embodiments of Formula (I), (I′), (II), or (II′), R¹² is substituted or unsubstituted 5-10 membered heteroaryl; and R¹³ is acyl. In some embodiments of Formula (I), (I′), (II), or (II′), R¹² is substituted or unsubstituted 4-10 membered heterocyclyl; and R¹³ is acyl. In some embodiments of Formula (I), (I′), (II), or (II′), R¹² is substituted or unsubstituted 3-10 membered carbocyclyl; and R¹³ is acyl. In some embodiments of Formula (I), (I′), (II), or (II′), R¹² is acyl; and R¹³ is acyl. In some embodiments of Formula (I), (I′), (II), or (II′), R¹² is a substituted or unsubstituted amide; and R¹³ is acyl. In some embodiments of Formula (I), (I′), (II), or (II′), R¹² is a substituted or unsubstituted ester; and R¹³ is acyl. In some embodiments of Formula (I), (I′), (II), or (II′), R¹² is —OH; and R¹³ is acyl. In some embodiments of Formula (I), (I′), (II), or (II′), R¹² is —SO₂R⁷; and R¹³ is acyl. In some embodiments of Formula (I), (I′), (II), or (II′), R¹² is —SOR⁷; and R¹³ is acyl. In some embodiments of Formula (I), (I′), (II), or (II′), R¹² is —SO₂NR⁷₂; and R¹³ is acyl. In some embodiments of Formula (I), (I′), (II), or (II′), R¹² is —OS₂R⁷; and R¹³ is acyl. In some embodiments of Formula (I), (I′), (II), or (II′), R¹² is —OSOR⁷; and R¹³ is acyl. In some embodiments of Formula (I), (I′), (II), or (II′), R¹² is —OS₂NR⁷₂; and R¹³ is acyl. In some embodiments of Formula (I), (I′), (II), or (II′), R¹² is —OSO₂OR⁷; and R¹³ is acyl. In some embodiments of Formula (I), (I′), (II), or (II′), R¹² is —O(SO)OR⁷; and R¹³ is acyl. In some embodiments of Formula (I), (I′), (II), or (II′), R¹² is —PO(OR⁷)₂; and R¹³ is acyl. In some embodiments of Formula (I), (I′), (II), or (II′), R¹² is —PO(NR⁷)₂; and R¹³ is acyl. In some embodiments of Formula (I), (I′), (II), or (II′), R¹² is —PO(NR⁷)(OR⁷); and R¹³ is acyl. In some embodiments of Formula (I), (I′), (II), or (II′), R¹² is —OPO(OR⁷)₂; and R¹³ is acyl. In some embodiments of Formula (I), (I′), (II), or (II′), R¹² is —OPO(NR⁷)₂; and R¹³ is acyl. In some embodiments of Formula (I), (I′), (II), or (II′), R¹² is —OPO(NR⁷)(OR⁷); and R¹³ is acyl. In some embodiments of Formula (I), (I′), (II), or (II′), R¹² is a nitrogen protecting group; and R¹³ is acyl. In some embodiments of Formula (I), (I′), (II), or (II′), R¹² is —OR⁷; and R¹³ is acyl.

In some embodiments of Formula (I), (I′), (II), or (II′), R¹² is hydrogen; and R¹³ is a substituted or unsubstituted amide. In some embodiments of Formula (I), (I′), (II), or (II′), R¹² is substituted or unsubstituted C₁₋₆ alkyl; and R¹³ is a substituted or unsubstituted amide. In some embodiments of Formula (I), (I′), (II), or (II′), R¹² is substituted or unsubstituted C₁₋₃ alkyl; and R¹³ is a substituted or unsubstituted amide. In some embodiments of Formula (I), (I′), (II), or (II′), R¹² is branched or unbranched substituted or unsubstituted C₁₋₆ alkyl; and R¹³ is a substituted or unsubstituted amide. In some embodiments of Formula (I), (I′), (II), or (II′), R¹² is substituted or unsubstituted methyl; and R³ is a substituted or unsubstituted amide. In some embodiments of Formula (I), (I′), (II), or (II′), R¹² is substituted or unsubstituted ethyl; and R¹³ is a substituted or unsubstituted amide. In some embodiments of Formula (I), (I′), (II), or (II′), R¹² is substituted or unsubstituted propyl; and R³ is a substituted or unsubstituted amide. In some embodiments of Formula (I), (I′), (II), or (II′), R¹² is substituted or unsubstituted butyl; and R¹³ is a substituted or unsubstituted amide. In some embodiments of Formula (I), (I′), (II), or (II′), R¹² is substituted or unsubstituted pentyl; and R¹³ is a substituted or unsubstituted amide. In some embodiments of Formula (I), (I′), (II), or (II′), R¹² is substituted or unsubstituted C₁₋₆ haloalkyl; and R¹³ is a substituted or unsubstituted amide. In some embodiments of Formula (I), (I′), (II), or (II′), R¹² is substituted or unsubstituted fluoroalkyl; and R¹³ is a substituted or unsubstituted amide. In some embodiments of Formula (I), (I′), (II), or (II′), R¹² is trifluoromethyl; and R¹³ is a substituted or unsubstituted amide. In some embodiments of Formula (I), (I′), (II), or (II′), R¹² is substituted or unsubstituted trifluoroethyl; and R¹³ is a substituted or unsubstituted amide. In some embodiments of Formula (I), (I′), (II), or (II′), R¹² is substituted or unsubstituted C₂₋₆ alkenyl; and R¹³ is a substituted or unsubstituted amide. In some embodiments of Formula (I), (I′), (II), or (II′), R¹² is substituted or unsubstituted C₂₋₆ alkenyl; and R¹³ is a substituted or unsubstituted amide. In some embodiments of Formula (I), (I′), (II), or (II′), R¹² is substituted or unsubstituted vinyl; and R¹³ is a substituted or unsubstituted amide. In some embodiments of Formula (I), (I′), (II), or (II′), R¹² is substituted or unsubstituted allyl; and R¹³ is a substituted or unsubstituted amide. In some embodiments of Formula (I), (I′), (II), or (II′), R¹² is substituted or unsubstituted propenyl; and R¹³ is a substituted or unsubstituted amide. In some embodiments of Formula (I), (I′), (II), or (II′), R¹² is substituted or unsubstituted butenyl; and R¹³ is a substituted or unsubstituted amide. In some embodiments of Formula (I), (I′), (II), or (II′), R¹² is substituted or unsubstituted C₂₋₆ alkynyl; and R¹³ is a substituted or unsubstituted amide. In some embodiments of Formula (I), (I′), (II), or (II′), R¹² is substituted or unsubstituted acetylenyl; and R¹³ is a substituted or unsubstituted amide. In some embodiments of Formula (I), (I′), (II), or (II′), R¹² is substituted or unsubstituted propargyl; and R¹³ is a substituted or unsubstituted amide. In some embodiments of Formula (I), (I′), (II), or (II′), R¹² is substituted or unsubstituted butynyl; and R¹³ is a substituted or unsubstituted amide. In some embodiments of Formula (I), (I′), (II), or (II′), R¹² is substituted or unsubstituted C₁₋₆ alkoxy; and R¹³ is a substituted or unsubstituted amide. In some embodiments of Formula (I), (I′), (II), or (II′), R¹² is CH₂—OMe; and R¹³ is a substituted or unsubstituted amide. In some embodiments of Formula (I), (I′), (II), or (II′), R¹² is CH₂-OEt; and R¹³ is a substituted or unsubstituted amide. In some embodiments of Formula (I), (I′), (II), or (II′), R¹²CH₂—OPr; and R¹³ is a substituted or unsubstituted amide. In some embodiments of Formula (I), (I′), (II), or (II′), R¹² is CH₂—OCF₃; and R¹³ is a substituted or unsubstituted amide. In some embodiments of Formula (I), (I′), (II), or (II′), R¹² is substituted or unsubstituted CH₂—OR⁷; and R¹³ is a substituted or unsubstituted amide. In some embodiments of Formula (I), (I′), (II), or (II′), R¹² is substituted or unsubstituted C₆₋₁₀ aryl; and R¹³ is a substituted or unsubstituted amide. In some embodiments of Formula (I), (I′), (II), or (II′), R¹² is substituted or unsubstituted 5-10 membered heteroaryl; and R¹³ is a substituted or unsubstituted amide. In some embodiments of Formula (I), (I′), (II), or (II′), R¹² is substituted or unsubstituted 4-10 membered heterocyclyl; and R¹³ is a substituted or unsubstituted amide. In some embodiments of Formula (I), (I′), (II), or (II′), R¹² is substituted or unsubstituted 3-10 membered carbocyclyl; and R¹³ is a substituted or unsubstituted amide. In some embodiments of Formula (I), (I′), (II), or (II′), R¹² is acyl; and R¹³ is a substituted or unsubstituted amide. In some embodiments of Formula (I), (I′), (II), or (II′), R¹² is a substituted or unsubstituted amide; and R¹³ is a substituted or unsubstituted amide. In some embodiments of Formula (I), (I′), (II), or (II′), R¹² is a substituted or unsubstituted ester; and R¹³ is a substituted or unsubstituted amide. In some embodiments of Formula (I), (I′), (II), or (II′), R¹² is —OH; and R¹³ is a substituted or unsubstituted amide. In some embodiments of Formula (I), (I′), (II), or (II′), R¹² is —SO₂R⁷; and R¹³ is a substituted or unsubstituted amide. In some embodiments of Formula (I), (I′), (II), or (II′), R¹² is —SOR⁷; and R¹³ is a substituted or unsubstituted amide. In some embodiments of Formula (I), (I′), (II), or (II′), R¹² is —SO₂NR⁷₂; and R¹³ is a substituted or unsubstituted amide. In some embodiments of Formula (I), (I′), (II), or (II′), R¹² is —OSO₂R⁷; and R¹³ is a substituted or unsubstituted amide. In some embodiments of Formula (I), (I′), (II), or (II′), R¹² is —OSOR⁷; and R¹³ is a substituted or unsubstituted amide. In some embodiments of Formula (I), (I′), (II), or (II′), R¹² is —OSO₂NR⁷₂; and R¹³ is a substituted or unsubstituted amide. In some embodiments of Formula (I), (I′), (II), or (II′), R¹² is —OSO₂OR⁷; and R¹³ is a substituted or unsubstituted amide. In some embodiments of Formula (I), (I′), (II), or (II′), R¹² is —O(SO)OR⁷; and R¹³ is a substituted or unsubstituted amide. In some embodiments of Formula (I), (I′), (II), or (II′), R¹² is —PO(OR⁷)₂; and R¹³ is a substituted or unsubstituted amide. In some embodiments of Formula (I), (I′), (II), or (II′), R¹² is —PO(NR⁷)₂; and R¹³ is a substituted or unsubstituted amide. In some embodiments of Formula (I), (I′), (II), or (II′), R¹² is —PO(NR⁷)(OR⁷); and R¹³ is a substituted or unsubstituted amide. In some embodiments of Formula (I), (I′), (II), or (II′), R¹² is —OPO(OR⁷)₂; and R¹³ is a substituted or unsubstituted amide. In some embodiments of Formula (I), (I′), (II), or (II′), R¹² is —OPO(NR⁷)₂; and R¹³ is a substituted or unsubstituted amide. In some embodiments of Formula (I), (I′), (II), or (II′), R¹² is —OPO(NR⁷)(OR⁷); and R¹³ is a substituted or unsubstituted amide. In some embodiments of Formula (I), (I′), (II), or (II′), R¹² is a nitrogen protecting group; and R¹³ is a substituted or unsubstituted amide. In some embodiments of Formula (I), (I′), (II), or (II′), R¹² is —OR⁷; and R¹³ is a substituted or unsubstituted amide.

In some embodiments of Formula (I), (I′), (II), or (II′), R¹² is hydrogen; and R¹³ is a substituted or unsubstituted ester. In some embodiments of Formula (I), (I′), (II), or (II′), R¹² is substituted or unsubstituted C₁₋₆ alkyl; and R¹³ is a substituted or unsubstituted ester. In some embodiments of Formula (I), (I′), (II), or (II′), R¹² is substituted or unsubstituted C₁₋₃ alkyl; and R¹³ is a substituted or unsubstituted ester. In some embodiments of Formula (I), (I′), (II), or (II′), R¹² is branched or unbranched substituted or unsubstituted C₁₋₆ alkyl; and R¹³ is a substituted or unsubstituted ester. In some embodiments of Formula (I), (I′), (II), or (II′), R¹² is substituted or unsubstituted methyl; and R¹³ is a substituted or unsubstituted ester. In some embodiments of Formula (I), (I′), (II), or (II′), R¹² is substituted or unsubstituted ethyl; and R¹³ is a substituted or unsubstituted ester. In some embodiments of Formula (I), (I′), (II), or (II′), R¹² is substituted or unsubstituted propyl; and R¹³ is a substituted or unsubstituted ester. In some embodiments of Formula (I), (I′), (II), or (II′), R¹² is substituted or unsubstituted butyl; and R¹³ is a substituted or unsubstituted ester. In some embodiments of Formula (I), (I′), (II), or (II′), R¹² is substituted or unsubstituted pentyl; and R¹³ is a substituted or unsubstituted ester. In some embodiments of Formula (I), (I′), (II), or (II′), R¹² is substituted or unsubstituted C₁₋₆ haloalkyl; and R¹³ is a substituted or unsubstituted ester. In some embodiments of Formula (I), (I′), (II), or (II′), R¹² is substituted or unsubstituted fluoroalkyl; and R¹³ is a substituted or unsubstituted ester. In some embodiments of Formula (I), (I′), (II), or (II′), R¹² is trifluoromethyl; and R¹³ is a substituted or unsubstituted ester. In some embodiments of Formula (I), (I′), (II), or (II′), R¹² is substituted or unsubstituted trifluoroethyl; and R¹³ is a substituted or unsubstituted ester. In some embodiments of Formula (I), (I′), (II), or (II′), R¹² is substituted or unsubstituted C₂₋₆ alkenyl; and R¹³ is a substituted or unsubstituted ester. In some embodiments of Formula (I), (I′), (II), or (II′), R¹² is substituted or unsubstituted C₂₋₆ alkenyl; and R¹³ is a substituted or unsubstituted ester. In some embodiments of Formula (I), (I′), (II), or (II′), R¹² is substituted or unsubstituted vinyl; and R¹³ is a substituted or unsubstituted ester. In some embodiments of Formula (I), (I′), (II), or (II′), R¹² is substituted or unsubstituted allyl; and R¹³ is a substituted or unsubstituted ester. In some embodiments of Formula (I), (I′), (II), or (II′), R¹² is substituted or unsubstituted propenyl; and R¹³ is a substituted or unsubstituted ester. In some embodiments of Formula (I), (I′), (II), or (II′), R¹² is substituted or unsubstituted butenyl; and R¹³ is a substituted or unsubstituted ester. In some embodiments of Formula (I), (I′), (II), or (II′), R¹² is substituted or unsubstituted C₂₋₆ alkynyl; and R¹³ is a substituted or unsubstituted ester. In some embodiments of Formula (I), (I′), (II), or (II′), R¹² is substituted or unsubstituted acetylenyl; and R¹³ is a substituted or unsubstituted ester. In some embodiments of Formula (I), (I′), (II), or (II′), R¹² is substituted or unsubstituted propargyl; and R¹³ is a substituted or unsubstituted ester. In some embodiments of Formula (I), (I′), (II), or (II′), R¹² is substituted or unsubstituted butynyl; and R¹³ is a substituted or unsubstituted ester. In some embodiments of Formula (I), (I′), (II), or (II′), R¹² is substituted or unsubstituted C₁₋₆ alkoxy; and R¹³ is a substituted or unsubstituted ester. In some embodiments of Formula (I), (I′), (II), or (II′), R¹² is CH₂—OMe; and R¹³ is a substituted or unsubstituted ester. In some embodiments of Formula (I), (I′), (II), or (II′), R¹² is CH₂—OEt; and R¹³ is a substituted or unsubstituted ester. In some embodiments of Formula (I), (I′), (II), or (II′), R¹²CH₂—OPr; and R¹³ is a substituted or unsubstituted ester. In some embodiments of Formula (I), (I′), (II), or (II′), R¹² is CH₂—OCF₃; and R¹³ is a substituted or unsubstituted ester. In some embodiments of Formula (I), (I′), (II), or (II′), R¹² is substituted or unsubstituted CH₂—OR⁷; and R¹³ is a substituted or unsubstituted ester. In some embodiments of Formula (I), (I′), (II), or (II′), R¹² is substituted or unsubstituted C₆₋₁₀ aryl; and R¹³ is a substituted or unsubstituted ester. In some embodiments of Formula (I), (I′), (II), or (II′), R¹² is substituted or unsubstituted 5-10 membered heteroaryl; and R¹³ is a substituted or unsubstituted ester. In some embodiments of Formula (I), (I′), (II), or (II′), R¹² is substituted or unsubstituted 4-10 membered heterocyclyl; and R¹³ is a substituted or unsubstituted ester. In some embodiments of Formula (I), (I′), (II), or (II′), R¹² is substituted or unsubstituted 3-10 membered carbocyclyl; and R¹³ is a substituted or unsubstituted ester. In some embodiments of Formula (I), (I′), (II), or (II′), R¹² is acyl; and R¹³ is a substituted or unsubstituted ester. In some embodiments of Formula (I), (I′), (II), or (II′), R¹² is a substituted or unsubstituted amide; and R¹³ is a substituted or unsubstituted ester. In some embodiments of Formula (I), (I′), (II), or (II′), R¹² is a substituted or unsubstituted ester; and R¹³ is a substituted or unsubstituted ester. In some embodiments of Formula (I), (I′), (II), or (II′), R¹² is —OH; and R¹³ is a substituted or unsubstituted ester. In some embodiments of Formula (I), (I′), (II), or (II′), R¹² is —SO₂R⁷; and R¹³ is a substituted or unsubstituted ester. In some embodiments of Formula (I), (I′), (II), or (II′), R¹² is —SOR⁷; and R¹³ is a substituted or unsubstituted ester. In some embodiments of Formula (I), (I′), (II), or (II′), R¹² is —SO₂NR⁷₂; and R¹³ is a substituted or unsubstituted ester. In some embodiments of Formula (I), (I′), (II), or (II′), R¹² is —OSO₂R⁷; and R¹³ is a substituted or unsubstituted ester. In some embodiments of Formula (I), (I′), (II), or (II′), R¹² is —OSOR⁷; and R¹³ is a substituted or unsubstituted ester. In some embodiments of Formula (I), (I′), (II), or (II′), R¹² is —OSO₂NR⁷₂; and R¹³ is a substituted or unsubstituted ester. In some embodiments of Formula (I), (I′), (II), or (II′), R¹² is —OSO₂OR⁷; and R¹³ is a substituted or unsubstituted ester. In some embodiments of Formula (I), (I′), (II), or (II′), R¹² is —O(SO)OR⁷; and R¹³ is a substituted or unsubstituted ester. In some embodiments of Formula (I), (I′), (II), or (II′), R¹² is —PO(OR⁷)₂; and R¹³ is a substituted or unsubstituted ester. In some embodiments of Formula (I), (I′), (II), or (II′), R¹² is —PO(NR⁷)₂; and R¹³ is a substituted or unsubstituted ester. In some embodiments of Formula (I), (I′), (II), or (II′), R¹² is —PO(NR⁷)(OR⁷); and R¹³ is a substituted or unsubstituted ester. In some embodiments of Formula (I), (I′), (II), or (II′), R¹² is —OPO(OR⁷)₂; and R¹³ is a substituted or unsubstituted ester. In some embodiments of Formula (I), (I′), (II), or (II′), R¹² is —OPO(NR⁷)₂; and R¹³ is a substituted or unsubstituted ester. In some embodiments of Formula (I), (I′), (II), or (II′), R¹² is —OPO(NR⁷)(OR⁷); and R¹³ is a substituted or unsubstituted ester. In some embodiments of Formula (I), (I′), (II), or (II′), R¹² is a nitrogen protecting group; and R¹³ is a substituted or unsubstituted ester. In some embodiments of Formula (I), (I′), (II), or (II′), R¹² is —OR⁷; and R¹³ is a substituted or unsubstituted ester.

In some embodiments of Formula (I), (I′), (II), or (II′), R¹² is hydrogen; and R¹³ is —OH. In some embodiments of Formula (I), (I′), (II), or (II′), R¹² is substituted or unsubstituted C₁₋₆ alkyl; and R¹³ is —OH. In some embodiments of Formula (I), (I′), (II), or (II′), R¹² is substituted or unsubstituted C₁₋₃ alkyl; and R¹³ is —OH. In some embodiments of Formula (I), (I′), (II), or (II′), R¹² is branched or unbranched substituted or unsubstituted C₁₋₆ alkyl; and R¹³ is —OH. In some embodiments of Formula (I), (I′), (II), or (II′), R¹² is substituted or unsubstituted methyl; and R¹³ is —OH. In some embodiments of Formula (I), (I′), (II), or (II′), R¹² is substituted or unsubstituted ethyl; and R¹³ is —OH. In some embodiments of Formula (I), (I′), (II), or (II′), R¹² is substituted or unsubstituted propyl; and R¹³ is —OH. In some embodiments of Formula (I), (I′), (II), or (II′), R¹² is substituted or unsubstituted butyl; and R¹³ is —OH. In some embodiments of Formula (I), (I′), (II), or (II′), R¹² is substituted or unsubstituted pentyl; and R¹³ is —OH. In some embodiments of Formula (I), (I′), (II), or (II′), R¹² is substituted or unsubstituted C₁₋₆ haloalkyl; and R¹³ is —OH. In some embodiments of Formula (I), (I′), (II), or (II′), R¹² is substituted or unsubstituted fluoroalkyl; and R¹³ is —OH. In some embodiments of Formula (I), (I′), (II), or (II′), R¹² is trifluoromethyl; and R¹³ is —OH. In some embodiments of Formula (I), (I′), (II), or (II′), R¹² is substituted or unsubstituted trifluoroethyl; and R¹³ is —OH. In some embodiments of Formula (I), (I′), (II), or (II′), R¹² is substituted or unsubstituted C₂₋₆ alkenyl; and R¹³ is —OH. In some embodiments of Formula (I), (I′), (II), or (II′), R¹² is substituted or unsubstituted C₂₋₆ alkenyl; and R¹³ is —OH. In some embodiments of Formula (I), (I′), (II), or (II′), R¹² is substituted or unsubstituted vinyl; and R¹³ is —OH. In some embodiments of Formula (I), (I′), (II), or (II′), R¹² is substituted or unsubstituted allyl; and R¹³ is —OH. In some embodiments of Formula (I), (I′), (II), or (II′), R¹² is substituted or unsubstituted propenyl; and R¹³ is —OH. In some embodiments of Formula (I), (I′), (II), or (II′), R¹² is substituted or unsubstituted butenyl; and R¹³ is —OH. In some embodiments of Formula (I), (I′), (II), or (II′), R¹² is substituted or unsubstituted C₂₋₆ alkynyl; and R¹³ is —OH. In some embodiments of Formula (I), (I′), (II), or (II′), R¹² is substituted or unsubstituted acetylenyl; and R¹³ is —OH. In some embodiments of Formula (I), (I′), (II), or (II′), R¹² is substituted or unsubstituted propargyl; and R¹³ is —OH. In some embodiments of Formula (I), (I′), (II), or (II′), R¹² is substituted or unsubstituted butynyl; and R¹³ is —OH. In some embodiments of Formula (I), (I′), (II), or (II′), R¹² is substituted or unsubstituted C₁₋₆ alkoxy; and R¹³ is —OH. In some embodiments of Formula (I), (I′), (II), or (II′), R¹² is CH₂—OMe; and R¹³ is —OH. In some embodiments of Formula (I), (I′), (II), or (II′), R¹² is CH₂—OEt; and R¹³ is —OH. In some embodiments of Formula (I), (I′), (II), or (II′), R¹²CH₂—OPr; and R¹³ is —OH. In some embodiments of Formula (I), (I′), (II), or (II′), R¹² is CH₂—OCF₃; and R¹³ is —OH. In some embodiments of Formula (I), (I′), (II), or (II′), R¹² is substituted or unsubstituted CH₂—OR⁷ and R¹³ is —OH. In some embodiments of Formula (I), (I′), (II), or (II′), R¹² is substituted or unsubstituted C₆₋₁₀ aryl; and R¹³ is —OH. In some embodiments of Formula (I), (I′), (II), or (II′), R¹² is substituted or unsubstituted 5-10 membered heteroaryl; and R¹¹ is —OH. In some embodiments of Formula (I), (I′), (II), or (II′), R¹² is substituted or unsubstituted 4-10 membered heterocyclyl; and R¹³ is —OH. In some embodiments of Formula (I), (I′), (II), or (II′), R¹² is substituted or unsubstituted 3-10 membered carbocyclyl; and R¹³ is —OH. In some embodiments of Formula (I), (I′), (II), or (II′), R¹² is acyl; and R¹³ is —OH. In some embodiments of Formula (I), (I′), (II), or (II′), R¹² is a substituted or unsubstituted amide; and R¹³ is —OH. In some embodiments of Formula (I), (I′), (II), or (II′), R¹² is a substituted or unsubstituted ester; and R¹³ is —OH. In some embodiments of Formula (I), (I′), (II), or (II′), R¹² is —OH; and R¹³ is —OH. In some embodiments of Formula (I), (I′), (II), or (II′), R¹² is —SO₂R⁷; and R¹³ is —OH. In some embodiments of Formula (I), (I′), (II), or (II′), R¹² is —SOR⁷; and R¹³ is —OH. In some embodiments of Formula (I), (I′), (II), or (II′), R¹² is —SO₂NR⁷₂; and R³ is —OH. In some embodiments of Formula (I), (I′), (II), or (II′), R¹² is —OSO₂R⁷; and R¹³ is —OH. In some embodiments of Formula (I), (I′), (II), or (II′), R¹² is —OSOR⁷; and R³ is —OH. In some embodiments of Formula (I), (I′), (II), or (II′), R¹² is —OSO₂NR⁷₂; and R¹³ is —OH. In some embodiments of Formula (I), (I′), (II), or (II′), R¹² is —OSO₂OR⁷; and R¹¹ is —OH. In some embodiments of Formula (I), (I′), (II), or (II′), R¹² is —O(SO)OR⁷; and R¹³ is —OH. In some embodiments of Formula (I), (I′), (II), or (II′), R¹² is —PO(OR⁷)₂; and R¹³ is —OH. In some embodiments of Formula (I), (I′), (II), or (II′), R¹² is —PO(NR⁷)₂; and R¹³ is —OH. In some embodiments of Formula (I), (I′), (II), or (II′), R¹² is —PO(NR)(OR⁷); and R³ is —OH. In some embodiments of Formula (I), (I′), (II), or (II′), R¹² is —OPO(OR⁷)₂; and R¹³ is —OH. In some embodiments of Formula (I), (I′), (II), or (II′), R¹² is —OPO(NR⁷)₂; and R³ is —OH. In some embodiments of Formula (I), (I′), (II), or (II′), R¹² is —OPO(NR⁷)(OR⁷); and R¹³ is —OH. In some embodiments of Formula (I), (I′), (II), or (II′), R¹² is a nitrogen protecting group; and R¹¹ is —OH. In some embodiments of Formula (I), (I′), (II), or (II′), R¹² is —OR⁷; and R¹³ is —OH.

In some embodiments of Formula (I), (I′), (II), or (II′), R¹² is hydrogen; and R¹³ is —SO₂R⁷. In some embodiments of Formula (I), (I′), (II), or (II′), R¹² is substituted or unsubstituted C₆ alkyl; and R¹³ is —SO₂R⁷. In some embodiments of Formula (I), (I′), (II), or (II′), R¹² is substituted or unsubstituted C₁₋₃ alkyl; and R¹³ is —SO₂R⁷. In some embodiments of Formula (I), (I′), (II), or (II′), R¹² is branched or unbranched substituted or unsubstituted C₁₋₆ alkyl; and R¹³ is —SO₂R⁷. In some embodiments of Formula (I), (I′), (II), or (II′), R¹² is substituted or unsubstituted methyl; and R¹³ is —SO₂R⁷. In some embodiments of Formula (I), (I′), (II), or (II′), R¹² is substituted or unsubstituted ethyl; and R¹³ is —SO₂R⁷In some embodiments of Formula (I), (I′), (II), or (II′), R¹² is substituted or unsubstituted propyl; and R¹³ is —SO₂R⁷. In some embodiments of Formula (I), (I′), (II), or (II′), R¹² is substituted or unsubstituted butyl; and R¹³ is —SO₂R⁷. In some embodiments of Formula (I), (I′), (II), or (II′), R¹² is substituted or unsubstituted pentyl; and R¹³ is —SO₂R⁷. In some embodiments of Formula (I), (I′), (II), or (II′), R¹² is substituted or unsubstituted C₁₋₆ haloalkyl; and R¹³ is —SO₂R⁷. In some embodiments of Formula (I), (I′), (II), or (II′), R¹² is substituted or unsubstituted fluoroalkyl; and R¹³ is —SO₂R⁷. In some embodiments of Formula (I), (I′), (II), or (II′), R¹² is trifluoromethyl; and R¹³ is —SO₂R⁷. In some embodiments of Formula (I), (I′), (II), or (II′), R¹² is substituted or unsubstituted trifluoroethyl; and R¹³ is —SO₂R⁷. In some embodiments of Formula (I), (I′), (II), or (II′), R¹² is substituted or unsubstituted C₂₋₆ alkenyl; and R¹³ is —SO₂R⁷. In some embodiments of Formula (I), (I′), (II), or (II′), R¹² is substituted or unsubstituted C₂₋₆ alkenyl; and R¹³ is —SO₂R⁷. In some embodiments of Formula (I), (I′), (II), or (II′), R¹² is substituted or unsubstituted vinyl; and R¹³ is —SO₂R⁷. In some embodiments of Formula (I), (I′), (II), or (II′), R¹² is substituted or unsubstituted allyl; and R¹³ is —SO₂R⁷. In some embodiments of Formula (I), (I′), (II), or (II′), R¹² is substituted or unsubstituted propenyl; and R¹³ is —SO₂R⁷. In some embodiments of Formula (I), (I′), (II), or (II′), R¹² is substituted or unsubstituted butenyl; and R¹³ is —SO₂R⁷. In some embodiments of Formula (I), (I′), (II), or (II′), R¹² is substituted or unsubstituted C₂₋₆ alkynyl; and R¹³ is —SO₂R⁷. In some embodiments of Formula (I), (I′), (II), or (II′), R¹² is substituted or unsubstituted acetylenyl; and R¹³ is —SO₂R⁷. In some embodiments of Formula (I), (I′), (II), or (II′), R¹² is substituted or unsubstituted propargyl; and R¹³ is —SO₂R⁷. In some embodiments of Formula (I), (I′), (II), or (II′), R¹² is substituted or unsubstituted butynyl; and R¹³ is —SO₂R⁷. In some embodiments of Formula (I), (I′), (II), or (II′), R¹² is substituted or unsubstituted C₁₋₆ alkoxy; and R¹³ is —SO₂R⁷. In some embodiments of Formula (I), (I′), (II), or (II′), R¹² is CH₂—OMe; and R¹³ is —SO₂R⁷. In some embodiments of Formula (I), (I′), (II), or (II′), R¹² is CH₂—OEt; and R¹³ is —SO₂R⁷. In some embodiments of Formula (I), (I′), (II), or (II′), R¹²CH₂—OPr; and R¹³ is —SO₂R⁷. In some embodiments of Formula (I), (I′), (II), or (II′), R¹² is CH₂—OCF₃; and R¹³ is —SO₂R⁷. In some embodiments of Formula (I), (I′), (II), or (II′), R¹² is substituted or unsubstituted CH₂—OR⁷; and R¹³ is —SO₂R⁷. In some embodiments of Formula (I), (I′), (II), or (II′), R¹² is substituted or unsubstituted C₆₋₁₀ aryl; and R¹³ is —SO₂R⁷. In some embodiments of Formula (I), (I′), (II), or (II′), R¹² is substituted or unsubstituted 5-10 membered heteroaryl; and R¹³ is —SO₂R⁷. In some embodiments of Formula (I), (I′), (II), or (II′), R¹² is substituted or unsubstituted 4-10 membered heterocyclyl; and R¹³ is —SO₂R⁷. In some embodiments of Formula (I), (I′), (II), or (II′), R¹² is substituted or unsubstituted 3-10 membered carbocyclyl; and R¹³ is —SO₂R⁷. In some embodiments of Formula (I), (I′), (II), or (II′), R¹² is acyl; and R¹³ is —SO₂R⁷. In some embodiments of Formula (I), (I′), (II), or (II′), R¹² is a substituted or unsubstituted amide; and R¹³ is —SO₂R⁷. In some embodiments of Formula (I), (I′), (II), or (II′), R¹² is a substituted or unsubstituted ester; and R¹³ is —SO₂R⁷. In some embodiments of Formula (I), (I′), (II), or (II′), R¹² is —OH; and R¹³ is —SO₂R⁷. In some embodiments of Formula (I), (I′), (II), or (II′), R¹² is —₂R⁷; and R¹³ is —SO₂R⁷. In some embodiments of Formula (I), (I′), (II), or (II′), R¹² is —SOR⁷; and R¹³ is —SO₂R⁷. In some embodiments of Formula (I), (I′), (II), or (II′), R¹² is —SO₂NR⁷₂; and R¹³ is —SO₂R⁷. In some embodiments of Formula (I), (I′), (II), or (II′), R¹² is —OSO₂R⁷; and R¹³ is —SO₂R⁷. In some embodiments of Formula (I), (I′), (II), or (II′), R¹² is —OSOR⁷; and R¹³ is —SO₂R⁷. In some embodiments of Formula (I), (I′), (II), or (II′), R¹² is —OSO₂NR⁷₂; and R¹³ is —SO₂R⁷. In some embodiments of Formula (I), (I′), (II), or (II′), R¹² is —SO₂OR⁷; and R¹³ is —SO₂R⁷. In some embodiments of Formula (I), (I′), (II), or (II′), R¹² is —O(SO)OR⁷; and R¹³ is —SO₂R⁷. In some embodiments of Formula (I), (I′), (II), or (II′), R¹² is —PO(OR⁷)₂; and R¹³ is —SO₂R⁷. In some embodiments of Formula (I), (I′), (II), or (II′), R¹² is —PO(NR⁷)₂; and R¹³ is —SO₂R⁷. In some embodiments of Formula (I), (I′), (II), or (II′), R¹² is —PO(NR⁷)(OR⁷); and R³ is —SO₂R⁷. In some embodiments of Formula (I), (I′), (II), or (II′), R¹² is —OPO(OR⁷)₂; and R¹³ is —SO₂R⁷. In some embodiments of Formula (I), (I′), (II), or (II′), R¹² is —OPO(NR⁷)₂; and R¹³ is —SO₂R⁷. In some embodiments of Formula (I), (I′), (II), or (II′), R¹² is —OPO(NR⁷)(OR⁷); and R¹³ is —SO₂R⁷. In some embodiments of Formula (I), (I′), (II), or (II′), R¹² is a nitrogen protecting group; and R¹³ is —SO₂R⁷. In some embodiments of Formula (I), (I′), (II), or (II′), R¹² is —OR⁷; and R¹³ is —SO₂R⁷

In some embodiments of Formula (I), (I′), (II), or (II′), R¹² is hydrogen; and R¹¹ is —SOR⁷. In some embodiments of Formula (I), (I′), (II), or (II′), R¹² is substituted or unsubstituted C₆ alkyl; and R¹³ is —SOR⁷. In some embodiments of Formula (I), (I′), (II), or (II′), R¹² is substituted or unsubstituted C₁₋₃ alkyl; and R¹³ is —SOR⁷. In some embodiments of Formula (I), (I′), (II), or (II′), R¹² is branched or unbranched substituted or unsubstituted C₁₋₆ alkyl; and R¹³ is —SOR⁷. In some embodiments of Formula (I), (I′), (II), or (II′), R¹² is substituted or unsubstituted methyl; and R¹³ is —SOR⁷. In some embodiments of Formula (I), (I′), (II), or (II′), R¹² is substituted or unsubstituted ethyl; and R¹³ is —SOR⁷. In some embodiments of Formula (I), (I′), (II), or (II′), R¹² is substituted or unsubstituted propyl; and R¹³ is —SOR⁷. In some embodiments of Formula (I), (I′), (II), or (II′), R¹² is substituted or unsubstituted butyl; and R¹³ is —SOR⁷. In some embodiments of Formula (I), (I′), (II), or (II′), R¹² is substituted or unsubstituted pentyl; and R¹³ is —SOR⁷. In some embodiments of Formula (I), (I′), (II), or (II′), R¹² is substituted or unsubstituted C₁₋₆ haloalkyl; and R¹³ is —SOR⁷. In some embodiments of Formula (I), (I′), (II), or (II′), R¹² is substituted or unsubstituted fluoroalkyl; and R¹³ is —SOR⁷. In some embodiments of Formula (I), (I′), (II), or (II′), R¹² is trifluoromethyl; and R¹³ is —SOR⁷. In some embodiments of Formula (I), (I′), (II), or (II′), R¹² is substituted or unsubstituted trifluoroethyl; and R¹³ is —SOR⁷. In some embodiments of Formula (I), (I′), (II), or (II′), R¹² is substituted or unsubstituted C₂₋₆ alkenyl; and R¹³ is —SOR⁷. In some embodiments of Formula (I), (I′), (II), or (II′), R¹² is substituted or unsubstituted C₂₋₆ alkenyl; and R¹³ is —SOR⁷. In some embodiments of Formula (I), (I′), (II), or (II′), R¹² is substituted or unsubstituted vinyl; and R¹³ is —SOR⁷. In some embodiments of Formula (I), (I′), (II), or (II′), R¹² is substituted or unsubstituted allyl; and R¹³ is —SOR⁷. In some embodiments of Formula (I), (I′), (II), or (II′), R¹² is substituted or unsubstituted propenyl; and R¹³ is —SOR⁷. In some embodiments of Formula (I), (I′), (II), or (II′), R¹² is substituted or unsubstituted butenyl; and R¹³ is —SOR⁷. In some embodiments of Formula (I), (I′), (II), or (II′), R¹² is substituted or unsubstituted C₂₋₆ alkynyl; and R¹³ is —SOR⁷. In some embodiments of Formula (I), (I′), (II), or (II′), R¹² is substituted or unsubstituted acetylenyl; and R¹³ is —SOR⁷. In some embodiments of Formula (I), (I′), (II), or (II′), R¹² is substituted or unsubstituted propargyl; and R¹³ is —SOR⁷. In some embodiments of Formula (I), (I′), (II), or (II′), R¹² is substituted or unsubstituted butynyl; and R¹³ is —SOR⁷. In some embodiments of Formula (I), (I′), (II), or (II′), R¹² is substituted or unsubstituted C₁₋₆ alkoxy; and R¹³ is —SOR⁷. In some embodiments of Formula (I), (I′), (II), or (II′), R¹² is CH₂—OMe; and R¹³ is —SOR⁷. In some embodiments of Formula (I), (I′), (II), or (II′), R¹² is CH₂—OEt; and R¹³ is —SOR⁷. In some embodiments of Formula (I), (I′), (II), or (II′), R¹²CH₂—OPr; and R¹³ is —SOR⁷. In some embodiments of Formula (I), (I′), (II), or (II′), R¹² is CH₂—OCF₃; and R¹³ is —SOR⁷. In some embodiments of Formula (I), (I′), (II), or (II′), R¹² is substituted or unsubstituted CH₂—OR⁷; and R¹³ is —SOR⁷. In some embodiments of Formula (I), (I′), (II), or (II′), R¹² is substituted or unsubstituted C₆₋₁₀ aryl; and R¹³ is —SOR⁷. In some embodiments of Formula (I), (I′), (II), or (II′), R¹² is substituted or unsubstituted 5-10 membered heteroaryl; and R¹³ is —SOR⁷. In some embodiments of Formula (I), (I′), (II), or (II′), R¹² is substituted or unsubstituted 4-10 membered heterocyclyl; and R¹³ is —SOR⁷. In some embodiments of Formula (I), (I′), (II), or (II′), R¹² is substituted or unsubstituted 3-10 membered carbocyclyl; and R¹³ is —SOR⁷. In some embodiments of Formula (I), (I′), (II), or (II′), R¹² is acyl; and R¹³ is —SOR⁷. In some embodiments of Formula (I), (I′), (II), or (II′), R¹² is a substituted or unsubstituted amide; and R¹³ is —SOR⁷. In some embodiments of Formula (I), (I′), (II), or (II′), R¹² is a substituted or unsubstituted ester; and R¹³ is —SOR⁷. In some embodiments of Formula (I), (I′), (II), or (II′), R¹² is —OH; and R¹³ is —SOR⁷. In some embodiments of Formula (I), (I′), (II), or (II′), R¹² is —SO₂R⁷; and R¹³ is —SOR⁷. In some embodiments of Formula (I), (I′), (II), or (II′), R¹² is —SOR⁷; and R¹³ is —SOR⁷. In some embodiments of Formula (I), (I′), (II), or (II′), R¹² is —SO₂NR⁷₂; and R¹³ is —SOR⁷. In some embodiments of Formula (I), (I′), (II), or (II′), R¹² is —OSO₂R⁷; and R¹³ is —SOR⁷. In some embodiments of Formula (I), (I′), (II), or (II′), R¹² is —OSOR⁷; and R¹³ is —SOR⁷. In some embodiments of Formula (I), (I′), (II), or (II′), R¹² is —OSO₂NR⁷₂; and R¹³ is —SOR⁷. In some embodiments of Formula (I), (I′), (II), or (II′), R¹² is —OSO₂OR⁷; and R¹³ is —SOR⁷. In some embodiments of Formula (I), (I′), (II), or (II′), R¹² is —O(SO)OR⁷; and R¹³ is —SOR⁷. In some embodiments of Formula (I), (I′), (II), or (II′), R¹² is —PO(OR⁷)₂; and R¹³ is —SOR⁷. In some embodiments of Formula (I), (I′), (II), or (II′), R¹² is —PO(NR⁷)₂; and R¹³ is —SOR⁷. In some embodiments of Formula (I), (I′), (II), or (II′), R¹² is —PO(NR⁷)(OR⁷); and R¹³ is —SOR⁷. In some embodiments of Formula (I), (I′), (II), or (II′), R¹² is —OPO(OR⁷)₂; and R¹³ is —SOR⁷. In some embodiments of Formula (I), (I′), (II), or (II′), R¹² is —OPO(NR⁷)₂; and R¹³ is —SOR⁷. In some embodiments of Formula (I), (I′), (II), or (II′), R¹² is —OPO(NR⁷)(OR⁷); and R¹³ is —SOR⁷. In some embodiments of Formula (I), (I′), (II), or (II′), R¹² is a nitrogen protecting group; and R¹³ is —SOR⁷. In some embodiments of Formula (I), (I′), (II), or (II′), R¹² is —OR⁷; and R¹³ is —SOR⁷.

In some embodiments of Formula (I), (I′), (II), or (II′), R¹² is hydrogen; and R¹³ is —SO₂NR⁷₂. In some embodiments of Formula (I), (I′), (II), or (II′), R¹² is substituted or unsubstituted C₁₋₆ alkyl; and R¹³ is —SO₂NR⁷₂. In some embodiments of Formula (I), (I′), (II), or (II′), R¹² is substituted or unsubstituted C₁₋₃ alkyl; and R¹³ is —SO₂NR⁷₂. In some embodiments of Formula (I), (I′), (II), or (II′), R¹² is branched or unbranched substituted or unsubstituted C₁₋₆ alkyl; and R¹³ is —SO₂NR⁷₂. In some embodiments of Formula (I), (I′), (II), or (II′), R¹² is substituted or unsubstituted methyl; and R¹³ is —SO₂NR⁷₂. In some embodiments of Formula (I), (I′), (II), or (II′), R¹² is substituted or unsubstituted ethyl; and R¹³ is —SO₂NR⁷₂. In some embodiments of Formula (I), (I′), (II), or (II′), R¹² is substituted or unsubstituted propyl; and R¹³ is —SO₂NR⁷₂. In some embodiments of Formula (I), (I′), (II), or (II′), R¹² is substituted or unsubstituted butyl; and R¹³ is —SO₂NR⁷₂. In some embodiments of Formula (I), (I′), (II), or (II′), R¹² is substituted or unsubstituted pentyl; and R¹³ is —SO₂NR⁷₂. In some embodiments of Formula (I), (I′), (II), or (II′), R¹² is substituted or unsubstituted C₁₋₆ haloalkyl; and R¹³ is —SO₂NR⁷₂. In some embodiments of Formula (I), (I′), (II), or (II′), R¹² is substituted or unsubstituted fluoroalkyl; and R¹³ is —SO₂NR⁷₂. In some embodiments of Formula (I), (I′), (II), or (II′), R¹² is trifluoromethyl; and R¹³ is —SO₂NR⁷₂. In some embodiments of Formula (I), (I′), (II), or (II′), R¹² is substituted or unsubstituted trifluoroethyl; and R¹³ is —SO₂NR⁷₂. In some embodiments of Formula (I), (I′), (II), or (II′), R¹² is substituted or unsubstituted C₂₋₆ alkenyl; and R¹³ is —SO₂NR⁷₂. In some embodiments of Formula (I), (I′), (II), or (II′), R¹² is substituted or unsubstituted C₂₋₆ alkenyl; and R¹³ is —SO₂NR⁷₂. In some embodiments of Formula (I), (I′), (II), or (II′), R¹² is substituted or unsubstituted vinyl; and R¹³ is —SO₂NR⁷₂. In some embodiments of Formula (I), (I′), (II), or (II′), R¹² is substituted or unsubstituted allyl; and R¹³ is —SO₂NR⁷₂. In some embodiments of Formula (I), (I′), (II), or (II′), R¹² is substituted or unsubstituted propenyl; and R¹³ is —SO₂NR⁷₂. In some embodiments of Formula (I), (I′), (II), or (II′), R¹² is substituted or unsubstituted butenyl; and R¹³ is —SO₂NR⁷₂. In some embodiments of Formula (I), (I′), (II), or (II′), R¹² is substituted or unsubstituted C₂₋₆ alkynyl; and R¹³ is —SO₂NR⁷₂. In some embodiments of Formula (I), (I′), (II), or (II′), R¹² is substituted or unsubstituted acetylenyl; and R¹³ is —SO₂NR⁷₂. In some embodiments of Formula (I), (I′), (II), or (II′), R¹² is substituted or unsubstituted propargyl; and R¹³ is —SO₂NR⁷₂. In some embodiments of Formula (I), (I′), (II), or (II′), R¹² is substituted or unsubstituted butynyl; and R¹³ is —SO₂NR⁷₂. In some embodiments of Formula (I), (I′), (II), or (II′), R¹² is substituted or unsubstituted C₁₋₆ alkoxy; and R¹³ is —SO₂NR⁷₂. In some embodiments of Formula (I), (I′), (II), or (II′), R¹² is CH₂—OMe; and R¹³ is —SO₂NR⁷₂. In some embodiments of Formula (I), (I′), (II), or (II′), R¹² is CH₂—OEt; and R¹³ is —SO₂NR⁷₂. In some embodiments of Formula (I), (I′), (II), or (II′), R¹²CH₂—OPr; and R¹³ is —SO₂NR⁷₂. In some embodiments of Formula (I), (I′), (II), or (II′), R¹² is CH₂—OCF₃; and R¹³ is —SO₂NR⁷₂. In some embodiments of Formula (I), (I′), (II), or (II′), R¹² is substituted or unsubstituted CH₂—OR⁷; and R¹³ is —SO₂NR⁷₂. In some embodiments of Formula (I), (I′), (II), or (II′), R¹² is substituted or unsubstituted C₆₋₁₀ aryl; and R¹³ is —SO₂NR⁷₂. In some embodiments of Formula (I), (I′), (II), or (II′), R¹² is substituted or unsubstituted 5-10 membered heteroaryl; and R¹³ is —SO₂NR⁷₂. In some embodiments of Formula (I), (I′), (II), or (II′), R¹² is substituted or unsubstituted 4-10 membered heterocyclyl; and R¹³ is —SO₂NR⁷₂. In some embodiments of Formula (I), (I′), (II), or (II′), R¹² is substituted or unsubstituted 3-10 membered carbocyclyl; and R¹³ is —SO₂NR⁷₂. In some embodiments of Formula (I), (I′), (II), or (II′), R¹² is acyl; and R¹³ is —SO₂NR⁷₂. In some embodiments of Formula (I), (I′), (II), or (II′), R¹² is a substituted or unsubstituted amide; and R¹³ is —SO₂NR⁷₂. In some embodiments of Formula (I), (I′), (II), or (II′), R¹² is a substituted or unsubstituted ester; and R¹³ is —SO₂NR⁷₂. In some embodiments of Formula (I), (I′), (II), or (II′), R¹² is —OH; and R¹³ is —SO₂NR⁷₂. In some embodiments of Formula (I), (I′), (II), or (II′), R¹² is —SO₂R⁷; and R¹³ is —SO₂NR⁷₂. In some embodiments of Formula (I), (I′), (II), or (II′), R¹² is —SOR⁷; and R¹³ is —SO₂NR⁷₂. In some embodiments of Formula (I), (I′), (II), or (II′), R¹² is —SO₂NR⁷₂; and R¹³ is —SO₂NR⁷₂. In some embodiments of Formula (I), (I′), (II), or (II′), R¹² is —OSO₂R⁷; and R¹³ is —SO₂NR⁷₂. In some embodiments of Formula (I), (I′), (II), or (II′), R¹² is —OSOR⁷; and R¹³ is —SO₂NR⁷₂. In some embodiments of Formula (I), (I′), (II), or (II′), R¹² is —OSO₂NR⁷₂; and R¹¹ is —SO₂NR⁷₂. In some embodiments of Formula (I), (I′), (II), or (II′), R¹² is —OSO₂OR⁷; and R¹³ is —SO₂NR⁷₂. In some embodiments of Formula (I), (I′), (II), or (II′), R¹² is —O(SO)OR⁷; and R¹³ is —SO₂NR⁷₂. In some embodiments of Formula (I), (I′), (II), or (II′), R¹² is —PO(OR⁷)₂; and R¹³ is —SO₂NR⁷₂. In some embodiments of Formula (I), (I′), (II), or (II′), R¹² is —PO(NR⁷)₂; and R¹³ is —SO₂NR⁷₂. In some embodiments of Formula (I), (I′), (II), or (II′), R¹² is —PO(NR⁷)(OR⁷); and R¹³ is —SO₂NR⁷₂. In some embodiments of Formula (I), (I′), (II), or (II′), R¹² is —OPO(OR⁷)₂; and R¹³ is —SO₂NR⁷₂. In some embodiments of Formula (I), (I′), (II), or (II′), R¹² is —OPO(NR⁷)₂; and R¹³ is —SO₂NR⁷₂. In some embodiments of Formula (I), (I′), (II), or (II′), R¹² is —OPO(NR⁷)(OR⁷); and R¹³ is —SO₂NR⁷₂. In some embodiments of Formula (I), (I′), (II), or (II′), R¹² is a nitrogen protecting group; and R¹³ is —SO₂NR⁷₂. In some embodiments of Formula (I), (I′), (II), or (II′), R¹² is —OR⁷; and R¹³ is —SO₂NR⁷₂.

In some embodiments of Formula (I), (I′), (II), or (II′), R¹² is hydrogen; and R¹³ is —OSO₂R⁷. In some embodiments of Formula (I), (I′), (II), or (II′), R¹² is substituted or unsubstituted C₁₋₆ alkyl; and R¹³ is —OSO₂R⁷. In some embodiments of Formula (I), (I′), (II), or (II′), R¹² is substituted or unsubstituted C₁₋₃ alkyl; and R¹³ is —OSO₂R⁷. In some embodiments of Formula (I), (I′), (II), or (II′), R¹² is branched or unbranched substituted or unsubstituted C₁₋₆ alkyl; and R¹³ is —OSO₂R⁷. In some embodiments of Formula (I), (I′), (II), or (II′), R¹² is substituted or unsubstituted methyl; and R¹³ is —OSO₂R⁷. In some embodiments of Formula (I), (I′), (II), or (II′), R¹² is substituted or unsubstituted ethyl; and R³ is —OSO₂R⁷. In some embodiments of Formula (I), (I′), (II), or (II′), R¹² is substituted or unsubstituted propyl; and R¹³ is —OSO₂R⁷. In some embodiments of Formula (I), (I′), (II), or (II′), R¹² is substituted or unsubstituted butyl; and R³ is —OSO₂R⁷. In some embodiments of Formula (I), (I′), (II), or (II′), R¹² is substituted or unsubstituted pentyl; and R¹³ is —OSO₂R⁷. In some embodiments of Formula (I), (I′), (II), or (II′), R¹² is substituted or unsubstituted C₁₋₆ haloalkyl; and R¹³ is —OSO₂R⁷. In some embodiments of Formula (I), (I′), (II), or (II′), R¹² is substituted or unsubstituted fluoroalkyl; and R³ is —OSO₂R⁷. In some embodiments of Formula (I), (I′), (II), or (II′), R¹² is trifluoromethyl; and R¹³ is —OSO₂R⁷. In some embodiments of Formula (I), (I′), (II), or (II′), R¹² is substituted or unsubstituted trifluoroethyl; and R¹³ is —OSO₂R⁷. In some embodiments of Formula (I), (I′), (II), or (II′), R¹² is substituted or unsubstituted C₂₋₆ alkenyl; and R³ is —OSO₂R⁷. In some embodiments of Formula (I), (I′), (II), or (II′), R¹² is substituted or unsubstituted C₂₋₆ alkenyl; and R¹³ is —OSO₂R⁷. In some embodiments of Formula (I), (I′), (II), or (II′), R¹² is substituted or unsubstituted vinyl; and R¹³ is —OSO₂R⁷. In some embodiments of Formula (I), (I′), (II), or (II′), R¹² is substituted or unsubstituted allyl; and R¹³ is —OSO₂R⁷. In some embodiments of Formula (I), (I′), (II), or (II′), R¹² is substituted or unsubstituted propenyl; and R¹³ is —OSO₂R⁷. In some embodiments of Formula (I), (I′), (II), or (II′), R¹² is substituted or unsubstituted butenyl; and R¹³ is —OSO₂R⁷. In some embodiments of Formula (I), (I′), (II), or (II′), R¹² is substituted or unsubstituted C₂₋₆ alkynyl; and R¹³ is —OSO₂R⁷. In some embodiments of Formula (I), (I′), (II), or (II′), R¹² is substituted or unsubstituted acetylenyl; and R¹³ is —OSO₂R⁷. In some embodiments of Formula (I), (I′), (II), or (II′), R¹² is substituted or unsubstituted propargyl; and R¹³ is —OSO₂R⁷. In some embodiments of Formula (I), (I′), (II), or (II′), R¹² is substituted or unsubstituted butynyl; and R¹³ is —OSO₂R⁷. In some embodiments of Formula (I), (I′), (II), or (II′), R¹² is substituted or unsubstituted C₁₋₆ alkoxy; and R¹³ is —OSO₂R⁷. In some embodiments of Formula (I), (I′), (II), or (II′), R¹² is CH₂—OMe; and R³ is —OSO₂R⁷. In some embodiments of Formula (I), (I′), (II), or (II′), R¹² is CH₂-OEt; and R¹³ is —OSO₂R⁷. In some embodiments of Formula (I), (I′), (II), or (II′), R¹²CH₂—OPr; and R³ is —OSO₂R⁷. In some embodiments of Formula (I), (I′), (II), or (II′), R¹² is CH₂—OCF₃; and R¹³ is —OSO₂R⁷. In some embodiments of Formula (I), (I′), (II), or (II′), R¹² is substituted or unsubstituted CH₂—OR⁷; and R¹³ is —OSO₂R⁷. In some embodiments of Formula (I), (I′), (II), or (II′), R¹² is substituted or unsubstituted C₆₋₁₀ aryl; and R¹³ is —OSO₂R⁷. In some embodiments of Formula (I), (I′), (II), or (II′), R¹² is substituted or unsubstituted 5-10 membered heteroaryl; and R¹³ is —OSO₂R⁷. In some embodiments of Formula (I), (I′), (II), or (II′), R¹² is substituted or unsubstituted 4-10 membered heterocyclyl; and R¹³ is —OSO₂R⁷. In some embodiments of Formula (I), (I′), (II), or (II′), R¹² is substituted or unsubstituted 3-10 membered carbocyclyl; and R¹³ is —OSO₂R⁷. In some embodiments of Formula (I), (I′), (II), or (II′), R¹² is acyl; and R¹³ is —OSO₂R⁷. In some embodiments of Formula (I), (I′), (II), or (II′), R¹² is a substituted or unsubstituted amide; and R¹³ is —OSO₂R⁷. In some embodiments of Formula (I), (I′), (II), or (II′), R¹² is a substituted or unsubstituted ester; and R¹³ is —OSO₂R⁷. In some embodiments of Formula (I), (I′), (II), or (II′), R¹² is —OH; and R¹³ is —OSO₂R⁷. In some embodiments of Formula (I), (I′), (II), or (II′), R¹² is —SO₂R⁷; and R¹³ is —OSO₂R⁷. In some embodiments of Formula (I), (I′), (II), or (II′), R¹² is —SOR⁷; and R¹³ is —OSO₂R⁷. In some embodiments of Formula (I), (I′), (II), or (II′), R¹² is —SO₂NR⁷₂; and R¹³ is —OSO₂R⁷. In some embodiments of Formula (I), (I′), (II), or (II′), R¹² is —OSO₂R⁷; and R¹³ is —OSO₂R⁷. In some embodiments of Formula (I), (I′), (II), or (II′), R¹² is —OSOR⁷; and R¹³ is —OSO₂R⁷. In some embodiments of Formula (I), (I′), (II), or (II′), R¹² is —OSO₂NR⁷₂; and R¹³ is —OSO₂R⁷. In some embodiments of Formula (I), (I′), (II), or (II′), R¹² is —OSO₂OR⁷; and R¹³ is —OSO₂R⁷. In some embodiments of Formula (I), (I′), (II), or (II′), R¹² is —O(SO)OR⁷; and R¹³ is —OSO₂R⁷. In some embodiments of Formula (I), (I′), (II), or (II′), R¹² is —PO(OR⁷)₂; and R¹³ is —OSO₂R⁷. In some embodiments of Formula (I), (I′), (II), or (II′), R¹² is —PO(NR⁷)₂; and R¹¹ is —OSO₂R⁷. In some embodiments of Formula (I), (I′), (II), or (II′), R¹² is —PO(NR⁷)(OR⁷); and R¹³ is —OSO₂R⁷. In some embodiments of Formula (I), (I′), (II), or (II′), R¹² is —OPO(OR⁷)₂; and R¹³ is —OSO₂R⁷. In some embodiments of Formula (I), (I′), (II), or (II′), R¹² is —OPO(NR⁷)₂; and R¹³ is —OSO₂R⁷. In some embodiments of Formula (I), (I′), (II), or (II′), R¹² is —OPO(NR⁷)(OR⁷); and R¹³ is —OSO₂R⁷. In some embodiments of Formula (I), (I′), (II), or (II′), R¹² is a nitrogen protecting group; and R¹³ is —OSO₂R⁷. In some embodiments of Formula (I), (I′), (II), or (II′), R¹² is —OR⁷; and R¹³ is —OSO₂R⁷.

In some embodiments of Formula (I), (I′), (II), or (II′), R¹² is hydrogen; and R¹³ is —OSOR⁷. In some embodiments of Formula (I), (I′), (II), or (II′), R¹² is substituted or unsubstituted C₁₋₆ alkyl; and R¹³ is —OSOR⁷. In some embodiments of Formula (I), (I′), (II), or (II′), R¹² is substituted or unsubstituted C₁₋₃ alkyl; and R¹³ is —OSOR⁷. In some embodiments of Formula (I), (I′), (II), or (II′), R¹² is branched or unbranched substituted or unsubstituted C₁₋₆ alkyl; and R¹³ is —OSOR⁷. In some embodiments of Formula (I), (I′), (II), or (II′), R¹² is substituted or unsubstituted methyl; and R¹³ is —OSOR⁷. In some embodiments of Formula (I), (I′), (II), or (II′), R¹² is substituted or unsubstituted ethyl; and R¹³ is —OSOR⁷. In some embodiments of Formula (I), (I′), (II), or (II′), R¹² is substituted or unsubstituted propyl; and R¹³ is —OSOR⁷. In some embodiments of Formula (I), (I′), (II), or (II′), R¹² is substituted or unsubstituted butyl; and R¹³ is —OSOR⁷. In some embodiments of Formula (I), (I′), (II), or (II′), R¹² is substituted or unsubstituted pentyl; and R¹³ is —OSOR⁷. In some embodiments of Formula (I), (I′), (II), or (II′), R¹² is substituted or unsubstituted C₁₋₆haloalkyl; and R¹³ is —OSOR⁷. In some embodiments of Formula (I), (I′), (II), or (II′), R¹² is substituted or unsubstituted fluoroalkyl; and R¹³ is —OSOR⁷. In some embodiments of Formula (I), (I′), (II), or (II′), R¹² is trifluoromethyl; and R¹³ is —OSOR⁷. In some embodiments of Formula (I), (I′), (II), or (II′), R¹² is substituted or unsubstituted trifluoroethyl; and R¹³ is —OSOR⁷. In some embodiments of Formula (I), (I′), (II), or (II′), R¹² is substituted or unsubstituted C₂₋₆ alkenyl; and R¹³ is —OSOR⁷. In some embodiments of Formula (I), (I′), (II), or (II′), R¹² is substituted or unsubstituted C₂₋₆ alkenyl; and R¹³ is —OSOR⁷. In some embodiments of Formula (I), (I′), (II), or (II′), R¹² is substituted or unsubstituted vinyl; and R¹³ is —OSOR⁷. In some embodiments of Formula (I), (I′), (II), or (II′), R¹² is substituted or unsubstituted allyl; and R¹³ is —OSOR⁷. In some embodiments of Formula (I), (I′), (II), or (II′), R¹² is substituted or unsubstituted propenyl; and R¹³ is —OSOR⁷. In some embodiments of Formula (I), (I′), (II), or (II′), R¹² is substituted or unsubstituted butenyl; and R¹³ is —OSOR⁷. In some embodiments of Formula (I), (I′), (II), or (II′), R¹² is substituted or unsubstituted C₂₋₆ alkynyl; and R¹³ is —OSOR⁷. In some embodiments of Formula (I), (I′), (II), or (II′), R¹² is substituted or unsubstituted acetylenyl; and R¹³ is —OSOR⁷. In some embodiments of Formula (I), (I′), (II), or (II′), R¹² is substituted or unsubstituted propargyl; and R¹³ is —OSOR⁷. In some embodiments of Formula (I), (I′), (II), or (II′), R¹² is substituted or unsubstituted butynyl; and R¹³ is —OSOR⁷. In some embodiments of Formula (I), (I′), (II), or (II′), R¹² is substituted or unsubstituted C₁₋₆ alkoxy; and R¹³ is —OSOR⁷. In some embodiments of Formula (I), (I′), (II), or (II′), R¹² is CH₂—OMe; and R¹³ is —OSOR⁷. In some embodiments of Formula (I), (I′), (II), or (II′), R¹² is CH₂—OEt; and R¹³ is —OSOR⁷. In some embodiments of Formula (I), (I′), (II), or (II′), R¹²CH—OPr; and R¹³ is —OSOR⁷. In some embodiments of Formula (I), (I′), (II), or (II′), R¹² is CH₂—OCF₃; and R¹³ is —OSOR⁷. In some embodiments of Formula (I), (I′), (II), or (II′), R¹² is substituted or unsubstituted CH₂—OR⁷; and R¹³ is —OSOR⁷. In some embodiments of Formula (I), (I′), (II), or (II′), R¹² is substituted or unsubstituted C₆₋₁₀ aryl; and R¹¹ is —OSOR⁷. In some embodiments of Formula (I), (I′), (II), or (II′), R¹² is substituted or unsubstituted 5-10 membered heteroaryl; and R¹³ is —OSOR⁷. In some embodiments of Formula (I), (I′), (II), or (II′), R¹² is substituted or unsubstituted 4-10 membered heterocyclyl; and R¹³ is —OSOR⁷. In some embodiments of Formula (I), (I′), (II), or (II′), R¹² is substituted or unsubstituted 3-10 membered carbocyclyl; and R¹³ is —OSOR⁷. In some embodiments of Formula (I), (I′), (II), or (II′), R¹² is acyl; and R³ is —OSOR⁷. In some embodiments of Formula (I), (I′), (II), or (II′), R¹² is a substituted or unsubstituted amide; and R¹³ is —OSOR⁷. In some embodiments of Formula (I), (I′), (II), or (II′), R¹² is a substituted or unsubstituted ester; and R¹³ is —OSOR⁷. In some embodiments of Formula (I), (I′), (II), or (II′), R¹² is —OH; and R¹³ is —OSOR⁷. In some embodiments of Formula (I), (I′), (II), or (II′), R¹² is —SO₂R⁷; and R¹³ is —OSOR⁷. In some embodiments of Formula (I), (I′), (II), or (II′), R¹² is —SOR⁷; and R¹³ is —OSOR⁷. In some embodiments of Formula (I), (I′), (II), or (II′), R¹² is —SO₂NR⁷₂; and R³ is —OSOR⁷. In some embodiments of Formula (I), (I′), (II), or (II′), R¹² is —OSO₂R⁷; and R¹³ is —OSOR⁷. In some embodiments of Formula (I), (I′), (II), or (II′), R¹² is —OSOR⁷; and R¹³ is —OSOR⁷. In some embodiments of Formula (I), (I′), (II), or (II′), R¹² is —OSO₂NR⁷₂; and R¹³ is —OSOR⁷. In some embodiments of Formula (I), (I′), (II), or (II′), R¹² is —OSO₂OR⁷; and R¹³ is —OSOR⁷. In some embodiments of Formula (I), (I′), (II), or (II′), R¹² is —O(SO)OR⁷; and R³ is —OSOR⁷. In some embodiments of Formula (I), (I′), (II), or (II′), R¹² is —PO(OR⁷)₂; and R¹³ is —OSOR⁷. In some embodiments of Formula (I), (I′), (II), or (II′), R¹² is —PO(NR⁷)₂; and R¹³ is —OSOR⁷. In some embodiments of Formula (I), (I′), (II), or (II′), R¹² is —PO(NR⁷)(OR⁷); and R¹³ is —OSOR⁷. In some embodiments of Formula (I), (I′), (II), or (II′), R¹² is —OPO(OR⁷)₂; and R¹³ is —OSOR⁷. In some embodiments of Formula (I), (I′), (II), or (II′), R¹² is —OPO(NR⁷)₂; and R¹³ is —OSOR⁷. In some embodiments of Formula (I), (I′), (II), or (II′), R¹² is —OPO(NR⁷)(OR⁷); and R¹³ is —OSOR⁷. In some embodiments of Formula (I), (I′), (II), or (II′), R¹² is a nitrogen protecting group; and R¹³ is —OSOR⁷. In some embodiments of Formula (I), (I′), (II), or (II′), R¹² is —OR⁷; and R¹³ is —OSOR⁷.

In some embodiments of Formula (I), (I′), (II), or (II′), R¹² is hydrogen; and R¹³ is —OSO₂NR⁷₂. In some embodiments of Formula (I), (I′), (II), or (II′), R¹² is substituted or unsubstituted C₁₋₆ alkyl; and R¹³ is —OSO₂NR⁷₂. In some embodiments of Formula (I), (I′), (II), or (II′), R¹² is substituted or unsubstituted C₁₋₃ alkyl; and R¹³ is —OSO₂NR⁷₂. In some embodiments of Formula (I), (I′), (II), or (II′), R¹² is branched or unbranched substituted or unsubstituted C₁₋₆ alkyl; and R¹³ is —OSO₂NR⁷₂. In some embodiments of Formula (I), (I′), (II), or (II′), R¹² is substituted or unsubstituted methyl; and R¹³ is —OSO₂NR⁷₂. In some embodiments of Formula (I), (I′), (II), or (II′), R¹² is substituted or unsubstituted ethyl; and R¹³ is —OSO₂NR⁷₂. In some embodiments of Formula (I), (I′), (II), or (II′), R¹² is substituted or unsubstituted propyl; and R¹³ is —OSO₂NR⁷₂. In some embodiments of Formula (I), (I′), (II), or (II′), R¹² is substituted or unsubstituted butyl; and R¹³ is —OSO₂NR⁷₂. In some embodiments of Formula (I), (I′), (II), or (II′), R¹² is substituted or unsubstituted pentyl; and R¹³ is —OSO₂NR⁷₂. In some embodiments of Formula (I), (I′), (II), or (II′), R¹² is substituted or unsubstituted C₁₋₆ haloalkyl; and R¹³ is —OSO₂NR⁷₂. In some embodiments of Formula (I), (I′), (II), or (II′), R¹² is substituted or unsubstituted fluoroalkyl; and R¹³ is —OSO₂NR⁷₂. In some embodiments of Formula (I), (I′), (II), or (II′), R¹² is trifluoromethyl; and R¹³ is —OSO₂NR⁷₂. In some embodiments of Formula (I), (I′), (II), or (II′), R¹² is substituted or unsubstituted trifluoroethyl; and R¹³ is —OSO₂NR⁷₂. In some embodiments of Formula (I), (I′), (II), or (II′), R¹² is substituted or unsubstituted C₂₋₆ alkenyl; and R¹³ is —OSO₂NR⁷₂. In some embodiments of Formula (I), (I′), (II), or (II′), R¹² is substituted or unsubstituted C₂₋₆ alkenyl; and R¹³ is —OSO₂NR⁷₂. In some embodiments of Formula (I), (I′), (II), or (II′), R¹² is substituted or unsubstituted vinyl; and R¹³ is —OSO₂NR⁷₂. In some embodiments of Formula (I), (I′), (II), or (II′), R¹² is substituted or unsubstituted allyl; and R¹³ is —OSO₂NR⁷₂. In some embodiments of Formula (I), (I′), (II), or (II′), R¹² is substituted or unsubstituted propenyl; and R¹³ is —OSO₂NR⁷₂. In some embodiments of Formula (I), (I′), (II), or (II′), R¹² is substituted or unsubstituted butenyl; and R¹³ is —OSO₂NR⁷₂. In some embodiments of Formula (I), (I′), (II), or (II′), R¹² is substituted or unsubstituted C₂₋₆ alkynyl; and R¹³ is —OSO₂NR⁷₂. In some embodiments of Formula (I), (I′), (II), or (II′), R¹² is substituted or unsubstituted acetylenyl; and R¹³ is —OSO₂NR⁷₂. In some embodiments of Formula (I), (I′), (II), or (II′), R¹² is substituted or unsubstituted propargyl; and R¹³ is —OSO₂NR⁷₂. In some embodiments of Formula (I), (I′), (II), or (II′), R¹² is substituted or unsubstituted butynyl; and R¹³ is —OSO₂NR⁷₂. In some embodiments of Formula (I), (I′), (II), or (II′), R¹² is substituted or unsubstituted C₁₋₆ alkoxy; and R¹³ is —OSO₂NR⁷₂. In some embodiments of Formula (I), (I′), (II), or (II′), R¹² is CH₂—OMe; and R¹³ is —OSO₂NR⁷₂. In some embodiments of Formula (I), (I′), (II), or (II′), R¹² is CH₂-OEt; and R¹³ is —OSO₂NR⁷₂. In some embodiments of Formula (I), (I′), (II), or (II′), R¹²CH₂—OPr; and R¹³ is —OSO₂NR⁷₂. In some embodiments of Formula (I), (I′), (II), or (II′), R¹² is CH₂—OCF₃; and R¹³ is —OSO₂NR⁷₂. In some embodiments of Formula (I), (I′), (II), or (II′), R¹² is substituted or unsubstituted CH₂—OR⁷; and R¹³ is —OSO₂NR⁷₂. In some embodiments of Formula (I), (I′), (II), or (II′), R¹² is substituted or unsubstituted C₆₋₁₀ aryl; and R¹³ is —OSO₂NR⁷₂. In some embodiments of Formula (I), (I′), (II), or (II′), R¹² is substituted or unsubstituted 5-10 membered heteroaryl; and R¹³ is —OSO₂NR⁷₂. In some embodiments of Formula (I), (I′), (II), or (II′), R¹² is substituted or unsubstituted 4-10 membered heterocyclyl; and R¹³ is —OSO₂NR⁷₂. In some embodiments of Formula (I), (I′), (II), or (II′), R¹² is substituted or unsubstituted 3-10 membered carbocyclyl; and R¹³ is —OSO₂NR⁷₂. In some embodiments of Formula (I), (I′), (II), or (II′), R¹² is acyl; and R¹³ is —OSO₂NR⁷₂. In some embodiments of Formula (I), (I′), (II), or (II′), R¹² is a substituted or unsubstituted amide; and R¹³ is —OSO₂NR⁷₂. In some embodiments of Formula (I), (I′), (II), or (II′), R¹² is a substituted or unsubstituted ester; and R¹³ is —OSO₂NR⁷₂. In some embodiments of Formula (I), (I′), (II), or (II′), R¹² is —OH; and R¹³ is —OSO₂NR⁷₂. In some embodiments of Formula (I), (I′), (II), or (II′), R¹² is —SO₂R⁷; and R¹³ is —OSO₂NR⁷₂. In some embodiments of Formula (I), (I′), (II), or (II′), R¹² is —SOR⁷; and R¹³ is —OSO₂NR⁷₂. In some embodiments of Formula (I), (I′), (II), or (II′), R¹² is —SO₂NR⁷₂; and R¹³ is —OSO₂NR⁷₂. In some embodiments of Formula (I), (I′), (II), or (II′), R¹² is —OSO₂R⁷; and R¹³ is —OSO₂NR⁷₂. In some embodiments of Formula (I), (I), (II), or (II), R¹² is —OSOR⁷; and R¹³ is —OSO₂NR⁷₂. In some embodiments of Formula (I), (I′), (II), or (II′), R¹² is —OSO₂NR⁷₂; and R¹³ is —OSO₂NR⁷₂. In some embodiments of Formula (I), (I′), (II), or (II′), R¹² is —OSO₂OR⁷; and R¹³ is —OSO₂NR⁷₂. In some embodiments of Formula (I), (I′), (II), or (II′), R¹² is —O(SO)OR⁷; and R¹³ is —OSO₂NR⁷₂. In some embodiments of Formula (I), (I′), (II), or (II′), R¹² is —PO(OR⁷)₂; and R¹¹ is —OSO₂NR⁷₂. In some embodiments of Formula (I), (I′), (II), or (II′), R¹² is —PO(NR⁷)₂; and R¹³ is —OSO₂NR⁷₂. In some embodiments of Formula (I), (I′), (II), or (II′), R¹² is —PO(NR⁷)(OR⁷); and R¹³ is —OSO₂NR⁷₂. In some embodiments of Formula (I), (I′), (II), or (II′), R¹² is —OPO(OR⁷)₂; and R¹³ is —OSO₂NR⁷₂. In some embodiments of Formula (I), (I′), (II), or (II′), R¹² is —OPO(NR⁷)₂; and R¹³ is —OSO₂NR⁷₂. In some embodiments of Formula (I), (I′), (II), or (II′), R¹² is —OPO(NR⁷)(OR⁷); and R¹³ is —OSO₂NR⁷₂. In some embodiments of Formula (I), (I′), (II), or (II′), R¹² is a nitrogen protecting group; and R¹³ is —OSO₂NR⁷₂. In some embodiments of Formula (I), (I′), (II), or (II′), R¹² is —OR⁷; and R¹³ is —OSO₂NR⁷₂.

In some embodiments of Formula (I), (I′), (II), or (II′), R¹² is hydrogen; and R¹³ is —OSO₂OR⁷. In some embodiments of Formula (I), (I′), (II), or (II′), R¹² is substituted or unsubstituted C₁₋₆ alkyl; and R¹³ is —OSO₂OR⁷. In some embodiments of Formula (I), (I′), (II), or (II′), R¹² is substituted or unsubstituted C₁₋₃ alkyl; and R¹³ is —OSO₂OR⁷. In some embodiments of Formula (I), (I′), (II), or (II′), R¹² is branched or unbranched substituted or unsubstituted C₁₋₆ alkyl; and R¹³ is —OSO₂OR⁷. In some embodiments of Formula (I), (I′), (II), or (II′), R¹² is substituted or unsubstituted methyl; and R¹³ is —OSO₂OR⁷. In some embodiments of Formula (I), (I′), (II), or (II′), R¹² is substituted or unsubstituted ethyl; and R¹³ is —OSO₂OR⁷. In some embodiments of Formula (I), (I′), (II), or (II′), R¹² is substituted or unsubstituted propyl; and R¹³ is —OSO₂OR⁷. In some embodiments of Formula (I), (I′), (II), or (II′), R¹² is substituted or unsubstituted butyl; and R¹³ is —OSO₂OR⁷. In some embodiments of Formula (I), (I′), (II), or (II′), R¹² is substituted or unsubstituted pentyl; and R¹³ is —OSO₂OR⁷. In some embodiments of Formula (I), (I′), (II), or (II′), R¹² is substituted or unsubstituted C₁₋₆ haloalkyl; and R¹³ is —OSO₂OR⁷. In some embodiments of Formula (I), (I′), (II), or (II′), R¹² is substituted or unsubstituted fluoroalkyl; and R¹³ is —OSO₂OR⁷. In some embodiments of Formula (I), (I′), (II), or (II′), R¹² is trifluoromethyl; and R¹³ is —OSO₂OR⁷. In some embodiments of Formula (I), (I′), (II), or (II′), R¹² is substituted or unsubstituted trifluoroethyl; and R¹³ is —OSO₂OR⁷. In some embodiments of Formula (I), (I′), (II), or (II′), R¹² is substituted or unsubstituted C₂₋₆ alkenyl; and R¹³ is —OSO₂OR⁷. In some embodiments of Formula (I), (I′), (II), or (II′), R¹² is substituted or unsubstituted C₂₋₆ alkenyl; and R¹³ is —OSO₂OR⁷. In some embodiments of Formula (I), (I′), (II), or (II′), R¹² is substituted or unsubstituted vinyl; and R¹³ is —OSO₂OR⁷. In some embodiments of Formula (I), (I′), (II), or (II′), R¹² is substituted or unsubstituted allyl; and R¹³ is —OSO₂OR⁷. In some embodiments of Formula (I), (I′), (II), or (II′), R¹² is substituted or unsubstituted propenyl; and R¹³ is —OSO₂OR⁷. In some embodiments of Formula (I), (I′), (II), or (II′), R¹² is substituted or unsubstituted butenyl; and R¹³ is —OSO₂OR⁷. In some embodiments of Formula (I), (I′), (II), or (II′), R¹² is substituted or unsubstituted C₂₋₆ alkynyl; and R¹³ is —OSO₂OR⁷. In some embodiments of Formula (I), (I′), (II), or (II′), R¹² is substituted or unsubstituted acetylenyl; and R¹³ is —OSO₂OR⁷. In some embodiments of Formula (I), (I′), (II), or (II′), R¹² is substituted or unsubstituted propargyl; and R¹³ is —OSO₂OR⁷. In some embodiments of Formula (I), (I′), (II), or (II′), R¹² is substituted or unsubstituted butynyl; and R¹³ is —OSO₂OR⁷. In some embodiments of Formula (I), (I′), (II), or (II′), R¹² is substituted or unsubstituted C₁₋₆ alkoxy; and R¹³ is —OSO₂OR⁷. In some embodiments of Formula (I), (I′), (II), or (II′), R¹² is CH₂—OMe; and R¹³ is —OSO₂OR⁷. In some embodiments of Formula (I), (I′), (II), or (II′), R¹² is CH₂-OEt; and R¹³ is —OSO₂R⁷. In some embodiments of Formula (I), (I′), (II), or (II′), R¹²CH₂—OPr; and R¹³ is —OSO₂R⁷. In some embodiments of Formula (I), (I′), (II), or (II′), R¹² is CH₂—OCF₃; and R¹³ is —OSO₂R⁷. In some embodiments of Formula (I), (I′), (II), or (II′), R¹² is substituted or unsubstituted CH₂—OR⁷; and R¹³ is —OSO₂R⁷. In some embodiments of Formula (I), (I′), (II), or (II′), R¹² is substituted or unsubstituted C₆₋₁₀ aryl; and R¹¹ is —OSO₂R⁷. In some embodiments of Formula (I), (I′), (II), or (II′), R¹² is substituted or unsubstituted 5-10 membered heteroaryl; and R¹³ is —OSO₂OR⁷. In some embodiments of Formula (I), (I′), (II), or (II′), R¹² is substituted or unsubstituted 4-10 membered heterocyclyl; and R¹³ is —OSO₂R⁷. In some embodiments of Formula (I), (I′), (II), or (II′), R¹² is substituted or unsubstituted 3-10 membered carbocyclyl; and R¹³ is —OSO₂OR⁷. In some embodiments of Formula (I), (I′), (II), or (II′), R¹² is acyl; and R¹³ is —OSO₂R⁷. In some embodiments of Formula (I), (I′), (II), or (II′), R¹² is a substituted or unsubstituted amide; and R¹³ is —OSO₂OR⁷. In some embodiments of Formula (I), (I′), (II), or (II′), R¹² is a substituted or unsubstituted ester; and R¹³ is —OSO₂OR⁷. In some embodiments of Formula (I), (I′), (II), or (II′), R¹² is —OH; and R¹³ is —OSO₂OR⁷. In some embodiments of Formula (I), (I′), (II), or (II′), R¹² is —SO₂R⁷; and R¹³ is —OS₂R⁷. In some embodiments of Formula (I), (I′), (II), or (II′), R¹² is —SOR⁷; and R¹³ is —OSO₂OR⁷. In some embodiments of Formula (I), (I′), (II), or (II′), R¹² is —SO₂NR⁷₂; and R¹³ is —OSO₂OR⁷. In some embodiments of Formula (I), (I′), (II), or (II′), R¹² is —OSO₂R⁷; and R¹³ is —OSO₂OR⁷. In some embodiments of Formula (I), (I′), (II), or (II′), R¹² is —OSOR⁷; and R¹³ is —OSO₂OR⁷. In some embodiments of Formula (I), (I′), (II), or (II′), R¹² is —OSO₂NR⁷₂; and R¹³ is —OSO₂OR⁷. In some embodiments of Formula (I), (I′), (II), or (II′), R¹² is —OSO₂OR⁷; and R³ is —OSO₂OR⁷. In some embodiments of Formula (I), (I′), (II), or (II′), R¹² is —O(SO)OR⁷; and R¹³ is —OSO₂OR⁷. In some embodiments of Formula (I), (I′), (II), or (II′), R¹² is —PO(OR⁷)₂; and R¹³ is —OSO₂OR⁷. In some embodiments of Formula (I), (I′), (II), or (II′), R¹² is —PO(NR⁷)₂; and R¹³ is —OSO₂OR⁷. In some embodiments of Formula (I), (I′), (II), or (II′), R¹² is —PO(NR⁷)(OR⁷); and R¹³ is —OSO₂OR⁷. In some embodiments of Formula (I), (I′), (II), or (II′), R¹² is —OPO(OR⁷)₂; and R¹³ is —OSO₂OR⁷. In some embodiments of Formula (I), (I′), (II), or (II′), R¹² is —OPO(NR⁷)₂; and R¹³ is —OSO₂OR⁷. In some embodiments of Formula (I), (I′), (II), or (II′), R¹² is —OPO(NR⁷)(OR⁷); and R¹³ is —OSO₂OR⁷. In some embodiments of Formula (I), (I′), (II), or (II′), R¹² is a nitrogen protecting group; and R¹³ is —OSO₂OR⁷. In some embodiments of Formula (I), (I′), (II), or (II′), R¹² is —OR⁷; and R¹³ is —OSO₂OR⁷

In some embodiments of Formula (I), (I′), (II), or (II′), R¹² is hydrogen; and R¹³ is —OR⁷. In some embodiments of Formula (I), (I′), (II), or (II′), R¹² is substituted or unsubstituted C₁₋₆ alkyl; and R¹³ is —OR⁷. In some embodiments of Formula (I), (I′), (II), or (II′), R¹² is substituted or unsubstituted C₁₋₃ alkyl; and R¹³ is —OR⁷. In some embodiments of Formula (I), (I′), (II), or (II′), R¹² is branched or unbranched substituted or unsubstituted C₁₋₆ alkyl; and R¹³ is —OR⁷. In some embodiments of Formula (I), (I′), (II), or (II′), R¹² is substituted or unsubstituted methyl; and R¹³ is —OR⁷. In some embodiments of Formula (I), (I′), (II), or (II′), R¹² is substituted or unsubstituted ethyl; and R¹³ is —OR⁷. In some embodiments of Formula (I), (I′), (II), or (II′), R¹² is substituted or unsubstituted propyl; and R¹³ is —OR⁷. In some embodiments of Formula (I), (I′), (II), or (II′), R¹² is substituted or unsubstituted butyl; and R¹³ is —OR⁷. In some embodiments of Formula (I), (I′), (II), or (II′), R¹² is substituted or unsubstituted pentyl; and R¹³ is —OR⁷. In some embodiments of Formula (I), (I′), (II), or (II′), R¹² is substituted or unsubstituted C₁₋₆ haloalkyl; and R¹³ is —OR⁷. In some embodiments of Formula (I), (I′), (II), or (II′), R¹² is substituted or unsubstituted fluoroalkyl; and R¹³ is —OR⁷. In some embodiments of Formula (I), (I′), (II), or (II′), R¹² is trifluoromethyl; and R¹³ is —OR⁷. In some embodiments of Formula (I), (I′), (II), or (II′), R¹² is substituted or unsubstituted trifluoroethyl; and R¹³ is —OR⁷. In some embodiments of Formula (I), (I′), (II), or (II′), R¹² is substituted or unsubstituted C₂₋₆ alkenyl; and R¹³ is —OR⁷. In some embodiments of Formula (I), (I′), (II), or (II′), R¹² is substituted or unsubstituted C₂₋₆ alkenyl; and R¹³ is —OR⁷. In some embodiments of Formula (I), (I′), (II), or (II′), R¹² is substituted or unsubstituted vinyl; and R¹³ is —OR⁷. In some embodiments of Formula (I), (I′), (II), or (II′), R¹² is substituted or unsubstituted allyl; and R¹¹ is —OR⁷. In some embodiments of Formula (I), (I′), (II), or (II′), R¹² is substituted or unsubstituted propenyl; and R¹³ is —OR⁷. In some embodiments of Formula (I), (I′), (II), or (II′), R¹² is substituted or unsubstituted butenyl; and R¹³ is —OR⁷. In some embodiments of Formula (I), (I′), (II), or (II′), R¹² is substituted or unsubstituted C₂₋₆ alkynyl; and R¹³ is —OR⁷. In some embodiments of Formula (I), (I′), (II), or (II′), R¹² is substituted or unsubstituted acetylenyl; and R¹³ is —OR⁷. In some embodiments of Formula (I), (I′), (II), or (II′), R¹² is substituted or unsubstituted propargyl; and R¹³ is —OR⁷. In some embodiments of Formula (I), (I′), (II), or (II′), R¹² is substituted or unsubstituted butynyl; and R¹³ is —OR⁷. In some embodiments of Formula (I), (I′), (II), or (II′), R¹² is substituted or unsubstituted C₁₋₆ alkoxy; and R¹³ is —OR⁷. In some embodiments of Formula (I), (I′), (II), or (II′), R¹² is CH₂—OMe; and R¹³ is —OR⁷. In some embodiments of Formula (I), (I′), (II), or (II′), R¹² is CH-OEt; and R¹³ is —OR⁷. In some embodiments of Formula (I), (I′), (II), or (II′), R¹²CH₂—OPr; and R¹³ is —OR⁷In some embodiments of Formula (I), (I′), (II), or (II′), R¹² is CH₂—OCF₃; and R¹³ is —OR⁷In some embodiments of Formula (I), (I′), (II), or (II′), R¹² is substituted or unsubstituted CH₂—OR⁷; and R¹³ is —OR⁷. In some embodiments of Formula (I), (I′), (II), or (II′), R¹² is substituted or unsubstituted C₆₋₁₀ aryl; and R¹³ is —OR⁷. In some embodiments of Formula (I), (I′), (II), or (II′), R¹² is substituted or unsubstituted 5-10 membered heteroaryl; and R¹³ is —OR⁷. In some embodiments of Formula (I), (I′), (II), or (II′), R¹² is substituted or unsubstituted 4-10 membered heterocyclyl; and R¹³ is —OR⁷. In some embodiments of Formula (I), (I′), (II), or (II′), R¹² is substituted or unsubstituted 3-10 membered carbocyclyl; and R¹³ is —OR⁷. In some embodiments of Formula (I), (I′), (II), or (II′), R¹² is acyl; and R¹³ is —OR⁷. In some embodiments of Formula (I), (I′), (II), or (II′), R¹² is a substituted or unsubstituted amide; and R¹³ is —OR⁷. In some embodiments of Formula (I), (I′), (II), or (II′), R¹² is a substituted or unsubstituted ester; and R¹³ is —OR⁷. In some embodiments of Formula (I), (I′), (II), or (II′), R¹² is —OH; and R¹³ is —OR⁷. In some embodiments of Formula (I), (I′), (II), or (II′), R¹² is —SO₂R⁷; and R¹³ is —OR⁷. In some embodiments of Formula (I), (I′), (II), or (II′), R¹² is —SOR⁷; and R¹³ is —OR⁷. In some embodiments of Formula (I), (I′), (II), or (II′), R¹² is —SO₂NR⁷₂; and R¹³ is —OR⁷. In some embodiments of Formula (I), (I′), (II), or (II′), R¹² is —OSO₂R⁷; and R¹³ is —OR⁷. In some embodiments of Formula (I), (I′), (II), or (II′), R¹² is —OSOR⁷; and R¹³ is —OR⁷. In some embodiments of Formula (I), (I′), (II), or (II′), R¹² is —OSO₂NR⁷₂; and R¹³ is —OR⁷. In some embodiments of Formula (I), (I′), (II), or (II′), R¹² is —OSO₂OR⁷; and R¹³ is —OR⁷. In some embodiments of Formula (I), (I′), (II), or (II′), R¹² is —O(SO)OR⁷; and R¹³ is —OR⁷. In some embodiments of Formula (I), (I′), (II), or (II′), R¹² is —PO(OR⁷)₂; and R¹³ is —OR⁷. In some embodiments of Formula (I), (I′), (II), or (II′), R¹² is —PO(NR⁷)₂; and R¹³ is —OR⁷. In some embodiments of Formula (I), (I′), (II), or (II′), R¹² is —PO(NR⁷)(OR⁷); and R¹³ is —OR⁷. In some embodiments of Formula (I), (I′), (II), or (II′), R¹² is —OPO(OR⁷)₂; and R¹³ is —OR⁷. In some embodiments of Formula (I), (I′), (II), or (II′), R¹² is —OPO(NR⁷)₂; and R¹³ is —OR⁷. In some embodiments of Formula (I), (I′), (II), or (II′), R¹² is —OPO(NR⁷)(OR⁷); and R¹³ is —OR⁷. In some embodiments of Formula (I), (I′), (II), or (II′), R¹² is a nitrogen protecting group; and R¹³ is —OR⁷. In some embodiments of Formula (I), (I′), (II), or (II′), R¹² is —OR⁷; and R¹³ is —OR⁷.

In some embodiments of Formula (I), (I′), (II), or (II′), R¹² and R¹³ are taken together with any intervening atoms to form a substituted or unsubstituted heteroaryl ring. In some embodiments of Formula (I), (I′), (II), or (II′), R¹² and R¹³ are taken together with any intervening atoms to form a substituted or unsubstituted heterocyclic ring. In some embodiments of Formula (I), (I′), (II), or (II′), the ring formed by R¹² and R¹³ is optionally substituted with 5 occurrences of R⁵. In some embodiments of Formula (I), (I′), (II), or (II′), the ring formed by R¹² and R¹³ is optionally substituted with 4 occurrences of R⁵. In some embodiments of Formula (I), (I′), (II), or (II′), the ring formed by R¹² and R¹³ is optionally substituted with 3 occurrences of R⁵. In some embodiments of Formula (I), (I′), (II), or (II′), the ring formed by R¹² and R¹³ is optionally substituted with 2 occurrences of R⁵. In some embodiments of Formula (I), (I′), (II), or (II′), the ring formed by R¹² and R¹³ is optionally substituted with 1 occurrence of R⁵. In some embodiments of Formula (I), (I′), (II), or (II′), the ring formed by R¹² and R¹³ is optionally substituted with 0 occurrences of R⁵.

In some embodiments of Formula (I), (I′), (II), or (II′), R¹¹ is hydrogen,

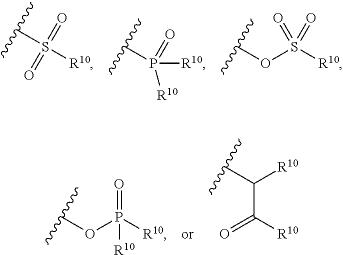

wherein

R¹⁰ is independently selected from hydrogen, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ alkynyl, substituted or unsubstituted C₁₋₆ alkoxy, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷.

In some embodiments of Formula (I), (I′), (II), or (II′), R¹¹ is hydrogen. In some embodiments of Formula (I), (I′), (II), or (II′), R¹¹ is

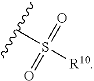

In some embodiments of Formula (I), (I′), (II), or (II′), R¹¹ is

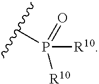

In some embodiments of Formula (I), (I′), (II), or (II′), R¹¹ is

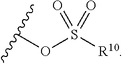

In some embodiments of Formula (I), (I′), (II), or (II′), R¹¹ is

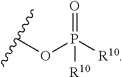

In some embodiments of Formula (I), (I′), (II), or (II′), R¹¹ is

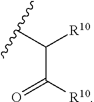

In some embodiments of Formula (I), (I′), (II), or (II′), R¹⁰ is independently hydrogen. In some embodiments of Formula (I), (I′), (II), or (II′), R¹⁰ is independently substituted or unsubstituted C₁₋₆ alkyl. In some embodiments of Formula (I), (I′), (II), or (II′), R¹⁰ is independently substituted or unsubstituted C₁₋₆ haloalkyl. In some embodiments of Formula (I), (I′), (II), or (II′), R¹⁰ is independently substituted or unsubstituted C₂₋₆ alkenyl. In some embodiments of Formula (I), (I′), (II), or (II′), R¹⁰ is independently substituted or unsubstituted C₂₋₆ alkynyl. In some embodiments of Formula (I), (I′), (II), or (II′), R¹⁰ is independently substituted or unsubstituted C₁₋₆ alkoxy. In some embodiments of Formula (I), (I′), (II), or (II′), R¹⁰ is independently an substituted or unsubstituted C₆₋₁₀ aryl ring. In some embodiments of Formula (I), (I′), (II), or (II′), R¹⁰ is independently an substituted or unsubstituted 5-10 membered heteroaryl ring. In some embodiments of Formula (I), (I′), (II), or (II′), R¹⁰ is independently an substituted or unsubstituted 4-10 membered heterocyclyl ring. In some embodiments of Formula (I), (I′), (II), or (II′), R¹⁰ is independently an substituted or unsubstituted 3-10 membered carbocyclyl ring. In some embodiments of Formula (I), (I′), (II), or (II′), R¹⁰ is independently nitro. In some embodiments of Formula (I), (I′), (II), or (II′), R¹⁰ is independently cyano. In some embodiments of Formula (I), (I′), (II), or (II′), R¹⁰ is independently acyl. In some embodiments of Formula (I), (I′), (II), or (II′), R¹⁰ is independently —NH₂. In some embodiments of Formula (I), (I′), (II), or (II′), R¹⁰ is independently —NHR⁷. In some embodiments of Formula (I), (I′), (II), or (II′), R¹⁰ is independently —N(R⁷)₂. In some embodiments of Formula (I), (I′), (II), or (II′), R¹⁰ is independently —OH. In some embodiments of Formula (I), (I′), (II), or (II′), R¹⁰ is independently —SH. In some embodiments of Formula (I), (I′), (II), or (II′), R¹⁰ is independently —SO₂R⁷. In some embodiments of Formula (I), (I′), (II), or (II′), R¹⁰ is independently —SOR⁷. In some embodiments of Formula (I), (I′), (II), or (II′), R¹⁰ is independently —SO₂NR⁷₂. In some embodiments of Formula (I), (I′), (II), or (II′), R¹⁰ is independently —SR⁷. In some embodiments of Formula (I), (I′), (II), or (II′), R¹⁰ is independently —OR⁷.

In some embodiments of Formula (I), (I′), (II), or (II′), at least one R⁷ is hydrogen. In some embodiments of Formula (I), (I′), (II), or (II′), at least one R⁷ is acyl. In some embodiments of Formula (I), (I′), (II), or (II′), at least one R⁷ is substituted or unsubstituted C₁₋₁₀ alkyl. In some embodiments of Formula (I), (I′), (II), or (II′), at least one R⁷ is substituted or unsubstituted C₆₋₁₀ aryl. In some embodiments of Formula (I), (I′), (II), or (II′), at least one R⁷ is independently substituted or unsubstituted 5-10 membered heteroaryl. In some embodiments of Formula (I), (I′), (II), or (II′), at least one R⁷ is substituted or unsubstituted 4-10 membered heterocyclyl. In some embodiments of Formula (I), (I′), (II), or (II′), at least one R⁷ is substituted or unsubstituted 3-10 membered carbocyclyl. In some embodiments of Formula (I), (I′), (II), or (II′), two R⁷ groups are taken together with any intervening atoms to form an substituted or unsubstituted heterocyclic ring.

In some embodiments of Formula (I), (I′), (II), or (II′), R¹¹ is hydrogen. In some embodiments of Formula (I), (I′), (II), or (II′), R¹¹ is

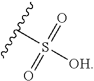 [SMILES: *S(=O)(=O)O]

In some embodiments of Formula (I), (I′), (II), or (II′), R¹¹ is

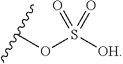 [SMILES: *OS(=O)(=O)O]

In some embodiments of Formula (I), (I′), (II), or (II′), R¹¹ is

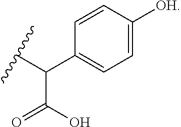 [SMILES: *C(C(=O)O)c1ccc(O)cc1]

In some embodiments of Formula (I), (I′), (II), or (II′), R¹¹ is not hydrogen. In some embodiments of Formula (I), (I′), (II), or (II′), R¹ is not

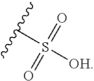 [SMILES: *S(=O)(=O)O]

In some embodiments of Formula (I), (I′), (II), or (II′), R¹ is not

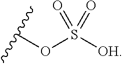 [SMILES: *OS(=O)(=O)O]

In some embodiments of Formula (I), (I′), (II), or (II′), R¹³ is not

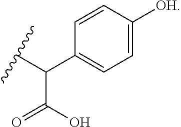 [SMILES: *C(C(=O)O)c1ccc(O)cc1]

In some embodiments of Formula (I), (I′), (II), or (II′), R¹² is H, and R¹³ is of the formula:

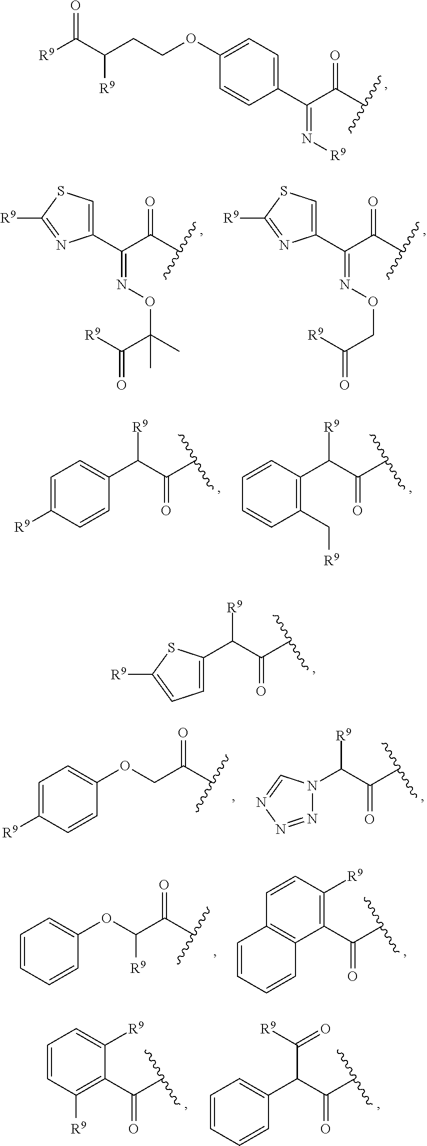

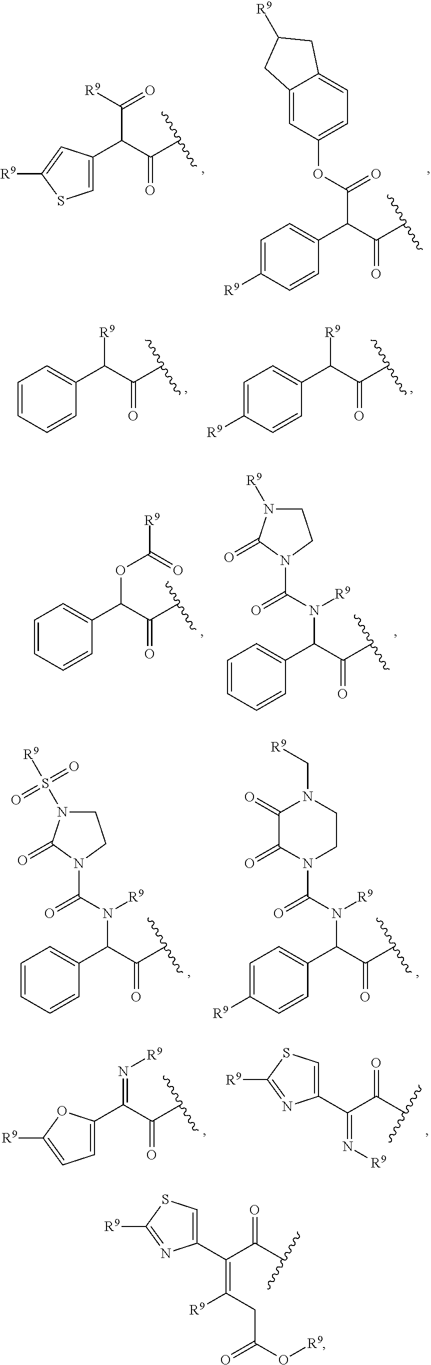

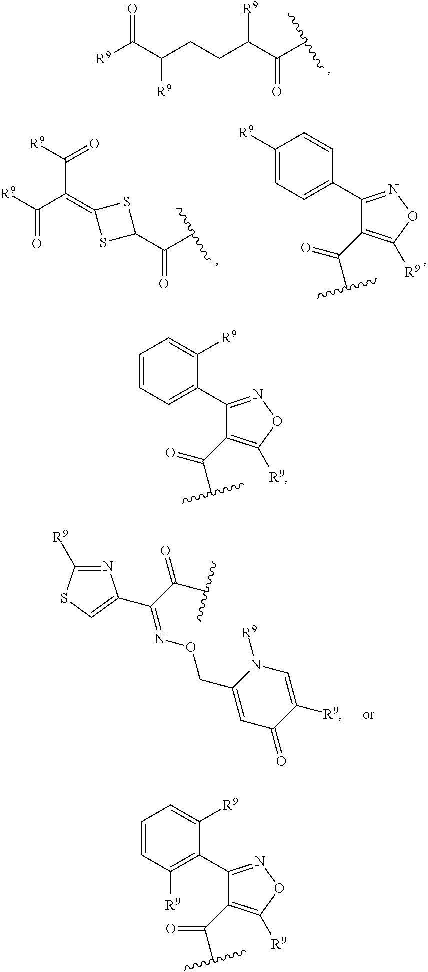

wherein:

R⁹ is independently selected from hydrogen, C₁₋₆ alkyl, C₁₋₆ haloalkyl, C₂₋₆ alkenyl, C₂₋₆ alkynyl, C₁₋₆ alkoxy, optionally substituted C₆₋₁₀ aryl, optionally substituted 5-10 membered heteroaryl, optionally substituted 4-10 membered heterocyclyl, optionally substituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷.

In some embodiments of Formula (I), (I′), (II), or (II′), R¹² is H, and R¹³ is

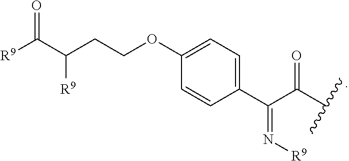

In some embodiments of Formula (I), (I′), (II), or (II′), R¹² is H, and R¹³ is of the formula:

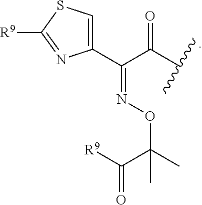

In some embodiments of Formula (I), (I′), (II), or (II′), R¹² is H, and R¹³ is

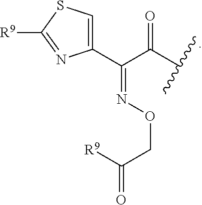

In some embodiments of Formula (I), (I′), (II), or (II′), R¹² is H, and R¹³ is

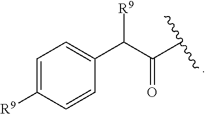

In some embodiments of Formula (I), (I′), (II), or (II′), R¹² is H, and R¹³ is

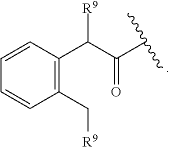

In some embodiments of Formula (I), (I′), (II), or (II′), R¹² is H, and R¹³ is

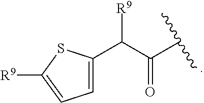

In some embodiments of Formula (I), (I′), (II), or (II′), R¹² is H, and R¹³ is

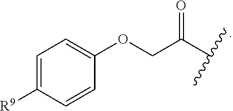

In some embodiments of Formula (I), (I′), (II), or (II′), R¹² is H, and R¹³ is

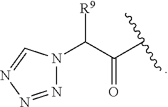

In some embodiments of Formula (I), (I′), (II), or (II′), R¹² is H, and R¹³ is

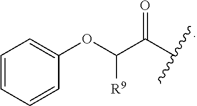

In some embodiments of Formula (I), (I′), (II), or (II′), R¹² is H, and R¹³ is

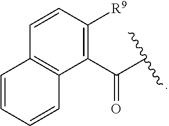

In some embodiments of Formula (I), (I′), (II), or (II′), R¹² is H, and R¹³ is

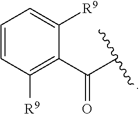

In some embodiments of Formula (I), (I′), (II), or (II′), R² is H, and R¹³ is

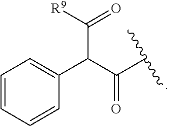

In some embodiments of Formula (I), (I′), (II), or (II′), R¹² is H, and R¹³ is

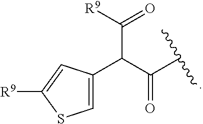

In some embodiments of Formula (I), (I′), (II), or (II′), R¹² is H, and R¹³ is

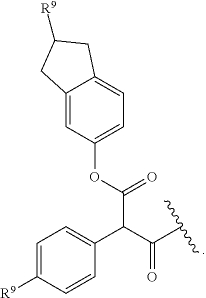

In some embodiments of Formula (I), (I′), (II), or (II′), R¹² is H, and R¹³ is

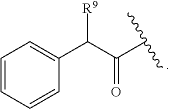

In some embodiments of Formula (I), (I′), (II), or (II′), R¹² is H, and R¹³ is

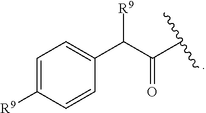

In some embodiments of Formula (I), (I′), (II), or (II′), R¹² is H, and R¹³ is

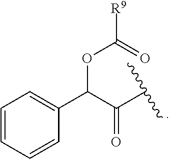

In some embodiments of Formula (I), (I′), (II), or (II′), R¹² is H, and R¹³ is

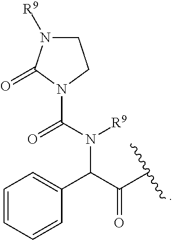

In some embodiments of Formula (I), (I′), (II), or (II′), R¹² is H, and R¹³ is

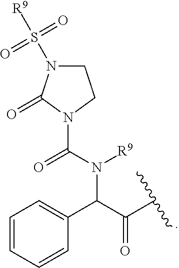

In some embodiments of Formula (I), (I′), (II), or (II′), R¹² is H, and R¹³ is

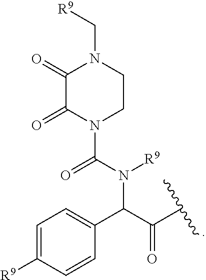

In some embodiments of Formula (I), (I′), (II), or (II′), R¹² is H, and R¹³ is

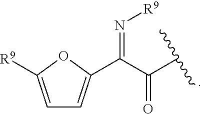

In some embodiments of Formula (I), (I′), (II), or (II′), R¹² is H, and R¹³ is

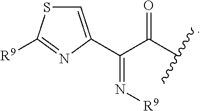

In some embodiments of Formula (I), (I′), (II), or (II′), R¹² is H, and R¹³ is

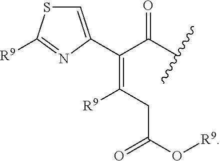

In some embodiments of Formula (I), (I′), (II), or (II′), R¹² is H, and R¹³ is

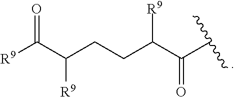

In some embodiments of Formula (I), (I′), (II), or (II′), R¹² is H, and R¹³ is

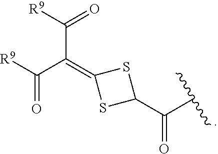

In some embodiments of Formula (I), (I′), (II), or (II′), R¹² is H, and R¹³ is

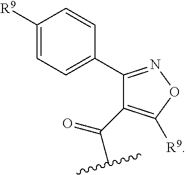

In some embodiments of Formula (I), (I′), (II), or (II′), R¹² is H, and R¹³ is

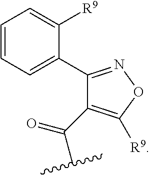

In some embodiments of Formula (I), (I′), (II), or (II′), R¹² is H, and R¹³ is

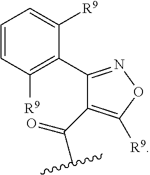

In some embodiments of Formula (I), (I′), (II), or (II′), R¹² is H, and R¹³ is

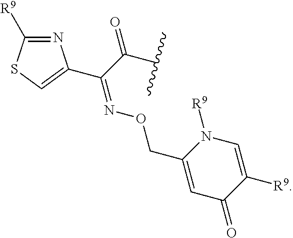

In some embodiments of Formula (I), (I′), (II), or (II′), at least one R⁹ is hydrogen. In some embodiments of Formula (I), (I′), (II), or (II′), at least one R⁹ is substituted or unsubstituted C₁₋₆ alkyl. In some embodiments of Formula (I), (I′), (II), or (II′), at least one R⁹ is substituted or unsubstituted C₁₋₆ haloalkyl. In some embodiments of Formula (I), (I′), (II), or (II′), at least one R⁹ is substituted or unsubstituted C₂₋₆ alkenyl. In some embodiments of Formula (I), (I′), (II), or (II′), at least one R⁹ is substituted or unsubstituted C₂₋₆ alkynyl. In some embodiments of Formula (I), (I′), (II), or (II′), at least one R⁹ is substituted or unsubstituted C₁₋₆ alkoxy. In some embodiments of Formula (I), (I′), (II), or (II′), at least one R⁹ is substituted or unsubstituted C₆₋₁₀ aryl. In some embodiments of Formula (I), (I′), (II), or (II′), at least one R⁹ is substituted or unsubstituted 5-10 membered heteroaryl. In some embodiments of Formula (I), (I′), (II), or (II′), at least one R⁹ is substituted or unsubstituted 4-10 membered heterocyclyl. In some embodiments of Formula (I), (I′), (II), or (II′), at least one R⁹ is substituted or unsubstituted 3-10 membered carbocyclyl. In some embodiments of Formula (I), (I′), (II), or (II′), at least one R⁹ is —NO₂. In some embodiments of Formula (I), (I′), (II), or (II′), at least one R⁹ is —CN. In some embodiments of Formula (I), (I′), (II), or (II′), at least one R⁹ is acyl. In some embodiments of Formula (I), (I′), (II), or (II′), at least one R⁹ is —NH₂. In some embodiments of Formula (I), (I′), (II), or (II′), at least one R⁹ is —NHR⁷. In some embodiments of Formula (I), (I′), (II), or (II′), at least one R⁹ is —N(R⁷)₂. In some embodiments of Formula (I), (I′), (II), or (II′), at least one R⁹ is —OH. In some embodiments of Formula (I), (I′), (II), or (II′), at least one R⁹ is —SH. In some embodiments of Formula (I), (I′), (II), or (II′), at least one R⁹ is —SO₂R⁷. In some embodiments of Formula (I), (I′), (II), or (II′), at least one R⁹ is —SOR⁷. In some embodiments of Formula (I), (I′), (II), or (II′), at least one R⁹ is —SO₂NR⁷₂. In some embodiments of Formula (I), (I′), (II), or (II′), at least one R⁹ is —SR⁷. In some embodiments of Formula (I), (I′), (II), or (II′), at least one R⁹ is —OR⁷.

In some embodiments of Formula (I), (I′), (II), or (II′), R¹³ is hydrogen.

In some embodiments of Formula (I), (I′), (II), or (II′), R¹³ is

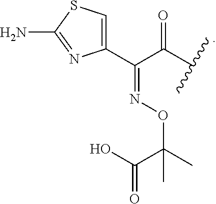 [SMILES: *C(=O)/C(=N\OC(C)(C)C(=O)O)c1csc(N)n1]

In some embodiments of Formula (I), (I′), (II), or (II′), R¹³ is

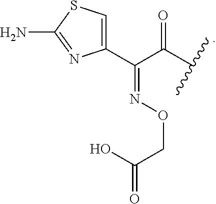 [SMILES: *C(=O)/C(=N\OCC(=O)O)c1csc(N)n1]

In some embodiments of Formula (I), (I′), (II), or (II′), R¹³ is

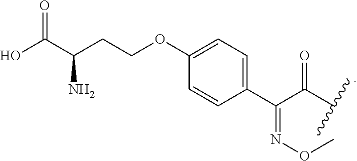 [SMILES: *C(=O)/C(=N\OC)c1ccc(OCC[C@@H](N)C(=O)O)cc1]

In some embodiments of Formula (I), (I′), (II), or (II′), R¹³ is

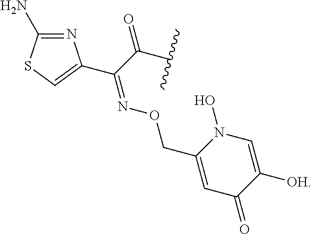 [SMILES: *C(=O)/C(=N\OCc1cc(=O)c(O)cn1O)c1csc(N)n1]

In some embodiments of Formula (I), (I′), (II), or (II′), R¹³ is not hydrogen.

In some embodiments of Formula (I), (I′), (II), or (II′), R¹³ is not

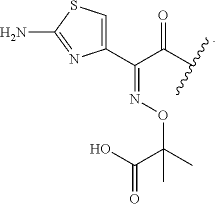 [SMILES: *C(=O)/C(=N\OC(C)(C)C(=O)O)c1csc(N)n1]

In some embodiments of Formula (I), (I′), (II), or (II′), R¹³ is not

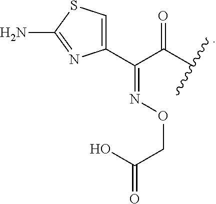 [SMILES: *C(=O)/C(=N\OCC(=O)O)c1csc(N)n1]

In some embodiments of Formula (I), (I′), (II), or (II′), R¹³ is not

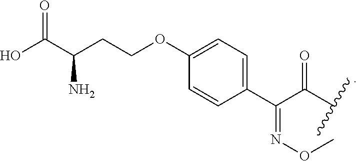 [SMILES: *C(=O)/C(=N\OC)c1ccc(OCC[C@@H](N)C(=O)O)cc1]

In some embodiments of Formula (I), (I′), (II), or (II′), R¹³ is not

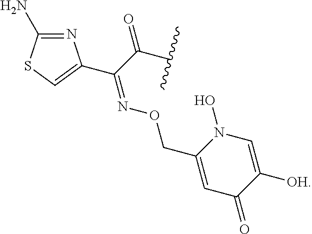 [SMILES: *C(=O)/C(=N\OCc1cc(=O)c(O)cn1O)c1csc(N)n1]

In some embodiments of Formula (I), (I′), (II), or (II′), R¹ is sterically larger than R².

In some embodiments of Formula (I), (I′), (II), or (II′), R¹ is sterically smaller than R².

### Pharmaceutical Compositions and Administration

Further provided are pharmaceutical compositions comprising a monobactam as described herein, or a pharmaceutically acceptable salt thereof, and optionally a pharmaceutically acceptable excipient.

The present disclosure provides pharmaceutical compositions comprising a monobactam described herein, e.g., a compound of Formula (I), (I′), (II), or (II′), or a pharmaceutically acceptable salt thereof, as described herein, and optionally a pharmaceutically acceptable excipient. It will be understood by one of ordinary skill in the art that the monobactams described herein, or salts thereof, may be present in various forms, such as an amorphous form, hydrates, solvates, or polymorphic forms. In certain embodiments, a provided composition comprises two or more compounds described herein. In certain embodiments, a monobactam described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting bacterial wall synthesis. In certain embodiments, the effective amount is an amount effective for inhibiting bacterial cell growth or proliferation. In certain embodiments, the effective amount is an amount effective to kill bacteria. In certain embodiments, the effective amount is an amount effective for treating a Gram-positive bacterial infection. In certain embodiments, the effective amount is an amount effective for treating a Gram-negative bacterial infection. In certain embodiments, the effective amount is a prophylactically effective amount. In certain embodiments, the effective amount is an amount effective to prevent a Gram positive bacterial infection. In certain embodiments, the effective amount is an amount effective to prevent a Gram negative bacterial infection.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in Formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the monobactam into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the monobactam as described herein. The amount of the monobactam is generally equal to the dosage of the monobactam which would be administered to a subject and/or a convenient fraction of such a dosage such as, for example, one-half or one-third of such a dosage.

Relative amounts of the monobactam, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) monobactam.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the monobactams, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable Formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the monobactam.

Solid dosage forms for oral administration include capsules, tablets, pills, powders, and granules. In such solid dosage forms, the monobactam is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical Formulating art. They may optionally comprise opacifying agents and can be of a composition that they release the monobactam(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The monobactam can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical Formulating art. In such solid dosage forms the monobactam can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the monobactam(s) only, or preferentially, in a certain part of the intestinal tract, optionally, in a delayed manner. Examples of embedding compositions which can be used include polymeric substances and waxes.

Dosage forms for topical and/or transdermal administration of a monobactam of this invention may include ointments, pastes, creams, lotions, gels, powders, solutions, sprays, inhalants and/or patches. Generally, the monobactam is admixed under sterile conditions with a pharmaceutically acceptable carrier and/or any needed preservatives and/or buffers as can be required. The use of transdermal patches is further contemplated, which often have the added advantage of providing controlled delivery of an monobactam to the body. Such dosage forms can be prepared, for example, by dissolving and/or dispensing the monobactam in the proper medium. Alternatively or additionally, the rate can be controlled by either providing a rate controlling membrane and/or by dispersing the monobactam in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable Formulations may, for example, comprise from about 1% to about 10% (w/w) monobactam, although the concentration of the monobactam can be as high as the solubility limit of the monobactam in the solvent. Formulations for topical administration may further comprise one or more of the additional ingredients described herein.

A pharmaceutical composition of the invention can be prepared, packaged, and/or sold in a Formulation suitable for pulmonary administration via the buccal cavity. Such a Formulation may comprise dry particles which comprise the monobactam and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the monobactam dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the monobactam may constitute 0.1 to 20% (w/w) of the composition. The propellant may further comprise additional ingredients such as a liquid non-ionic and/or solid anionic surfactant and/or a solid diluent (which may have a particle size of the same order as particles comprising the monobactam).

Pharmaceutical compositions of the invention formulated for pulmonary delivery may provide the monobactam in the form of droplets of a solution and/or suspension. Such Formulations can be prepared, packaged, and/or sold as aqueous and/or dilute alcoholic solutions and/or suspensions, optionally sterile, comprising the monobactam, and may conveniently be administered using any nebulization and/or atomization device. Such Formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another Formulation suitable for intranasal administration is a coarse powder comprising the monobactam and having an average particle from about 0.2 to 500 micrometers. Such a Formulation is administered. by rapid inhalation through the nasal passage from a container of the powder held close to the nares.

Formulations for nasal administration may, for example, comprise from about as little as 0.1% (w/w) and as much as 100% (w/w) of the monobactam, and may comprise one or more of the additional ingredients described herein. A pharmaceutical composition can be prepared, packaged, and/or sold in a Formulation for buccal administration. Such Formulations may, for example, be in the form of tablets and/or lozenges made using conventional methods, and may contain, for example, 0.1 to 20% (w/w) monobactam, the balance comprising an orally dissolvable and/or degradable composition and, optionally, one or more of the additional ingredients described herein. Alternately, Formulations for buccal administration may comprise a powder and/or an aerosolized and/or atomized solution and/or suspension comprising the monobactam. Such powdered, aerosolized, and/or aerosolized Formulations, when dispersed, may have an average particle and/or droplet size in the range from about 0.1 to about 200 nanometers, and may further comprise one or more of the additional ingredients described herein.

A pharmaceutical composition can be prepared, packaged, and/or sold in a Formulation for ophthalmic administration. Such Formulations may, for example, be in the form of eye drops including, for example, a 0.1/1.0% (w/w) solution and/or suspension of the monobactam in an aqueous or oily liquid carrier. Such drops may further comprise buffering agents, salts, and/or one or more other of the additional ingredients described herein. Other opthalmically-administrable Formulations which are useful include those which comprise the monobactam in microcrystalline form and/or in a liposomal preparation. Ear drops and/or eye drops are contemplated as being within the scope of this invention.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Monobactams provided herein are typically formulated in dosage unit form for ease of administration and uniformity of dosage. It will be understood, however, that the total daily amount of the monobactam will be decided by the attending physician within the scope of sound medical judgment. The specific therapeutically effective dose level for any particular subject will depend upon a variety of factors including the disease, disorder, or condition being treated and the severity of the disorder; the activity of the specific monobactam employed; the specific composition employed; the age, body weight, general health, sex and diet of the subject; the time of administration, route of administration, and rate of excretion of the specific monobactam employed; the duration of the treatment; drugs used in combination or coincidental with the specific monobactam employed; and like factors well known in the medical arts.

The monobactams and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general the most appropriate route of administration will depend upon a variety of factors including the nature of the agent, the therapeutic regimen, and/or the condition of the subject. Oral administration is the preferred mode of administration. However, in certain embodiments, the subject may not be in a condition to tolerate oral administration, and thus intravenous, intramuscular, and/or rectal administration are also preferred altermative modes of adminsitration.

The exact amount of a monobactam required to achieve an effective amount will vary from subject to subject, depending, for example, on species, age, and general condition of a subject, severity of the side effects or disorder, identity of the particular monobactam(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a monobactam for administration one or more times a day to a 70 kg adult human may comprise about 0.1 mg to about 3000 mg, about 0.1 mg to about 2000 mg, about 0.1 mg to about 1000 mg, about 0.1 mg to about 100 mg, about 1 mg to about 100 mg, or about 10 mg to about 100 mg, of a monobactam per unit dosage form.

In certain embodiments, the monobactams may be administered at dosage levels sufficient to deliver from about 0.001 mg/kg to about 100 mg/kg, from about 0.01 mg/kg to about 100 mg/kg, from about 0.1 mg/kg to about 100 mg/kg, from about 0.5 mg/kg to about 100 mg/kg, from about 10 mg/kg to about 100 mg/kg, from about 20 mg/kg to about 100 mg/kg, and from about 25 mg/kg to about 100 mg/kg, of subject body weight per day, one or more times a day, to obtain the desired therapeutic effect.

It will be also appreciated that a monobactam or composition, as described herein, can be administered in combination with one or more additional therapeutically active agents. The monobactam or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the monobactam with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

In any of the above described methods, one or more additional therapeutic agents (also referred to as the “agent”) may be administered concurrently with, prior to, or subsequent to, the monobactam. The agent may be added at the same time as the monobactam (simultaneous administration), before or after administration of the monobactam (sequential administration), or any combination thereof. For example, in certain embodiments, the agent is administered first, followed by simultaneous administration of the agent and the monobactam. In certain embodiments, the monobactam is administered first, followed by simultaneous administration of the agent and the monobactam. In any of the above embodiments, either the agent or the monobactam may be further administered alone after the simultaneous administration.

Exemplary additional therapeutically active agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins.

Therapeutically active agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

In certain embodiments, the additional therapeutically agent is an antibiotic. Exemplary antibiotics include, but are not limited to, penicillins (e.g., penicillin, amoxicillin), cephalosporins (e.g., cephalexin), macrolides (e.g., erythromycin, clarithormycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, doxycline, aureomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, and quinupristin/dalfoprisin (Synercid™).

Also encompassed by the invention are kits (e.g., pharmaceutical packs). The kits provided may comprise a pharmaceutical composition or monobactam and a container (e.g., a vial, ampule, bottle, syringe, and/or dispenser package, or other suitable container). In some embodiments, provided kits may optionally further include a second container comprising a pharmaceutical excipient for dilution or suspension of an pharmaceutical composition or monobactam. In some embodiments, the pharmaceutical composition or monobactam provided in the container and the second container are combined to form one unit dosage form.

### Method of Treatment

Further provided are methods of using monobactams for the treatment of infectious diseases, for example, bacterial infections. Monobactams are known to exhibit anti-bacterial activity; see, for example, Dudley et al., Clin. Infect. Dis. (2013) 9:1301-1309, and Walker et al., Ann. Pharmacother. (2012) 46:1537-1546.

Thus, as generally described herein, provided is a method of treating a infectious disease comprising administering an effective amount of a monobactam, or a pharmaceutically acceptable salt thereof, to a subject in need thereof. Such a method can be conducted in vivo (i.e., by administration to a subject) or in vitro (e.g., upon contact with the pathogen, tissue, or cell culture). Treating, as used herein, encompasses therapeutic treatment and prophylactic treatment.

In certain embodiments, the effective amount is a therapeutically effective amount. For example, in certain embodiments, the method slows the progress of an infectious disease in the subject. In certain embodiments, the method improves the condition of the subject suffering from an infectious disease. In certain embodiments, the subject has a suspected or confirmed infectious disease. In certain embodiments, the subject has been exposed to bacteria in the surrounding enviroment. In certain embodiments, the subject has been exposed to bacteria originating from an animal vector. In certain embodiments, the subject has been exposed to bacteria originating from other persons with an infectious disease.

In certain embodiments, the effective amount is a prophylatically effective amount. For example, in certain embodiments, the method prevents or reduces the likelihood of an infectious disease, e.g., in certain embodiments, the method comprises administering a monobactam to a subject in need thereof in an amount sufficient to prevent or reduce the likelihood of an infectious disease. In certain embodiments, the subject is at risk of an infectious disease (e.g., has been exposed to another subject who has a suspected or confirmed infectious disease or has been exposed or thought to be exposed to a pathogen).

In another aspect, provided is an in vitro method of inhibiting pathogenic growth comprising contacting an effective amount of the monobactam with a pathogen (e.g., a bacteria) in a cell culture.

As used herein, “infectious disease” and “microbial infection” are used interchangeably, and refer to an infection with a pathogen, such as a bacterium. In certain embodiments, the infectious disease is caused by a pathogen resistant to other treatments. In certain embodiments, the infectious disease is caused by a pathogen that is multi-drug tolerant or resistant, e.g., the infectious disease is caused by a pathogen that neither grows nor dies in the presence of or as a result of other treatments.

In certain embodiments, the infectious disease is a bacterial infection. For example, in certain embodiments, provided is a method of treating a bacterial infection comprising administering an effective amount of a monobactam, or a pharmaceutically acceptable salt thereof, to a subject in need thereof.

In certain embodiments, the monobactam has a mean inhibitory concentration (MIC), with respect to a particular bacteria, of less than 50 μg/mL, less than 25 μg/mL, less than 20 μg/mL, less than 10 μg/mL, less than 5 μg/mL, or less than 1 μg/mL.

In certain embodiments, the bacteria is susceptible (e.g., responds to) or resistant to known monobactams such as aztreonam, carumonam, SQ26,445, SQ26,180, tigemonam, or nocardicin A; see Scheme 1 for a listing of known monobactams. In certain embodiments, the bactera is resistant to a known monobactam. For example, in certain embodiments, the bacteria is aztreonam resistant.

In certain embodiments, the bacterial infection is resistant to other antibiotics (e.g., non-monobactam) therapy. For example, in certain embodiments, the pathogen is penam resistant. In certain embodiments, the pathogen is cephem resistant. In certain embodiments, the pathogen is carbapenem resistant. In certain embodiments, the pathogen is penem resistant For example, in certain embodiments, the pathogen is vancomycin resistant (VR).

In certain embodiments, the pathogen is a methicillin-resistant (MR), e.g., in certain embodiments, the bacterial infection is an methicillin-resistant S. aureus infection (a MRSA infection).

In certain embodiments, the bacteria has an efflux (e.g., mef, msr) genotype. In certain embodiments, the bacteria has a methylase (e.g., erm) genotype. In certain embodiments, the bacteria has a constitutive genotype. In certain embodiments, the bacteria has an inducible genotype. In certain embodiments, the bacteria expresses an enzyme capable of inactivating a beta-lactam antibiotic. In certain embodiments, the bacteria expresses a beta-lactamase. In some embodiments, the bacteria expresses a group 1, group 2, group 3, or group 4 beta-lactamase. In some embodiments, the bacteria expresses a class A, B, C, or D beta-lactamase.

Exemplary bacterial infections include, but are not limited to, infections with a Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/Chlorobi/Bacteroidetes (FCB), phylum Fusobacteria, phylum Gemmatimonadest, phylum Ntrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacterial infection is an infection with a Gram positive bacteria.

In certain embodiments, the Gram positive bacteria is a bacteria of the phylum Firmicutes.

In certain embodiments, the bacteria is a member of the phylum Firmicutes and the genus Enterococcus, i.e., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus.

In certain embodiments, the bacteria is a member of the phylum Firmicutes and the genus Staphylococcus, i.e., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In certain embodiments, the Staphylococcus infection is an S. aureus infection. In certain embodiments, the S. aureus has an efflux (e.g., mef, msr) genotype. In certain embodiments, the S. aureus has a methylase (e.g., erm) genotype.

In certain embodiments, the bacteria is a member of the phylum Firmicutes and the genus Bacillus, i.e., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis. In certain embodiments, the Bacillus infection is a B. subtilis infection. In certain embodiments, the B. subtilis has an efflux (e.g., mef, msr) genotype. In certain embodiments, the B. subtilis has a methylase (e.g., erm) genotype.

In certain embodiments, the bacteria is a member of the phylum Firmicutes and the genus Strepococcus, i.e., the bacterial infection is a Strepococcus infection. Exemplary Strepococcus bacteria include, but are not limited to, S. agalactiae, S. anginosus, S. bovis, S. canis, S. constellatus, S. dysgalactiae, S. equinus, S. iniae, S. intermedius, S. mitis, S. mutans, S. oralis, S. parasanguinis, S. peroris, S. pneumoniae, S. pyogenes, S. ratti, S. salivarius, S. thermophilus, S. sanguinis, S. sobrinus, S. suis, S. uberis, S. vestibularis, S. viridans, and S. zooepidemicus. In certain embodiments, the Strepococcus infection is an S. pyogenes infection. In certain embodiments, the Strepococcus infection is an S. pneumoniae infection. In certain embodiments, the S. pneumoniae has an efflux (e.g., mef, msr) genotype. In certain embodiments, the S. pneumoniae has a methylase (e.g., erm) genotype.

In certain embodiments, the bacteria is a member of the Gram positive bacterium of one of the following genera: Actinomyces, Bacillus, Clostridium, Corynebacterium, Enterococcus, Erysipelothrix, Listeria, Peptostreptococcus, Staphylococcus, or Streptococcus.

In certain embodiments, the bacteria is a member of the Gram positive bacterium of one of the following species: Actinomyces israelii, Bacillus anthracis, Clostridium difficile, Clostridium perfringens, Corynebacterium diphtheriae, Enterococcus faecalis, Enterococcus faecium, Listeria monocytogenes, Staphylococcus aureus, Staphylococcus epidermidis, Streptococcus agalactiae, Streptococcus bovis, Streptococcus pneumoniae, Streptococcus pyogenes, or Viridans streptococci.

In certain embodiments, the bacterial infection is an infection with a Gram negative bacteria.

In certain embodiments, the Gram negtive bacteria is a bacteria of the phylum Proteobacteria and the genus Escherichia. i.e., the bacterial infection is an Escherichia infection. Exemplary Escherichia bacteria include, but are not limited to, E. albertii, E. blattae, E. coli, E. fergusonii, E. hermannii, and E. vulneris. In certain embodiments, the Escherichia infection is an E. coli infection.

In certain embodiments, the Gram negtive bacteria is a bacteria of the phylum Proteobacteria and the genus Haemophilus. i.e., the bacterial infection is an Haemophilus infection. Exemplary Haemophilus bacteria include, but are not limited to, H. aegyptius, H. aphrophilus, H. avium, H. ducreyi, H. felis, H. haemolyticus, H. influenzae, H. parainfluenzae, H. paracuniculus, H. parahaemolyticus, H. pittmaniae, Haemophilus segnis, and H. somnus. In certain embodiments, the Escherichia infection is an H. influenzae infection.

In certain embodiments, the Gram negative bacteria is a bacteria of one of the following genera: Acinetobacter, Aeromonas, Bacteroides, Burkholderia, Citrobacter, Enterobacter, Escherichia, Fusobacterium, Haemophilus, Klebsiella, Moraxella, Morganella, Mycoplasma, Neisseria, Pantoea, Pasteurella, Plesiomonas, Porphyromonas, Prevotella, Proteus, Providencia, Pseudomonas, Salmonella, Serratia, Shigella, Spirillum, Stenotrophomonas, Streptobacillus, Treponema, or Yersinia.

In certain embodiments, the Gram negative bacteria is a bacteria of one of the following species: Acinetobacter baumannii, Aeromonas hydrophila, Arizona hinshawii, Bacteroides fragilis, Branhamella catarrhalis, Burkholderia cepacia, Citrobacter diversus, Citrobacterfreundii, Enterobacter aerogenes, Enterobacter cloacae, Escherichia coli, Fusobacterium nucleatum, Haemophilus influenzae, Haemophilus parainfluenzae, Klebsiella oxytoca, Klebsiella pneumoniae, Moraxella catarrhalis, Morganella morganii, Neisseria gonorrhoeae, Neisseria meningitidis, Pantoea agglomerans, Pasteurella multocida, Plesiomonas shigelloides, Prevotella melaninogenica, Proteus mirabilis, Proteus rettgeri, Proteus vulgaris, Pseudomonas aeruginosa, Pseudomonas diminuta, Pseudomonas fluorescens, Pseudomonas stutzeri, Salmonella enterica, Salmonella enteritidis, Salmonella typhi, Serratia marcescens, Spirillum minus, Stenotrophomonas maltophilia, Streptobacillus moniliformis, Treponema pallidum, or Yersinia enterocolitica.

### Synthetic Methods

En route to the monobactams, the synthetic methodology utilizes a diastereoselective glycine aldol reaction with aldehyde and ketone substrates to generate diastereomerically enriched amino amides and derivatives thereof with high diastereoselectivity. The glycine aldol methodology works on a wide variety of substrates including both aldehyde and ketone electrophiles (see Scheme 2 and 3).

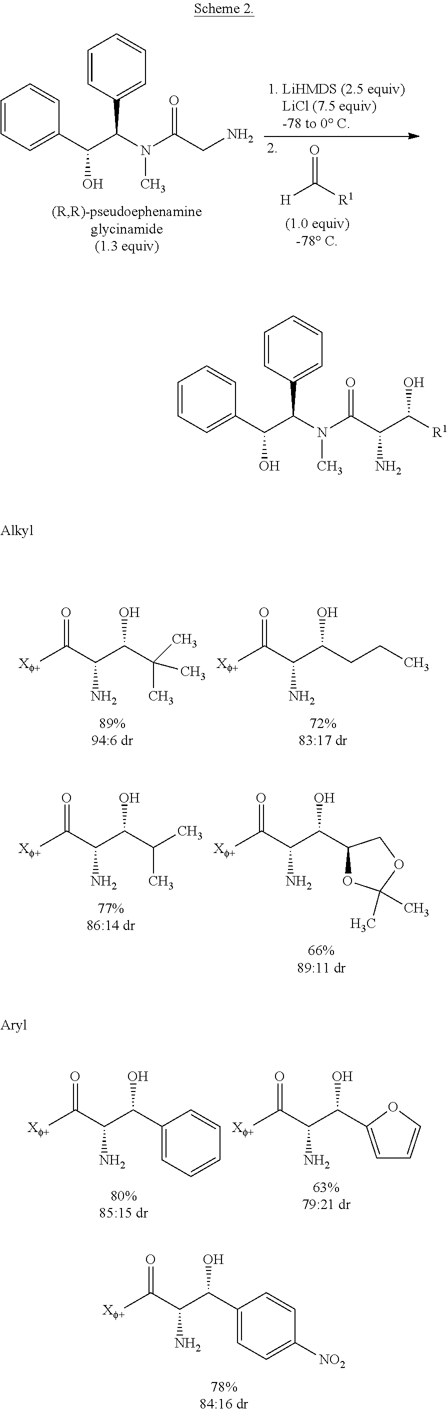

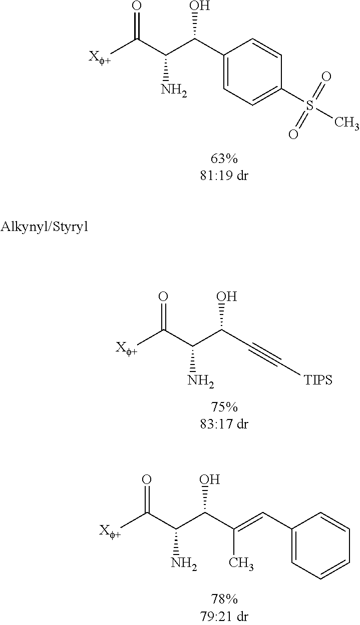

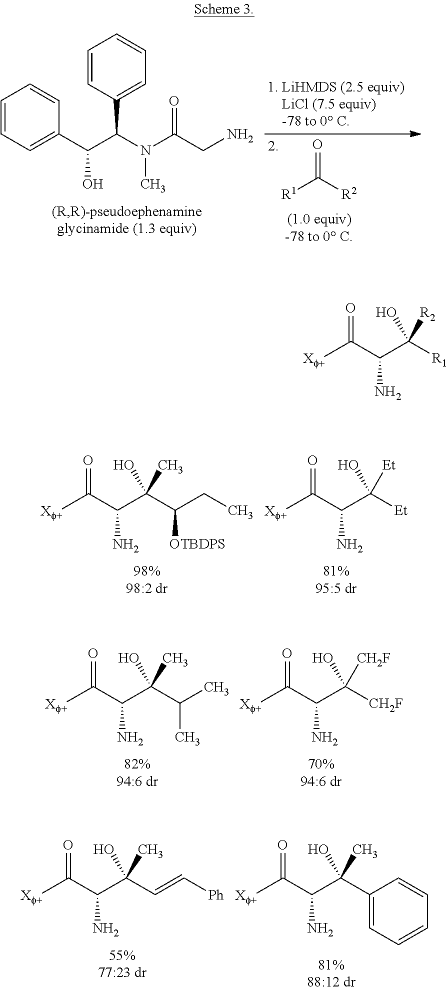

In some embodiments, R¹ is a sterically larger group than R² resulting in reaction outcomes as detailed below (see Scheme 4).

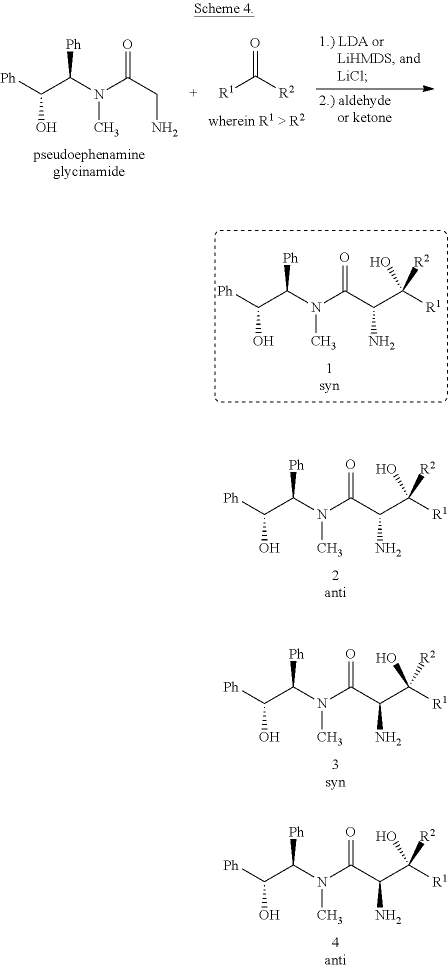

The general scheme for the reaction and the possible functionality of the chiral auxillary and intermediate is captured below (see Scheme 5).

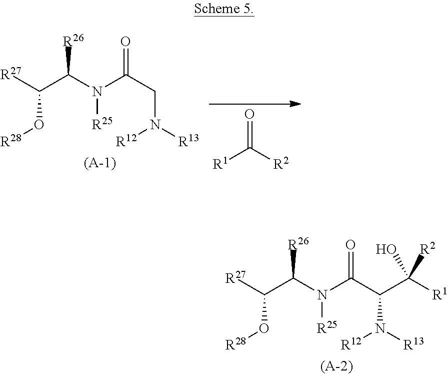

wherein:

R²⁵ is substituted or unsubstituted alkyl, substituted or unsubstituted aryl, substituted or unsubstituted heteroaryl, or a nitrogen protecting group;

R²⁶ and R²⁷ are each independently substituted or unsubstituted alkyl, substituted or unsubstituted, or substituted or unsubstituted heteroaryl; and

R²⁸ is hydrogen, substituted or unsubstituted alkyl, or an oxygen protecting group.

In certain embodiments, R²⁵ is optionally substituted C₁₋₆ alkyl. In certain embodiments, R² is unsubstituted C₁₋₆ alkyl. In certain embodiments, R² is methyl. In certain embodiments, R² is ethyl. In certain embodiments, R² is a nitrogen protecting group. In certain embodiments, R²⁵ is methyl, ethyl, or a nitrogen protecting group.

In certain embodiments, R²⁶ and R²⁷ are each independently optionally substituted alkyl. In certain embodiments, at least one of R²⁶ and R²⁷ is independently optionally substituted C₁₋₆ alkyl. In certain embodiments, at least one of R²⁶ and R²⁷ is unsubstituted alkyl. In certain embodiments, at least one of R²⁶ and R²⁷ is methyl. In certain embodiments, at least one of R²⁶ and R²⁷ is ethyl.

In certain embodiments, R²⁶ and R²⁷ are each independently optionally substituted aryl. In certain embodiments, at least one of R²⁶ and R²⁷ is independently optionally substituted aryl. In certain embodiments, at least one of R²⁶ and R²⁷ is independently optionally substituted phenyl. In certain embodiments, at least one of R²⁶ and R²⁷ is independently unsubstituted phenyl. In certain embodiments, both R²⁶ and R²⁷ are optionally substituted phenyl. In certain embodiments, both R²⁶ and R²⁷ are unsubstituted phenyl. In certain embodiments, R²⁶ is optionally substituted C₁₋₆ alkyl and R²⁷ is optionally substituted aryl. In certain embodiments, R²⁶ is unsubstituted C₁₋₆ alkyl and R²⁷ is optionally substituted phenyl. In certain embodiments, R² is methyl and R² is phenyl.

In certain embodiments, R²⁶ and R²⁷ are each independently optionally substituted heteroaryl, e.g., pyridinyl. In certain embodiments, at least one of R²⁶ and R²⁷ is independently optionally substituted heteroaryl. In certain embodiments, both R²⁶ and R²⁷ are optionally substituted heteroaryl. In certain embodiments, R²⁶ is optionally substituted C₁₋₆ alkyl and R²⁷ is optionally substituted heteroaryl.

In certain embodiments, R¹² is hydrogen. In certain embodiments, R¹³ is hydrogen. In certain embodiments, both R¹² and R¹³ are hydrogen.

In certain embodiments, R²⁸ is hydrogen. In certain embodiments, R²⁸ is optionally substituted alkyl, e.g. C₁₋₆ alkyl. In certain embodiments, R²⁸ is unsubstituted C₁₋₆ alkyl. In certain embodiments, R²⁸ is an oxygen protecting group.

In certain embodiments, the compound of Formula (A-1) is of the Formula:

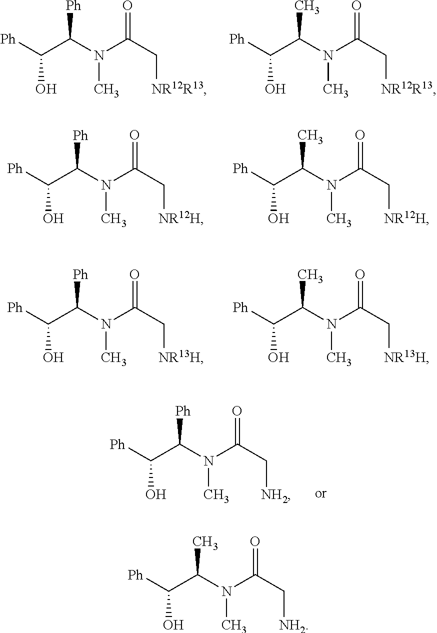

In certain embodiments, an organic base is present in the aldol reaction of Scheme 5. In certain embodiments, the organic base is a Group IA or Group IIA hydroxide or alkoxide, organo lithium, organo sodium, or organo magnesium. In certain embodiments, the base is organo lithium. In certain embodiments, the base is LDA. In certain embodiments, the base is LiHMDS. In certain embodiments, the base is NaHMDS. In certain embodiments, a Group IA or Group IIA halide salt is present in the aldol reaction of Scheme 5. In certain embodiments, a Group IA or Group IIA halide salt is present with the organic base in the aldol reaction of Scheme 5. In certain embodiments, the halide salt is a lithium halide. In certain embodiments, the halide salt is LiCl.

As depicted in Scheme 4, the glycine aldol reaction provides four diastereomeric products. These products are typically enriched in the stereoisomer 1 over all other stereoisomers. In certain embodiments, the aldol reaction of Scheme 4 provides a ratio of the stereoisomer 1 over all other stereoisomers of about 1:1 to about 100:1. In certain embodiments, the aldol reaction of Scheme 4 provides a ratio of the stereoisomer 1 over all other stereoisomers of about 1:1 to about 10:1. In certain embodiments, the aldol reaction of Scheme 4 provides a ratio of the stereoisomer 1 over all other stereoisomers of about 5:1 to about 10:1. In certain embodiments, the aldol reaction of Scheme 4 provides a ratio of the stereoisomer 1 over all other stereoisomers of about 10:1 to about 100:1. In certain embodiments, the aldol reaction of Scheme 4 provides a ratio of the stereoisomer 1 over all other stereoisomers of about 30:1 to about 100:1. In certain embodiments, the aldol reaction of Scheme 4 provides a ratio of the stereoisomer 1 over all other stereoisomers of about 50:1 to about 100:1. In certain embodiments, the aldol reaction of Scheme 4 provides a ratio of the stereoisomer 1 over all other stereoisomers of about 80:1 to about 100:1. In certain embodiments, the aldol reaction of Scheme 4 provides a ratio of the stereoisomer 1 over all other stereoisomers of about 90:1 to about 100:1.

Conversion of the diastereomerically enriched products from the aforementioned glycine aldol reaction into monobactams relies upon several transformations. Non-limiting examples of such transformations are depicted below (see Schemes 6-16).

As depicted in Scheme 6, following the enantioselective glycine aldol reaction, the chiral auxillary is converted to the corresponding carboxylic acid or ester (A-3) via conditions Si. Introduction of the amide function through conditions S2 leads to compounds of Formula (A-4). The carbinol functionality is converted into a leaving group under conditions S3, leading to subsequent lactamization and production of compounds of Formula (A-5).

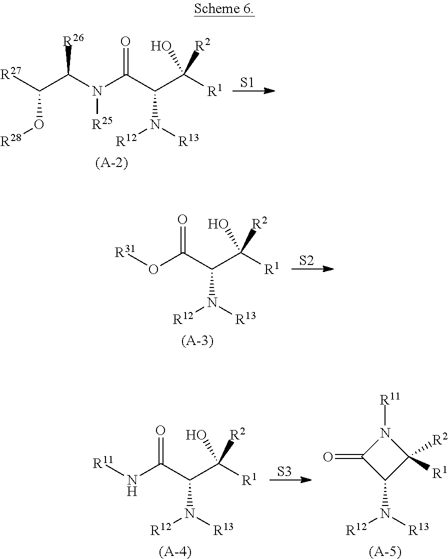

As depicted in Scheme 7, following the enantioselective glycine aldol reaction, the chiral auxillary is converted to the corresponding carboxylic acid or ester (A-7) via conditions S4. Introduction of the amide function through conditions S5 leads to compounds of Formula (A-8). The carbinol functionality is converted into a leaving group under conditions S6, leading to subsequent lactamization and production of compounds of Formula (A-9). The free N—H group is optionally functionalized through reaction with leaving group-conjugates of R¹³ under conditions S7 to produce compounds of Formula (A-10). In some embodiments, the leaving group conjugate of R³ is an acyl halide. In some embodiments, the leaving group conjugate of R³ is an sulfonyl halide. In some embodiments, the leaving group conjugate of R¹³ is an acid anhydride. In some embodiments, the leaving group conjugate of R³ is an sulfonyl anhydride. In some embodiments, the leaving group conjugate of R¹³ is an alkyl halide. In some embodiments, the leaving group conjugate of R¹³ is a carboxylic acid treated with an amide coupling reagent. In some embodiments, conditions S7 are basic.

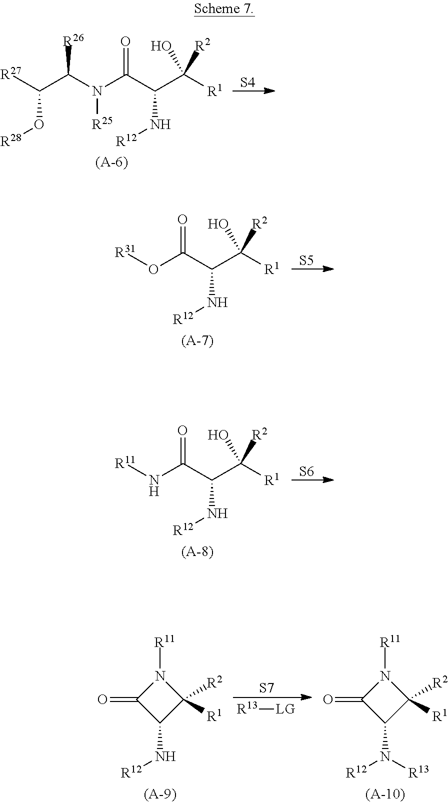

As depicted in Scheme 8, following the enantioselective glycine aldol reaction, the chiral auxillary is converted to the corresponding carboxylic acid or ester (A-12) via conditions S8. Introduction of the amide function through conditions S9 leads to compounds of Formula (A-13). The carbinol functionality is converted into a leaving group under conditions S10, leading to subsequent lactamization and production of compounds of Formula (A-9). The free N—H group is optionally functionalized through reaction with leaving group-conjugates of R¹² under conditions S11 to produce compounds of Formula (A-15). In some embodiments, the leaving group conjugate of R¹² is an acyl halide. In some embodiments, the leaving group conjugate of R¹² is an sulfonyl halide. In some embodiments, the leaving group conjugate of R¹² is an acid anhydride. In some embodiments, the leaving group conjugate of R¹² is an sulfonyl anhydride. In some embodiments, the leaving group conjugate of R¹² is an alkyl halide. In some embodiments, the leaving group conjugate of R¹² is a carboxylic acid treated with an amide coupling reagent. In some embodiments, conditions S11 are basic. The second free N—H group is optionally functionalized through reaction with leaving group-conjugates of R¹³ under conditions S12 to produce compounds of Formula (A-16).

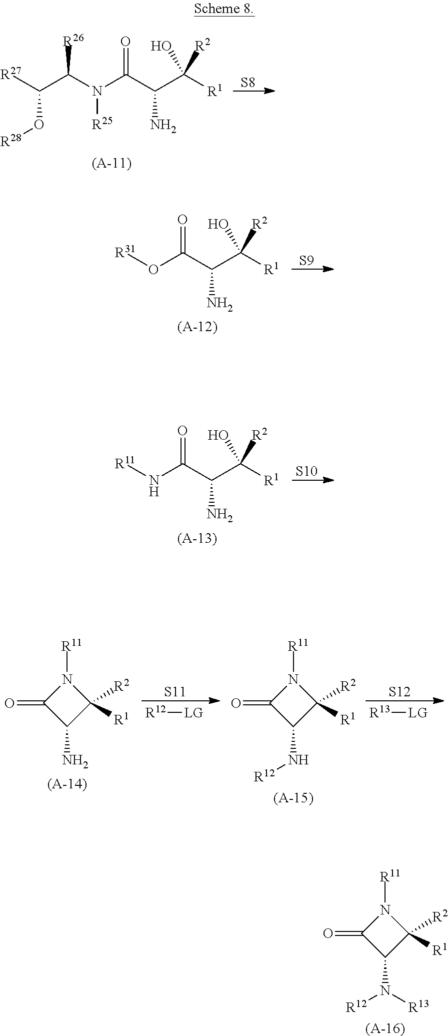

As depicted in Scheme 9, following the enantioselective glycine aldol reaction, the chiral auxillary is converted to the corresponding carboxylic acid or ester (A-18) via conditions S13. The free N—H is protected under conditions S14 to afford protected-amines of Formula (A-19). Introduction of a protected-amide function through conditions S15 leads to compounds of Formula (A-20). The carbinol functionality is converted into a leaving group under conditions S16, leading to subsequent lactamization and production of compounds of Formula (A-21). Deprotection of the amine protecting group PG¹ under conditions S17 leads to compounds of Formula (A-22). The free N—H group is optionally functionalized through reaction with leaving group-conjugates of R¹² under conditions S18 to produce compounds of Formula (A-20). The second free N—H group is optionally functionalized through reaction with leaving group-conjugates of R¹³ under conditions S19 to produce compounds of Formula (A-24). Deprotection of the amine protecting group PG² under conditions S20 leads to compounds of Formula (A-25). The free N—H group is optionally functionalized through reaction with leaving group-conjugates of R¹¹ under conditions S21 to produce compounds of Formula (A-26).

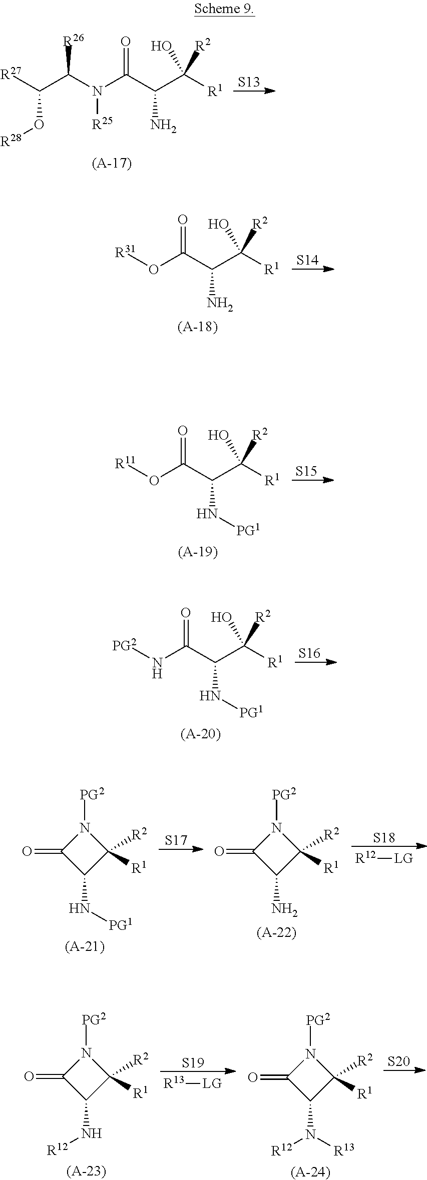

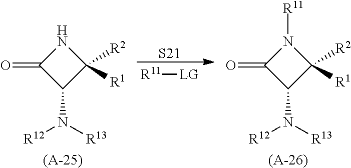

As depicted in Scheme 10, following the enantioselective glycine aldol reaction, the chiral auxiliary is converted to the corresponding carboxylic acid or ester (A-28) via conditions S22. Introduction of a protected-hydroxyamide function through conditions S23 leads to compounds of Formula (A-29). The carbinol functionality is converted into a leaving group under conditions S24, leading to subsequent lactamization and production of compounds of Formula (A-30). The free N—H group is optionally functionalized through reaction with leaving group-conjugates of R¹² under conditions S25 to produce compounds of Formula (A-31). The second free N—H group is optionally functionalized through reaction with leaving group-conjugates of R¹³ under conditions S26 to produce compounds of Formula (A-32). Deprotection of the oxygen protecting group PG³ under conditions S27 leads to compounds of Formula (A-33). The free O—H group is optionally functionalized through reaction with leaving group-conjugates of R³² under conditions S28 to produce compounds of Formula (A-34).

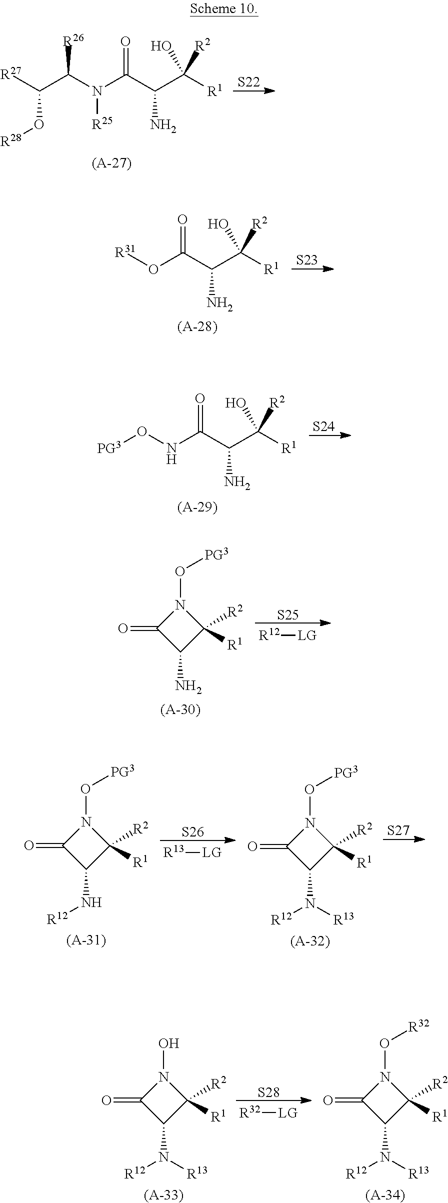

Alternatively, as depicted in Scheme 11, hydroxyamide-protected lactams of Formula (A-35) are converted into the free N—H lactams of Formula (A-36) under conditions S29. The free N—H group is optionally functionalized through reaction with leaving group-conjugates of R¹¹ under conditions S30 to produce compounds of Formula (A-37).

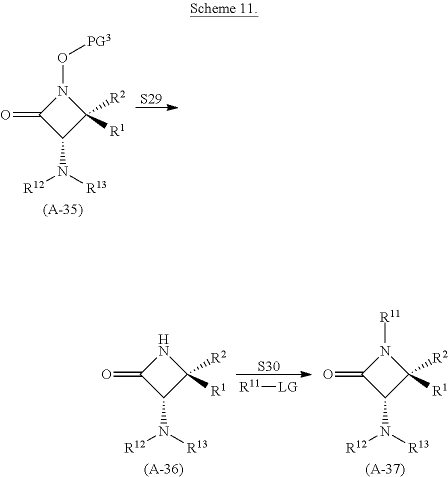

In certain embodiments, contacting compound of Formula (A-2), (A-6), (A-17), or (A-27) with water or an alcohol under either acidic or basic conditions provides carboxylic acids or esters of Formula (A-3), (A-7), (A-18), or (A-28) as shown in Scheme 12.

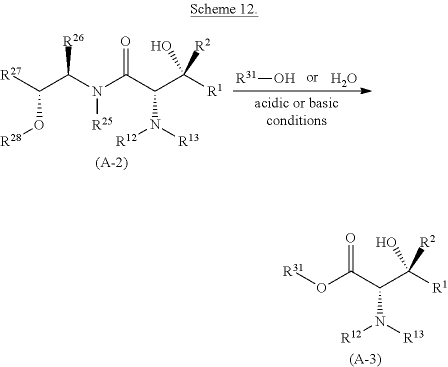

Alternatively, contacting a compound of Formula (A-2), (A-6), (A-17), or (A-27) with a hydroxylamine derivative under either acidic or basic conditions provides hydroxamic acids such as those of Formula (A-3*) as shown in Scheme 12a.

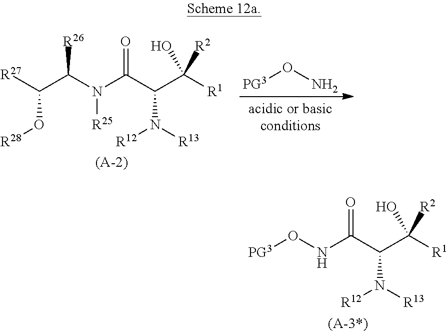

In certain embodiments, a compound of Formula (A-2), (A-6), (A-17), or (A-27) is contacted with an alcohol and R³¹ is C₁₋₆ alkyl. In certain embodiments, a compound of Formula (A-2), (A-6), (A-17), or (A-27) is contacted with an alcohol, and the alcohol is methanol, ethanol, propanol, isopropanol, butanol, or t-butanol. In certain embodiments, a compound of Formula (A-2), (A-6), (A-17), or (A-27) is contacted with water and R³¹ is hydrogen. In certain embodiments, the conditions are acidic. In certain embodiments, the acid is either inorganic or organic. In certain embodiments, the acid is inorganic. In certain embodiments, the acid is organic. In certain embodiments, the acid is hydrobromic acid, hydrochloric acid, hydrofluoric acid, hydroiodic acid, nitric acid, perchloric acid, phosphoric acid, sodium bisulfate, or sulfuric acid. In certain embodiments, the acid is acetic, benzenesulfonic, benzoic, citric, ethanesulfonic, fumaric, gluconic, lactic, maleic, malic, mandelic, methanesulfonic, mucic, p-toluenesulfonic, oxalic, pamoic, pantothenic, succinic, or trifluoromethane sulfonic. In certain embodiments, the conditions are basic. In certain embodiments, the base is either inorganic or organic. In certain embodiments, the base is inorganic. In certain embodiments, the base is organic. In certain embodiments, the base is a metal acetate, alkoxide, amide, amidine, carbonate, hydroxide, peroxide, phenoxide, phosphate, sulfate, or sulfide. In certain embodiments, the base is sodium hydroxide. In certain embodiments, the base is potassium hydroxide. In certain embodiments, the base is barium hydroxide. In certain embodiments, the base is tin hydroxide. In certain embodiments, the base potassium trimethylsilanolate. In certain embodiments, the base is sodium trimethylsilanolate. In certain embodiments, the base is 1,1,3,3-tetramethylguanidine, 1,4-diazabicyclo[2.2.2]octane, 1,8-bis(dimethylamino)naphthalene, 1,8-diazabicycloundec-7-ene, ammonia, diisopropylamine, imidazole, N,N-diisopropylethylamine, piperidine, pyridine, pyrrolidine, or triethylamine. In some embodiments, the solvent is a protic solvent. In some embodiments, the solvent is R³¹—OH or water. In certain embodiments, the reaction is carried out in a solvent mixture. In certain embodiments, the solvent mixture is THF and R³¹—OH or water. In certain embodiments, the solvent mixture is 1:1 THF:methanol. In certain embodiments, the base is sodium hydroxide and the solvent is water. In certain embodiments, the base is potassium hydroxide and the solvent is water. In certain embodiments, the base is barium hydroxide and the solvent is water. In certain embodiments, the base is tin hydroxide and the solvent is water.

Specific examples of the hydrolysis of the chiral auxillary from glycine aldol products such as compounds of Formula (A-2), (A-6), (A-17), or (A-27) are provided below (Scheme 13).

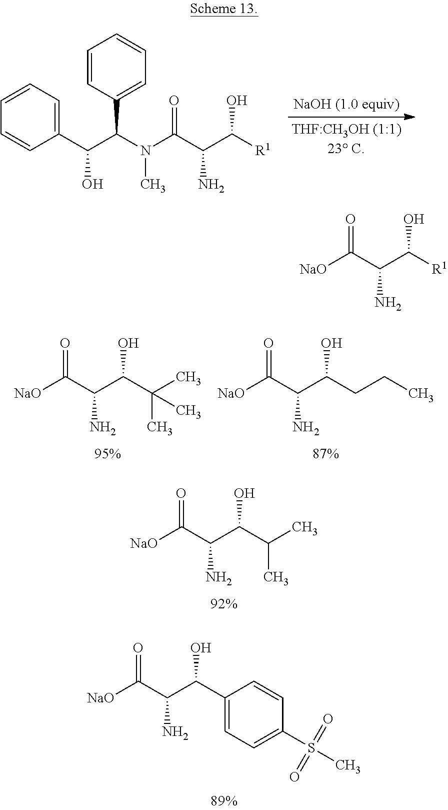

Strongly basic conditions can frequently lead to degradation of the desired intermediates en route to the targeted monobactams (see Scheme 14). Therefore, alternative conditions have been developed to enable the hydrolysis of such sensitive synthetic intermediates (e.g. situations where neither R¹ nor R² are not hydrogen, see Scheme 15). In certain embodiments, a compound of Formula (A-2), (A-6), (A-17), or (A-27) is contacted with phosgene or triphosgene to provide a compound of Formula (A-37) as shown in Scheme 15. In certain embodiments, a base is added. In certain embodiments, the base is organic. In certain embodiments, the base is 1,1,3,3-tetramethylguanidine, 1,4-diazabicyclo[2.2.2]octane, 1,8-bis(dimethylamino)naphthalene, 1,8-diazabicycloundec-7-ene, ammonia, diisopropylamine, imidazole, N,N-diisopropylethylamine, piperidine, pyridine, pyrrolidine, or triethylamine.

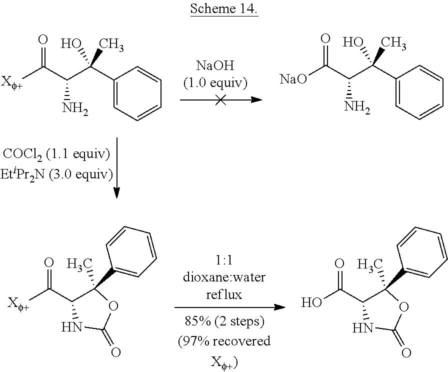

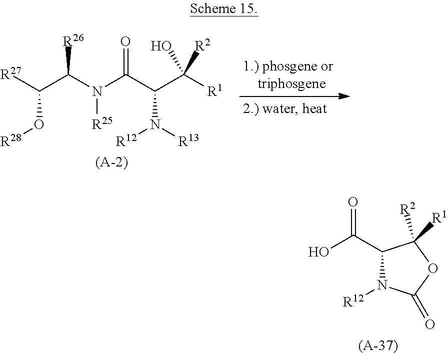

Protection of the free N—H group of intermediates such as compounds of Formula (A-18) can be carried out with a wide variety of N-protecting groups, as described herein (see Definitions). Conditions for such protections are varied and familiar to those skilled in the art. Specific conditions pertaining to individual protecting groups of interest described in detail in Protecting Groups in Organic Synthesis, T. W. Greene and P. G. M. Wuts, 3^{rd} edition, John Wiley & Sons, 1999. A non-limiting example of N—H protection of glycine aldol products is provided below (Scheme 16). Likewise, conditions for deprotection of O-PG and N-PG intermediates such as compounds of Formula (A-21), (A-25), and (A-32) are familiar to those skilled in the art and can also be found in the aforemented reference.

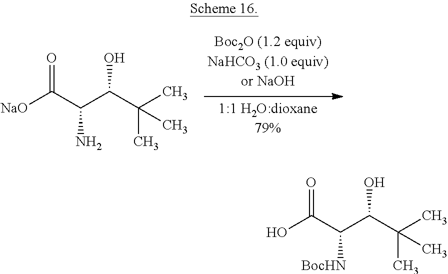

Available conditions for amide coupling reactions to produce compounds of Formula (A-4), (A-8), (A-13), (A-20), or (A-29) are numerous and familiar to those skilled in the art. Non-limiting examples include coupling with carbodiimides, such as N,N′-dicyclohexylcarbodiimide or N,N′-diisopropylcarbodiimide, activation of the acid with phosphinic chlorides such as bis(2-oxo-3-oxazolidinyl)phosphinic chloride (BOP—Cl) or phosphonic anhydrides (propylphosphonic anhydride, T3P), use of activated 1-hydroxybenzotriazole (HOBt), such as O-phosphonylated variants including (benzotriazol-1-yloxy)tris(dimethylamino)phosphonium hexafluorophosphate (BOP reagent) or (benzotriazol-1-yl-oxytripyrrolidinophosphonium hexafluorophosphate) (PyBOP reagent), conversion of the acid to an intermediate acyl halide (via, for example, phosphoryl or thionyl chloride) or anhydride (via, for example, pivalic anhydride) and subsequent amidation under basic conditions, or direct amidation with amino-metal complexes (e.g. aluminum amides via treatment of the amine with, for example, dimethylaluminum chloride). Numerous reviews are available on the process, including J. W. Bode (2006), “Emerging methods in amide- and peptide-bond formation.” Curr. Opin. Drug Discov. Devel. 9(6), 765-775, and E. Valeur (2009), “Amide bond formation: beyond the myth of coupling reagents.” Chem. Soc. Rev. 38(2), 606-631, which are incorporated herein by reference.

Conversion of the carbinol into a leaving group in compounds of Formula (A-4), (A-8), (A-13), (A-20), or (A-29) for the purpose of lactam cyclization can be accomplished using a wide variety of methods. Under certain conditions, overall inversion of the carbinol stereocenter may be realized, while under alternative conditions, the outcome may be overall retention of the stereochemistry at this center in the newly formed lactam. In all of the provided examples, replacement of one or more of the reagents with a variety of alternatives is well known to one skilled in the art (e.g. nBu₃P instead of PPh₃. In some embodiments, compounds of Formula (A-4), (A-8), (A-13), (A-20), or (A-29) are cyclized under acidic conditions. In some embodiments, compounds of Formula (A-4), (A-8), (A-13), (A-20), or (A-29) are cyclized through an intramolecular displacement of an activated carbinol. In some embodiments, the carbinol is activated in an additional step. In some embodiments, the carbinol of compounds of Formula (A-4), (A-8), (A-13), (A-20), or (A-29) is converted into a halide. In some embodiments, the carbinol of compounds of Formula (A-4), (A-8), (A-13), (A-20), or (A-29) is converted into a bromide. In some embodiments, the carbinol of compounds of Formula (A-4), (A-8), (A-13), (A-20), or (A-29) is converted into an iodide. In some embodiments, the carbinol of compounds of Formula (A-4), (A-8), (A-13), (A-20), or (A-29) is converted into a substituted or unsubstituted alkyl sulfonate. In some embodiments, the carbinol of compounds of Formula (A-4), (A-8), (A-13), (A-20), or (A-29) is converted into a substituted or unsubstituted aryl sulfonate. In some embodiments, the carbinol of compounds of Formula (A-4), (A-8), (A-13), (A-20), or (A-29) is converted into a methyl sulfonate. In some embodiments, the carbinol of compounds of Formula (A-4), (A-8), (A-13), (A-20), or (A-29) is converted into a trifluoromethane sulfonate. In some embodiments, the carbinol of compounds of Formula (A-4), (A-8), (A-13), (A-20), or (A-29) is converted into a toluene sulfonate. In some embodiments, the carbinol of compounds of Formula (A-4), (A-8), (A-13), (A-20), or (A-29) is converted into a nitrobenzene sulfonate. In some embodiments, the carbinol of compounds of Formula (A-4), (A-8), (A-13), (A-20), or (A-29) is converted into a sulfate group. In some embodiments, when the carbinol of compounds of Formula (A-4), (A-8), (A-13), (A-20), or (A-29) is converted into a halide, conditions S3, S6, S10, S16, or S24 comprise a phosphoryl halide. In some embodiments, when the carbinol of compounds of Formula (A-4), (A-8), (A-13), (A-20), or (A-29) is converted into a halide, conditions S3, S6, S10, S16, or S24 comprise a sulfuryl halide. In some embodiments, when the carbinol of compounds of Formula (A-4), (A-8), (A-13), (A-20), or (A-29) is converted into a halide, conditions S3, S6, S10, S16, or S24 comprise a tetrahalomethane and a phosphine. In some embodiments, the tetrahalomethane is carbon tetrachloride or carbon tetrabromide and the phosphine is triphenyl phosphine. In some embodiments, when the carbinol of compounds of Formula (A-4), (A-8), (A-13), (A-20), or (A-29) is converted into a sulfonate, conditions S3, S6, S10, S16, or S24 comprise a sulfonyl halide. In some embodiments, when the carbinol of compounds of Formula (A-4), (A-8), (A-13), (A-20), or (A-29) is converted into a sulfonate, conditions S3, S6, S10, S16, or S24 comprise a sulfonyl anhydride. In some embodiments, when the carbinol of compounds of Formula (A-4), (A-8), (A-13), (A-20), or (A-29) is converted into a sulfate group, conditions S3, S6, S10, S16, or S24 comprise potassium tetraborate.

In some embodiments, conditions S3, S6, S10, S16, or S24 are neutral. In some embodiments, conditions S3, S6, S10, S16, or S24 comprise addition of a base. In certain embodiments of conditions S3, S6, S10, S16, or S24, the base is either inorganic or organic. In certain embodiments of conditions S3, S6, S10, S16, or S24, the base is inorganic. In certain embodiments of conditions S3, S6, S10, S16, or S24, the base is organic. In certain embodiments of conditions S3, S6, S10, S16, or S24, the base is a metal acetate, alkoxide, amide, amidine, carbonate, hydroxide, phenoxide, or phosphate. In certain embodiments of conditions S3, S6, S10, S16, or S24, the base is sodium, potassium, or caesium carbonate. In certain embodiments of conditions S3, S6, S10, S16, or S24, the base is sodium, potassium, or caesium bicarbonate. In certain embodiments of conditions S3, S6, S10, S16, or S24, the base is 1,1,3,3-tetramethylguanidine, 1,4-diazabicyclo[2.2.2]octane, 1,8-bis(dimethylamino)naphthalene, 1,8-diazabicycloundec-7-ene, ammonia, diisopropylamine, imidazole, N,N-diisopropylethylamine, piperidine, pyridine, pyrrolidine, or triethylamine. In some embodiments of conditions S3, S6, S10, S16, or S24, the solvent is a polar protic solvent. In some embodiments of conditions S3, S6, S10, S16, or S24, the solvent is a polar aprotic solvent. In some embodiments of conditions S4, the reaction is performed in the absence of solvent. In some embodiments, conditions S3, S6, S10, S16, or S24 comprise a catalyst. In some embodiments of conditions S3, S6, S10, S16, or S24, the catalyst is an iodide salt.

In some embodiments, the hydroxyl moiety of a compound of Formula (A-4), (A-8), (A-13), (A-20), or (A-29) is converted into a leaving group in situ. In some embodiments, the hydroxyl moiety of a compound of Formula (A-4), (A-8), (A-13), (A-20), or (A-29) is converted into a leaving group in situ using an azodicarboxylate and an aryl or alkyl phosphine. In some embodiments, the azodicarboxylate is diethyl azodicarboxylate and the phosphine is triphenyl phosphine. In some embodiments, the hydroxyl moiety of a compound of Formula (A-4), (A-8), (A-13), (A-20), or (A-29) is converted into a leaving group in situ using sulfur trioxide pyridine complex.

In some embodiments, the properties of the R¹², R¹³, or PG¹ groups present during the cyclization of compounds of Formula (A-4), (A-8), or (A-20) are important for determining the success of the reactions resulting in lactams of Formula (A-5), (A-9), or (A-21). In some embodiments, R¹² is an electron withdrawing substituent. In some embodiments, R¹³ is an electron withdrawing substituent. In some embodiments, both R¹² and R¹³ are electron withdrawing substituents. In some embodiments, PG¹ is an electron withdrawing protecting group.

Removal of the O-PG³ group in compounds of Formula (A-35) can be accomplished through a variety of means. Several non-limiting examples of conditions S29 include catalytic hydrogenolysis using a heterogeneous palladium or nickel catalyst and molecular hydrogen, a dissolving metal reduction using lithium, sodium, or potassium metal, a metal hydride reduction using a reagent such as lithium aluminum hydride, or a single-electron reduction using a reagent such as titanium (III) chloride. In some embodiments, S29 is a two-step sequence, the first step comprising removing the —PG³ group, and the second step comprising removing the resulting —OH group of the hydroxamate. In some embodiments, when S29 is a two-step sequence, the sequence comprises removing the —PG³ group with palladium on carbon and molecular hydrogen followed by removing the resulting —OH group of the hydroxamate with titanium (III) chloride. In some embodiments of compounds of Formula (A-35), —PG³ is benzyl.

Leaving group conjugates of R¹² and R¹³ include, but are not limited to, halides, sulfonates, or phosphonates. In some embodiments, the leaving group conjugate of R¹² is a halide of the Formula XR¹², wherein X is a halide. In some embodiments, the leaving group conjugate of R¹³ is a halide of the Formula or XR¹³, wherein X is a halide. In some embodiments, the leaving group conjugate of R¹² is a sulfonate of the Formula R²⁹SO₂OR¹², wherein R²⁹ is C₁₋₆ alkyl, or optionally substituted phenyl. In some embodiments, the leaving group conjugate of R¹² is a sulfonate of the Formula R²⁹SO₂XR¹³, wherein R²⁹ is C₁₋₆ alkyl, or optionally substituted phenyl.

### EXEMPLIFICATION

In order that the invention described herein may be more fully understood, the following examples are set forth. It should be understood that these examples are for illustrative purposes only and are not to be construed as limiting this invention in any manner.

### General Experimental Procedures

All reactions were performed in flame-dried glassware fitted with rubber septa under a positive pressure of argon, unless otherwise noted. Air- and moisture-sensitive liquids were transferred via syringe or stainless steel cannula. Solutions were concentrated by rotary evaporation below 35° C. Analytical thin-layer chromatography (TLC) was performed using glass plates pre-coated with silica gel (0.25-mm, 60-A pore size, 230-400 mesh, Merck KGA) impregnated with a fluorescent indicator (254 nm). TLC plates were visualized by exposure to ultraviolet light (UV), then were stained by submersion in a 10% solution of phosphomolybdic acid (PMA) in ethanol, followed by brief heating on a hot plate. Flash column chromatography was performed as described by W. C. Still, M. Kahn, A. Mitra, J. Org. Chem. 1978, 43, 2923-2925 employing silica gel (60 Å, standard grade) purchased from Dynamic Adsorbents.

Materials:

Commercial solvents and reagents were used as received with the following exceptions. Hexamethyldisilazine (HMDS) was distilled from calcium hydride under an atmosphere of dinitrogen at 760 mmHg. Dichloromethane, ethyl ether, dioxane, and tetrahydrofuran were purified by passage through Al₂O₃ under argon by the method of A. B. Pangborn, M. A. Giardello, R. H. Grubbs, R. K. Rosen, F. J. Timmers, Organometallics. 1996, 15, 1518-1520. Aldehydes and ketones for use as electrophiles in aldol reactions were fractionally distilled immediately prior to use. The molarity of solutions of n-butyllithium was determined by titration against diphenylacetic acid as an indicator (average of three determinations). W. G. Kofron, L. M. Baclawski, J. Org. Chem. 1976, 41, 1879-1880.

Instrumentation:

Proton nuclear magnetic resonance (1H NMR) spectra were recorded on Varian MERCURY 400 (400 MHz), Varian INOVA 500 (500 MHz), or Varian INOVA 600 (600 MHz) NMR spectrometers at 23° C. Proton chemical shifts are expressed in parts per million (ppm, δ scale) and are referenced to residual protium in the NMR solvent (CHCl₃: δ 7.26, D₂HCOD: δ 3.31). Data are represented as follows: chemical shift, multiplicity (s=singlet, d=doublet, t=triplet, q=quartet, dd=doublet of doublets, dt=doublet of triplets, sxt=sextet, m=multiplet, br=broad, app=apparent), integration, and coupling constant (J) in Hertz (Hz). HPLC retention times were acquired using a Beckman System Gold instrument equipped with a Chiracel OD-H column (5 mm particle size, 4.6 mm×250 mm). High-resolution mass spectra were obtained at the Harvard University Mass Spectrometry Facility using a Bruker microTOF-QII mass spectrometer. LC-MS analysis, including collection of low resolution mass spectrometry data, was performed on an Agilent 1260 Infinity instrument equipped with a 6120 quadrupole LC-MS.

### General Procedure for Aldolization of Pseudoephenamine Glycinamide with Ketones

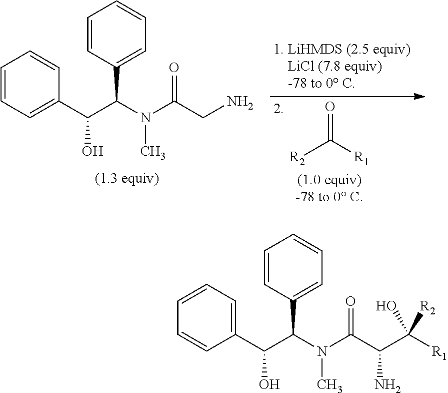

A 25-mL round-bottom flask equipped with a stir bar was charged with anhydrous lithium chloride (331 mg, 7.80 mmol, 7.8 equiv). The reaction vessel was heated with a gentle flame under vacuum (0.1 mmHg) for 2 minutes. After cooling to 23° C. in vacuo, the flask was backfilled with argon and (R,R)-pseudoephenamine glycinamide (370 mg, 1.30 mmol, 1.3 equiv) was added. Tetrahydrofuran (6.5 mL) was added by syringe and the reaction mixture was stirred at ambient temperature until pseudoephenamine glycinamide had dissolved (˜5 minutes); lithium chloride does not completely dissolve. The resulting suspension was cooled to −78° C. in a dry ice-acetone cooling bath and a freshly-prepared solution of lithium hexamethyldisilazide in tetrahydrofuran (1.0 M, 2.5 mL, 2.5 mmol, 2.5 equiv) was added dropwise. After 5 minutes, the reaction vessel was transferred to an ice-water bath and stirring continued for 25 minutes. The vessel was re-cooled to −78° C., and a solution of ketone in tetrahydrofuran (1.0 M, 1.0 mL, 1.0 mmol, 1.0 equiv) was added dropwise. After 30 minutes at −78° C., the reaction vessel was transferred to an ice-water cooling bath and stirring continued at to 0° C. Once the ketone was completely consumed as indicated by TLC (usually <30 minutes), the mixture was partitioned between half-saturated aqueous ammonium chloride solution (20 mL) and ethyl acetate (25 mL). The layers were separated, and the aqueous layer was extracted with ethyl acetate (2×25 mL). The combined organic extracts were washed with saturated aqueous sodium chloride solution (45 mL) and dried over sodium sulfate. The dried solution was filtered, and the filtrate was concentrated. The diastereomeric ratio of the crude product was determined by NMR or HPLC analysis (vide infra). The residue was purified by flash column chromatography on silica gel.

### Example 1

### Aldolization of (R,R)-pseudoephenamine glycinamide with 2-fluoro-4-(methylsulfonyl)benzaldehyde

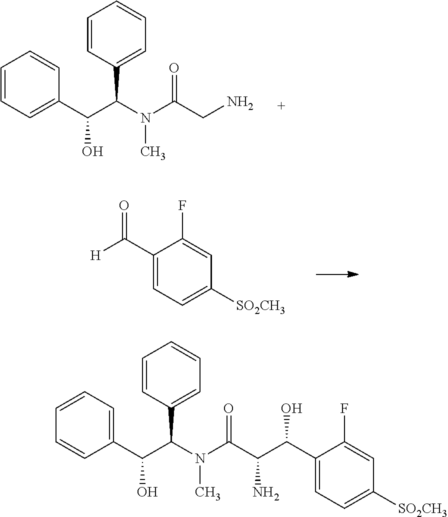

A 25-mL round-bottom flask equipped with a stir bar was charged with anhydrous lithium chloride (331 mg, 7.80 mmol, 7.8 equiv). The vessel was heated with a gentle flame under vacuum (0.1 mmHg) for 2 minutes. After cooling to 23° C. in vacuo, the flask was backfilled with argon and (R,R)-pseudoephenamine glycinamide (370 mg, 1.30 mmol, 1.3 equiv) was added. Tetrahydrofuran (6.5 mL) was added by syringe and the reaction mixture was stirred at 23° C. until pseudoephenamine glycinamide had dissolved (˜5 minutes); lithium chloride does not completely dissolve. The resulting suspension was cooled to −78° C. in a dry ice-acetone cooling bath and a freshly-prepared solution of lithium hexamethyldisilazide in tetrahydrofuran (1.0 M, 2.5 mL, 2.5 mmol, 2.5 equiv) was added dropwise. After 5 minutes, the reaction vessel was transferred to an ice-water bath and stirring was continued for 25 minutes. The vessel was re-cooled to −78° C., and a solution of aldehyde in tetrahydrofuran (1.0 M, 1.0 mL, 1.0 mmol, 1.0 equiv) was added dropwise. Once the aldehyde was completely consumed as indicated by TLC (usually <30 minutes), a half-saturated aqueous ammonium chloride solution (0.10 mL) was added and the vessel was allowed to warm to 23° C. The mixture was partitioned between half-saturated aqueous ammonium chloride solution (20 mL) and ethyl acetate (25 mL). The layers were separated, and the aqueous layer was extracted with ethyl acetate (2×25 mL). The combined organic extracts were washed with saturated aqueous sodium chloride solution (45 mL) and dried over sodium sulfate. The dried solution was filtered, and the filtrate was concentrated. The residue was purified by flash column chromatography (1→5% methanol-ethyl acetate+0.5% saturated aqueous ammonium hydroxide solution, followed by a second column of 3→8% methanol-dichloromethane+0.5% saturated aqueous ammonium hydroxide solution) to provide the title compound as a white solid (315 mg, 64%). ¹H NMR (4:1 ratio of rotamers; major rotamer reported, 500 MHz, CD₃OD), δ: 7.72-7.69 (m, 1H), 7.61 (dd, 1H, J=8.0, 1.6 Hz), 7.35-7.30 (m, 4H), 7.23-7.15 (m, 6H), 7.09-7.07 (m, 1H), 5.98 (d, 1H, J=9.9 Hz), 5.27 (d, 1H, J=9.9 Hz), 4.95 (d, 1H, J=7.2 Hz), 4.06 (d, 1H, J=7.2 Hz), 3.02 (s, 3H), 2.80 (s, 3H).

### Example 2

### Aldolization of (R,R)-pseudoephenamine glycinamide with 1,3-difluoropropan-2-one

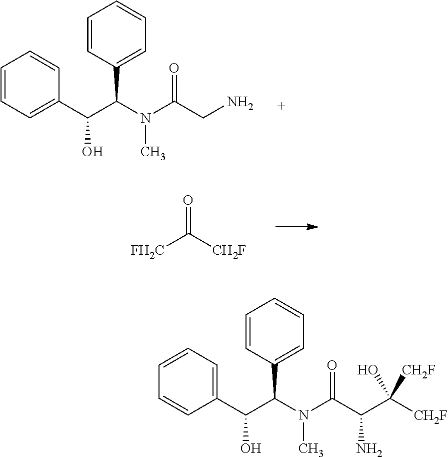

The general procedure for aldolization of (R,R)-pseudoephenamine glycinamide with ketones was followed. The diastereomeric ratio of the crude product residue was determined to be 94:6 by HPLC analysis (Agilent Extend-C18, 85:15→65:35 water:acetonitrile+0.1% trifluoroacetic acid, 0.5 mL/min, λ=220 nm, t_{R} (minor)=12.9 min, t_{R} (major)=15.3 min). The residue was purified by flash column chromatography (1→3% methanol-dichloromethane+0.5% saturated aqueous ammonium hydroxide solution) to provide the title compound as a white foam (264 mg, 70%). ¹H NMR (3:1 ratio of rotamers; asterisk (*) denotes minor rotamer peaks, 500 MHz, CD₃OD), δ: 7.41 (d, 2H, J=8.3 Hz), 7.38* (d, 2H, J=8.3 Hz), 7.31 (d, 2H, J=6.8 Hz), 7.25-7.16 (m, 6H), 6.02 (d, 1H, J=9.3 Hz), 5.55* (d, 1H, J=9.7 Hz), 5.42 (d, 1H, J=9.3 Hz), 5.32* (d, 1H, J=9.7 Hz), 4.78-4.60* (m, 3H), 4.60-4.54 (m, 1H), 4.50-4.44 (m, 2H), 4.39-4.34 (m, 1H), 4.27-4.25* (m, 2H), 4.08 (s, 1H), 3.16 (s, 3H), 3.03* (s, 3H). HRMS (ESI): Calcd for (C₂₀H₂₄F₂N₂O₃+Na)⁺: 401.1647. Found: 401.1634.

### Example 3

### One Pot, Two Step Synthesis of N-Boc Acids from Aldol Adducts

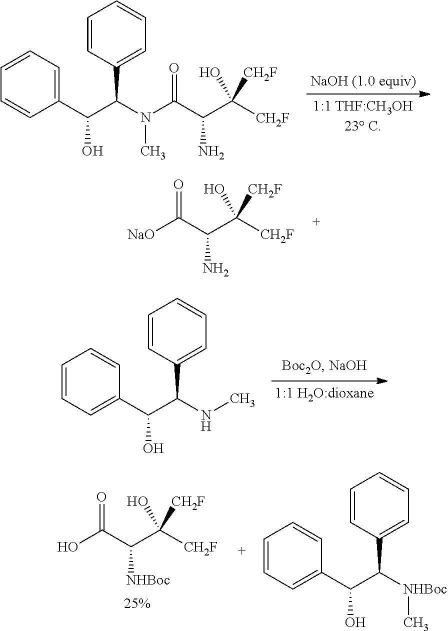

A 25-mL round bottom flask equipped with a stir bar was charged with aldol adduct (616 mg, 1.63 mmol, 1 equiv). A 1:1 mixture of tetrahydrofuran:methanol (6.4 mL) was added, followed by aqueous sodium hydroxide solution (1.0 M, 1.63 mL, 1.63 mmol, 1 equiv). Reaction progress was monitored by the consumption of starting material by TLC (10% methanol in dichloromethane+0.5% saturated aqueous ammonium hydroxide solution). After 4 d, the reaction mixture was concentrated to dryness and the residue was dissolved in a 1:1 mixture of water:1,4-dioxane (15 mL). The reaction vessel was placed in an ice water cooling bath. Aqueous sodium hydroxide solution (1.0 M, 4.88 mL, 4.88 mmol, 3 equiv) was added, followed by di-tert-butyl dicarbonate (1.13 mL, 4.88 mmol, 3 equiv). The cooling bath was removed after 5 minutes and the vessel continued to stir at 23° C. Reaction progress was monitored by the consumption of starting material by LC-MS analysis of aliquots removed from the reaction mixture. After 16 h, water (30 mL) was added and the mixture was washed with three portions of ether (30 mL). The ethereal extracts were combined and back-extracted with 0.5 M aqueous sodium hydroxide solution (20 mL). The ethereal extracts were dried over sodium sulfate and filtered. The filtrate was concentrated to provide N-Boc-pseudoephenamine in quantitative yield.

The basic aqueous phases were combined, the resulting solution was cooled in an ice-water cooling bath, and 1 M aqueous hydrochloric acid solution was added dropwise until the pH of the solution was ˜2. The acidified aqueous phase was then extracted with ethyl acetate (3×25 mL). The organic extracts were combined and were dried over sodium sulfate. The dried organic solution was filtered and the filtrate was concentrated to provide crude N-Boc-protected acid. The crude material was purified via flash column chromatography (2→5% methanol-dichloromethane+1% acetic acid) to give N-Boc-protected acid (110 mg, 25%) as an off-white foam. ¹H NMR (500 MHz, CD₃OD), δ: 4.66-4.46 (m, 3H), 4.41 (m, 1H), 4.37 (s, 1H), 1.46 (s, 9H). HRMS (ESI): Calcd for (C₁₀H₁₇F₂NO₅+Na)⁺: 292.0967. Found: 292.0954.

### Example 4

### Synthesis of Exemplary Monobactam

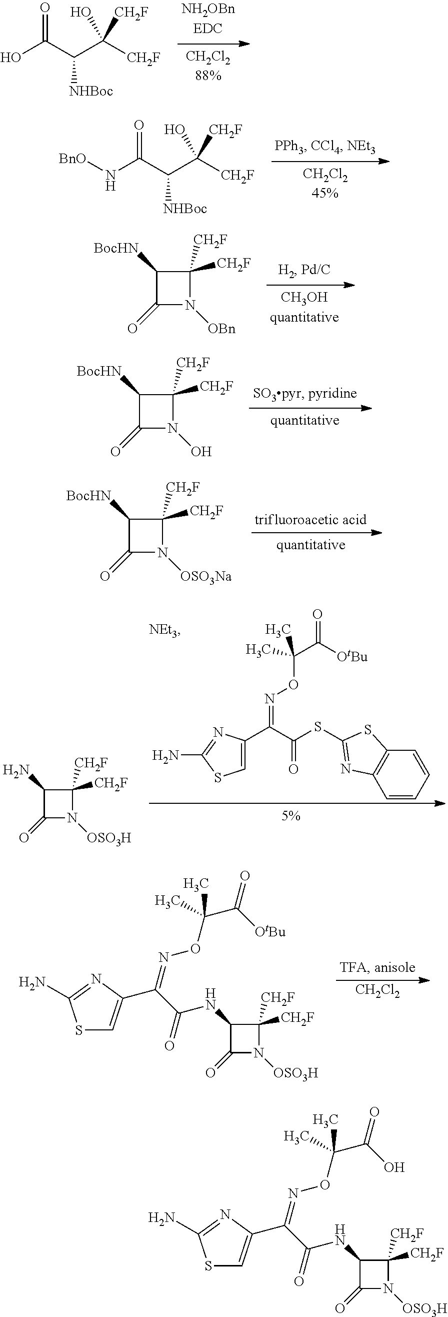

### Step 1. Synthesis of Hydroxamate

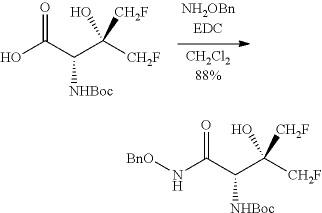

To a 25-mL round bottom flask equipped with a stir bar was added the N-Boc acid (100 mg, 0.371 mmol, 1 equiv) and dry dichloromethane (3.7 mL). O-Benzylhydroxylamine (0.052 mL, 0.427 mmol, 1.15 equiv) was added, followed by EDC (78 mg, 0.409 mmol, 1.1 equiv) in one portion. Reaction progress was monitored by the consumption of starting material by LC-MS analysis of aliquots removed from the reaction mixture. After 14 h, additional dichloromethane (10 mL) was added and the reaction mixture was washed sequentially with water (15 mL) and saturated aqueous sodium chloride solution (15 mL). The organic phase was dried over sodium sulfate and was filtered. The filtrate was concentrated to provide crude material which was purified via flash column chromatography (20→40% ethyl acetate-hexanes) to provide the hydroxamate (123 mg, 88%) as a white foam. ¹H NMR (600 MHz, CD₃OD), δ: 7.43 (d, 2H, J=6.5 Hz), 7.38-7.34 (m, 3H), 4.47-4.37 (m, 4H), 4.21 (s, 1H), 1.45 (s, 9H). [*Note: Two protons are hidden under the residual water peak at δ 4.87.] HRMS (ESI): Calcd for (C₁₇H₂₄F₂N₂O₅—H): 373.1581. Found: 373.1562.

### Step 2. Synthesis of 3-lactam

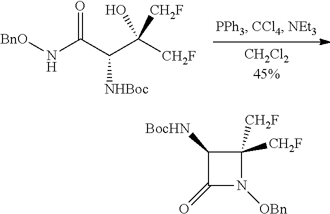

A 25-mL round bottom flask equipped with a stir bar was charged with hydroxamate (123 mg, 0.329 mmol, 1 equiv). Dry acetonitrile (3.3 mL) was added, followed by carbon tetrachloride (0.190 mL, 1.971 mmol, 6 equiv), triphenylphosphine (172 mg, 0.657 mmol, 2 equiv) and triethylamine (0.105 mL, 0.756 mmol, 2.3 equiv). The reaction mixture continued to stir at 23° C. After 2 h, the reaction mixture had darkened to brown in color. Reaction progress was monitored by the consumption of starting material by LC-MS analysis of aliquots removed from the reaction mixture. Upon complete consumption of the starting material, the reaction mixture was concentrated in vacuo. The residue was purified via flash column chromatography (20% ethyl acetate-hexanes) to provide the desired β-lactam (53 mg, 45%). ¹H NMR (500 MHz, CD₃OD), δ: 7.45-7.38 (m, 5H), 5.01-4.96 (m, 2H), 4.84-4.70 (m, 3H), 4.62-4.43 (m, 2H), 1.44 (s, 9H). HRMS (ESI): Calcd for (C₁₇H₂₂F₂N₂O₄+Na)⁺: 379.1440. Found: 379.1437.

### Step 3. Synthesis of N-hydroxyl β-lactam

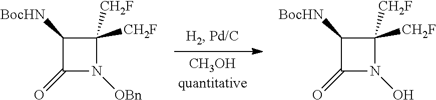

To a 10-mL round bottom flask equipped with a stir bar and charged with β-lactam (53 mg, 0.149 mmol, 1 equiv) was added methanol (1.49 mL), followed by 10% palladium on carbon (16 mg). The reaction flask was then equipped with a balloon of hydrogen. An atmosphere of hydrogen was introduced by briefly evacuating the flask and refilling with hydrogen gas. This process was repeated three times and the reaction mixture was stirred under an atmosphere of hydrogen at 23° C. Reaction progress was monitored by the consumption of starting material by LC-MS analysis of aliquots removed from the reaction mixture. After 3 h, the reaction mixture was filtered through a pad of celite on a fritted filter in order to remove the palladium. The methanolic filtrate was concentrated in vacuo to provide the N-hydroxyl β-lactam in quantitative yield. ¹H NMR (600 MHz, CD₃OD), δ: 4.86-4.71 (m, 4H), 4.63 (s, 1H), 1.45 (s, 9H). HRMS (ESI): Calcd for (C₁₀H₁₆F₂N₂O₄—H): 265.1000. Found: 265.1042.

### Step 4. O-Sulfonation

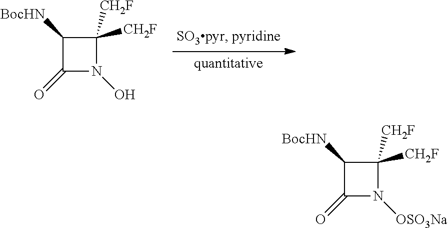

A 10-mL round bottom flask equipped with a stir bar was charged with N-hydroxyl β-lactam (27 mg, 0.101 mmol, 1 equiv). Pyridine (0.254 mL) was added and the reaction vessel was placed in an ice water cooling bath. Sulfur trioxide-pyridine complex (21 mg, 0.132 mmol, 1.3 equiv) was added. The cooling bath was removed after 5 minutes and the vessel continued to stir at 23° C. Reaction progress was monitored by the consumption of starting material by LC-MS analysis of aliquots removed from the reaction mixture. After 3 h, the reaction mixture was concentrated in vacuo to remove the pyridine and redissolved in methanol (2 mL). The O-sulfonic acid β-lactam solution in methanol was stirred with Dowex Marathon C Ion exchange beads for 15 min. The methanolic solution was filtered and the filtrate was concentrated to provide the O-sulfonic acid β-lactam in quantitative yield. 1H NMR (600 MHz, CD₃OD), δ: 4.97-4.67 (m, 5H), 1.45 (s, 9H). LRMS (ESI): Calcd for (C₁₀H₁₆F₂N₂O₇S—H): 345.1. Found: 345.0.

### Step 5. Synthesis of Monocyclic β-Lactam Core: Removal of the N-Boc Carbonate

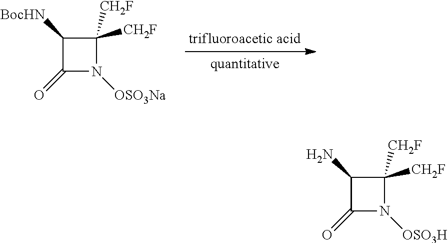

A 10-mL round bottom flask equipped with a stir bar was charged with β-lactam (37 mg, 0.101 mmol, 1 equiv), followed by dichloromethane (0.300 mL). The reaction vessel was placed in an ice water cooling bath and a 1:1 mixture of trifluoroacetic acid-dichloromethane (0.700 mL) was added. Reaction progress was monitored by the consumption of starting material by LC-MS analysis of aliquots removed from the reaction mixture. After 20 min at 0° C., the reaction mixture was concentrated to provide the core in quantitative yield as a white solid. LRMS (ESI): Calcd for (C₅H₈F₂N₂O₅S—H): 245.0. Found: 245.1.

### Step 6. Acylation of N-3 Amine

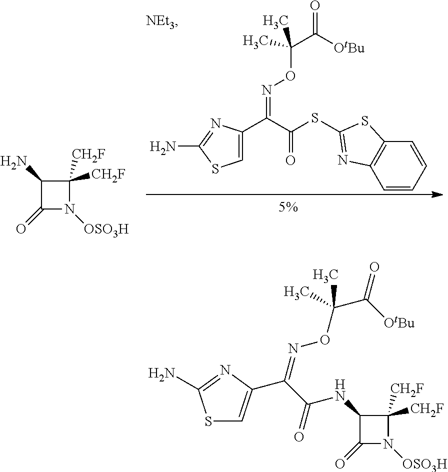

A 10-mL round bottom flask equipped with a stir bar was charged with core (25 mg, 0.101 mmol, 1 equiv), followed by wet tetrahydrofuran (0.400 mL). The reaction vessel was placed in an ice water cooling bath. Triethylamine (0.42 mL, 0.305 mmol, 3 equiv) was added and the cooling bath was removed after 5 minutes. The vessel continued to stir at 23° C. as (Z)-tert-butyl 2-(((1-(2-aminothiazol-4-yl)-2-(benzo[d]thiazol-2-ylthio)-2-oxoethylidene)amino)oxy)-2-methylpropanoate (73 mg, 0.152 mmol, 1.5 equiv) was added portion-wise. Reaction progress was monitored by the consumption of the core by LC-MS analysis of aliquots removed from the reaction mixture. After 2.5 h, ethyl acetate (10 mL) and water (10 mL) were added and the layers were separated. The organic layer was washed with 0.5 M aqueous hydrochloric acid solution (2×10 mL) and the aqueous extracts were combined and concentrated with no additional heating on the rotovap bath. The crude material was purified via HPLC (Agilent Extend-C18, 90:10→70:30 water-acetonitrile+0.1% formic acid, then 100% acetonitrile+0.1% formic acid) to provide the coupled product (3 mg, 5%) as an off-white solid. 1H NMR (600 MHz, CD₃OD), δ: 7.14 (s, 1H), 5.33 (s, 1H), 5.00-4.83 (m, 4H), 1.60 (s, 3H), 1.59 (s, 3H), 1.47 (s, 9H). HRMS (ESI): Calcd for (C₁₈H₂₅F₂N₅O₉S₂— H): 556.0989. Found: 556.0985.

### Step 7. Saponification of t-Butyl Ester

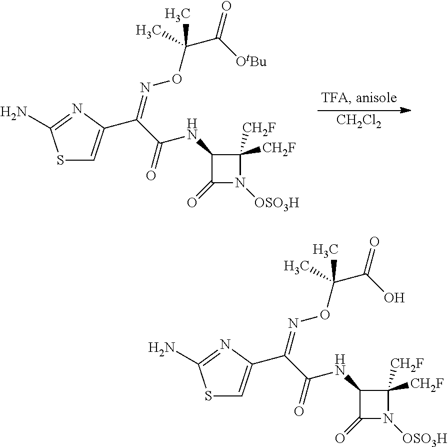

A 10-mL round bottom flask equipped with a stir bar was charged with the coupled product (3 mg, 5.38 μmol, 1 equiv), followed by dry dichloromethane (0.100 mL) and anisole (0.053 mL, 0.484 mmol, 90 equiv). The reaction vessel was placed in an ice water cooling bath. Trifluoroacetic acid (0.083 mL, 1.076 mmol, 200 equiv) was added and the vessel continued to stir at 0° C. as reaction progress was monitored by the consumption of the starting material by LC-MS analysis of aliquots removed from the reaction mixture. After 30 min, additional trifluoroacetic acid (0.5 mL) was added and the reaction vessel continued to stir at 0° C. After 45 additional minutes, toluene (10 mL) was added and the reaction mixture was concentrated without additional heating on the rotovap bath. Water (2 mL) and hexanes (2 mL) were added and then layers were separated. An HPLC sample was prepared from the aqueous phase and the crude material was purified via HPLC (Agilent Extend-C18, 90:10→70:30 water-acetonitrile+0.1% formic acid, then 100% acetonitrile+0.1% formic acid). Note: Care must be taken upon concentration of the aqueous fractions. Labile hydrolysis of the O-sulfonic acid occurs upon concentration of the HPLC fractions with formic acid present. Therefore, toluene was added to all desired fractions and the fractions were concentrated without additional heating on the rotovap bath. The final product was isolated as an off-white solid (1.2 mg, 45%). 1H NMR (600 MHz, CD₃OD), δ: 6.92 (s, 1H), 5.32 (s, 1H), 4.99-4.73 (m, 4H), 1.60 (s, 6H). HRMS (ESI): Calcd for (C₁₄H₁₇F₂N₅O₉S₂— H): 500.0358. Found: 500.0864.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I) or (I′): 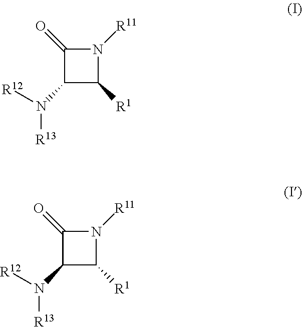
or a pharmaceutically acceptable salt thereof,
wherein
R¹ is selected from C₁₋₁₀ alkyl, C₁₋₁₀ haloalkyl, C₂₋₁₀ alkenyl, C₂₋₁₀ alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, or 3-10 membered carbocyclylalkyl, each of which is optionally substituted with 0, 1, 2, 3, 4, or 5 occurrences of R⁵;
R⁵ is independently selected from halogen, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ alkynyl, substituted or unsubstituted C₁₋₆ alkoxy, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷ or —SR⁷;
R⁷ is independently selected from hydrogen, acyl, substituted or unsubstituted C₁₋₁₀ alkyl, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, or two R⁷ groups are taken together with any intervening atoms to form a substituted or unsubstituted heterocyclic ring.
R¹¹ is hydrogen, C₁₋₆ alkyl, C₁₋₆ haloalkyl, C₂₋₆ alkenyl, C₂₋₆ alkynyl, C₁₋₆ alkoxy, C₆₋₁₀ aryl, 5-10 membered heteroaryl, 4-10 membered heterocyclyl, 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵; and
R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, C₆₋₁₀ aryl, 5-10 membered heteroaryl, 4-10 membered heterocyclyl, 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), a nitrogen protecting group, or R¹² and R¹³ are taken together with any intervening atoms to form a heteroaryl or heterocyclic ring, each of which is optionally substituted with 0, 1, 2, 3, 4, or 5 occurrences of R⁵.
with the exclusion of known monobactams included in Scheme 1.

2. The compound of claim 1, wherein the compound is of Formula (I-a) or (I-a′):

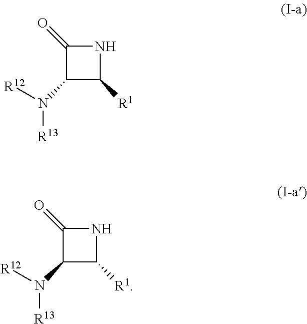

3. The compound of claim 1, wherein the compound is of Formula (I-b) or (I-b′):

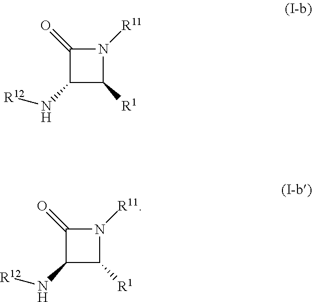

4. The compound of claim 1, wherein the compound is of Formula (I-c) or (I-c′):

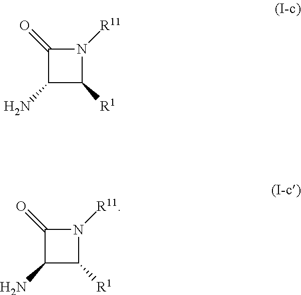

5. The compound of claim 1, wherein R¹ is substituted or unsubstituted C₆₋₁₀ aryl, substituted or unsubstituted 5-10 membered heteroaryl wherein linkage occurs through a carbon atom of the heteroaryl ring, substituted or unsubstituted 4-10 membered heterocyclyl wherein linkage occurs through a carbon atom of the heterocyclyl ring, or substituted or unsubstituted 3-10 membered carbocyclyl provided R¹ is not unsubstituted phenyl, phenyl substituted with 1 occurrence of R⁵, phenyl substituted with 2 occurrences of R⁵, unsubstituted or substituted thiophene, unsubstituted or unsubstituted furan, unsubstituted or substituted isoxazole, unsubstituted or substituted pyrazole, unsubstituted or substituted pyridine, unsubstituted or substituted dioxolane, or unsubstituted or substituted cyclohexyl.

6. The compound of claim 1, wherein R¹ is not unsubstituted or substituted alkyl.

7. A compound of Formula (II) or (II′): 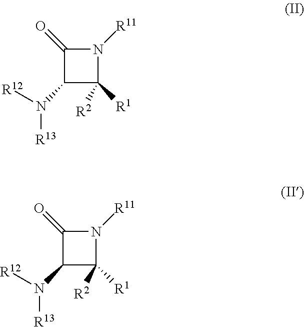
or a pharmaceutically acceptable salt thereof,
wherein
R¹ and R² are independently selected from C₁₋₁₀ alkyl, C₁₋₁₀ haloalkyl, C₂₋₁₀ alkenyl, C₂₋₁₀alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, 3-10 membered carbocyclylalkyl, or R¹ and R² are taken together with any intervening atoms to form a carbocyclic or heterocyclic ring, each of which is optionally substituted with 0, 1, 2, 3, 4, or 5 occurrences of R⁵;
R⁵ is independently selected from halogen, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ alkynyl, substituted or unsubstituted C₁₋₆ alkoxy, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷;
R⁷ is independently selected from hydrogen, acyl, substituted or unsubstituted C₁₋₁₀ alkyl, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, or two R⁷ groups are taken together with any intervening atoms to form a substituted or unsubstituted heterocyclic ring.
R¹¹ is hydrogen, C₁₋₆ alkyl, C₁₋₆ haloalkyl, C₂₋₆ alkenyl, C₂₋₆ alkynyl, C₁₋₆ alkoxy, C₆₋₁₀ aryl, 5-10 membered heteroaryl, 4-10 membered heterocyclyl, 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵; and
R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, C₆₋₁₀ aryl, 5-10 membered heteroaryl, 4-10 membered heterocyclyl, 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), a nitrogen protecting group, or R¹² and R¹³ are taken together with any intervening atoms to form a heteroaryl or heterocyclic ring, each of which is optionally substituted with 0, 1, 2, 3, 4, or 5 occurrences of R⁵.

8. The compound of claim 7, with the exclusion of any monobactam depicted in Table 1.

9. The compound of claim 7, wherein the compound is of Formula (II-a) or (II-a′):

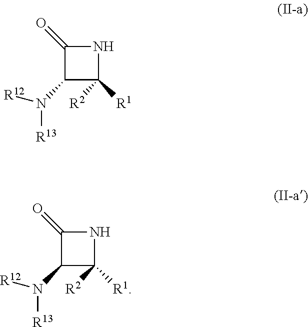

10. The compound of claim 7, wherein the compound is of Formula (II-b) or (II-b′):

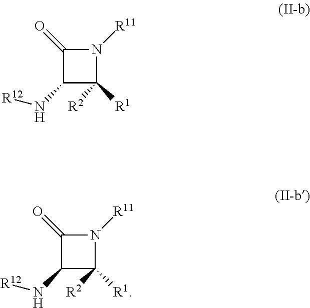

11. The compound of claim 7, wherein the compound is of Formula (II-c) or (II-c′):

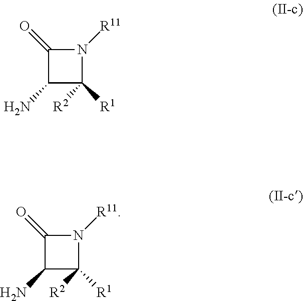

12. The compound of claim 7, wherein R¹ or R² are substituted or unsubstituted C₆-10 aryl, substituted or unsubstituted 5-10 membered heteroaryl wherein linkage occurs through a carbon atom of the heteroaryl ring, substituted or unsubstituted 4-10 membered heterocyclyl wherein linkage occurs through a carbon atom of the heterocyclyl ring, or substituted or unsubstituted 3-10 membered carbocyclyl.

13. (canceled)

14. The compound of claim 7, wherein R² is substituted or unsubstituted C₁₋₃ alkyl, substituted or unsubstituted C₁₋₃ haloalkyl, substituted or unsubstituted C₆ aryl, substituted or unsubstituted C₁₋₆ alkoxy, substituted or unsubstituted 5-10 membered heteroaryl wherein linkage occurs through a carbon atom of the heteroaryl ring, substituted or unsubstituted 4-10 membered heterocyclyl wherein linkage occurs through a carbon atom of the heterocyclyl ring, or substituted or unsubstituted 3-6 membered carbocyclyl.

15-16. (canceled)

17. The compound of claim 7, wherein R¹ and R² are taken together to form an substituted or unsubstituted carbocyclic ring or substituted or unsubstituted heterocyclic ring.

18-26. (canceled)

27. The compound of claim 1, wherein R¹¹ is:
hydrogen, 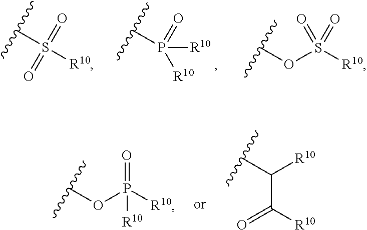
wherein:
R¹⁰ is independently selected from hydrogen, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ alkynyl, substituted or unsubstituted C₁₋₆ alkoxy, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷.

28-31. (canceled)

32. The compound of claim 1, wherein R¹² is hydrogen; and R¹³ is 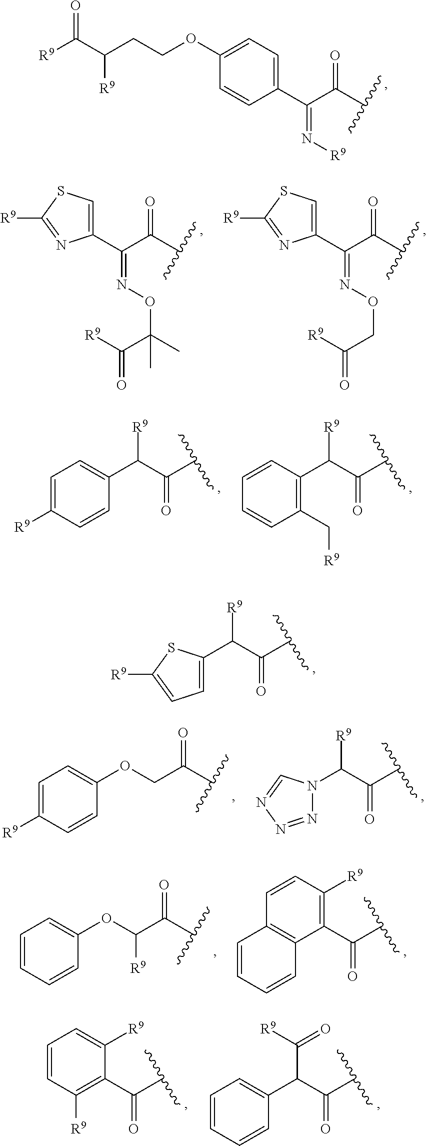 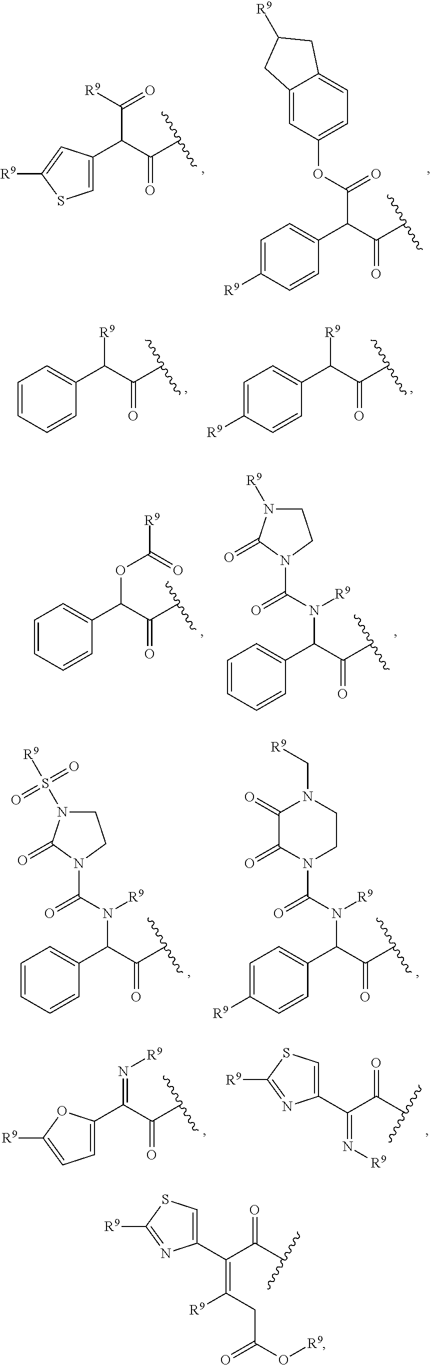 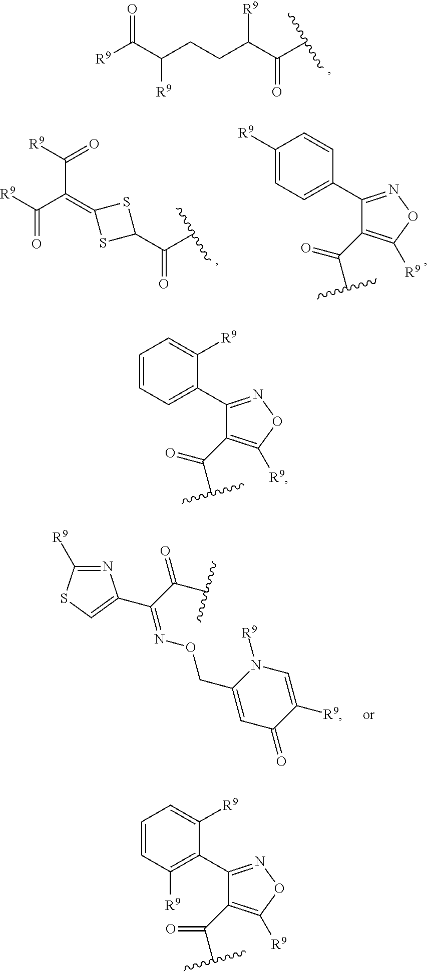
wherein:
R⁹ is independently selected from hydrogen, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ alkynyl, substituted or unsubstituted C₁₋₆ alkoxy, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷.

33-36. (canceled)

37. A pharmaceutical composition comprising a monobactam of claim 1, or pharmaceutically acceptable salt thereof, and a pharmaceutically acceptable excipient.

38. A method of treating an infectious disease comprising administering an effective amount of a monobactam of claim 1, or pharmaceutically acceptable salt thereof, to a subject in need thereof.

39-45. (canceled)

46. A kit comprising a container, a pharmaceutical composition of a compound of claim 1, and instructions for use in a patient.

47. (canceled)

48. A method of preparing a compound of Formula (I), (I′), (II), or (II′), 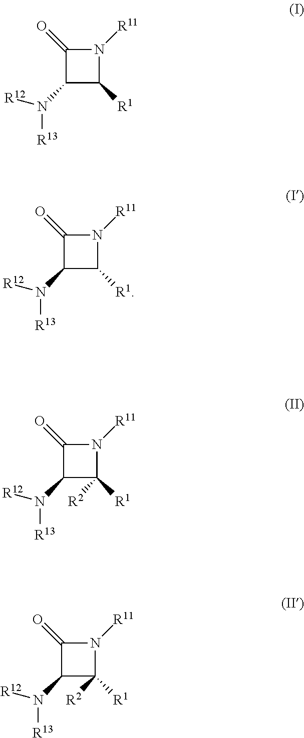
or salt thereof, the method comprising contacting a compound of Formula: 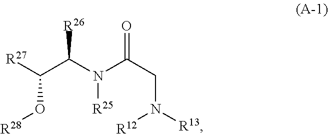
or salt thereof,
with an aldehyde or ketone 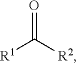
or salt thereof,
under suitable conditions to affect an asymmetric glycine aldol reaction that produces a compound of Formula: 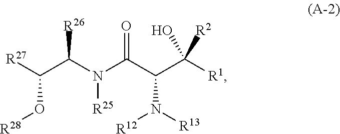
or salt thereof,
wherein:
R¹ and R² are independently selected from C₁₋₁₀ alkyl, C₁₋₁₀ haloalkyl, C₂₋₁₀ alkenyl, C₂₋₁₀alkynyl, C₆₋₁₀ aryl, C₆₋₁₀ aralkyl, C₁₋₁₀ alkoxy, 5-10 membered heteroaryl, 5-10 membered heteroaralkyl, 4-10 membered heterocyclyl, 4-10 membered heterocyclylalkyl, 3-10 membered carbocyclyl, 3-10 membered carbocyclylalkyl, or R¹ and R² are taken together with any intervening atoms to form a carbocyclic or heterocyclic ring, each of which is optionally substituted with 0, 1, 2, 3, 4, or 5 occurrences of R⁵;
R⁵ is independently selected from halogen, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ alkynyl, substituted or unsubstituted C₁₋₆ alkoxy, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, nitro, cyano, acyl, —NH₂, —NHR⁷, —N(R⁷)₂, —OH, —SH, —SO₂R⁷, —SOR⁷, —SO₂NR⁷₂, —OR⁷, or —SR⁷;
R⁷ is independently selected from hydrogen, acyl, substituted or unsubstituted C₁₋₁₀alkyl, substituted or unsubstituted C₆₋₁₀ aryl, substituted or unsubstituted 5-10 membered heteroaryl, substituted or unsubstituted 4-10 membered heterocyclyl, substituted or unsubstituted 3-10 membered carbocyclyl, or two R⁷ groups are taken together with any intervening atoms to form an substituted or unsubstituted heterocyclic ring;
R¹¹ is hydrogen, C₁₋₆ alkyl, C₁₋₆ haloalkyl, C₂₋₆ alkenyl, C₂₋₆ alkynyl, C₁₋₆ alkoxy, C₆₋₁₀ aryl, 5-10 membered heteroaryl, 4-10 membered heterocyclyl, 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), or a nitrogen protecting group, each of which is optionally substituted with 0, 1, 2, 3, 4, or 5 occurrences of R⁵;
R¹² and R¹³ are each independently hydrogen, C₁₋₆ alkyl, C₁₋₆ haloalkyl, C₂₋₆ alkenyl, C₂₋₆ alkynyl, C₁₋₆ alkoxy, C₆₋₁₀ aryl, 5-10 membered heteroaryl, 4-10 membered heterocyclyl, 3-10 membered carbocyclyl, acyl, —OH, —OR⁷, —SO₂R⁷, —SOR⁷, —SO₂NR⁷₂, —OSO₂R⁷, —OSOR⁷, —OSO₂NR⁷₂, —OSO₂OR⁷, —O(SO)OR⁷, —PO(OR⁷)₂, —PO(NR⁷)₂, —PO(NR⁷)(OR⁷), —OPO(OR⁷)₂, —OPO(NR⁷)₂, —OPO(NR⁷)(OR⁷), a nitrogen protecting group, or R¹² and R¹³ are taken together with any intervening atoms to form a heteroaryl, or heterocyclic ring, each of which is optionally substituted with 0, 1, 2, 3, 4, or 5 occurrences of R⁵;
R²⁵ is substituted or unsubstituted alkyl, substituted or unsubstituted aryl, substituted or unsubstituted heteroaryl, or a nitrogen protecting group;
R²⁶ and R²⁷ are each independently substituted or unsubstituted alkyl, substituted or unsubstituted aryl, or substituted or unsubstituted heteroaryl; and
R²⁸ is hydrogen, substituted or unsubstituted alkyl, or an oxygen protecting group, hydrolyzing a compound of Formula: 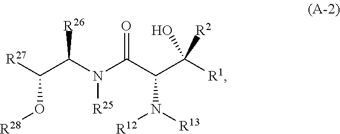
or salt thereof,
under suitable conditions to produce a compound of Formula: 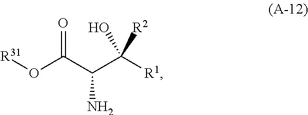
or salt thereof,
wherein:
R³¹ is hydrogen,
amidating a compound of Formula 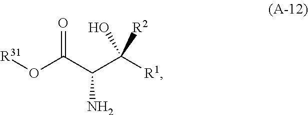
or salt thereof,
with an amine of Formula H₂N—R¹¹, or salt thereof, under suitable conditions to produce a compound of Formula: 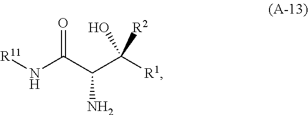
or salt thereof,
cyclizing a compound of Formula: 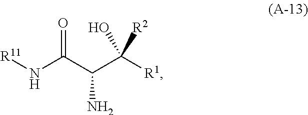
or salt thereof,
under suitable conditions to produce a compound of Formula: 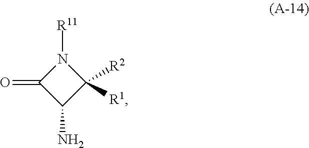
or salt thereof, and
optionally amidating, sulfonylating, or alkylating a compound of Formula: 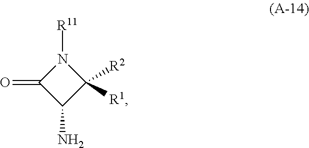
or salt thereof,
independently with LG-R¹² or LG-R¹³, wherein LG is a leaving group, to produce a compound of Formula (I), (I′), (II), or (II′).

49-54. (canceled)
